(19) United States
(12) Patent Application Publication
(10) Pub. No.: US 2022/0144759 A1
(43) Pub. Date: May 12, 2022

(54) COMPOUND

(71) Applicant: Sumitomo Chemical Company, Limited, Tokyo (JP)

(72) Inventors: Yuji ASATSU, Osaka-shi, Osaka (JP); Shoichi OZAWA, Tama-City, Tokyo (JP); Ai KOBASHI, Niihama-shi, Ehime (JP)

(73) Assignee: Sumitomo Chemical Company, Limited, Tokyo (JP)

(21) Appl. No.: 17/433,999

(22) PCT Filed: Feb. 21, 2020
(86) PCT No.: PCT/JP2020/007178

§ 371 Date: Aug. 25, 2021

(30) Foreign Application Priority Data
Feb. 28, 2019 (JP) 2019-035793
Jul. 8, 2019 (JP) 2019-126917

(51) Int. Cl. C07C 255/46; C07D 205/04; C07D 295/155; C07D 207/06; C07D 215/12; C07D 319/06; C07D 211/18; C07C 255/31; C07C 317/30; C07C 323/28; C07C 323/30; C07C 317/32; C07C 317/18

(52) U.S. Cl. CPC C07C 255/46; C07D 205/04; C07D 295/155; C07D 207/06; C07D 215/12; C07D 319/06; G02B 1/04; C07C 255/31; C07C 317/30; C07C 323/28; C07C 323/30; C07C 317/32; C07C 317/18; C07D 211/18

(57) ABSTRACT

A compound having a molecular weight of 3000 or less and a partial structure represented by Formula (X) is provided:

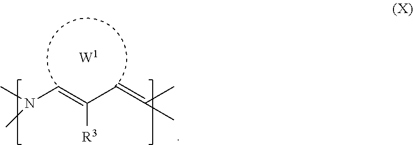

In Formula (X), a ring W¹ represents a ring structure having at least one double bond as a constituent element of the ring and having no aromaticity; and R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent.

### TECHNICAL FIELD

The present invention relates to a compound.

### BACKGROUND ART

In the related art, an ultraviolet absorber has been used in various applications and products in order to protect human bodies and resin materials from deterioration due to ultraviolet rays. The ultraviolet absorber is roughly divided into an inorganic ultraviolet absorber and an organic absorber. While the inorganic ultraviolet absorber has excellent durability such as light resistance and heat resistance, it tends to be inferior in absorption wavelength control and compatibility with an organic material. On the other hand, although the organic ultraviolet absorber is inferior in durability to the inorganic ultraviolet absorber, the organic ultraviolet absorber can control an absorption wavelength, compatibility with an organic material, and the like from the degree of freedom of a molecular structure in the organic ultraviolet absorber, and is used in a wide range of fields such as sunscreens, paints, optical materials, building materials, and automobile materials.

Examples of the organic ultraviolet absorber generally include compounds having a triazole skeleton, a benzophenone skeleton, a triazine skeleton, and a cyanoacrylate skeleton. However, since many of the organic ultraviolet absorbers having a skeleton have a maximum absorption wavelength (Amax) of 360 nm or less, the organic ultraviolet absorbers cannot efficiently absorb an ultraviolet to near-ultraviolet region having a wavelength of 380 to 400 nm, and it is necessary to increase the amount used to sufficiently absorb light in this region. In addition, in a case where many compounds having a skeleton have a broad absorption spectrum and sufficiently absorb light having a wavelength of 380 to 400 nm, there is a problem that absorption occurs not only in a wavelength region of 380 to 400 nm but also in light having a wavelength of 420 nm or more, and thereby a composition containing an ultraviolet absorber is colored.

As means for solving the above problems, for example, Patent Document 1 proposes a compound having a merocyanine skeleton as represented by the following formula as an organic ultraviolet absorber. Patent Document 1 discloses that a film containing a compound having a merocyanine skeleton represented by the following formula has low light transmittance in the vicinity of a wavelength of 390 nm.

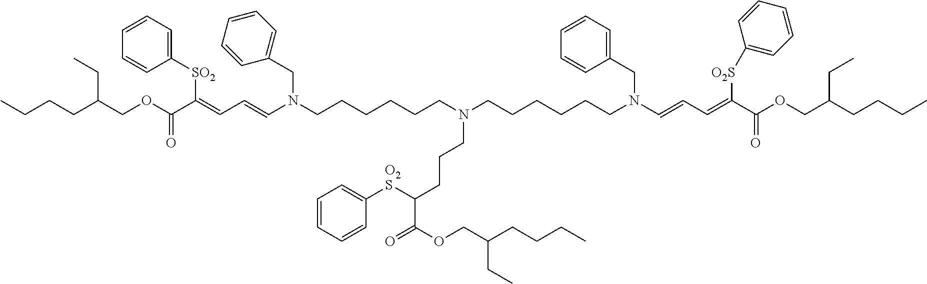 [SMILES: CCCCC(CC)COC(=O)/C(=C/C=C/N(CCCCCCN(CCCCCCN(/C=C/C=C(/C(=O)OCC(CC)CCCC)S(=O)(=O)c1ccccc1)Cc1ccccc1)CCCC(C(=O)OCC(CC)CCCC)S(=O)(=O)c1ccccc1)Cc1ccccc1)S(=O)(=O)c1ccccc1]

### PRIOR ART DOCUMENTS

### Patent Documents

Patent Document 1: JP-A-2010-111823

### SUMMARY OF THE INVENTION

### Problems to be Solved by the Invention

However, a compound having a merocyanine skeleton has low durability (particularly weather resistance), and thus is difficult to apply to applications requiring severe weather resistance.

An object of the present invention is to provide a novel compound having a merocyanine skeleton which efficiently absorbs light having a wavelength of 380 to 400 nm and can be used as an ultraviolet to near-ultraviolet absorber having good weather resistance.

### Means for Solving the Problems

The present invention includes the following inventions.

[1] A compound having a molecular weight of 3000 or less and a partial structure represented by Formula (X):

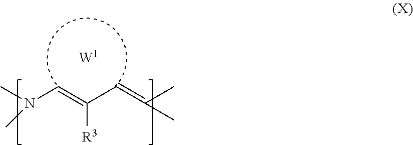

[in Formula (X), a ring W¹ represents a ring structure having at least one double bond as a constituent element of the ring and having no aromaticity;

R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{3A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—; and

R^{1A}, R^{2A}, R^{3A}, R^{4A}, R^{5A}, R^{6A}, R^{7A}, R^{8A}, R^{9A}, R^{10A}, and R^{11A} each independently represent a hydrogen atom or an alkyl group having 1 to 6 carbon atoms].

[2] The compound according to [1], wherein the compound having a molecular weight of 3000 or less and a partial structure represented by Formula (X) is any of compounds from a compound represented by Formula (I) to a compound represented by Formula (VIII):

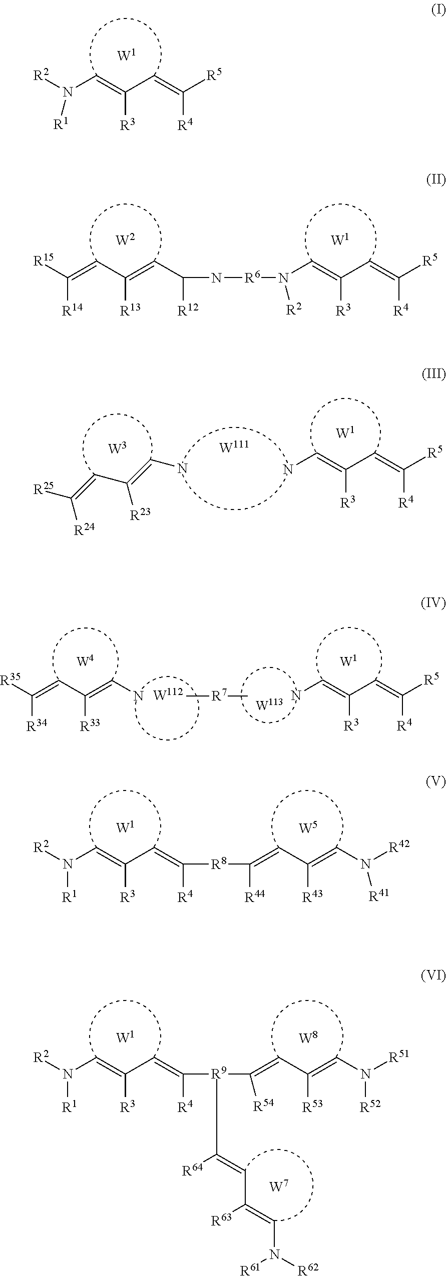

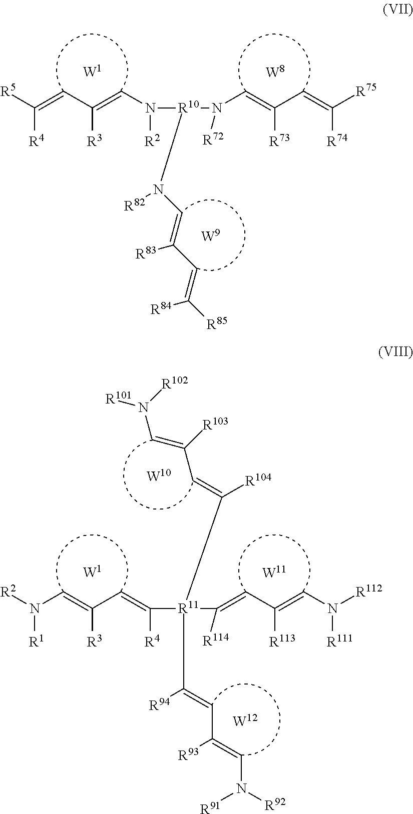

[in Formula (I) to Formula (VIII),

the ring W¹ and R³ have the same meaning as described above;

a ring W², a ring W³, a ring W⁴, a ring W⁵, a ring W⁶, a ring W⁷, a ring W⁸, a ring W⁹, a ring W¹⁰, a ring W¹¹, and a ring W¹² each independently represent a ring structure having at least one double bond as a constituent element of the ring;
a ring W¹¹¹ represents a ring having at least two nitrogen atoms as constituent elements;
a ring W¹¹² and a ring W¹¹³ each independently represent a ring having at least one nitrogen atom as a constituent element;

R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², and R¹¹² each independently represent a hydrogen atom, a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{12A}—, —SO₂—, —CO—, —O—, —COO—, —OCO—, —CONR^{13A}—, —NR^{14A}—CO—, —S—, —SO—, —CF₂— or —CHF—;

R¹³, R²³, R³³, R⁴³, R⁵³, R⁶³, R⁷¹, R⁸³, R⁹³, R¹⁰³, and R¹¹³ each independently represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—;

R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, and R^{14A} each independently represent a hydrogen atom or an alkyl group having 1 to 6 carbon atoms;

R⁴, R¹⁴, R²⁴, R³⁴, R⁴⁴, R⁵⁴, R⁶⁴, R⁷⁴, R⁸⁴, R⁹⁴, R¹⁰⁴, R¹¹⁴, R⁵, R¹⁵, R²⁵, R³⁵, R⁷⁵, and R⁸⁵ each independently represent an electron-withdrawing group;

R¹ and R² may be bonded to each other to form a ring;

R⁴¹ and R⁴² may be bonded to each other to form a ring;

R⁵¹ and R⁵² may be bonded to each other to form a ring;

R⁶¹ and R⁶² may be bonded to each other to form a ring;

R⁹¹ and R⁹² may be bonded to each other to form a ring;

R¹⁰¹ and R¹⁰² may be bonded to each other to form a ring;

R¹¹¹ and R¹¹² may be bonded to each other to form a ring;

R² and R³ may be bonded to each other to form a ring;

R¹² and R¹³ may be bonded to each other to form a ring;

R⁴² and R⁴³ may be bonded to each other to form a ring;

R⁵² and R⁵³ may be bonded to each other to form a ring;

R⁶² and R⁶³ may be bonded to each other to form a ring;

R⁷² and R⁷³ may be bonded to each other to form a ring;

R⁸² and R⁸³ may be bonded to each other to form a ring;

R⁹² and R⁹³ may be bonded to each other to form a ring;

R¹⁰² and R¹⁰³ may be bonded to each other to form a ring;

R¹¹² and R¹¹³ may be bonded to each other to form a ring;

R⁴ and R⁵ may be bonded to each other to form a ring;

R¹⁴ and R¹⁵ may be bonded to each other to form a ring;

R²⁴ and R²⁵ may be bonded to each other to form a ring;

R³⁴ and R³⁵ may be bonded to each other to form a ring;

R⁷⁴ and R⁷⁵ may be bonded to each other to form a ring;

R⁸⁴ and R⁶⁵ may be bonded to each other to form a ring;

R⁶ and R⁸ each independently represent a divalent linking group;

R⁷ represents a single bond or a divalent linking group;

R⁹ and R¹⁰ each independently represent a trivalent linking group; and

R¹¹ represents a tetravalent linking group].

[3] The compound according to [2], wherein at least one selected from R⁴ and R⁵ is a nitro group, a cyano group, a halogen atom, —OCF₃, —SCF₃, —SF₅, —SF₃, a fluoroalkyl group, a fluoroaryl group, —CO—O—R²²², —SO₂-R²²², or —CO—R²²² (R²²² represents a hydrogen atom, an alkyl group having 1 to 25 carbon atoms which may have a substituent, or an aromatic hydrocarbon group having 6 to 18 carbon atoms which may have a substituent).

[4] The compound according to [2] or [3], wherein at least one selected from R⁴ and R⁵ is a nitro group, a cyano group, a fluorine atom, a chlorine atom, —OCF₃, —SCF₃, a fluoroalkyl group, —CO—O—R²²², or —SO₂-R²²² (R²²² represents a hydrogen atom, an alkyl group having 1 to 25 carbon atoms which may have a substituent, or an aromatic hydrocarbon group having 6 to 18 carbon atoms which may have a substituent).

[5] The compound according to any one of [2] to [4], wherein at least one selected from R⁴ and R⁵ is a cyano group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent).

[6] The compound according to any one of [2] to [5], wherein at least one selected from R⁴ and R⁵ is a cyano group.

[7] The compound according to any of [2] to [6], wherein R⁴ is a cyano group, R⁵ is a cyano group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent).

[8] The compound according to any one of [2] to [7], wherein both R⁴ and R⁵ are a cyano group.

[9] The compound according to any one of [2] to [8], wherein R¹ and R² are each independently an aliphatic hydrocarbon group having 1 to 25 carbon atoms which may have a substituent.

[10] The compound according to any of [2] to [8], wherein R¹ and R² are linked to each other to form a ring.

[11] The compound according to [10], wherein the ring formed by linking R¹ and R² to each other is an aliphatic ring.

[12] The compound according to any one of [2] to [11], wherein the ring W², the ring W³, the ring W⁴, the ring W⁵, the ring W⁶, the ring W⁷, the ring W⁸, the ring W⁹, the ring W¹⁰, the ring W¹¹, and the ring W¹² are each independently a ring having no aromaticity.

[13] The compound according to any one of [2] to [12], wherein the ring W², the ring W³, the ring W⁴, the ring W⁵, the ring W⁶, the ring W⁷, the ring W⁸, the ring W⁹, the ring W¹⁰, the ring W¹¹, and the ring W¹² are each independently a 5 to 7-membered ring structure.

[14] The compound according to [13], wherein the ring W², the ring W³, the ring W⁴, the ring W⁵, the ring W⁶, the ring W⁷, the ring W⁸, the ring W⁹, the ring W¹⁰, the ring W¹¹, and the ring W¹² are each independently a 6-membered ring structure.

[15] The compound according to any one of [1] to [14], wherein R³ is a nitro group, a cyano group, a halogen atom, —OCF₃, —SCF₃, —SF₅, —SF₃, a fluoroalkyl group, a fluoroaryl group, —CO—O—R^{11A}, or —SO₂-R^{112A} (R^{111A} and R^{112A} each independently represent an alkyl group having 1 to 24 carbon atoms which may have a halogen atom).

[16] The compound according to any one of [1] to [15], wherein R³ is a cyano group, a fluorine atom, a chlorine atom, —OCF₃, —SCF₃, a fluoroalkyl group, —CO—O—R^{111A}, or —SO₂—R^{112A} (R^{111A} and R^{112A} each independently represent an alkyl group having 1 to 24 carbon atoms which may have a halogen atom).

[17] The compound according to any one of [1] to [16], wherein R³ is a cyano group.

[18] The compound according to any one of [1] to [17], wherein the ring W¹ is a 5 to 7-membered ring.

[19] The compound according to [18], wherein the ring W¹ is a 6-membered ring.

[20] The compound according to any one of [1] to [19], wherein a gram absorption coefficient ε at λₘₐₓ is 0.5 or more,

(λmax represents a maximum absorption wavelength [nm] in a compound having a molecular weight of 3000 or less and a partial structure represented by Formula (X)).

[21] The compound according to any one of [1] to [20], which satisfies Formula (B),

ε(λmax)/ε(λmax+30 nm)≥5 (B),

[ε(λmax) represents a gram absorption coefficient at a maximum absorption wavelength [nm] in a compound having a molecular weight of 3000 or less and a partial structure represented by Formula (X); and

ε(λmax+30 nm) represents a gram absorption coefficient at a wavelength [nm] of (maximum absorption wavelength+30 nm) of a compound having a molecular weight of 3000 or less and a partial structure represented by Formula (X)].

[22] A composition containing the compound according to any one of [1] to [21].

[23] A molded article formed from a composition containing the compound according to [22].

[24] A spectacle lens composition containing the compound according to any one of [1] to [21].

[25] A spectacle lens formed from the spectacle lens composition according to [24].

[26] A method for producing a compound represented by Formula (I):

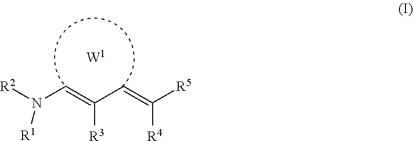

[in Formula (I), the ring W¹, R¹, R², R³, R⁴, and R⁵ have the same meaning as described above],

the method including a step of reacting a compound represented by Formula (I-1):

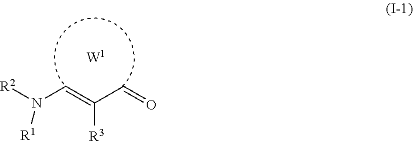

[in Formula (I-1),

a ring W¹ represents a ring structure having at least one double bond as a constituent element of the ring and having no aromaticity;

R¹ and R² each independently represent a hydrogen atom, a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{12A}—, —SO₂—, —CO—, —O—, —COO—, —OCO—, —CONR^{13A}—, —NR^{14A}—CO—, —S—, —SO—, —CF₂— or —CHF—;

R¹ and R² may be linked to each other to form a ring;

R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{3A}—, —NR^{9A}—CO—S—, —CS—, —O—CS—, —CS—O—, —NR^{10A}—CS—, —NR^{11A}CS—S—, —S—CS—, —CS—S—, —S—CS—S—, —SO—, or —SO₂—;

R² and R³ may be bonded to each other to form a ring; and

R^{1A}, R^{2A}, R^{3A}, R^{4A}, R^{5A}, R^{6A}, R^{7A}, R^{8A}, R_{9A}, R^{10A}, R^{11A}, R^{12A}, R^{13A}, and R^{14A} each independently represent a hydrogen atom or an alkyl group having 1 to 6 carbon atoms], with a compound represented by Formula (I-2):

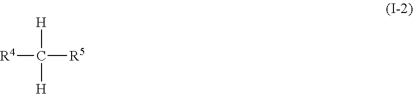

[in Formula (I-2), R⁴ and R⁵ each independently represent an electron-withdrawing group; and R⁴ and R⁵ may be bonded to each other to form a ring].

[27] The production method according to [26], further including a step of reacting a compound represented by Formula (I-3):

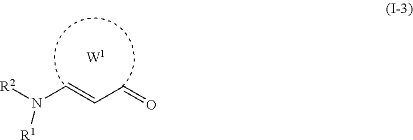

[in Formula (I-3), the ring W¹, R¹, and R² have the same meaning as described above], with a compound represented by Formula (I-4):

R³-E₁ (I-4)

[in Formula (I-4), R³ has the same meaning as described above; and E₁ represents a leaving group], to obtain the compound represented by Formula (I-1).

[28] The production method according to [27], further including a step of reacting a compound represented by Formula (I-5):

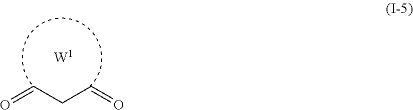

[in Formula (I-5), a ring W¹ has the same meaning as described above], with a compound represented by Formula (I-6):

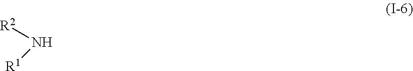

[in Formula (I-6), R¹ and R² have the same meaning as described above], to obtain the compound represented by Formula (I-3).

[29] A method for producing a compound represented by Formula (I):

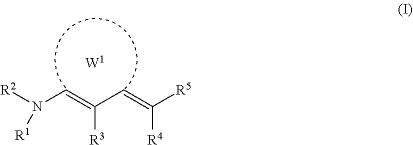

[in Formula (I), the ring W¹, R¹, R², R³, R⁴, and R⁵ have the same meaning as described above; and R² and R³ may be bonded to each other to form a ring],

the method including a step of reacting a compound represented by Formula (I-7):

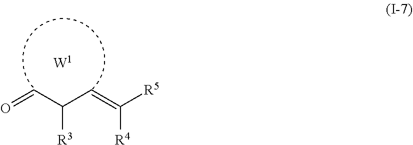

[in Formula (I-7),

a ring W¹ represents a ring structure having at least one double bond as a constituent element of the ring and having no aromaticity;

R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—;

R^{1A}, R^{2A}, R^{3A}, R^{4A}, R^{5A}, R^{6A}, R^{7A}, R^{8A}, R^{9A}, R^{10A}, and R^{11A} each independently represent a hydrogen atom or an alkyl group having 1 to 6 carbon atoms;

R⁴ and R⁵ each independently represent an electron-withdrawing group; and

R⁴ and R⁵ may be bonded to each other to form a ring], with a compound represented by Formula (I-6):

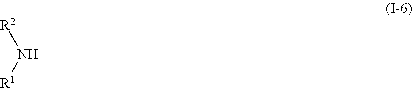

[in Formula (I-6),

R¹ and R² each independently represent a hydrogen atom, a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{12A}—, —SO₂—, —CO—, —O—, —COO—, —OCO—, —CONR^{13A}—, —NR^{14A}—CO—, —S—, —SO—, —SO₂—, —CF₂— or —CHF—;

R¹ and R² may be linked to each other to form a ring; and

R^{12A}, R^{13A}, and R^{14A} each independently represent a hydrogen atom or an alkyl group having 1 to 6 carbon atoms].

[30] The production method according to [29], further including a step of reacting a compound represented by Formula (I-8):

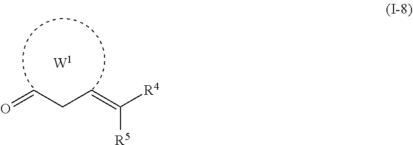

[in Formula (I-8), the ring W¹, R⁴, and R⁵ have the same meaning as described above], with a compound represented by Formula (I-4):

R³-E₁ (I-4)

[in Formula (I-4), R³ has the same meaning as described above; and E₁ represents a leaving group] to obtain the compound represented by Formula (I-7).

[31] The production method according to [30], further including a step of reacting a compound represented by Formula (I-5):

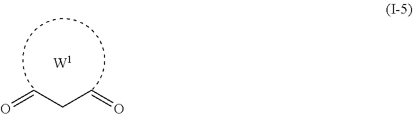

[in Formula (I-5), a ring W¹ has the same meaning as described above], with a compound represented by Formula (I-2):

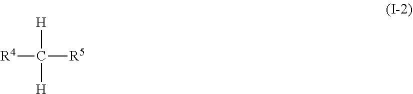

[in Formula (I-2), R⁴ and R⁵ have the same meaning as described above], to obtain the compound represented by Formula (I-8).

[32] The production method according to [26], further including a step of reacting a compound represented by Formula (I-5-1):

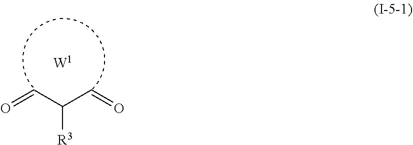

[in Formula (I-5-1), the ring W¹ and R³ have the same meaning as described above], with a compound represented by Formula (I-6):

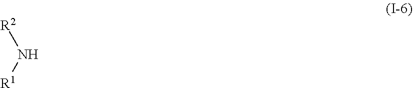

[in Formula (I-6), R¹ and R² have the same meaning as described above], to obtain the compound represented by Formula (I-1).

[33] The production method according to [29], further including a step of reacting a compound represented by Formula (I-5-1):

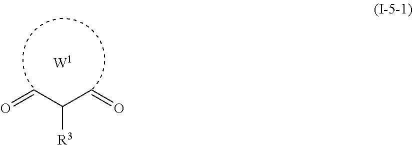

[in Formula (I-5-1), the ring W¹ and R³ have the same meaning as described above], with a compound represented by Formula (I-2):

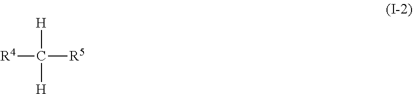

[in Formula (I-2), R⁴ and R⁵ have the same meaning as described above], to obtain the compound represented by Formula (I-7).

[34] The production method according to [32] or [33], further including a step of reacting a compound represented by Formula (I-5):

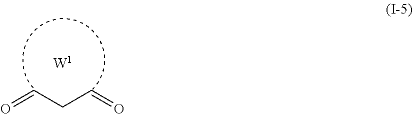

[in Formula (I-5), a ring W¹ has the same meaning as described above], with a compound represented by Formula (I-4):

R³-E₁ (I-4)

[in Formula (I-4), R³ has the same meaning as described above; and E₁ represents a leaving group], to obtain the compound represented by Formula (I-5-1).

### Effect of the Invention

The present invention provides a novel compound having a merocyanine skeleton having high absorption selectivity to short-wavelength visible light having a wavelength of 380 to 400 nm. In addition, the compound of the present invention has good weather resistance.

### MODE FOR CARRYING OUT THE INVENTION

<Compound (X)>

The compound of the present invention is a compound having a molecular weight of 3000 or less and a partial structure represented by Formula (X) (hereinafter, may be referred to as a compound (X)).

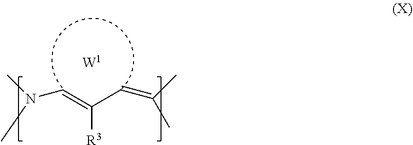

[in Formula (X), a ring W¹ represents a ring structure having at least one double bond as a constituent element of the ring and having no aromaticity;

R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—; and

R^{1A}, R^{2A}, R^{3A}, R^{4A}, R^{5A}, R^{6A}, R^{7A}, R^{8A}, R^{9A}, R^{10A}, and R^{11A} each independently represent a hydrogen atom or an alkyl group having 1 to 6 carbon atoms].

In the present specification, the number of carbon atoms does not include the number of carbon atoms of the substituent, and refers to the number of carbon atoms before substitution when —CH₂— or —CH═ is substituted, for example, as described above.

The ring W¹ is not particularly limited as long as it is a ring having one or more double bonds as a constituent element of the ring and is a ring having no aromaticity. The ring W¹ may be a single ring or a fused ring.

The ring W¹ may be a heterocyclic ring containing a hetero atom (for example, an oxygen atom, a sulfur atom, a nitrogen atom, or the like) as a constituent element of the ring, or may be an aliphatic hydrocarbon ring composed of a carbon atom and a hydrogen atom.

The ring W¹ has one or more double bonds as a constituent element of the ring, but the number of double bonds contained in the ring W¹ is usually 1 to 4, preferably 1 to 3, more preferably 1 or 2, and still more preferably 1.

The ring W¹ is usually a ring having 5 to 18 carbon atoms, and is preferably a 5 to 7-membered ring structure, and more preferably a 6-membered ring structure.

The ring W¹ is preferably a single ring.

The ring W¹ may have a substituent. Examples of the substituent include a halogen atom such as a fluorine atom, a chlorine atom, a bromine atom, or an iodine atom; an alkyl group having 1 to 12 carbon atoms, such as a methyl group, an ethyl group, a propyl group, a butyl group, a pentyl group, a hexyl group, a heptyl group, an octyl group, or a nonyl group; a halogenated alkyl group having 1 to 12 carbon atoms, such as a fluoromethyl group, a difluoromethyl group, a trifluoromethyl group, a 2-fluoroethyl group, a 2,2-difluoroethyl group, a 2,2,2-trifluoroethyl group, a 1,1,2,2-tetrafluoroethyl group, and a 1,1,2,2,2-pentafluoroethyl group; an alkoxy group having 1 to 12 carbon atoms, such as a methoxy group, an ethoxy group, a propoxy group, a butoxy group, a pentyloxy group, or a hexyloxy group; an alkylthio group having 1 to 12 carbon atoms, such as a methylthio group, an ethylthio group, a propylthio group, a butylthio group, a pentylthio group, and a hexylthio group; a fluorinated alkoxy group having 1 to 12 carbon atoms, such as a monofluoromethoxy group, a difluoromethoxy group, a trifluoromethoxy group, a 2-fluoroethoxy group, and a 1,1,2,2,2-pentafluoroethoxy group; an amino group which may be substituted with an alkyl group having 1 to 6 carbon atoms such as an amino group, a methylamino group, an ethylamino group, a dimethylamino group, a diethylamino group, or a methylethyl group; an alkylcarbonyloxy group having 2 to 12 carbon atoms, such as a methylcarbonyloxy group and an ethylcarbonyloxy group; an alkylsulfonyl group having 1 to 12 carbon atoms, such as a methylsulfonyl group and an ethylsulfonyl group; an arylsulfonyl group having 6 to 12 carbon atoms, such as a phenylsulfonyl group; a cyano group; a nitro group; a hydroxyl group; a thiol group; a carboxy group; —SF₃; and —SF₅.

The substituent which the ring W¹ may have is preferably an alkyl group having 1 to 12 carbon atoms, an alkoxy group having 1 to 12 carbon atoms, an alkylthio group having 1 to 12 carbon atoms, or an amino group which may be substituted with an alkyl group having 1 to 6 carbon atoms.

Examples of the ring W¹ include groups described below.

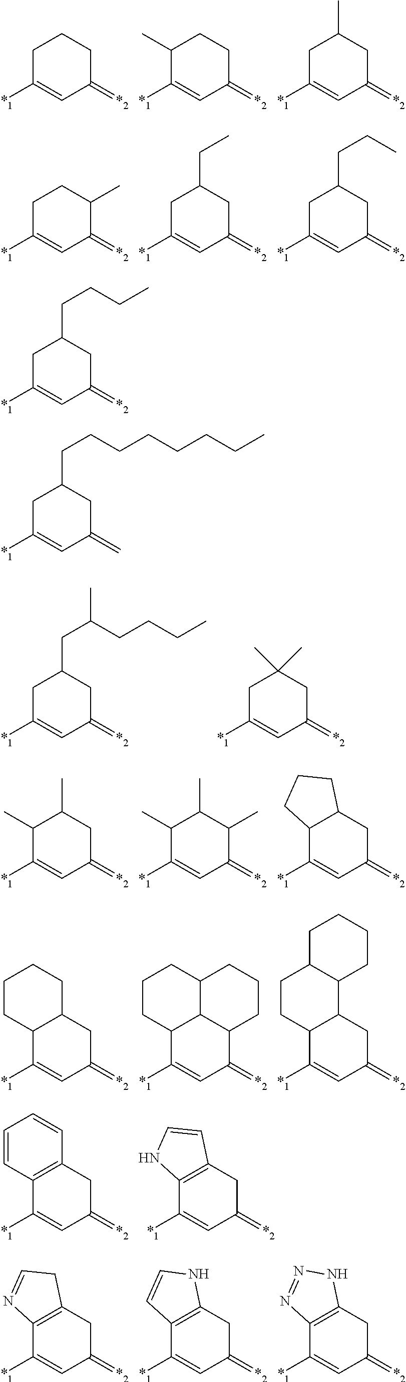

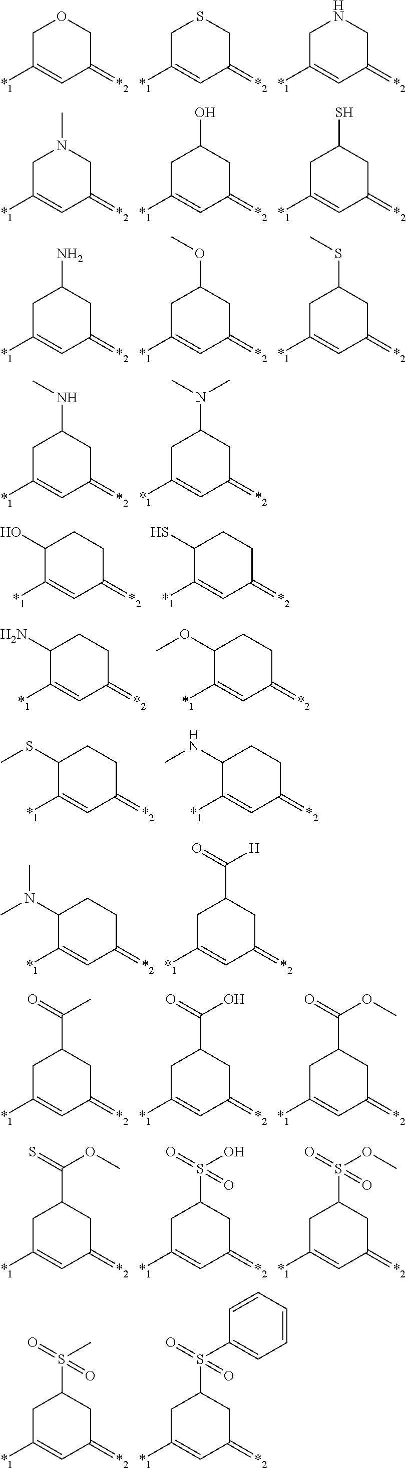

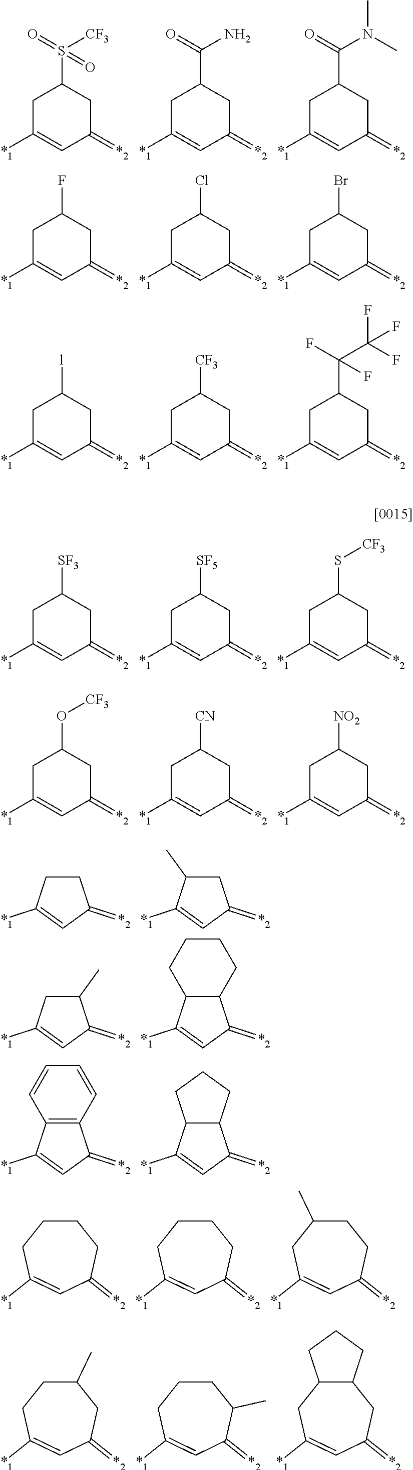

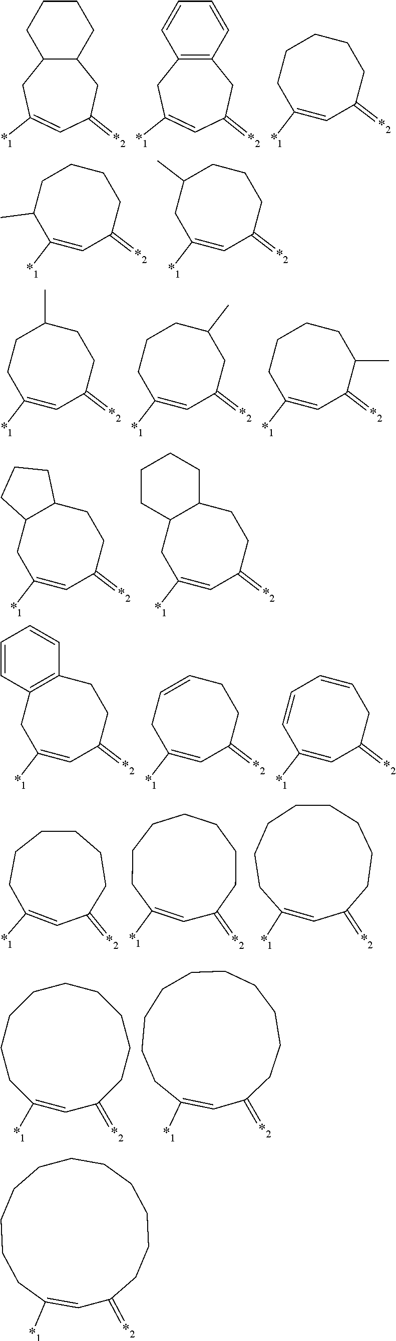

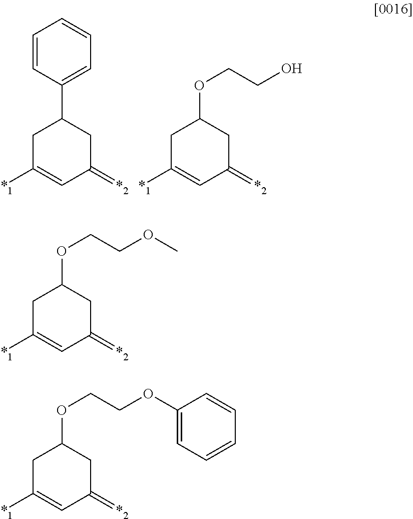

[in the formula, *1 represents a bond to a nitrogen atom, and *2 represents a bond to a carbon atom].

Examples of the heterocyclic group represented by R³ include aliphatic heterocyclic groups having 3 to 16 carbon atoms and aromatic heterocyclic groups having 3 to 16 carbon atoms, such as a pyridyl group, a pyrrolidyl group, a tetrahydrofurfuryl group, a tetrahydrothiophene group, a pyrrole group, a furyl group, a thiopheno group, a piperidine group, a tetrahydropyranyl group, a tetrahydrothiopyranyl group, a thiopyranyl group, an imidazolino group, a pyrazole group, an oxazole group, a thiazolyl group, a dioxanyl group, a morpholino group, a thiazinyl group, a triazole group, a tetrazole group, a dioxolanyl group, a pyridazinyl group, a pyrimidinyl group, a pyrazinyl group, an indolyl group, an isoindolyl group, a benzoimidazolyl group, a purinyl group, a benzotriazolyl group, a quinolinyl group, an isoquinoxalinyl group, a quinazolinyl group, a quinoxalinyl group, a cinnolinyl group, a pteridinyl group, a benzopyranyl group, an anthryl group, an acridinyl group, a xanthenyl group, a carbazolyl group, a tetrasenyl group, a porfinyl group, a chlorinyl group, corinyl group, an adenyl group, a guanyl group, a cytosyl group, a timyl group, an uracil group, a quinolyl group, a thiophenyl group, an imidazolyl group, an oxazolyl group, and a thiazolyl group, and a pyrrolidyl group, a piperidyl group, a tetrahydrofurfuryl group, a tetrahydropyranyl group, a tetrahydrothiopheno group, a tetrahydrothiopyranyl group or a pyridill group is preferable.

Examples of the aliphatic hydrocarbon group having 1 to 25 carbon atoms represented by R³ include a linear or branched alkyl group having 1 to 25 carbon atoms such as a methyl group, an ethyl group, an n-propyl group, an isopropyl group, an n-butyl group, a tert-butyl group, a sec-butyl group, an n-pentyl group, an isopentyl group, an n-hexyl group, an isohexyl group, an n-octyl group, an isooctyl group, an n-nonyl group, an isononyl group, an n-decyl group, an isodecyl group, an n-dodecyl group, an isododecyl group, an undecyl group, a lauryl group, a myristyl group, a cetyl group, and a stearyl group; a cycloalkyl group having 3 to 25 carbon atoms such as a cyclopropyl group, a cyclobutyl group, a cyclopentyl group and a cyclohexyl group; and a cycloalkylalkyl group having 4 to 25 carbon atoms such as a cyclohexylmethyl group.

The aliphatic hydrocarbon group having 1 to 25 carbon atoms represented by R³ is preferably an alkyl group having 1 to 15 carbon atoms, and more preferably an alkyl group having 1 to 12 carbon atoms.

Examples of the substituent that the aliphatic hydrocarbon group represented by R³ may include a halogen atom, a hydroxyl group, a nitro group, a cyano group, and —SO₃H.

—CH₂— or —CH═ contained in the aliphatic hydrocarbon group having 1 to 25 carbon atoms represented by R³ may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—.

When —CH₂— or —CH═ contained in the aliphatic hydrocarbon group having 1 to 25 carbon atoms is substituted, —O—, —S—, —CO—O—, or —SO₂— is preferably substituted.

When —CH₂— or —CH═ contained in the aliphatic hydrocarbon group having 1 to 25 carbon atoms is substituted with —O—, the aliphatic hydrocarbon group is preferably an alkoxy group represented by —O—R′ (R′ is an alkyl group having 1 to 24 carbon atoms which may have a halogen atom). In addition, it may be a polyalkyleneoxy group such as a polyethyleneoxy group or a polypropyleneoxy group. Examples of the alkoxy group represented by —O—R′ include a methoxy group, an ethoxy group, an —OCF₃ group, a polyethyleneoxy group, and a polypropyleneoxy group.

When —CH₂— or —CH═ contained in the aliphatic hydrocarbon group having 1 to 25 carbon atoms is substituted with —S—, the aliphatic hydrocarbon group is preferably an alkylthio group represented by —S—R′ (R′ is an alkyl group having 1 to 24 carbon atoms which may have a halogen atom). In addition, it may be a polyalkylenethio group such as a polyethylenethio group or a polypropylenethio group. Examples of the alkylthio group represented by —S—R′ include a methylthio group, an ethylthio group, A-SCF₃ group, a polyethylene thio group, and a polypropylene thio group.

When —CH₂— or —CH═ contained in the aliphatic hydrocarbon group having 1 to 25 carbon atoms is substituted with —COO—, the aliphatic hydrocarbon group is preferably a group represented by —COO—R′ (R′ is an alkyl group having 1 to 24 carbon atoms which may have a halogen atom).

When —CH₂— or —CH═ contained in the aliphatic hydrocarbon group having 1 to 25 carbon atoms is substituted with —SO₂—, the aliphatic hydrocarbon group is preferably a group represented by —SO₂—R′ (R′ is an alkyl group having 1 to 24 carbon atoms which may have a halogen atom), and may be a —SO₂CHF₂ group, a —SO₂CH₂F group, or the like.

Examples of the alkyl group having 1 to 6 carbon atoms represented by R^{1A}, R^{2A}, R^{3A}, R^{4A}, R^{5A}, R^{6A}, R^{7A}, R^{8A}, R^{9A}, R^{10A}, and R^{11A} include linear or branched alkyl groups having 1 to 6 carbon atoms such as a methyl group, an ethyl group, an n-propyl group, an isopropyl group, an n-butyl group, a tert-butyl group, a sec-butyl group, an n-pentyl group, an n-hexyl group, and a 1-methylbutyl group.

Examples of the aromatic hydrocarbon group having 6 to 18 carbon atoms represented by R³ include an aryl group having 6 to 18 carbon atoms such as a phenyl group, a naphthyl group, an anthracenyl group, a tetracenyl group, a pentacenyl group, a phenanthryl group, a chrysenyl group, a triphenylenyl group, a tetraphenyl group, a pyrenyl group, a perylenyl group, a coronenyl group, and a biphenyl group; and an aralkyl group having 7 to 18 carbon atoms such as a benzyl group, a phenylethyl group, and a naphthylmethyl group, and an aryl group having 6 to 18 carbon atoms is preferable, and a phenyl group or a benzyl group is more preferable.

Examples of the substituent that the aromatic hydrocarbon group having 6 to 18 carbon atoms represented by R³ may have include a halogen atom; a hydroxyl group; a thiol group; an amino group; a nitro group; a cyano group; and A-SO₃H group.

—CH₂— or —CH═ contained in the aromatic hydrocarbon group having 6 to 18 carbon atoms represented by R³ may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—.

When —CH₂— or —CH═ contained in the aromatic hydrocarbon group having 6 to 18 carbon atoms is substituted, it is preferably substituted with —O— or —SO₂—.

When —CH₂— or —CH═ contained in the aromatic hydrocarbon group having 6 to 18 carbon atoms is substituted with —O—, the aromatic hydrocarbon group is an aryloxy group having 6 to 17 carbon atoms such as a phenoxy group; a phenoxyethyl group, a phenoxydiethylene glycol group, an arylalkoxy group of a phenoxypolyalkylene glycol group, and the like are preferable.

When —CH₂— or —CH═ contained in the aromatic hydrocarbon group having 6 to 18 carbon atoms is substituted with —SO₂—, the aromatic hydrocarbon group is preferably a group represented by —SO₂—R″ (R″ represents an aryl group having 6 to 17 carbon atoms or an aralkyl group having 7 to 17 carbon atoms).

Examples of the halogen atom represented by R³ include a fluorine atom, a chlorine atom, a bromine atom, and an iodine atom.

R³ is preferably a nitro group; a cyano group; a halogen atom; —OCF₃; —SCF₃; —SF₅; —SF₃; a fluoroalkyl group (preferably, 1 to 25 carbon atoms); a fluoroaryl group (preferably, 6 to 18 carbon atoms); —CO—O—R^{111A} or —SO₂—R^{112A}(R^{111A} and R^{112A} each independently represent an alkyl group having 1 to 24 carbon atoms which may have a halogen atom), more preferably a cyano group; a fluorine atom; a chlorine atom; —OCF₃; —SCF₃; a fluoroalkyl group (preferably, 1 to 12 carbon atoms); —CO—O—R^{111A} or —SO₂—R^{112A}(R^{111A} and R^{112A} each independently represent an alkyl group having 1 to 24 carbon atoms which may have a halogen atom), and particularly preferably a cyano group.

The molecular weight of the compound (X) is preferably 2500 or less, more preferably 2000 or less, still more preferably 1500 or less, and particularly preferably 1000 or less.

In addition, it is preferably 100 or more, 150 or more, and 200 or more.

The compound (X) may be a copolymer as long as it has a molecular weight of 3000 or less, and is preferably a monomer.

The compound (X) preferably exhibits a maximum absorption wavelength at a wavelength of 370 nm or more and 420 nm or less. When the compound (X) has a maximum absorption wavelength at a wavelength of 370 nm or more and 420 nm or less, ultraviolet to near-ultraviolet light having a wavelength in a range of 380 nm or more and 400 nm or less can be efficiently absorbed. The maximum absorption wavelength (λmax) of the compound (X) is preferably a wavelength of 375 nm or more and 415 nm or less, more preferably a wavelength of 375 nm or more and 410 nm or less, and still more preferably a wavelength of 380 nm or more and 400 nm or less.

The gram absorption coefficient ε at λmax of the compound (X) is preferably 0.5 or more, more preferably 0.75 or more, and particularly preferably 1.0 or more. The upper limit is not particularly limited, and is generally 10 or less. λmax represents the maximum absorption wavelength of the compound (X).

When the gram absorption coefficient F at λmax of the compound (X) is 0.5 or more, ultraviolet to near-ultraviolet light in a wavelength range of 380 to 400 nm can be efficiently absorbed even with a small addition amount.

In the compound (X), ε(λmax)/e (λmax+30 nm) is preferably 5 or more, more preferably 10 or more, and particularly preferably 20 or more. The upper limit is not particularly limited, and is generally 1000 or less. ε (λmax) represents a gram absorption coefficient at a maximum absorption wavelength [nm] of the compound (X), and ε(λmax+30 nm) represents a gram absorption coefficient at a wavelength [nm] of (maximum absorption wavelength [nm]+30 nm) of the compound (X).

When ε(λmax)/ε(λmax+30 nm) is 5 or more, secondary absorption at a wavelength of 420 nm or more can be minimized, so that coloring is less likely to occur.

The unit of the gram absorption coefficient is L/(g cm).

The compound (X) is preferably any of a compounds represented by Formula (I) to Formula (VIII), and more preferably a compound represented by Formula (I).

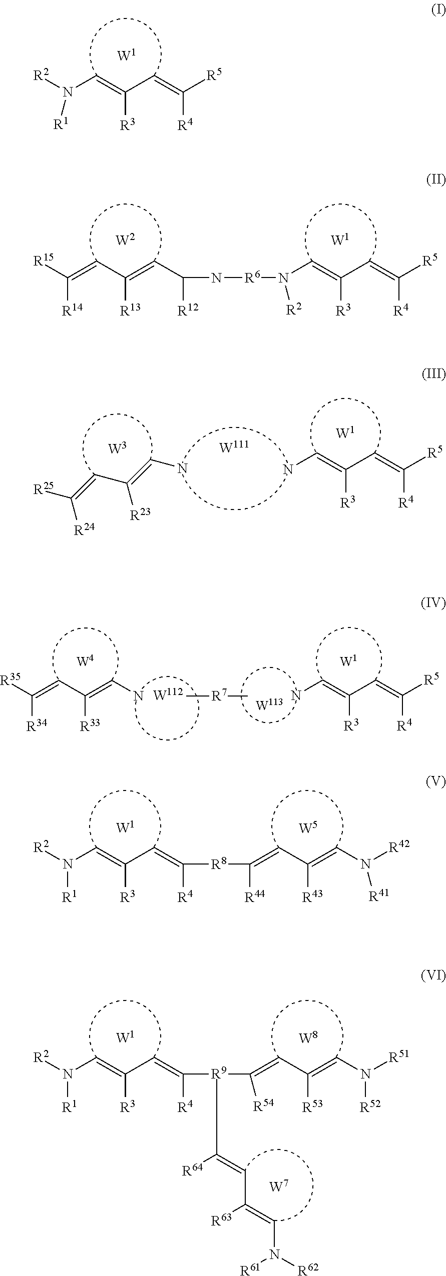

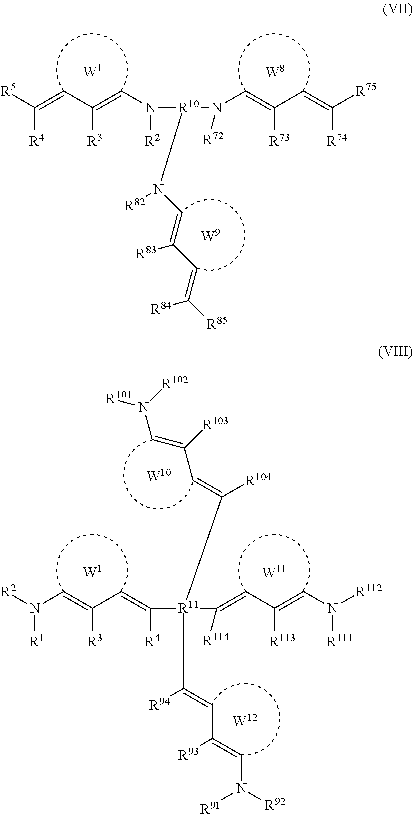

[in Formula (I) to Formula (VIII),

the ring W¹ and R³ have the same meaning as described above;

a ring W², a ring W³, a ring W⁴, a ring W⁵, a ring W⁶, a ring W⁷, a ring W⁸, a ring W⁹, a ring W¹⁰, a ring W¹¹, and a ring W¹² each independently represent a ring structure having at least one double bond as a constituent element of the ring;

a ring W¹¹¹ represents a ring having at least two nitrogen atoms as constituent elements;

a ring W¹¹² and a ring W¹¹³ each independently represent a ring having at least one nitrogen atom as a constituent element;

R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², and R¹¹² each independently represent a hydrogen atom, a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{12A}—, —SO₂—, —CO—, —O—, —COO—, —OCO—, —CONR^{13A}—, —NR^{14A}—CO—, —S—, —SO—, —CF₂— or —CHF—;

R¹³, R²³, R³³, R⁴³, R⁵³, R⁶³, R⁷³, R⁸³, R⁹³, R¹⁰³, and R¹¹³ each independently represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—;

R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, and R^{14A} each independently represent a hydrogen atom or an alkyl group having 1 to 6 carbon atoms;

R⁴, R¹⁴, R²⁴, R³⁴, R⁴⁴, R⁵⁴, R⁶⁴, R⁷⁴, R⁸⁴, R⁹⁴, R¹⁰⁴, R¹¹⁴, R⁵, R¹⁵, R²⁵, R³⁵, R⁷⁵, and R⁸⁵ each independently represent an electron-withdrawing group;

R¹ and R² may be bonded to each other to form a ring;

R⁴¹ and R⁴² may be bonded to each other to form a ring;

R⁵¹ and R⁵² may be bonded to each other to form a ring;

R⁶¹ and R⁶² may be bonded to each other to form a ring;

R⁹¹ and R⁹² may be bonded to each other to form a ring;

R¹⁰¹ and R¹⁰² may be bonded to each other to form a ring;

R¹¹¹ and R¹¹² may be bonded to each other to form a ring;

R² and R³ may be bonded to each other to form a ring;

R¹² and R¹³ may be bonded to each other to form a ring;

R⁴² and R⁴³ may be bonded to each other to form a ring;

R⁵² and R⁵³ may be bonded to each other to form a ring;

R⁶² and R⁶³ may be bonded to each other to form a ring;

R⁷² and R⁷³ may be bonded to each other to form a ring;

R⁸² and R⁸³ may be bonded to each other to form a ring;

R⁹² and R⁹³ may be bonded to each other to form a ring;

R¹⁰² and R¹⁰³ may be bonded to each other to form a ring;

R¹¹² and R¹¹³ may be bonded to each other to form a ring;

R⁴ and R⁵ may be bonded to each other to form a ring;

R¹⁴ and R¹⁵ may be bonded to each other to form a ring;

R²⁴ and R²⁵ may be bonded to each other to form a ring;

R³⁴ and R³⁵ may be bonded to each other to form a ring;

R⁷⁴ and R⁷⁵ may be bonded to each other to form a ring;

R⁸⁴ and R⁸⁵ may be bonded to each other to form a ring;

R⁶ and R⁸ each independently represent a divalent linking group;

R⁷ represents a single bond or a divalent linking group;

R⁹ and R¹⁰ each independently represent a trivalent linking group; and

R¹¹ represents a tetravalent linking group].

A ring W², a ring W³, a ring W⁴, a ring W⁵, a ring W⁶, a ring W⁷, a ring W⁸, a ring W⁹, a ring W¹⁰, a ring W¹¹, and a ring W¹² are not particularly limited as long as they are each independently a ring having one or more double bonds as a constituent element of the ring. The rings W² to W¹² each may be a single ring or a fused ring. In addition, the rings W² to W¹² may be an aliphatic ring or an aromatic ring.

The rings W² to W¹² may be a heterocyclic ring containing a hetero atom (for example, an oxygen atom, a sulfur atom, a nitrogen atom, or the like) as a constituent element of the ring.

The rings W² to W¹² have one or more double bonds as a constituent element of the ring, but the number of double bonds contained in the rings W² to W¹² each independently 1 to 4 in usual, preferably 1 to 3, more preferably 1 or 2, and still more preferably 1.

The rings W² to W¹² are each independently usually a ring having 5 to 18 carbon atoms, and is preferably a 5 to 7-membered ring structure, and more preferably a 6-membered ring structure.

The rings W² to W¹² are each independently preferably a single ring. The rings W² to W¹² are each independently preferably a ring having no aromaticity.

The rings W² to W¹² may have a substituent. Examples of the substituent include the same substituents that the ring W¹ may have.

The substituent which the rings W² to W¹² may have is preferably an alkyl group having 1 to 12 carbon atoms, an alkoxy group having 1 to 12 carbon atoms, an alkylthio group having 1 to 12 carbon atoms, or an amino group which may be substituted with an alkyl group having 1 to 6 carbon atoms.

Specific examples of the rings W² to W¹² include the same as specific examples of the ring W¹.

The ring W¹¹¹ is a ring containing two nitrogen atoms as constituent elements of the ring. The ring W¹¹¹ may be a single ring or a fused ring, and is preferably a single ring.

The ring W¹¹¹ is usually a 5 to 10-membered ring, preferably a 5 to 7-membered ring, and more preferably a 5-membered ring or a 6-membered ring.

The ring W¹¹¹ may have a substituent. Examples of the substituent that the ring W¹¹¹ may have include a hydroxyl group; a thiol group; an aldehyde group; an alkyl group having 1 to 6 carbon atoms, such as a methyl group or an ethyl group; an alkoxy group having 1 to 6 carbon atoms, such as a methoxy group and an ethoxy group; an alkylthio group having 1 to 6 carbon atoms such as a methylthio group and an ethylthio group; an amino group which may be substituted with an alkyl group having 1 to 6 carbon atoms such as an amino group, a methylamino group, a dimethylamino group, or a methylethyl group; —CONR^{1f}R^{2f} (R^{1f} and R^{2f} each independently represent a hydrogen atom or an alkyl group having 1 to 6 carbon atoms); —COSR^{3f} (R^{3f} represents an alkyl group having 1 to 6 carbon atoms); —CSSR^{4f} (R^{4f} represents an alkyl group having 1 to 6 carbon atoms); —CSOR^{5f} (R^{5f} represents an alkyl group having 1 to 6 carbon atoms); and —SO₂R^{6f} (R^{5f} represents an aryl group having 6 to 12 carbon atoms or an alkyl group having 1 to 6 carbon atoms which may have a fluorine atom).

Examples of the ring W¹¹¹ include rings described below.

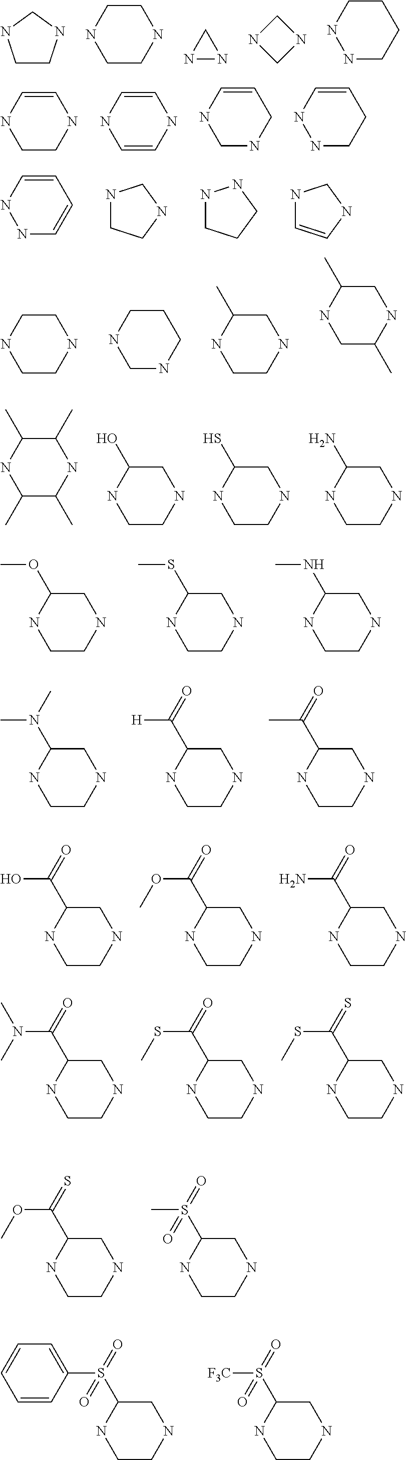

The rings W¹¹² and W¹¹³ are each independently a ring containing one nitrogen atom as a constituent element of the ring. The rings W¹¹² and W¹¹³ may be each independently a single ring or a fused ring, and are preferably a single ring.

The rings W¹¹² and W¹¹³ are each independently usually a 5 to 10-membered ring, preferably a 5 to 7-membered ring, and more preferably a 5-membered ring or a 6-membered ring.

The rings W¹¹² and W¹¹³ may have a substituent. Examples of the substituent that the rings W¹¹² and W¹¹³ may have include the same substituent as the substituent of the ring W¹.

Examples of the rings W¹¹² and W¹¹³ include rings described below.

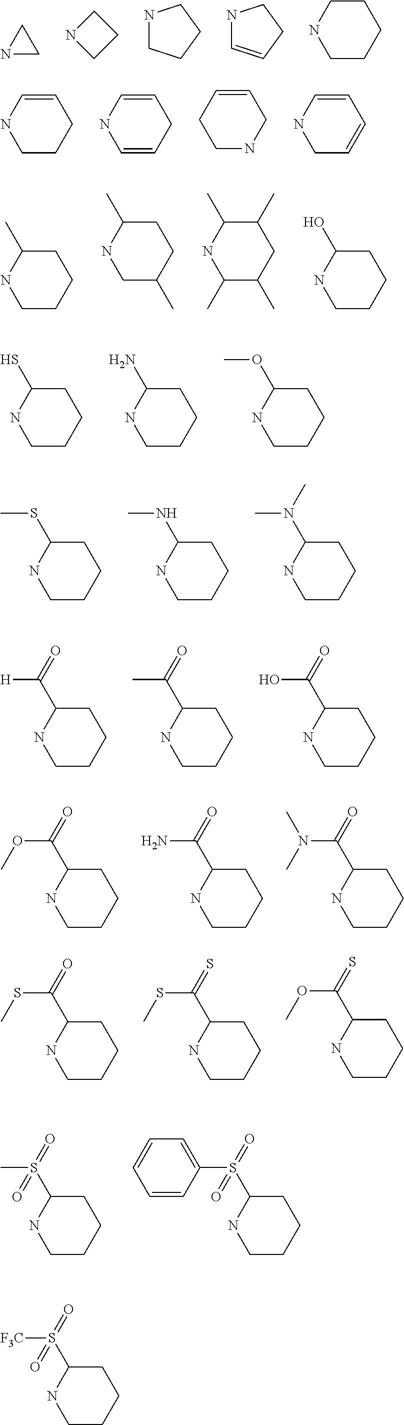

Examples of the electron-withdrawing group represented by R⁴, R¹⁴, R²⁴, R³⁴, R⁴⁴, R⁵⁴, R⁶⁴, R⁷⁴, R⁸⁴, R⁹⁴, R¹⁰⁴, R¹¹⁴, R⁵, R¹⁵, R²⁵, R³⁵, R⁷⁵, and R⁸⁵ include a halogen atom, a nitro group, a cyano group, a carboxy group, a halogenated alkyl group, a halogenated aryl group, —OCF₃, —SCF₃, —SF₅, —SF₃, —SO₃H, —SO₂H, —SO₂CF₃, —SO₂CHF₂, —SO₂CH₂F, and a group represented by Formula (X-1).

*—X¹—R²²² (X-1)

[in Formula (X-1),

X¹ represents —CO—, —COO—, —OCO—, —CS—, —CSS—, —COS—, —CSO—, —SO₂—, —NR²²³CO—, or —CONR²²⁴—;

R²²² represents a hydrogen atom, an alkyl group having 1 to 25 carbon atoms which may have a substituent, or an aromatic hydrocarbon group having 6 to 18 carbon atoms which may have a substituent;

R²²³ and R²²⁴ each independently represent a hydrogen atom, an alkyl group having 1 to 6 carbon atoms, or a phenyl group; and

* represents a bond].

Examples of the halogen atom include a fluorine atom, a chlorine atom, a bromine atom, and an iodine atom.

Examples of the halogenated alkyl group include a fluoroalkyl group such as a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, and a perfluorohexyl group, and a perfluoroalkyl group is preferable. The number of carbon atoms of the halogenated alkyl group is usually 1 to 25, and preferably 1 to 12. The halogenated alkyl group may be linear or branched.

Examples of the halogenated aryl group include a fluorophenyl group, a chlorophenyl group, and a bromophenyl group, and the halogenated aryl group is preferably a fluoroaryl group, and more preferably a perfluoroaryl group. The number of carbon atoms of the aryl group containing a halogen atom is usually 6 to 18, and preferably 6 to 12.

X¹ is preferably —COO— or —SO₂—.

Examples of the alkyl group having 1 to 25 carbon atoms represented by R²²² include linear or branched alkyl groups having 1 to 25 carbon atoms such as a methyl group, an ethyl group, an n-propyl group, an isopropyl group, an n-butyl group, a tert-butyl group, a sec-butyl group, an n-pentyl group, an n-hexyl group, a 1-methylbutyl group, a 3-methylbutyl group, a n-octyl group, a n-decyl group, and a 2-hexyl-octyl group. R²²² is preferably an alkyl group having 1 to 12 carbon atoms.

Examples of the substituent that the alkyl group having 1 to 25 carbon atoms represented by R²²² may have include a halogen atom and a hydroxy group.

Examples of the aromatic hydrocarbon group having 6 to 18 carbon atoms represented by R²²² include an aryl group having 6 to 18 carbon atoms such as a phenyl group, a naphthyl group, an anthracenyl group, and a biphenyl group; and an aralkyl group having 7 to 18 carbon atoms such as a benzyl group, a phenylethyl group, and a naphthylmethyl group.

Examples of the substituent that the aromatic hydrocarbon group having 6 to 18 carbon atoms represented by R²²² may have include a halogen atom and a hydroxy group.

Examples of the alkyl group having 1 to 6 carbon atoms represented by R²²³ and R²²⁴ include linear or branched alkyl groups having 1 to 6 carbon atoms such as a methyl group, an ethyl group, an n-propyl group, an isopropyl group, an n-butyl group, a tert-butyl group, a sec-butyl group, an n-pentyl group, an n-hexyl group, a 1-methylbutyl group, and a 3-methylbutyl group.

The electron-withdrawing group represented by R⁴, R¹⁴, R²⁴, R³⁴, R⁴⁴, R⁵⁴, R⁶⁴, R⁷⁴, R⁸⁴, R⁹⁴, R¹⁰⁴, R¹¹⁴, R⁵, R¹⁵, R²⁵, R³⁵, R⁷⁵, and R⁸⁵ is preferably each independently a nitro group, a cyano group, a halogen atom, —OCF₃, —SCF₃, —SF₅, —SF₃, a fluoroalkyl group (preferably, 1 to 25 carbon atoms), a fluoroaryl group (preferably, 6 to 18 carbon atoms), —CO—O—R²²², —SO₂—R²²², or —CO—R²²² (R²²² represents a hydrogen atom, an alkyl group having 1 to 25 carbon atoms which may have a substituent, or an aromatic hydrocarbon group having 6 to 18 carbon atoms which may have a substituent),

more preferably a nitro group, a cyano group, a fluorine atom, a chlorine atom, —OCF₃, —SCF₃, a fluoroalkyl group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), and still more preferably a cyano group.

At least one of R⁴ and R⁵ is preferably a cyano group, R⁴ is more preferably a cyano group, and R⁵ is more preferably a cyano group, —CO—O—R²²² or —SO₂—R²²² (R²²² each independently represents a hydrogen atom, an alkyl group having 1 to 25 carbon atoms which may have a halogen atom, or an aromatic hydrocarbon group having 6 to 18 carbon atoms which may have a halogen atom).

R⁴ and R⁵ may be bonded to each other to form a ring. The ring formed by bonding R⁴ and R⁵ to each other may be a single ring or a fused ring, but is preferably a single ring. The ring formed by bonding R⁴ and R⁵ to each other may contain a hetero atom (nitrogen atom, oxygen atom, or sulfur atom) or the like as a constituent element of the ring.

The ring formed by bonding R⁴ and R⁵ to each other is usually a 3 to 10-membered ring, preferably a 5 to 7-membered ring, and more preferably a 5-membered ring or a 6-membered ring.

Examples of the ring formed by bonding R⁴ and R⁵ to each other include the structures described below.

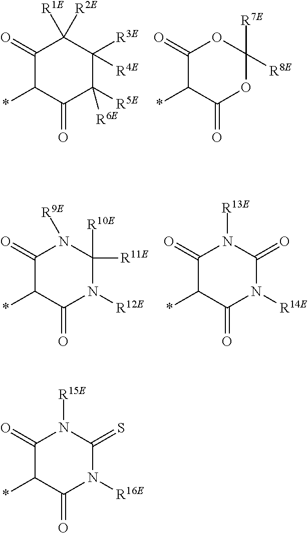

[in the formula, * represents a bond to a carbon atom; and R^{1E} to R^{16E} each independently represent a hydrogen atom or a substituent].

The ring formed by bonding R⁴ and R⁵ to each other may have a substituent (R^{1E} to R^{16E} in the above formula). Examples of the substituent include the same substituents that the ring W¹ may have. The R^{1E} to R^{16E} are each independently preferably an alkyl group having 1 to 12 carbon atoms, more preferably an alkyl group having 1 to 6 carbon atoms, and still more preferably a methyl group.

Examples of the ring formed by bonding R¹⁴ and R¹⁵ to each other include the same ring as the ring formed by bonding R⁴ and R⁵ to each other.

Examples of the ring formed by bonding R²⁴ and R²⁵ to each other include the same ring as the ring formed by bonding R⁴ and R⁵ to each other.

Examples of the ring formed by bonding R³⁴ and R³⁵ to each other include the same ring as the ring formed by bonding R⁴ and R⁵ to each other.

Examples of the ring formed by bonding R⁷⁴ and R⁷⁵ to each other include the same ring as the ring formed by bonding R⁴ and R⁵ to each other.

Examples of the ring formed by bonding R⁸⁴ and R⁸⁵ to each other include the same ring as the ring formed by bonding R⁴ and R⁵ to each other.

Examples of the heterocyclic group represented by R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², R¹¹², R¹³, R²³, R³³, R⁴³, R⁵³, R⁶³, R⁷³, R⁸³, R⁹³, R¹⁰³, and R¹¹³ include the same heterocyclic group as that represented by R³, and the heterocyclic group is preferably a pyrrolidyl group, a piperidyl group, a tetrahydrofurfuryl group, a tetrahydropyranyl group, a tetrahydrothiopheno group, a tetrahydrothiopyranyl group or a pyridyl group.

Examples of the aliphatic hydrocarbon group having 1 to 25 carbon atoms represented by R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², R¹¹², R¹³, R²³, R³³, R⁴³, R⁵³, R⁶³, R⁷³, R⁸³, R⁹³, R¹⁰³, and R¹¹³ include the same aliphatic hydrocarbon group as the aliphatic hydrocarbon group having 1 to 25 carbon atoms represented by R³.

The aliphatic hydrocarbon group having 1 to 25 carbon atoms is preferably an alkyl group having 1 to 15 carbon atoms, and more preferably an alkyl group having 1 to 12 carbon atoms.

Examples of the substituent that the aliphatic hydrocarbon group represented by R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², R¹¹², R¹³, R²³, R³³, R⁴³, R⁵³, R⁶³, R⁷³, R⁸³, R⁹³, R¹⁰³, and R¹¹³ may include a halogen atom, a hydroxyl group, a nitro group, a cyano group, —SO₃H, and the like.

In addition, —CH₂— or —CH═ contained in the aliphatic hydrocarbon group having 1 to 25 carbon atoms represented by R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², and R¹¹² may be substituted with —NR^{12A}—, —SO₂—, —CO—, —O—, —COO—, —OCO—, —CONR^{13A}—, —NR^{14A}—CO—, —S—, —SO—, —CF₂— or —CHF—. —CH₂— or —CH═ contained in the aliphatic hydrocarbon group having 1 to 25 carbon atoms represented by R¹³, R²³, R³³, R⁴³, R⁵³, R⁶³, R⁷³, R⁸³, R⁹³, R¹⁰³, and R¹¹³ may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO— or —SO₂—.

When —CH₂— or —CH═ contained in the aliphatic hydrocarbon group having 1 to 25 carbon atoms is substituted, —O—, —S—, —CO—O—, or —SO₂— is preferably substituted.

When —CH₂— or —CH═ contained in the aliphatic hydrocarbon group having 1 to 25 carbon atoms is substituted with —O—, the aliphatic hydrocarbon group is preferably an alkoxy group represented by —O—R′ (R′ is an alkyl group having 1 to 24 carbon atoms which may have a halogen atom). In addition, it may be a polyalkyleneoxy group such as a polyethyleneoxy group or a polypropyleneoxy group. Examples of the alkoxy group represented by —O—R′ include a methoxy group, an ethoxy group, and an —OCF₃ group.

When —CH₂— or —CH═ contained in the aliphatic hydrocarbon group having 1 to 25 carbon atoms is substituted with —S—, the aliphatic hydrocarbon group is preferably an alkylthio group represented by —S—R′ (R′ is an alkyl group having 1 to 24 carbon atoms which may have a halogen atom). In addition, it may be a polyalkylenethio group such as a polyethylenethio group or a polypropylenethio group. Examples of the alkylthio group represented by —S—R′ include a methylthio group, an ethylthio group, A-SCF₃ group, a polyethylene thio group, and a polypropylene thio group.

When —CH₂— or —CH═ contained in the aliphatic hydrocarbon group having 1 to 25 carbon atoms is substituted with —COO—, the aliphatic hydrocarbon group is preferably a group represented by —COO—R′ (R′ is an alkyl group having 1 to 24 carbon atoms which may have a halogen atom).

When —CH₂— or —CH═ contained in the aliphatic hydrocarbon group having 1 to 25 carbon atoms is substituted with —SO₂—, the aliphatic hydrocarbon group is preferably a group represented by —SO₂—R′ (R′ is an alkyl group having 1 to 24 carbon atoms which may have a halogen atom), and may be a —SO₂CHF₂ group, a —SO₂CH₂F group, or the like.

Examples of the alkyl group having 1 to 6 carbon atoms represented by R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, and R^{14A} include the same alkyl group as the alkyl group having 1 to 6 carbon atoms represented by R^{1A}.

Examples of the aromatic hydrocarbon group having 6 to 18 carbon atoms represented by R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², R¹¹², R¹³, R²³, R³³, R⁴³, R⁵³, R⁶³, R⁷³, R⁸³, R⁹³, R¹⁰³, and R¹¹³ include the same aromatic hydrocarbon group as the aromatic hydrocarbon group having 6 to 18 carbon atoms represented by R¹, and the aromatic hydrocarbon group is preferably an aryl group having 6 to 18 carbon atoms, and more preferably a phenyl group or a benzyl group.

Examples of the substituent that the aromatic hydrocarbon group having 6 to 18 carbon atoms may have include a halogen atom; a hydroxyl group; a thiol group; an amino group; a nitro group; a cyano group; and A-SO₃H group. —CH₂— or —CH═ contained in the aromatic hydrocarbon group having 6 to 18 carbon atoms represented by R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², and R¹¹² may be substituted with —NR^{12A}—, —SO₂—, —CO—, —O—, —COO—, —OCO—, —CONR^{13A}—, —NR^{14A}—CO—, —S—, —SO—, —CF₂— or —CHF—. —CH₂— or —CH═ contained in the aromatic hydrocarbon group having 6 to 18 carbon atoms represented by R¹³, R²³, R³³, R⁴³, R⁵³, R⁶³, R⁷³, R⁸³, R⁹³, R¹⁰³, and R¹¹³ may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO— or —SO₂—.

When —CH₂— or —CH═ contained in the aromatic hydrocarbon group having 6 to 18 carbon atoms is substituted, it is preferably substituted with —O— or —SO₂—.

When —CH₂— or —CH═ contained in the aromatic hydrocarbon group having 6 to 18 carbon atoms is substituted with —O—, the aromatic hydrocarbon group is an aryloxy group having 6 to 17 carbon atoms such as a phenoxy group; a phenoxyethyl group, a phenoxydiethylene glycol group, an arylalkoxy group of a phenoxypolyalkylene glycol group, and the like are preferable.

When —CH₂— or —CH═ contained in the aromatic hydrocarbon group having 6 to 18 carbon atoms is substituted with —SO₂—, the aromatic hydrocarbon group is preferably a group represented by —SO₂—R″ (R″ represents an aryl group having 6 to 17 carbon atoms or an aralkyl group having 7 to 17 carbon atoms).

Examples of the alkyl group having 1 to 6 carbon atoms represented by R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, and R^{14A} include the same alkyl group as the alkyl group having 1 to 6 carbon atoms represented by R^{1A}.

R² and R³ may be linked to each other to form a ring. A double bond constituting the ring W¹ is included as a constituent element of the ring formed by linking R² and R³. That is, the ring formed by linking R² and R³ and the ring W¹ form a fused ring. Specific examples of the fused ring formed by the ring formed by linking R² and R³ and the ring W¹ include the ring structures described below.

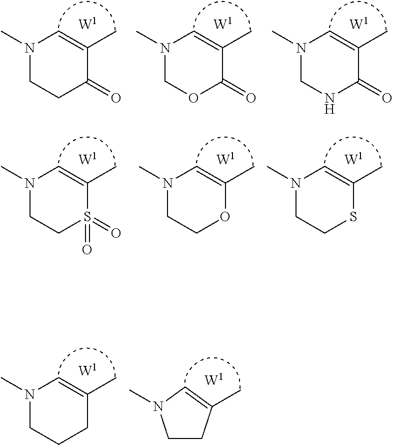

The ring formed by bonding R¹² and R¹³ to each other contains a double bond constituting the ring W² as a constituent element of the ring formed by connecting R¹² and R¹³. That is, the ring formed by bonding R¹² and R¹³ to each other and the ring W² form a fused ring. Specific examples thereof include the same fused ring formed by the ring formed by linking R² and R³ and the ring W¹.

The ring formed by bonding R⁴² and R⁴³ to each other contains a double bond constituting the ring W⁵ as a constituent element of the ring formed by connecting R⁴² and R⁴¹. That is, the ring formed by bonding R⁴² and R⁴³ to each other and the ring W⁵ form a fused ring. Specific examples thereof include the same fused ring formed by the ring formed by linking R² and R³ and the ring W¹.

The ring formed by bonding R⁵² and R⁵³ to each other contains a double bond constituting the ring W⁶ as a constituent element of the ring formed by connecting R⁵² and R⁵³. That is, the ring formed by bonding R⁵² and R⁵³ to each other and ring W⁶ form a fused ring. Specific examples thereof include the same fused ring formed by the ring formed by linking R² and R³ and the ring W¹.

The ring formed by bonding R⁶² and R⁶³ to each other contains a double bond constituting the ring W⁷ as a constituent element of the ring formed by connecting R⁶² and R⁶³. That is, the ring formed by bonding R⁶² and R⁶³ to each other and ring W⁷ form a fused ring. Specific examples thereof include the same fused ring formed by the ring formed by linking R² and R³ and the ring W¹.

The ring formed by bonding R⁷² and R⁷³ to each other contains a double bond constituting the ring W⁸ as a constituent element of the ring formed by connecting R⁷² and R⁷³. That is, the ring formed by bonding R⁷² and R²³ to each other and ring W⁸ form a fused ring. Specific examples thereof include the same fused ring formed by the ring formed by linking R² and R³ and the ring W¹.

The ring formed by bonding R⁸² and R⁸³ to each other contains a double bond constituting the ring W⁹ as a constituent element of the ring formed by connecting R⁸² and R⁸³. That is, the ring formed by bonding R⁸² and R⁸³ to each other and ring W⁹ form a fused ring. Specific examples thereof include the same fused ring formed by the ring formed by linking R² and R³ and the ring W¹.

The ring formed by bonding R⁹² and R⁹³ to each other contains a double bond constituting the ring W¹² as a constituent element of the ring formed by connecting R⁹² and R⁹³. That is, the ring formed by bonding R⁹² and R⁹³ to each other and ring W¹² form a fused ring. Specific examples thereof include the same fused ring formed by the ring formed by linking R² and R³ and the ring W¹.

The ring formed by bonding R¹⁰² and R¹⁰³ to each other contains a double bond constituting the ring W¹⁰ as a constituent element of the ring formed by connecting R¹⁰² and R¹⁰³. That is, the ring formed by bonding R¹⁰² and R¹⁰³ to each other and ring W¹⁰ form a fused ring. Specific examples thereof include the same fused ring formed by the ring formed by linking R² and R³ and the ring W¹.

The ring formed by bonding R¹¹² and R¹¹³ to each other contains a double bond constituting the ring W¹¹ as a constituent element of the ring formed by connecting R¹¹² and R¹¹³. That is, the ring formed by bonding R¹¹² and R¹¹³ to each other and ring W¹¹ form a fused ring. Specific examples thereof include the same fused ring formed by the ring formed by linking R² and R³ and the ring W¹.

R¹ and R² may be bonded to each other to form a ring. The ring formed by bonding R¹ and R² to each other contains one nitrogen atom as a constituent element of the ring. The ring formed by bonding R¹ and R² to each other may be a single ring or a fused ring, but is preferably a single ring. The ring formed by bonding R¹ and R² to each other may contain a hetero atom (nitrogen atom, oxygen atom, or sulfur atom) or the like as a constituent element of the ring. The ring formed by bonding R¹ and R² to each other is preferably an aliphatic ring, and more preferably an aliphatic ring having no unsaturated bond.

The ring formed by bonding R¹ and R² to each other is usually a 3 to 10-membered ring, preferably a 5 to 7-membered ring, and more preferably a 5-membered ring or a 6-membered ring.

The ring formed by bonding R¹ and R² to each other may have a substituent, and examples thereof include the same substituents as those of the rings W² to W¹².

Examples of the ring formed by bonding R¹ and R² to each other include the rings described below.

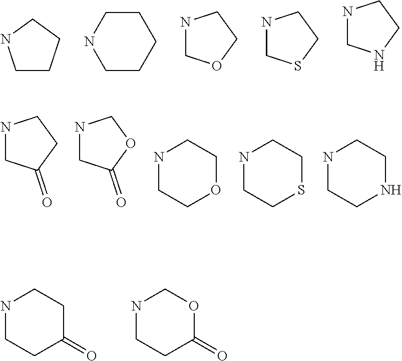

Examples of the ring formed by bonding R⁴¹ and R⁴² to each other include the same ring as the ring formed by bonding R¹ and R² to each other.

Examples of the ring formed by bonding R⁵¹ and R⁵² to each other include the same ring as the ring formed by bonding R¹ and R² to each other.

Examples of the ring formed by bonding R⁶¹ and R⁶² to each other include the same ring as the ring formed by bonding R¹ and R² to each other.

Examples of the ring formed by bonding R⁹¹ and R⁹² to each other include the same ring as the ring formed by bonding R¹ and R² to each other.

Examples of the ring formed by bonding R¹⁰¹ and R¹⁰² to each other include the same ring as the ring formed by bonding R¹ and R² to each other.

Examples of the ring formed by bonding R¹¹¹ and R¹¹² to each other include the same ring as the ring formed by bonding R¹ and R² to each other.

A divalent linking group represented by R⁶, R⁷, and R⁸ represents a divalent aliphatic hydrocarbon group having 1 to 18 carbon atoms which may have a substituent or a divalent aromatic hydrocarbon group having 6 to 18 carbon atoms which may have a substituent. —CH₂— contained in the divalent aliphatic hydrocarbon group and the divalent aromatic hydrocarbon group may be substituted with —O—, —S—, —NR^{1B}— (R^{1B} represents a hydrogen atom or an alkyl group having 1 to 6 carbon atoms), —CO—, —SO₂—, —SO—, or —PO₃—.

Examples of the substituent that the divalent aliphatic hydrocarbon group and the divalent aromatic hydrocarbon group may have include a halogen atom, a hydroxyl group, a carboxy group, and an amino group.

The divalent linking groups represented by R⁶, R⁷, and R⁸ are each independently preferably a divalent aliphatic hydrocarbon group having 1 to 18 carbon atoms which may have a substituent, and more preferably a divalent aliphatic hydrocarbon group having 1 to 12 carbon atoms which may have a substituent.

Specific examples of the divalent linking group represented by R⁶, R⁷, and R⁸ include the following linking groups. In the formula * represents a bond.

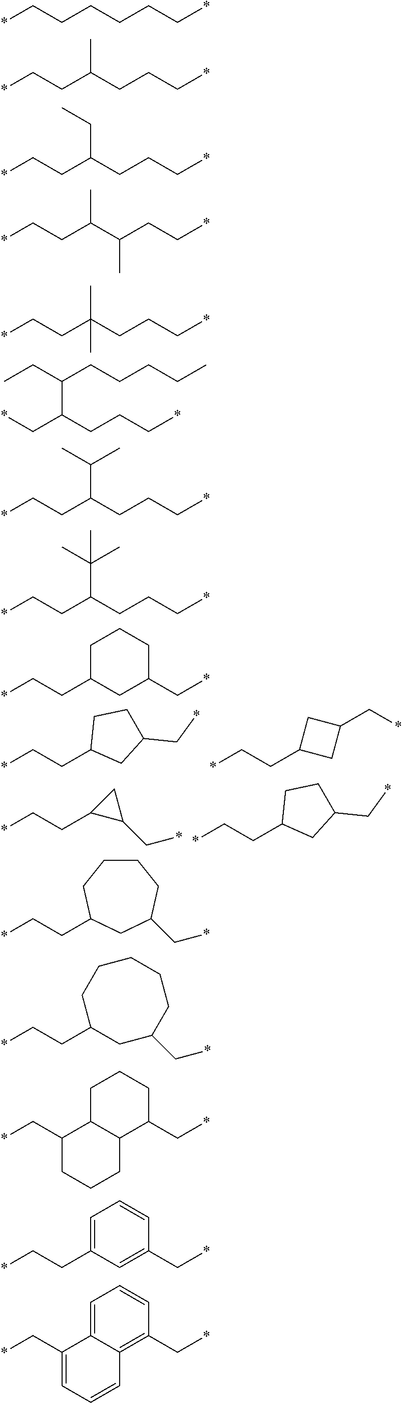

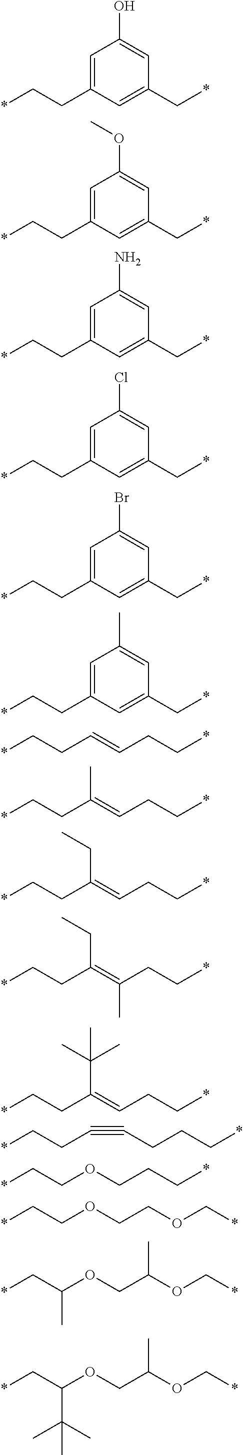

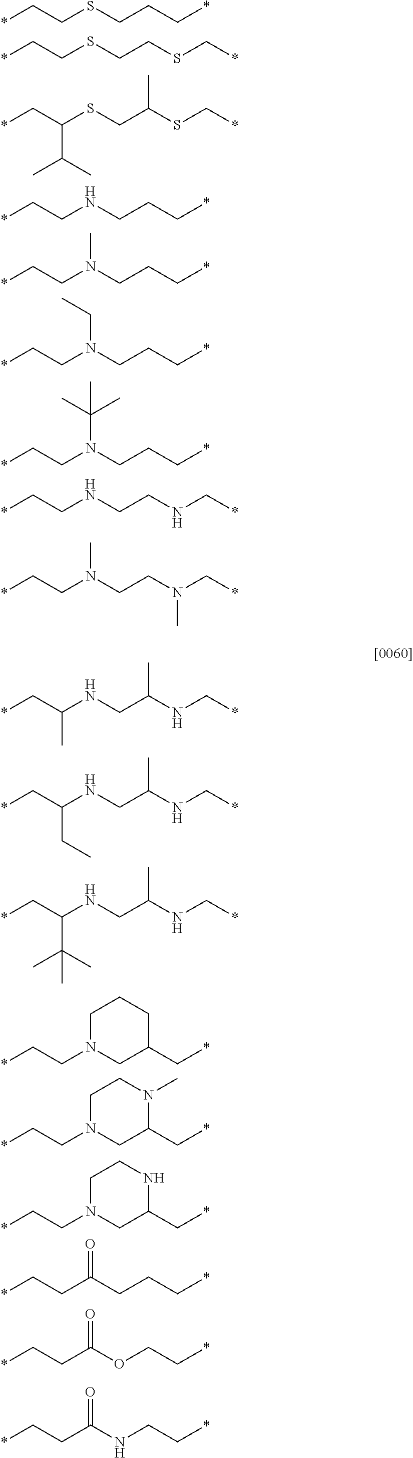

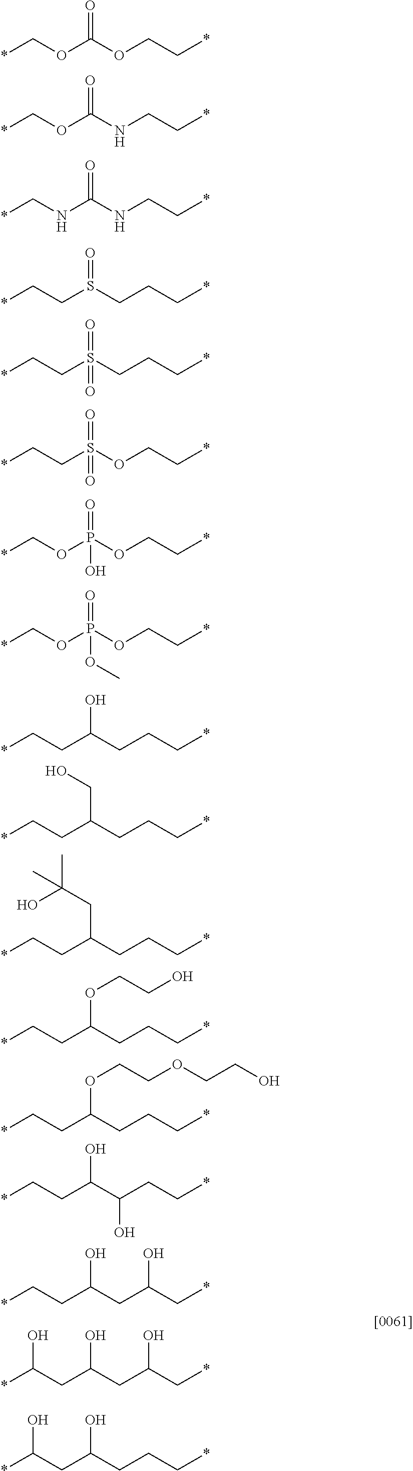

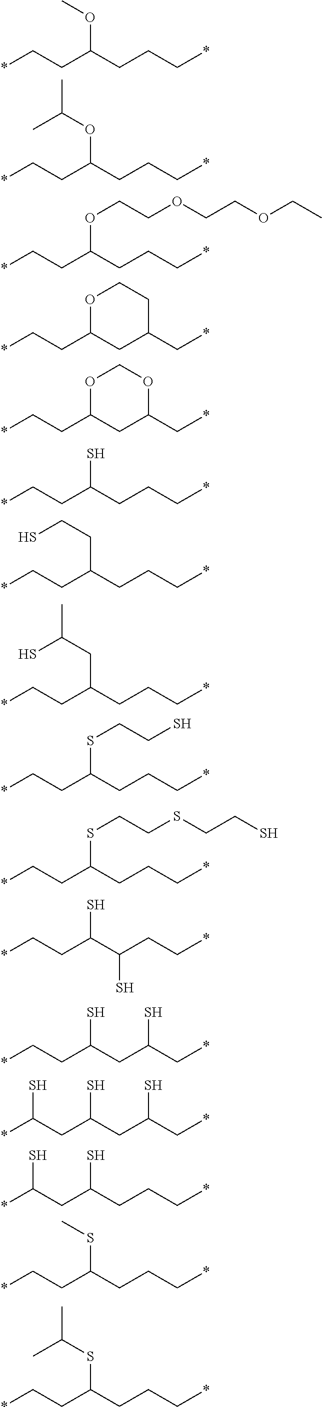

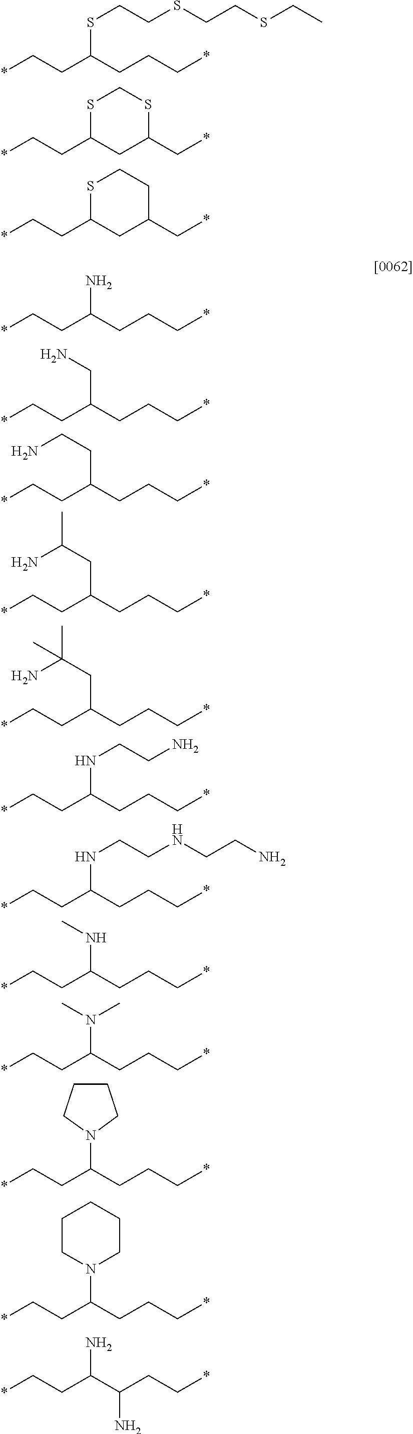

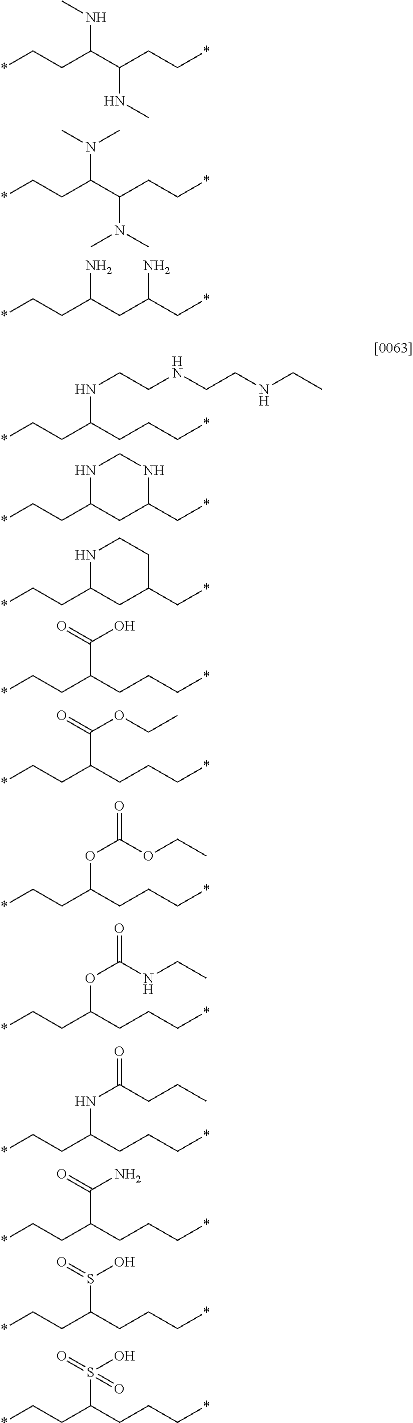

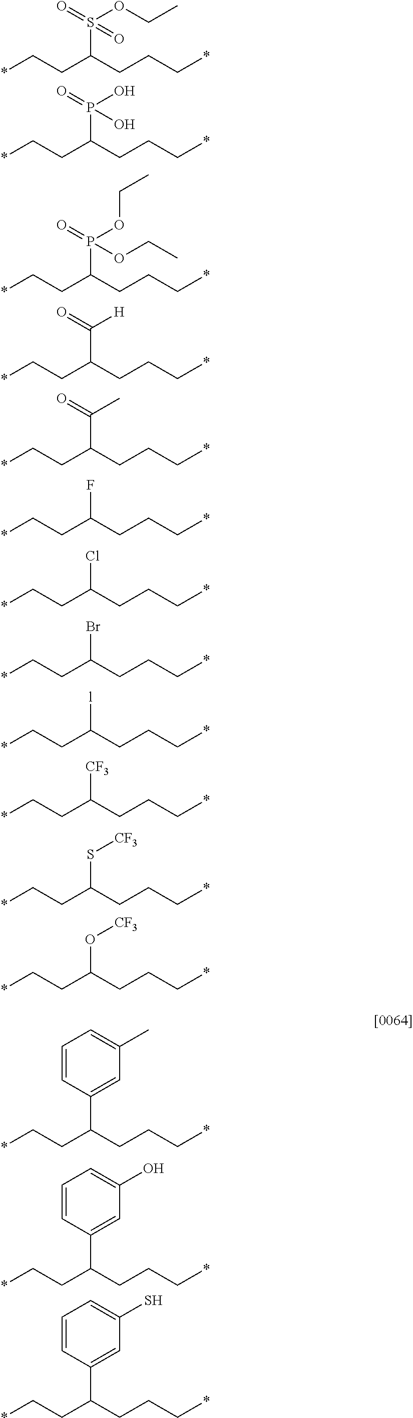

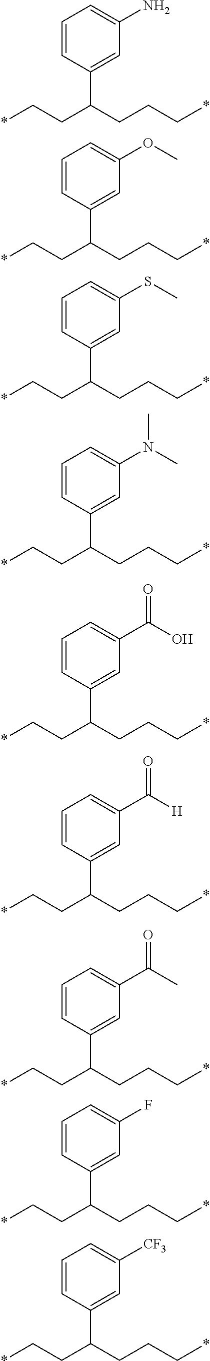

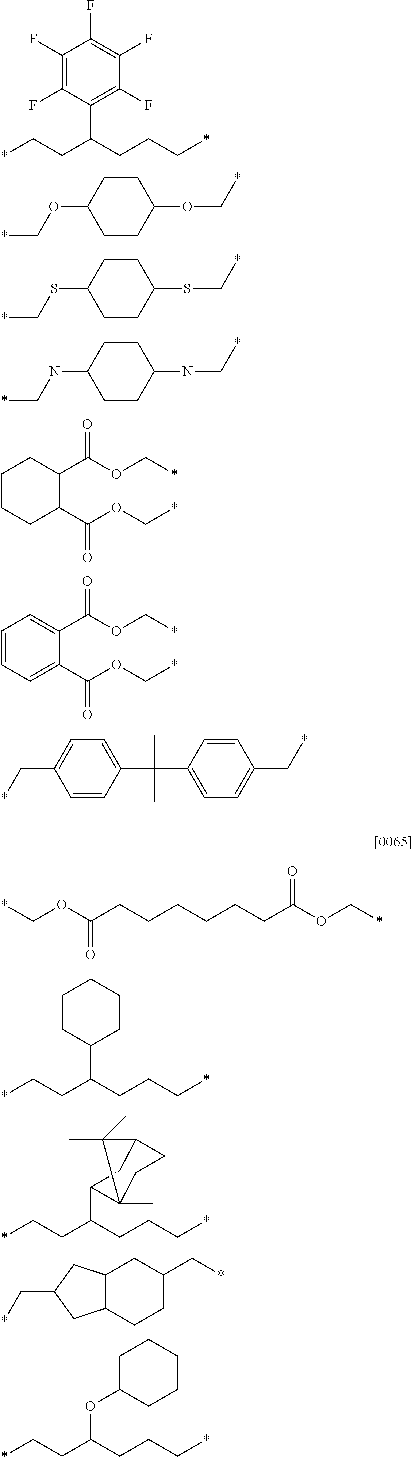

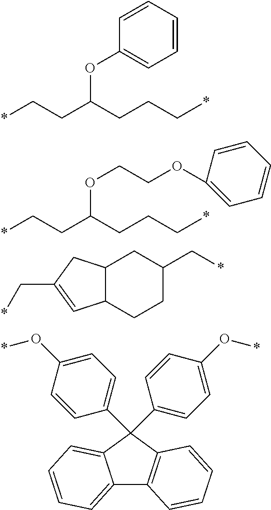

The divalent linking groups represented by R⁶ and R⁷ are each independently preferably a divalent aliphatic hydrocarbon group having 1 to 18 carbon atoms which may have a substituent or a linking group represented by the following formula, and more preferably a divalent aliphatic hydrocarbon group having 1 to 12 carbon atoms which may have a substituent or a linking group represented by the following formula.

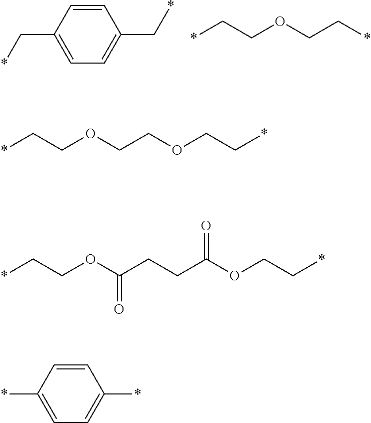

R⁸ is preferably a divalent aliphatic hydrocarbon group having 1 to 18 carbon atoms which may have a substituent or a linking group represented by the following formula.

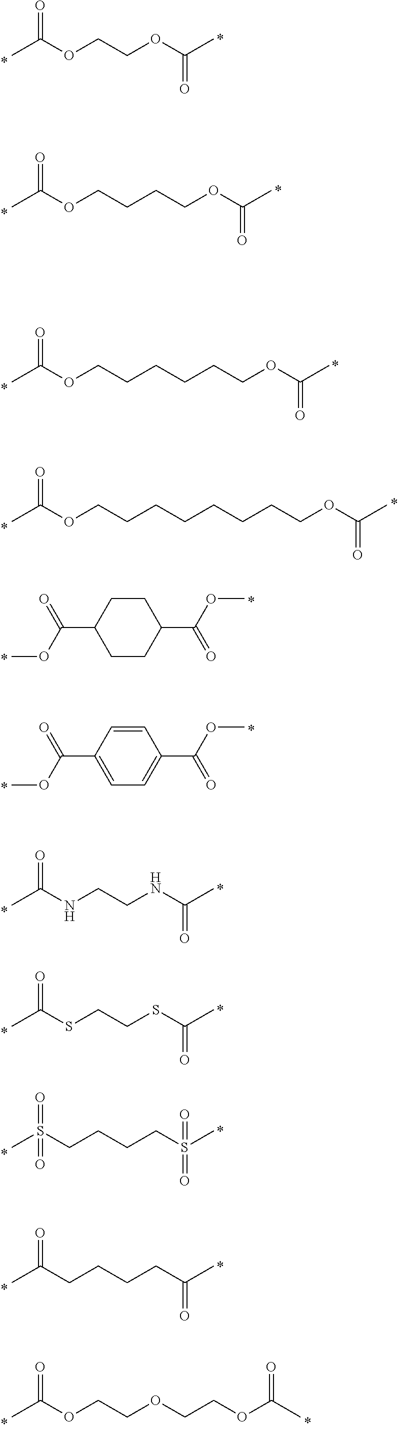

Examples of the trivalent linking group represented by R⁹ and R¹⁰ each independently include a trivalent aliphatic hydrocarbon group having 1 to 18 carbon atoms which may have a substituent or a trivalent aromatic hydrocarbon group having 6 to 18 carbon atoms which may have a substituent. —CH₂— contained in the trivalent aliphatic hydrocarbon group may be substituted with —O—, —S—, —CS—, —CO—, —SO—, or —NR^{11B}— (R^{11B} represents a hydrogen atom or an alkyl group having 1 to 6 carbon atoms).

Examples of the substituent that the trivalent aliphatic hydrocarbon group and the trivalent aromatic hydrocarbon group may have include a halogen atom, a hydroxyl group, a carboxy group, and an amino group.

It is preferable that the trivalent linking groups represented by R⁹ and R¹⁰ are each independently a trivalent aliphatic hydrocarbon group having 1 to 12 carbon atoms which may have a substituent.

Specific examples of the trivalent linking group represented by R⁹ and R¹⁰ include the following linking groups.

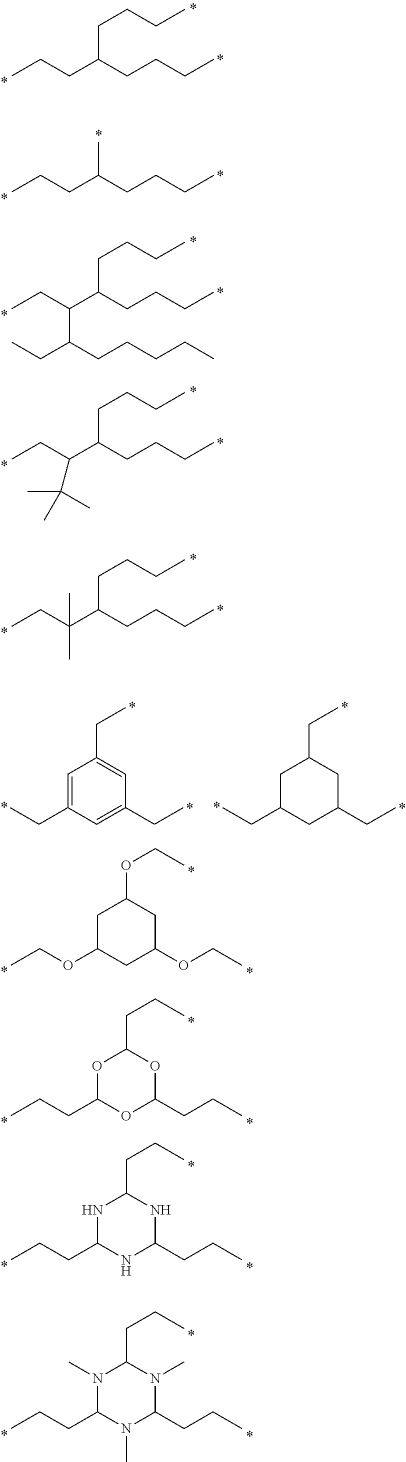

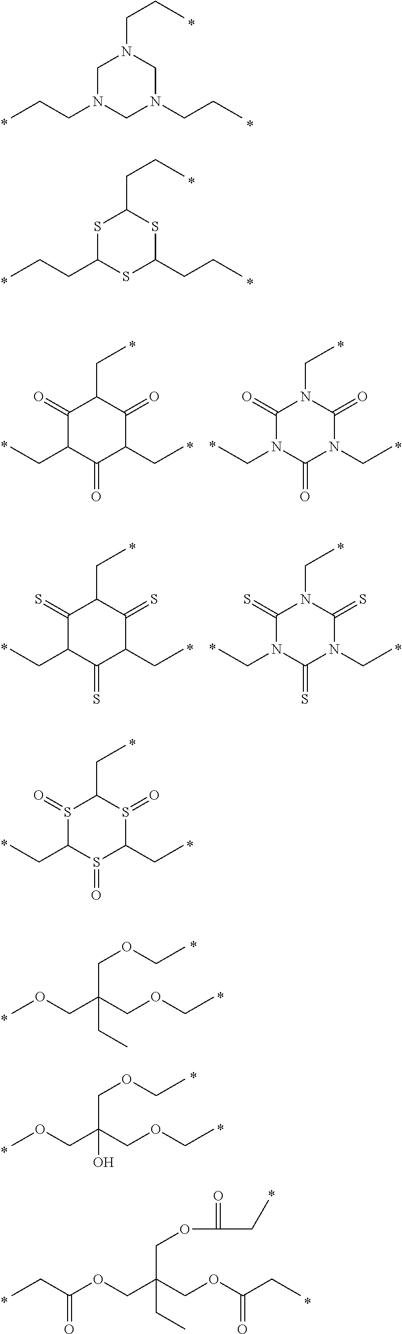

Examples of the tetravalent linking group represented by R¹¹ include a tetravalent aliphatic hydrocarbon group having 1 to 18 carbon atoms which may have a substituent or a tetravalent aromatic hydrocarbon group having 6 to 18 carbon atoms which may have a substituent. —CH₂— contained in the tetravalent aliphatic hydrocarbon group may be substituted with —O—, —S—, —CS—, —CO—, —SO—, or —NR^{11C}— (R^{11C} represents a hydrogen atom or an alkyl group having 1 to 6 carbon atoms).

Examples of the substituent that the tetravalent aliphatic hydrocarbon group and the tetravalent aromatic hydrocarbon group may have include a halogen atom, a hydroxyl group, a carboxy group, and an amino group.

It is preferable that the tetravalent linking groups represented by R¹¹ are each independently a tetravalent aliphatic hydrocarbon group having 1 to 12 carbon atoms which may have a substituent.

Specific examples of the tetravalent linking group represented by R¹¹ include the following linking groups.

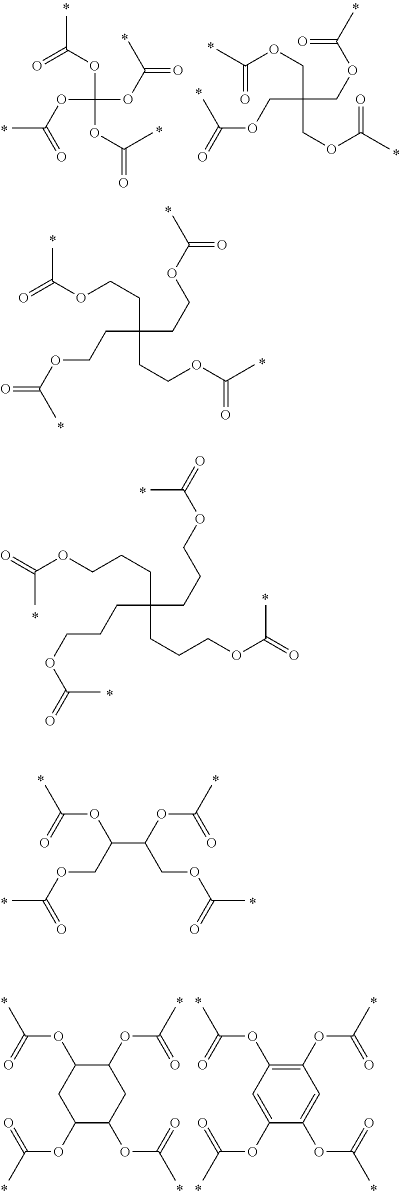

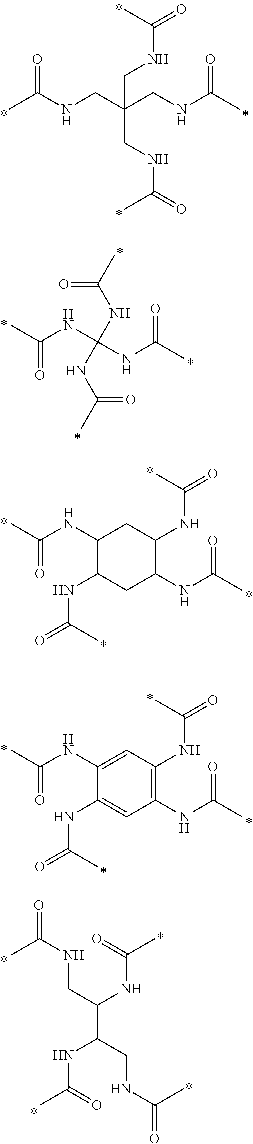

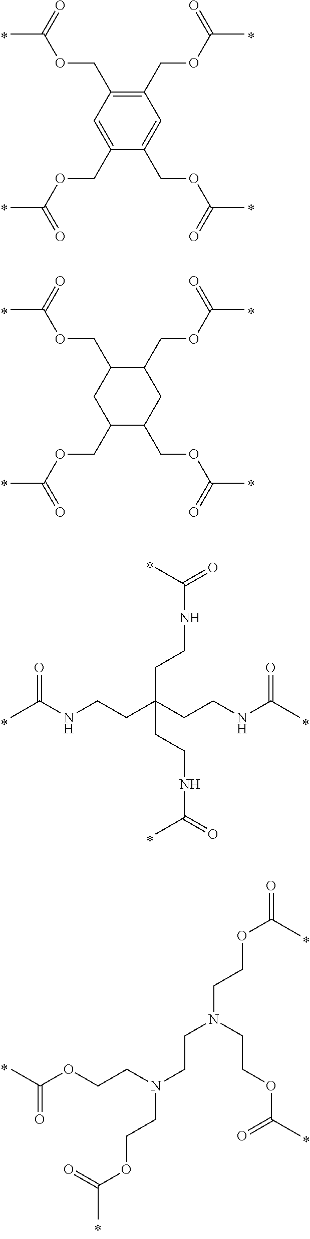

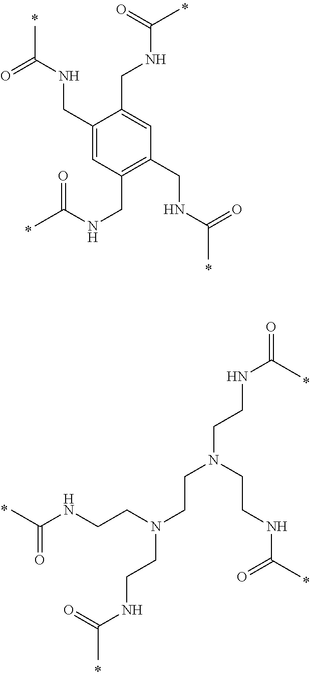 [SMILES: CC(=O)NCc1cc(CNC(C)=O)c(CNC(C)=O)cc1CNC(C)=O ; CC(=O)NCCN(CCNC(C)=O)CCN(CCNC(C)=O)CCNC(C)=O]

R¹ is preferably an alkyl group having 1 to 15 carbon atoms, and more preferably an alkyl group having 1 to 10 carbon atoms.

R² is preferably an alkyl group having 1 to 15 carbon atoms, and more preferably an alkyl group having 1 to 10 carbon atoms.

R¹ and R² are preferably linked to each other to form a ring, more preferably an aliphatic ring, still more preferably an aliphatic ring having no unsaturated bond, and particularly preferably a pyrrolidine ring or a piperidine ring structure.

R³ is preferably a nitro group; a cyano group; a halogen atom; —OCF₃, —SCF₃, —SF₅, —SF₃, a fluoroalkyl group (preferably, 1 to 25 carbon atoms), a fluoroaryl group (preferably, 6 to 18 carbon atoms), —CO—O—R^{111A} or —SO₂—R^{112A}(R^{111A} and R^{112A} each independently represent an alkyl group having 1 to 24 carbon atoms), more preferably a cyano group, a fluorine atom, a chlorine atom, —OCF₃, —SCF₃, a fluoroalkyl group, —CO—O—R^{111A} or —SO₂—R^{112A} (R^{111A} and R^{112A} each independently represent an alkyl group having 1 to 24 carbon atoms which may have a halogen atom), still more preferably a cyano group or a fluorine atom, and particularly preferably a cyano group.

R⁴ and R⁵ are preferably each independently a nitro group, a cyano group, a halogen atom, —OCF₃, —SCF₃, —SF₅, —SF₃, a fluoroalkyl group, a fluoroaryl group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent),

more preferably a nitro group, a cyano group, a fluorine atom, a chlorine atom, —OCF₃, —SCF₃, a fluoroalkyl group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent),

still more preferably a cyano group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), and particularly preferably a cyano group.

At least one of R⁴ and R⁵ is preferably a cyano group, R⁴ is more preferably a cyano group, and R⁵ is more preferably a cyano group, —CO—O—R²²² or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent).

R⁴ and R⁵ preferably have the same structure.

R⁴ and R⁵ are each preferably a cyano group.

R⁴¹, R⁵¹, R⁶¹, R⁹¹, R¹⁰¹, and R¹¹¹ are each independently preferably an alkyl group having 1 to 15 carbon atoms, and more preferably an alkyl group having 1 to 10 carbon atoms.

R¹², R⁴², R⁵², R⁶², R⁷², R⁸², R⁹², R¹⁰² and R¹¹² are each independently preferably an alkyl group having 1 to 15 carbon atoms, and more preferably an alkyl group having 1 to 10 carbon atoms.

R⁴¹ and R⁴² are preferably linked to each other to form a ring, more preferably an aliphatic ring, still more preferably an aliphatic ring having no unsaturated bond, and particularly preferably a pyrrolidine ring or a piperidine ring structure.

R⁵¹ and R⁵² are preferably linked to each other to form a ring, more preferably an aliphatic ring, still more preferably an aliphatic ring having no unsaturated bond, and particularly preferably a pyrrolidine ring or a piperidine ring structure.

R⁶¹ and R⁶² are preferably linked to each other to form a ring, more preferably an aliphatic ring, still more preferably an aliphatic ring having no unsaturated bond, and particularly preferably a pyrrolidine ring or a piperidine ring structure.

R⁹¹ and R³² are preferably linked to each other to form a ring, more preferably an aliphatic ring, still more preferably an aliphatic ring having no unsaturated bond, and particularly preferably a pyrrolidine ring or a piperidine ring structure.

R¹⁰¹ and R¹⁰² are preferably linked to each other to form a ring, more preferably an aliphatic ring, still more preferably an aliphatic ring having no unsaturated bond, and particularly preferably a pyrrolidine ring or a piperidine ring structure.

R¹¹¹ and R¹¹² are preferably linked to each other to form a ring, more preferably an aliphatic ring, still more preferably an aliphatic ring having no unsaturated bond, and particularly preferably a pyrrolidine ring or a piperidine ring structure.

R¹³, R²³, R³³, R⁴³, R⁵³, R⁶³, R⁷³, R⁸³, R⁹³, R¹⁰³, and R¹¹³ are each independently preferably a nitro group; a cyano group; a halogen atom; —OCF₃; —SCF₃; —SF₅; —SF₃; a fluoroalkyl group having 1 to 25 carbon atoms; a fluoroaryl group having 6 to 18 carbon atoms; —CO—O—R^{111A} or —SO₂—R^{112A}(R^{111A} and R^{112A} each independently represent an alkyl group having 1 to 24 carbon atoms which may have a halogen atom),

more preferably a cyano group; a fluorine atom; a chlorine atom; —OCF₃; —SCF₃; a fluoroalkyl group having 1 to 12 carbon atoms; —CO—O—R^{111A} or —SO₂—R^{112A} (R^{111A} and R^{112A} each independently represent an alkyl group having 1 to 24 carbon atoms which may have a halogen atom), and particularly preferably a cyano group.

R¹⁴, R²⁴, R³⁴, R⁴⁴, R⁵⁴, R⁶⁴, R⁷⁴, R⁸⁴, R⁹⁴, R¹⁰⁴, R¹¹⁴, R¹⁵, R²⁵, R³⁵, R⁷⁵, and R⁹⁵ are each independently preferably a nitro group, a cyano group, a halogen atom, —OCF₃, —SCF₃, —SF₅, —SF₃, —CO—O—R²²², —SO₂—R²²² (R²²² represents an alkyl group having 1 to 25 carbon atoms which may have a halogen atom), a fluoroalkyl group having 1 to 25 carbon atoms, or a fluoroaryl group having 6 to 18 carbon atoms, more preferably a nitro group, a cyano group, a fluorine atom, a chlorine atom, —OCF₃, —SCF₃, a fluoroalkyl group, —CO—O—R²²² or —SO₂—R²²² (R²²² represents an alkyl group having 1 to 25 carbon atoms which may have a halogen atom), still more preferably a cyano group, —CO—O—R²²² or —SO₂—R²²² (R²²² represents an alkyl group having 1 to 25 carbon atoms which may have a halogen atom), and particularly preferably a cyano group.

It is preferable that R¹⁴ and R¹⁵ have the same structure.

It is preferable that R²⁴ and R²⁵ have the same structure.

It is preferable that R³⁴ and R³⁵ have the same structure.

It is preferable that R⁷⁴ and R⁷⁵ have the same structure.

It is preferable that R⁸⁴ and R⁸⁵ have the same structure.

The compound represented by Formula (I) is more preferably any of a compound represented by Formula (I-1A), a compound represented by Formula (I-2A), and a compound represented by Formula (I-3A).

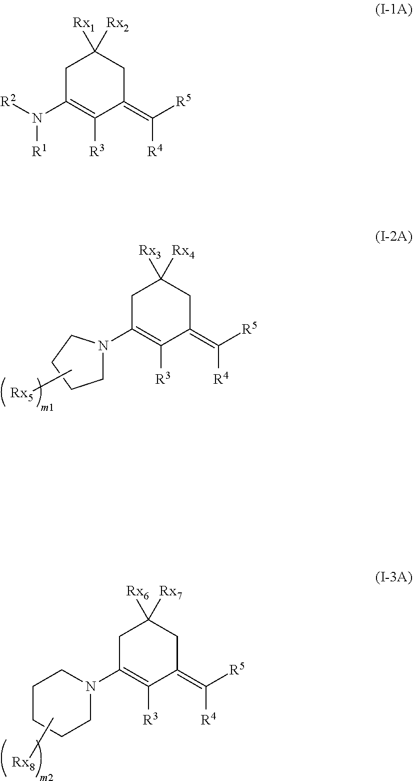

[in the formula, R¹, R², R³, R⁴, and R⁵ have the same meaning as described above;

Rx₁, Rx₂, Rx₃, Rx₄, Rx₅, Rx₆, Rx₇, and Rx₈ each independently represent a hydrogen atom or a substituent; and

m1 represents an integer of 0 to 4, and m2 represents an integer of 0 to 5].

Examples of the substituents represented by Rx₁ to Rx₈ include the same substituents as those that the ring W¹ may have.

m1 and m2 are each independently preferably 0 or 1.

The compound represented by Formula (II) is preferably a compound represented by Formula (II-A).

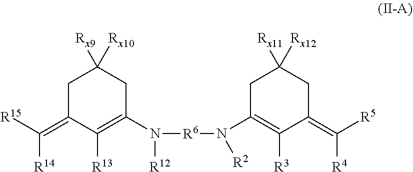

[in the formula, R², R³, R⁴, R⁵, R⁶, R¹², R¹³, R¹⁴, and R¹⁵ have the same meaning as described above; and

Rₓ₉, Rₓ₁₀, Rₓ₁₁, and Rₓ₁₂ each independently represent a hydrogen atom or a substituent].

Examples of the substituents represented by Rₓ₉ to Rₓ₁₂ include the same substituents as those that the ring W¹ may have.

The compound represented by Formula (III) is preferably a compound represented by Formula (III-A).

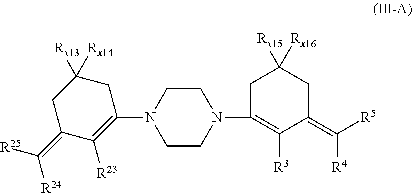

[in the formula, R³, R⁴, R⁵, R²³, R²⁴, and R²⁵ have the same meaning as described above; and

Rₓ₁₃, Rₓ₁₄, Rₓ₁₅, and Rₓ₁₆ each independently represent a hydrogen atom or a substituent].

Examples of the substituents represented by Rₓ₁₃ to Rₓ₁₆ include the same substituents as those that the ring W¹ may have.

Examples of the compound represented by Formula (I) (hereinafter, may be referred to as a compound (I)) include the following compounds.

The compound (I) is preferably compounds represented by Formula (1-1) to Formula (1-4), Formula (1-7), Formula (1-8), Formula (1-10), Formula (1-12), Formula (1-20) to Formula (1-25), Formula (1-54) to Formula (1-57), Formula (1-59), Formula (1-63) to Formula (1-68), Formula (1-70) to Formula (1-78), Formula (1-80), Formula (1-124) to Formula (1-132), Formula (1-135), Formula (1-137) to Formula (1-142), Formula (1-158) to Formula (1-172), and Formula (1-218) to Formula (1-229), is more preferably compounds represented by Formula (1-1), Formula (1-2), Formula (1-4), Formula (1-7), Formula (1-10), Formula (1-12), Formula (1-20), Formula (1-22), Formula (1-54) to Formula (1-56), Formula (1-59), Formula (1-63) to Formula (1-65), Formula (1-66), Formula (1-71), Formula (1-124), Formula (1-125), Formula (1-126), Formula (1-128), Formula (1-131), Formula (1-158), Formula (1-160), Formula (1-164), Formula (1-169), and Formula (1-218) to Formula (1-227), and still more preferably compounds represented by Formula (1-54) to Formula (1-56), Formula (1-59), Formula (1-64), Formula (1-125), and Formula (1-218) to Formula (1-229).

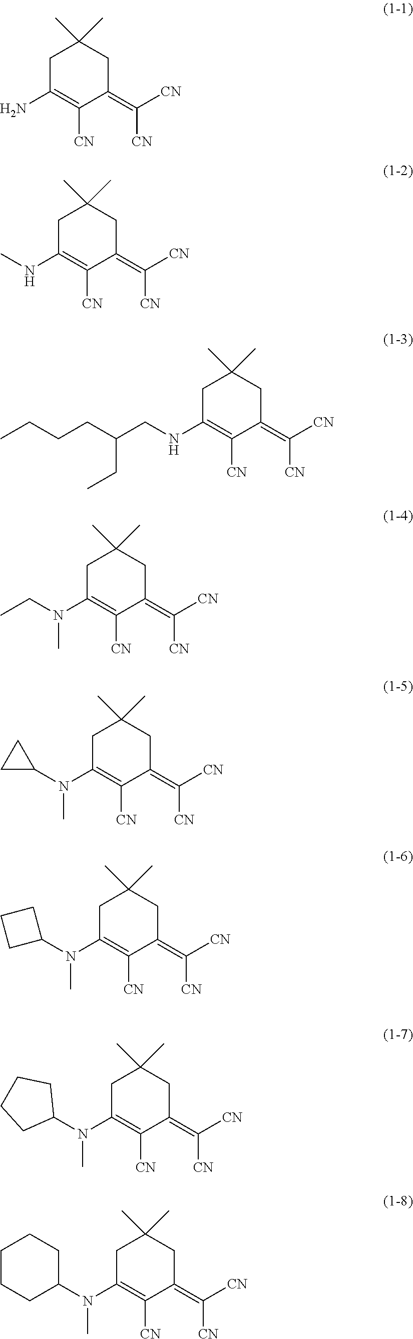

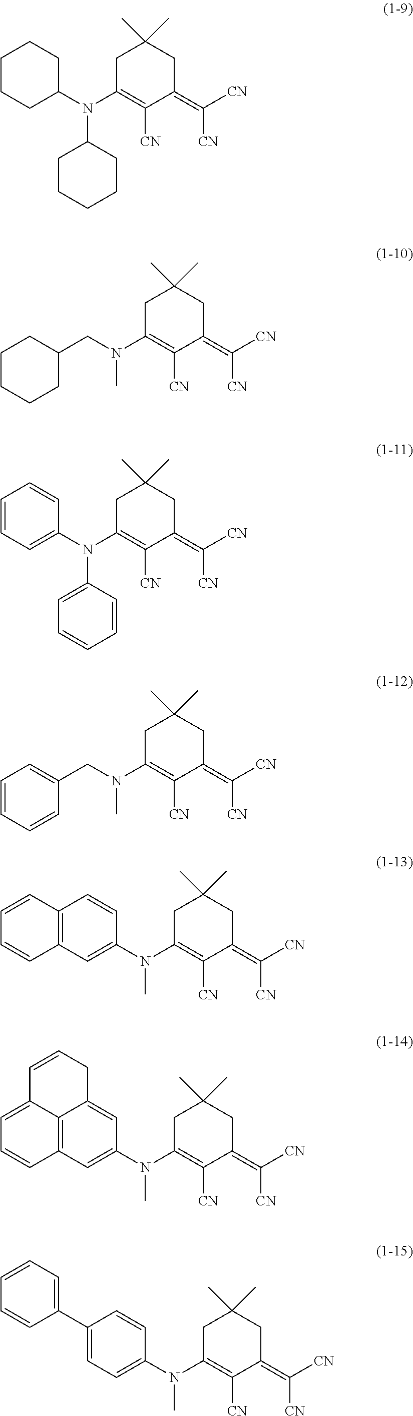

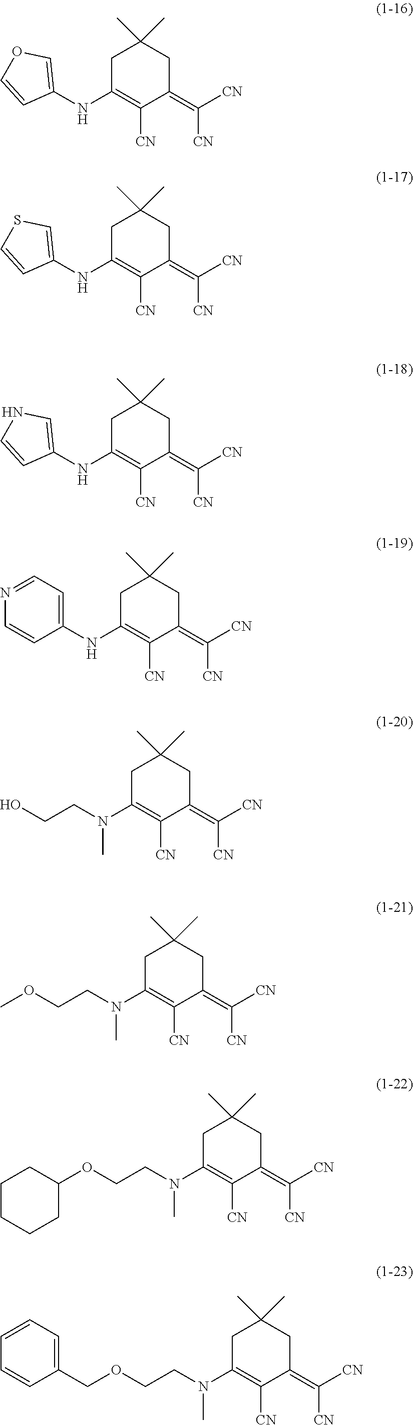

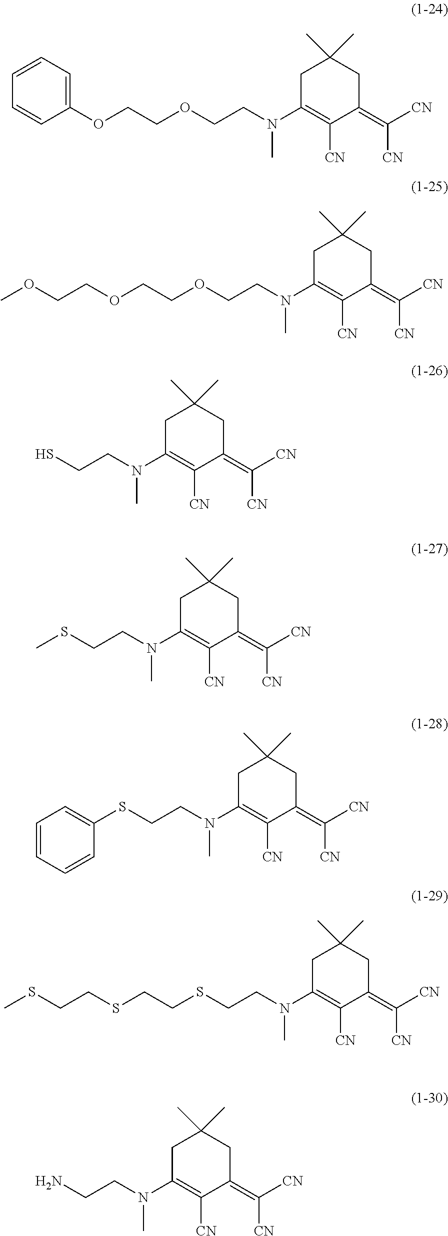

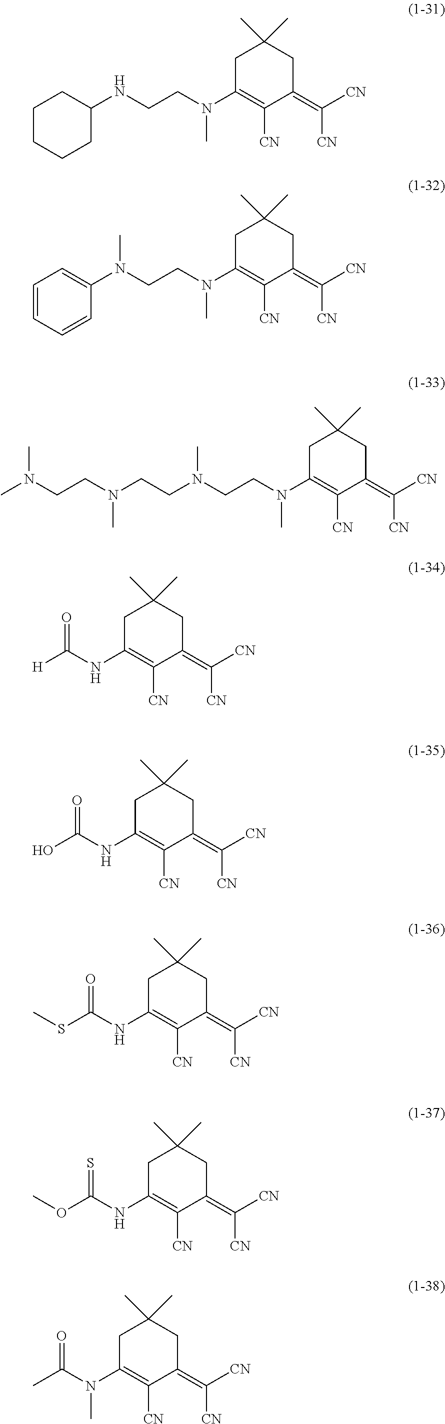

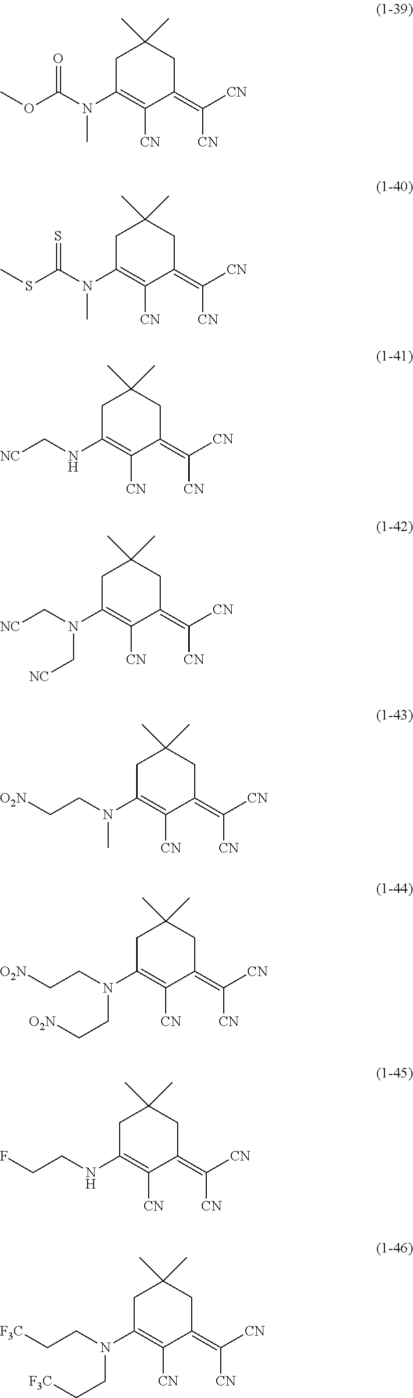

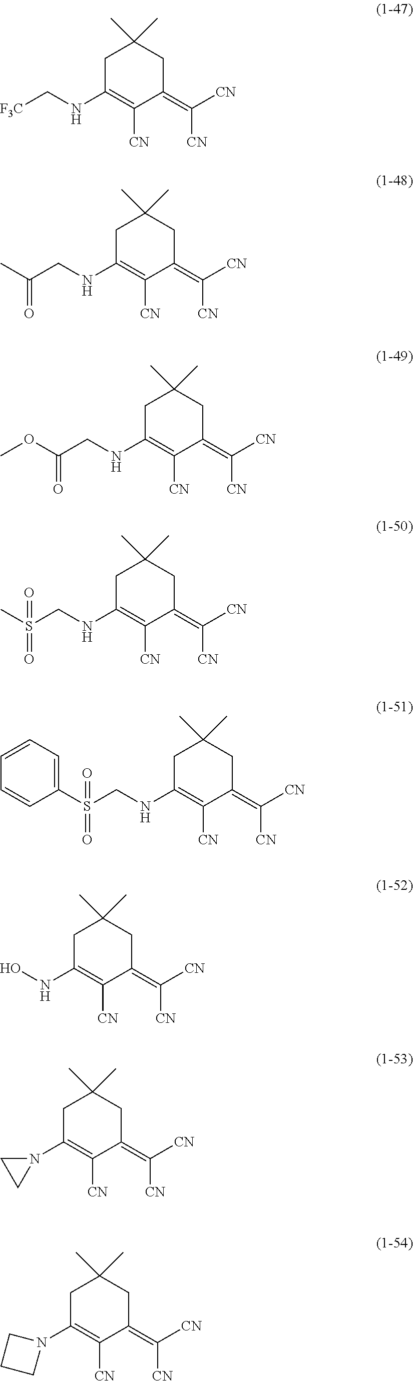

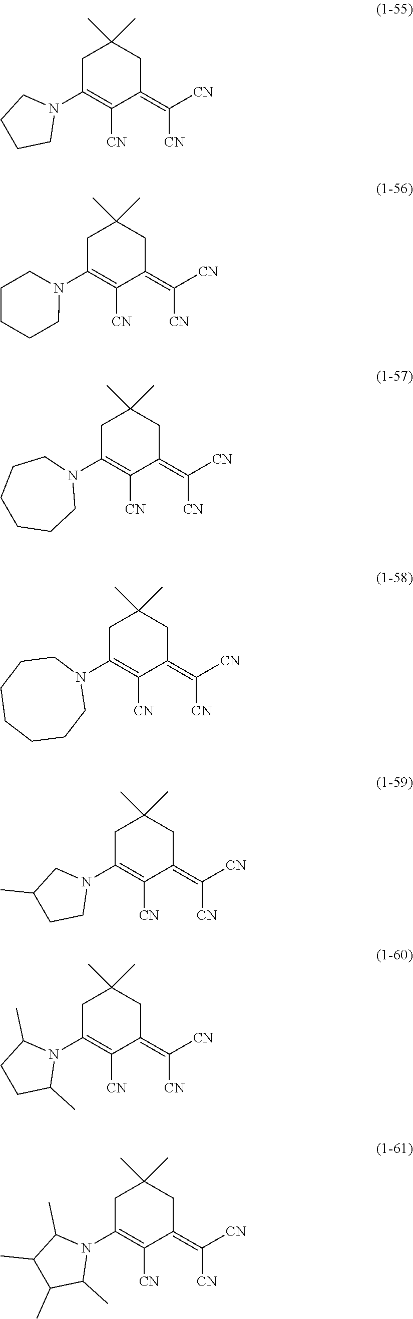

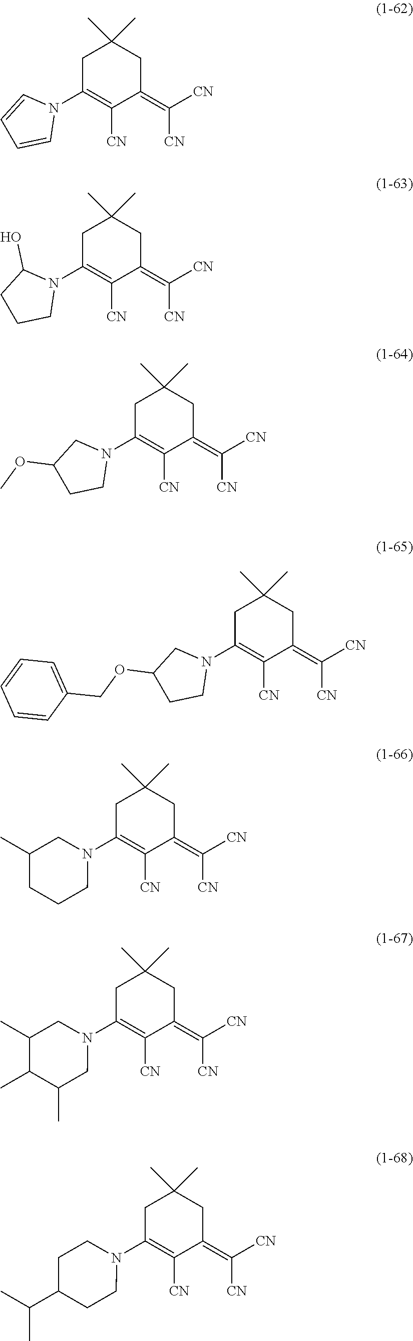

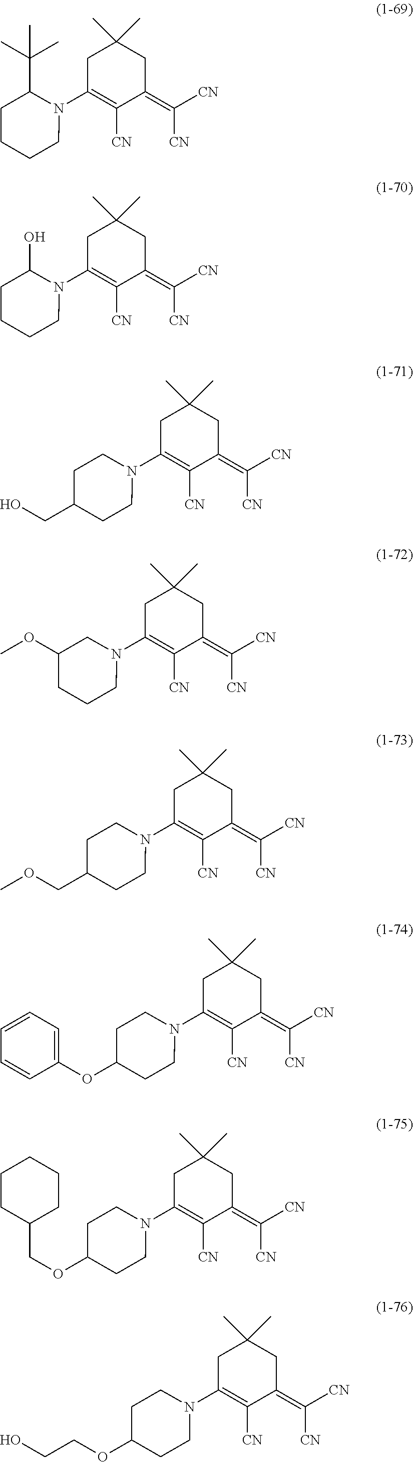

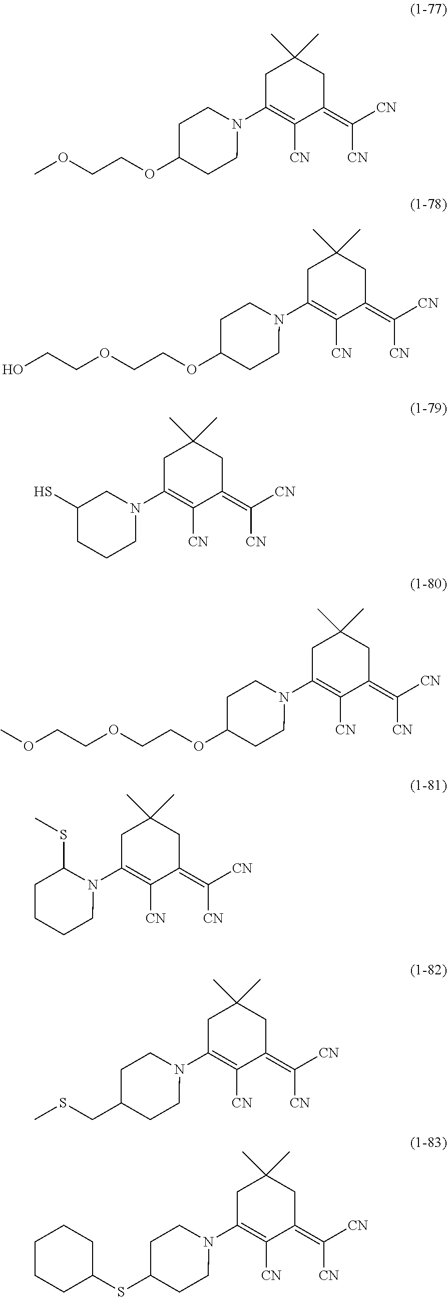

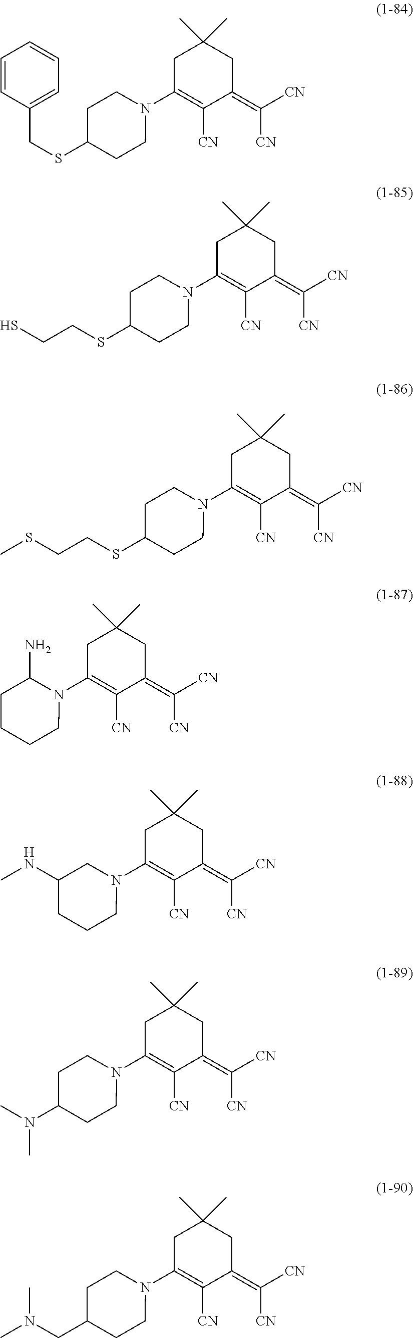

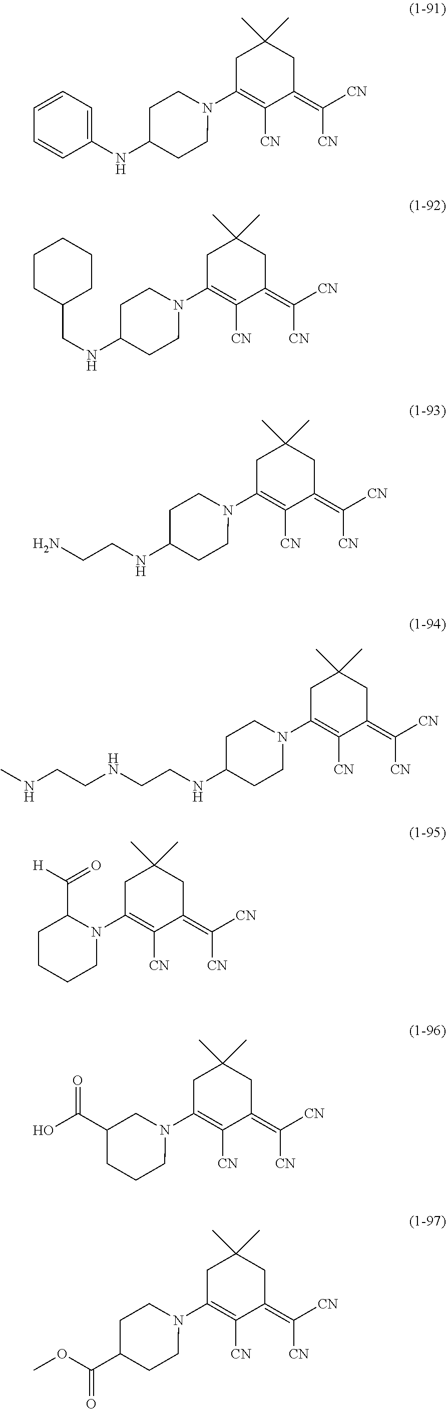

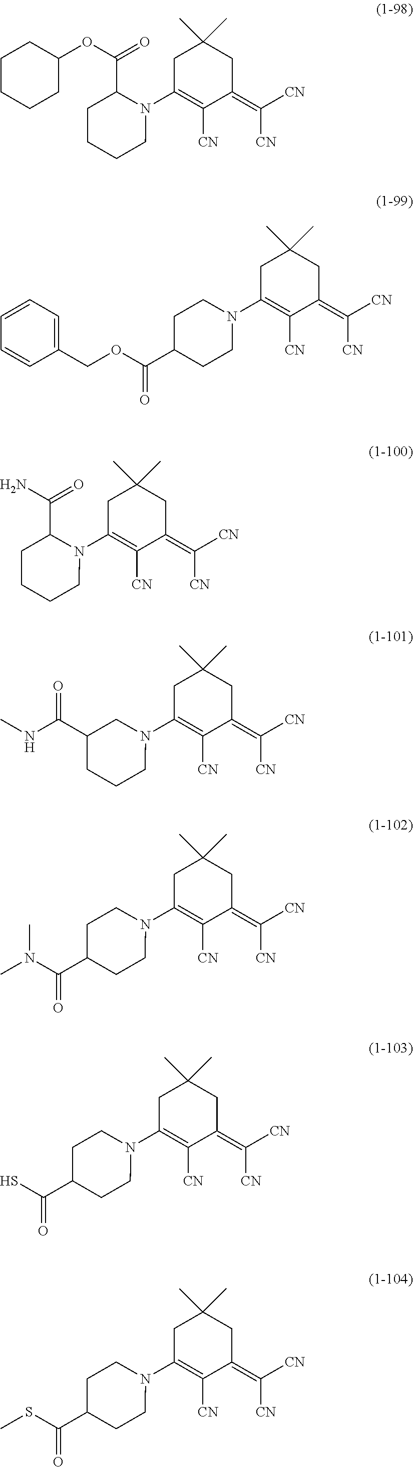

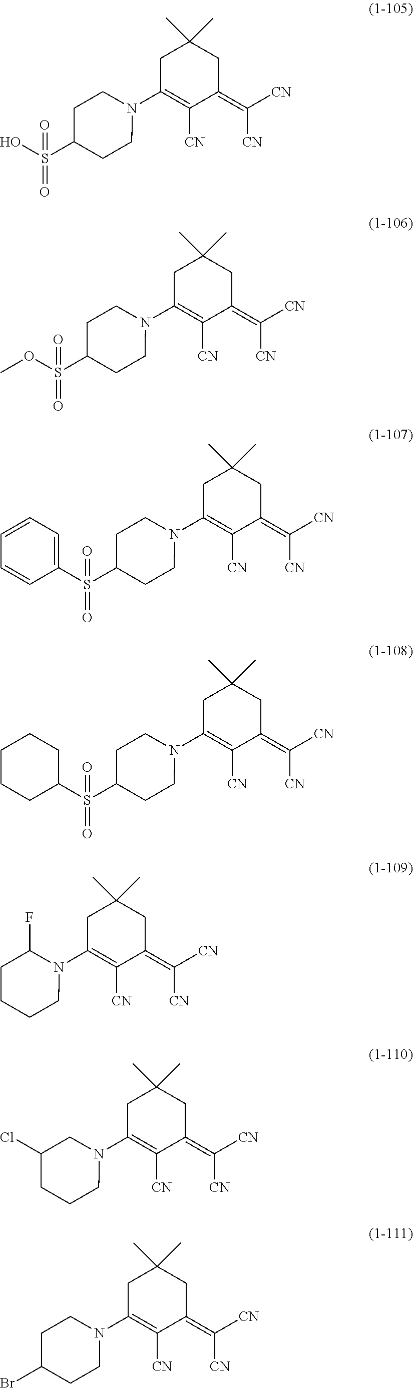

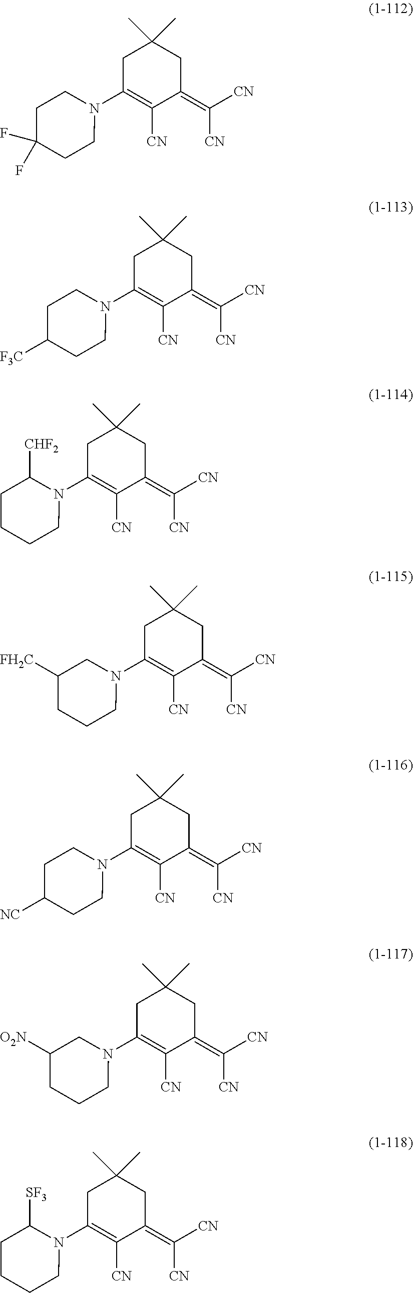

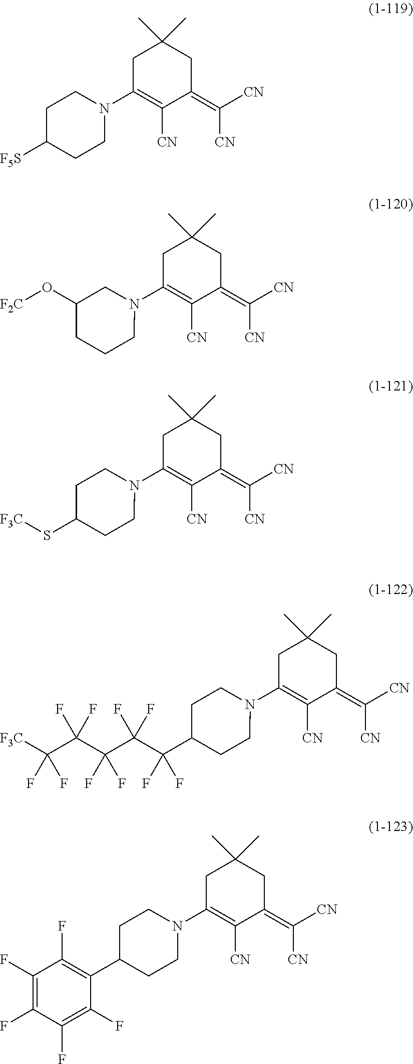

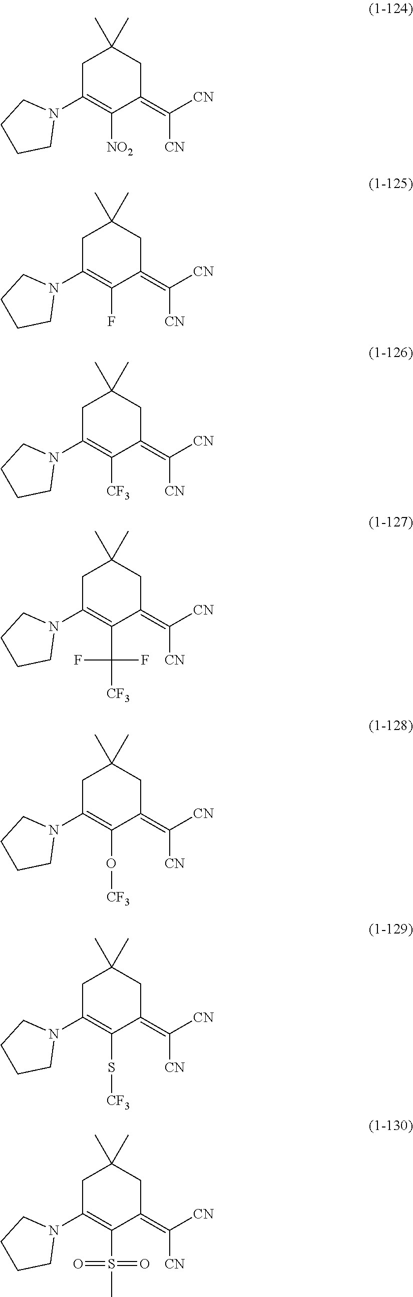

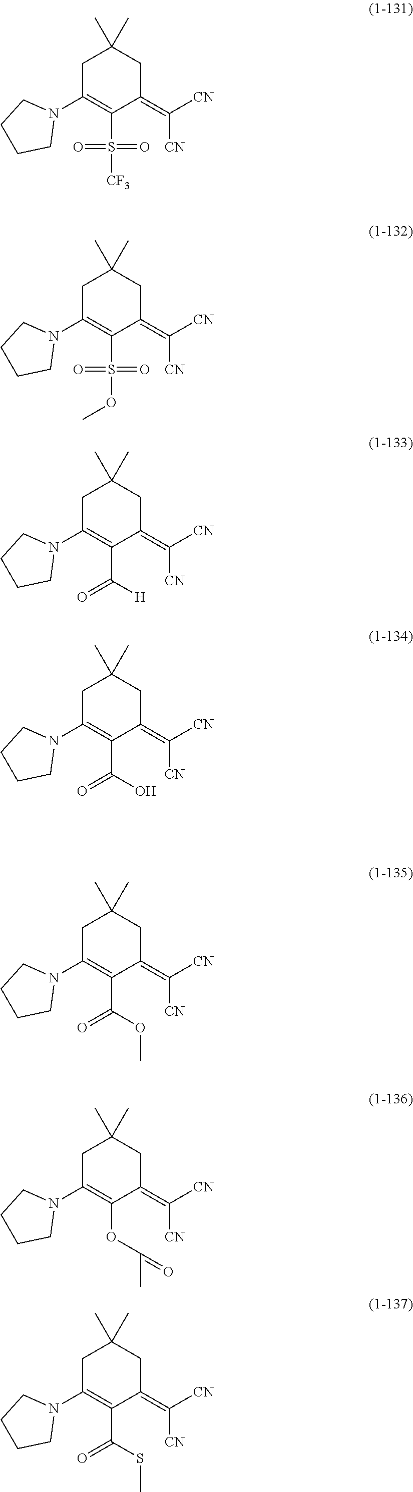

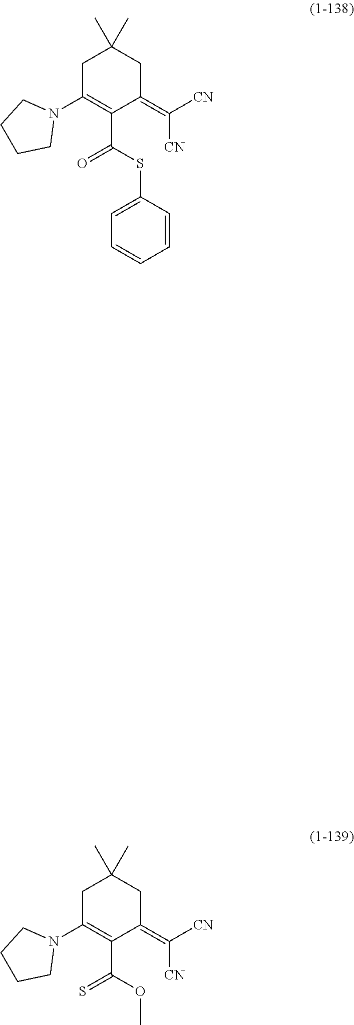 [SMILES: CC1(C)CC(=C(C#N)C#N)C(C(=O)Sc2ccccc2)=C(N2CCCC2)C1 ; COC(=S)C1=C(N2CCCC2)CC(C)(C)CC1=C(C#N)C#N]

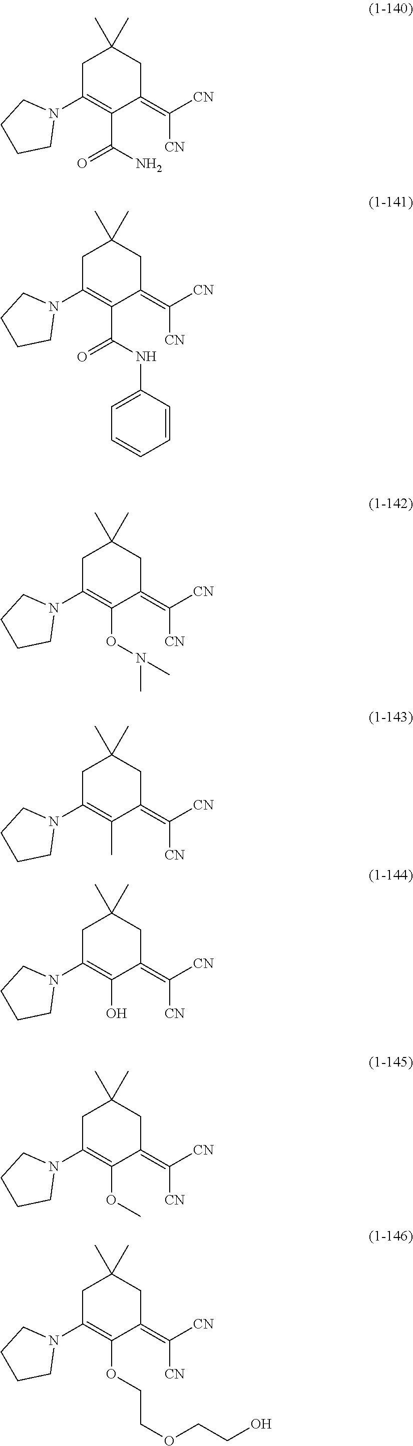

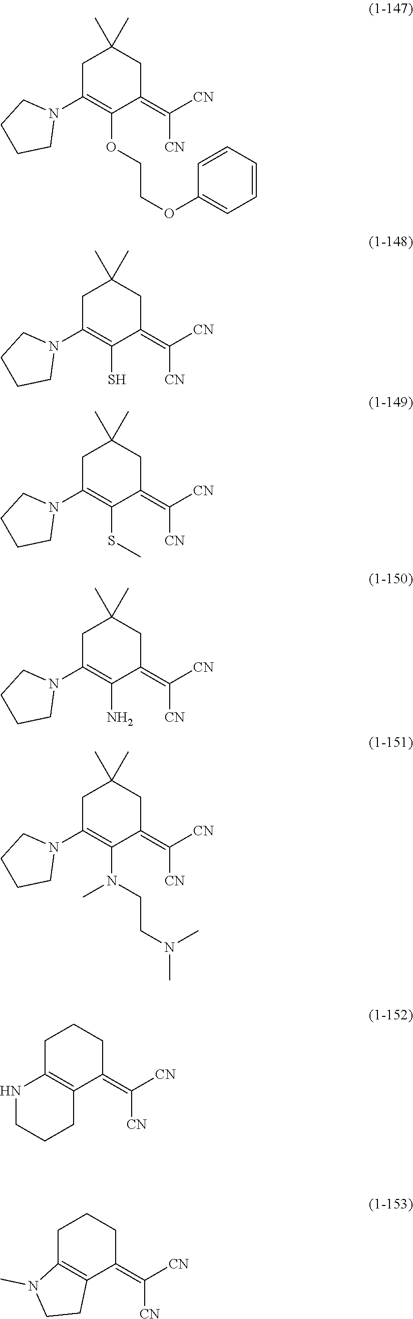

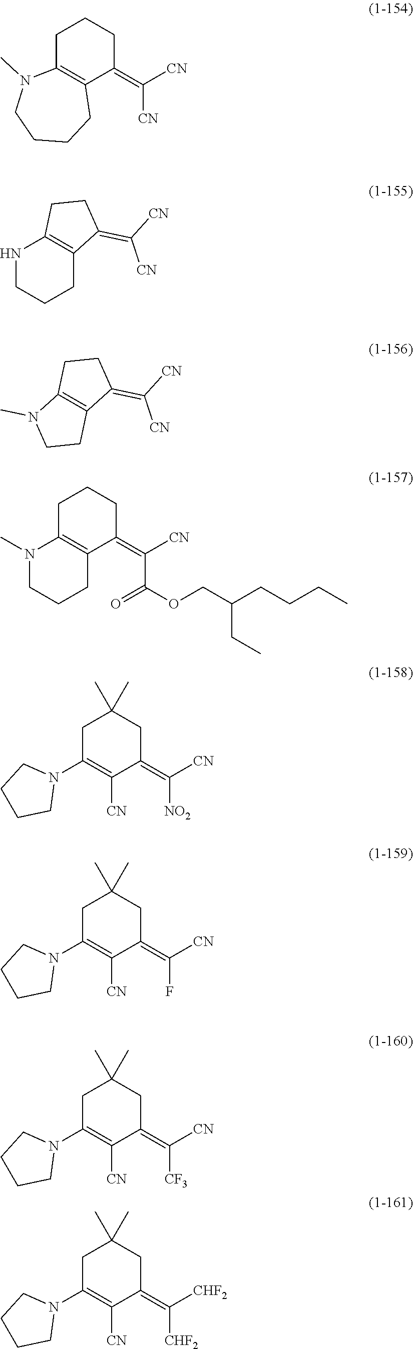

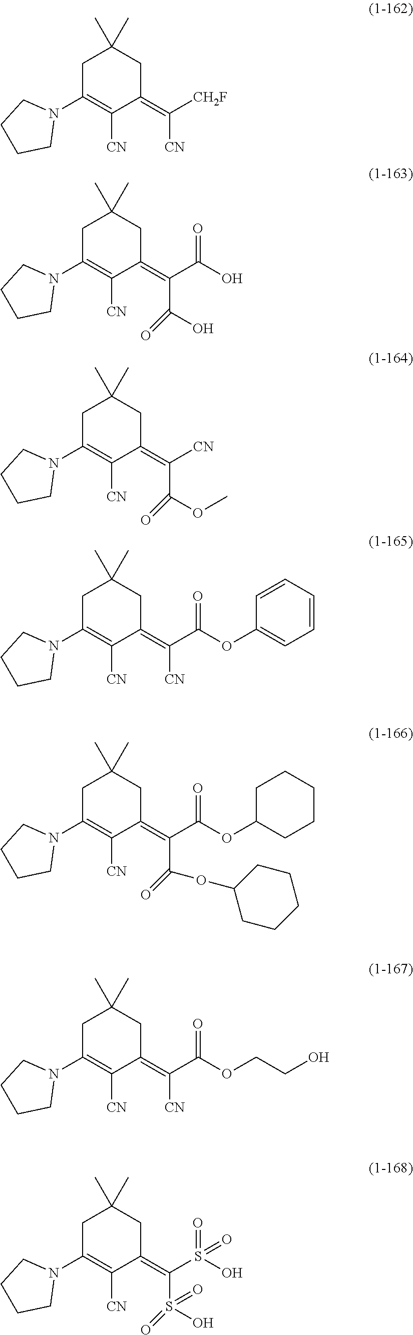

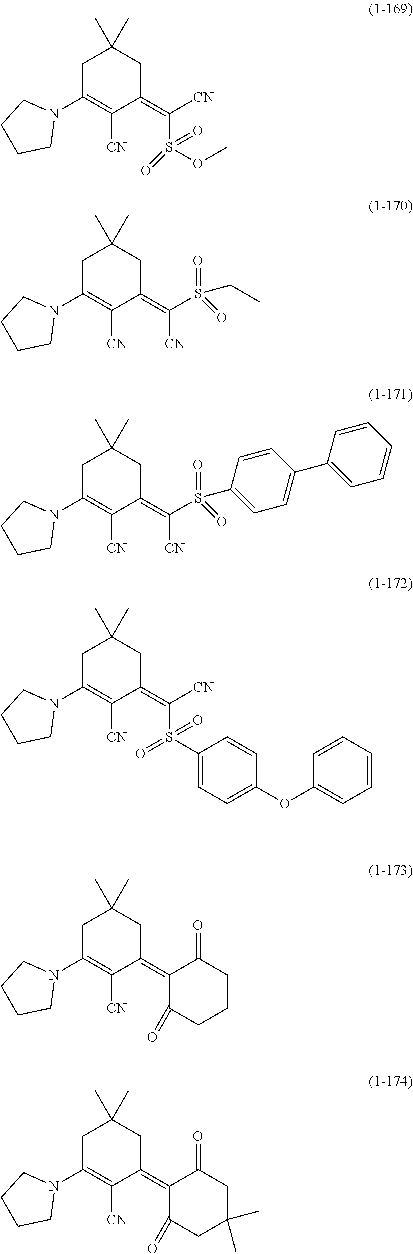 [SMILES: COS(=O)(=O)/C(C#N)=C1/CC(C)(C)CC(N2CCCC2)=C1C#N ; CCS(=O)(=O)/C(C#N)=C1\CC(C)(C)CC(N2CCCC2)=C1C#N ; CC1(C)CC(N2CCCC2)=C(C#N)/C(=C(\C#N)S(=O)(=O)c2ccc(-c3ccccc3)cc2)C1 ; CC1(C)CC(N2CCCC2)=C(C#N)/C(=C(/C#N)S(=O)(=O)c2ccc(Oc3ccccc3)cc2)C1 ; CC1(C)CC(=C2C(=O)CCCC2=O)C(C#N)=C(N2CCCC2)C1 ; CC1(C)CC(=O)C(=C2CC(C)(C)CC(N3CCCC3)=C2C#N)C(=O)C1]

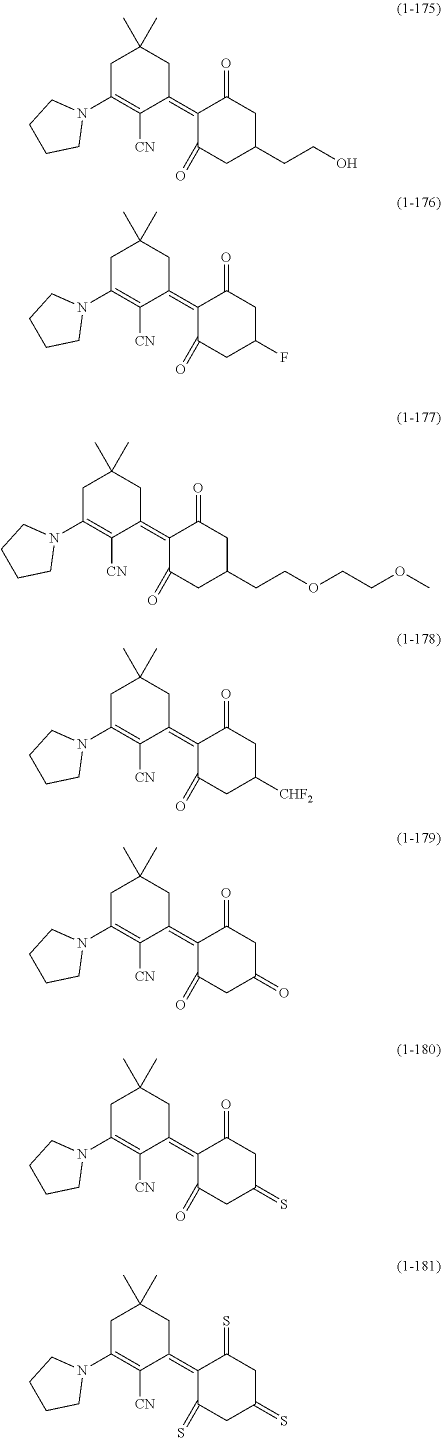

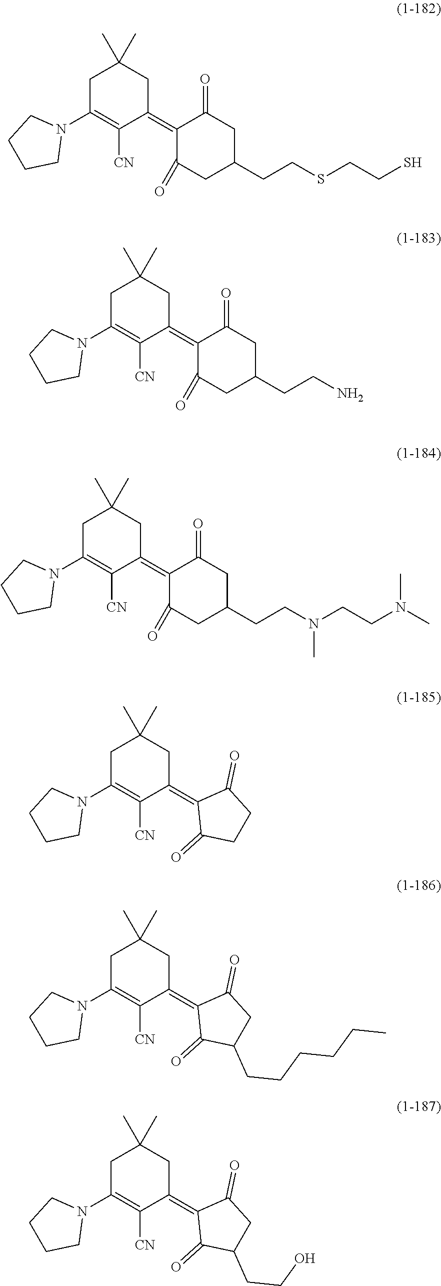 [SMILES: CC1(C)CC(=C2C(=O)CC(CCSCCS)CC2=O)C(C#N)=C(N2CCCC2)C1 ; CC1(C)CC(=C2C(=O)CC(CCN)CC2=O)C(C#N)=C(N2CCCC2)C1 ; CN(C)CCN(C)CCC1CC(=O)C(=C2CC(C)(C)CC(N3CCCC3)=C2C#N)C(=O)C1 ; CC1(C)CC(=C2C(=O)CCC2=O)C(C#N)=C(N2CCCC2)C1 ; CCCCCCC1CC(=O)/C(=C2\CC(C)(C)CC(N3CCCC3)=C2C#N)C1=O ; CC1(C)CC(N2CCCC2)=C(C#N)/C(=C2/C(=O)CC(CCO)C2=O)C1]

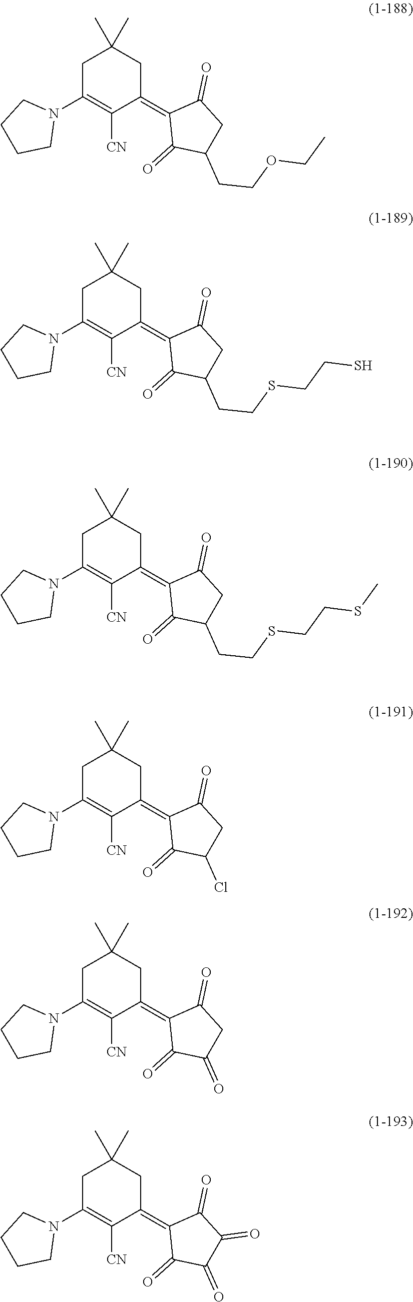 [SMILES: CCOCCC1CC(=O)/C(=C2\CC(C)(C)CC(N3CCCC3)=C2C#N)C1=O ; CC1(C)CC(N2CCCC2)=C(C#N)/C(=C2/C(=O)CC(CCSCCS)C2=O)C1 ; CSCCSCCC1CC(=O)/C(=C2\CC(C)(C)CC(N3CCCC3)=C2C#N)C1=O ; CC1(C)CC(N2CCCC2)=C(C#N)/C(=C2/C(=O)CC(Cl)C2=O)C1 ; CC1(C)CC(N2CCCC2)=C(C#N)/C(=C2/C(=O)CC(=O)C2=O)C1 ; CC1(C)CC(=C2C(=O)C(=O)C(=O)C2=O)C(C#N)=C(N2CCCC2)C1]

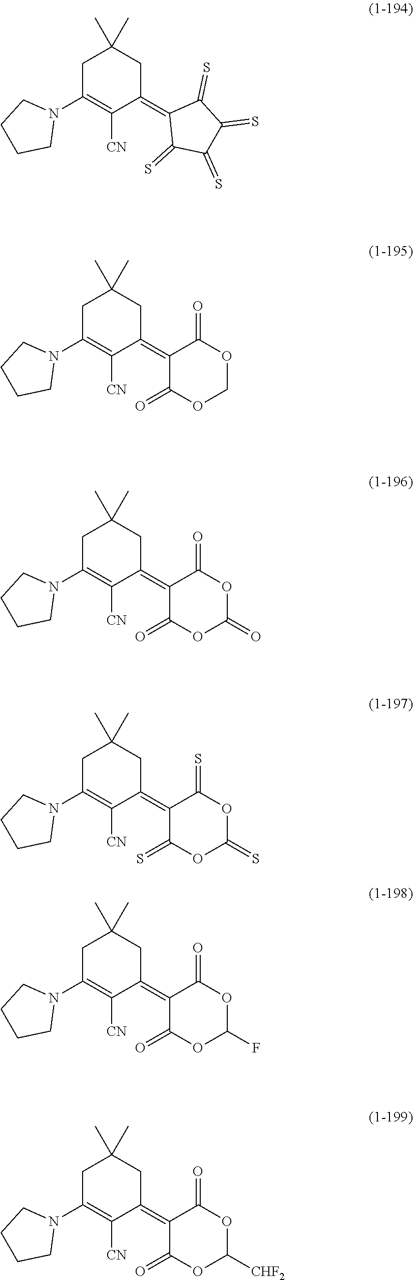

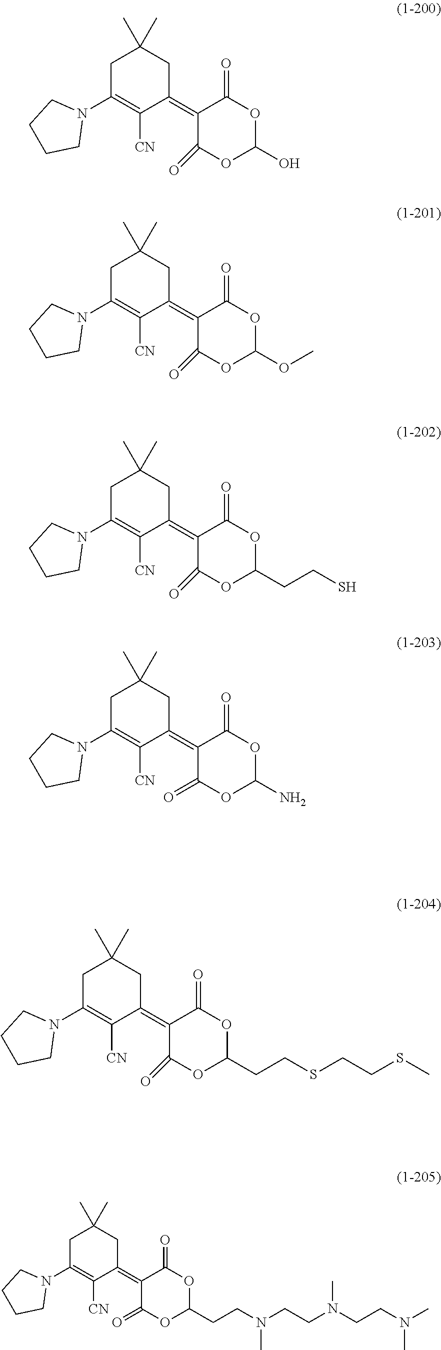 [SMILES: CC1(C)CC(=C2C(=O)OC(O)OC2=O)C(C#N)=C(N2CCCC2)C1 ; COC1OC(=O)C(=C2CC(C)(C)CC(N3CCCC3)=C2C#N)C(=O)O1 ; CC1(C)CC(=C2C(=O)OC(CCS)OC2=O)C(C#N)=C(N2CCCC2)C1 ; CC1(C)CC(=C2C(=O)OC(N)OC2=O)C(C#N)=C(N2CCCC2)C1 ; CSCCSCCC1OC(=O)C(=C2CC(C)(C)CC(N3CCCC3)=C2C#N)C(=O)O1 ; CN(C)CCN(C)CCN(C)CCC1OC(=O)C(=C2CC(C)(C)CC(N3CCCC3)=C2C#N)C(=O)O1]

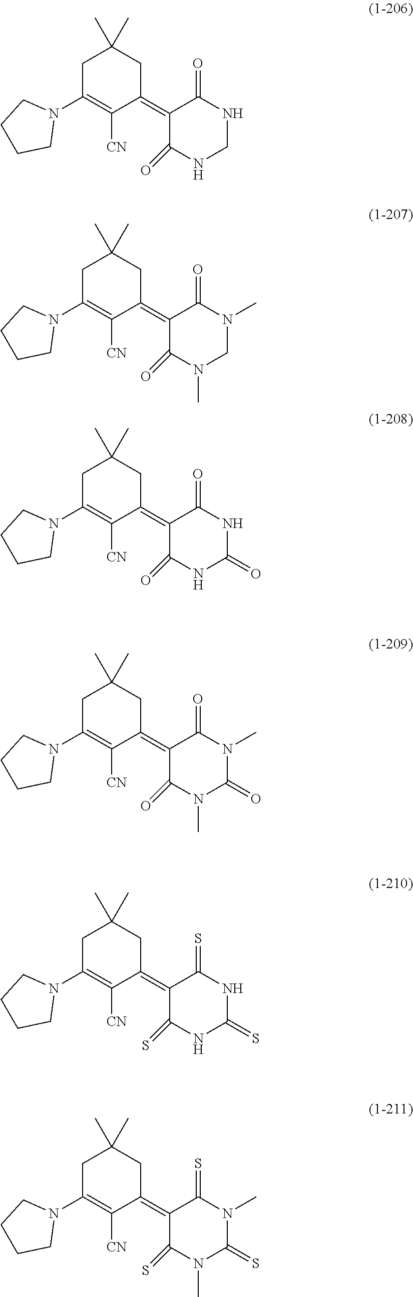 [SMILES: CC1(C)CC(=C2C(=O)NCNC2=O)C(C#N)=C(N2CCCC2)C1 ; CN1CN(C)C(=O)C(=C2CC(C)(C)CC(N3CCCC3)=C2C#N)C1=O ; CC1(C)CC(=C2C(=O)NC(=O)NC2=O)C(C#N)=C(N2CCCC2)C1 ; CN1C(=O)C(=C2CC(C)(C)CC(N3CCCC3)=C2C#N)C(=O)N(C)C1=O ; CC1(C)CC(=C2C(=S)NC(=S)NC2=S)C(C#N)=C(N2CCCC2)C1 ; CN1C(=S)C(=C2CC(C)(C)CC(N3CCCC3)=C2C#N)C(=S)N(C)C1=S]

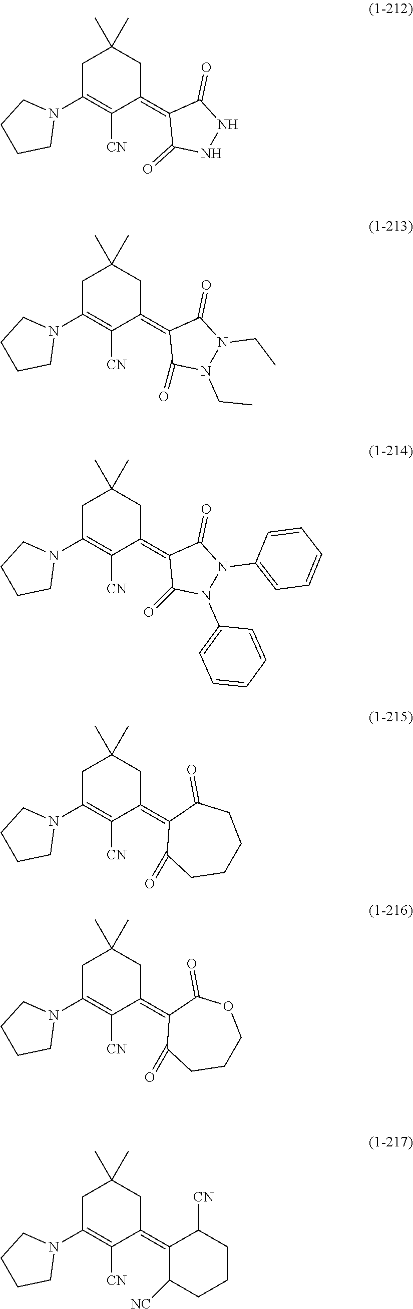

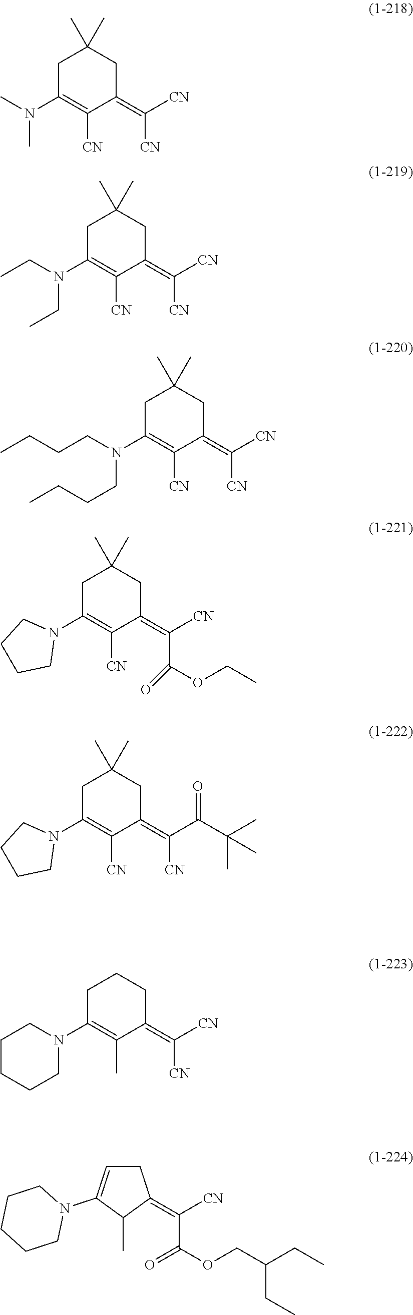

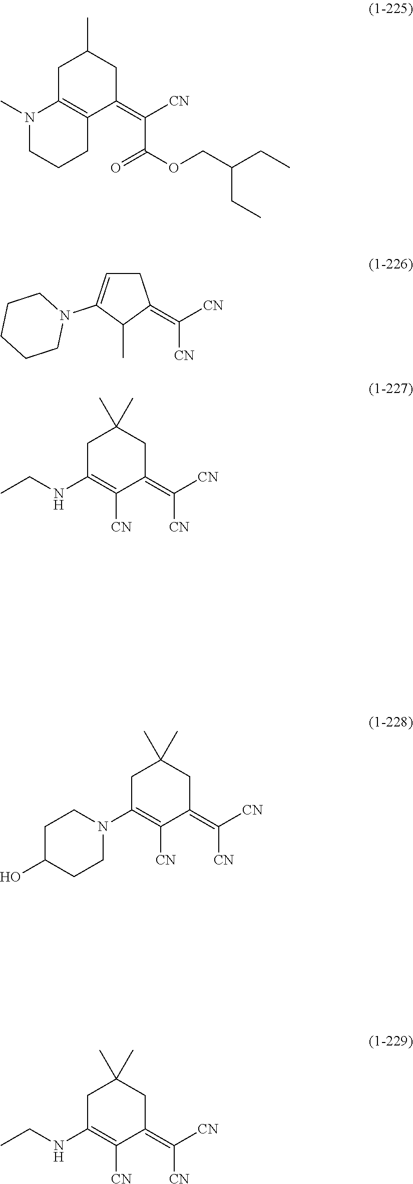

Examples of the compound represented by Formula (II) (hereinafter, may be referred to as a compound (II)) include the following compounds.

The compound (II) is preferably compounds represented by Formula (2-1), Formula (2-2), Formula (2-5) to Formula (2-12), Formula (2-24) to Formula (2-28), Formula (2-32), Formula (2-33), Formula (2-38) to Formula (2-44), Formula (2-70), Formula (2-71), and Formula (2-103) to Formula (2-106), and more preferably compounds represented by Formula (2-1), Formula (2-2), Formula (2-5) to Formula (2-10), or Formula (2-103) to Formula (2-106).

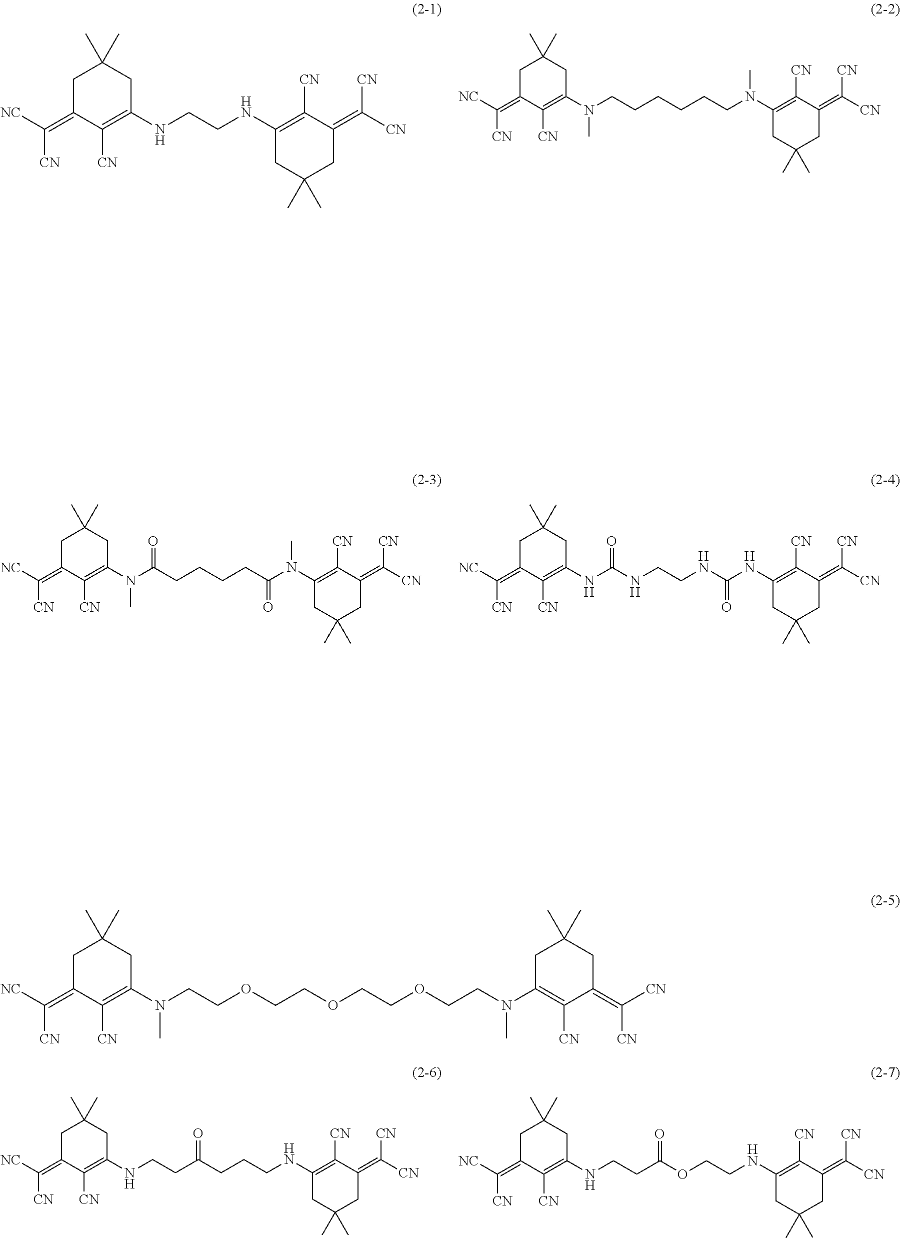

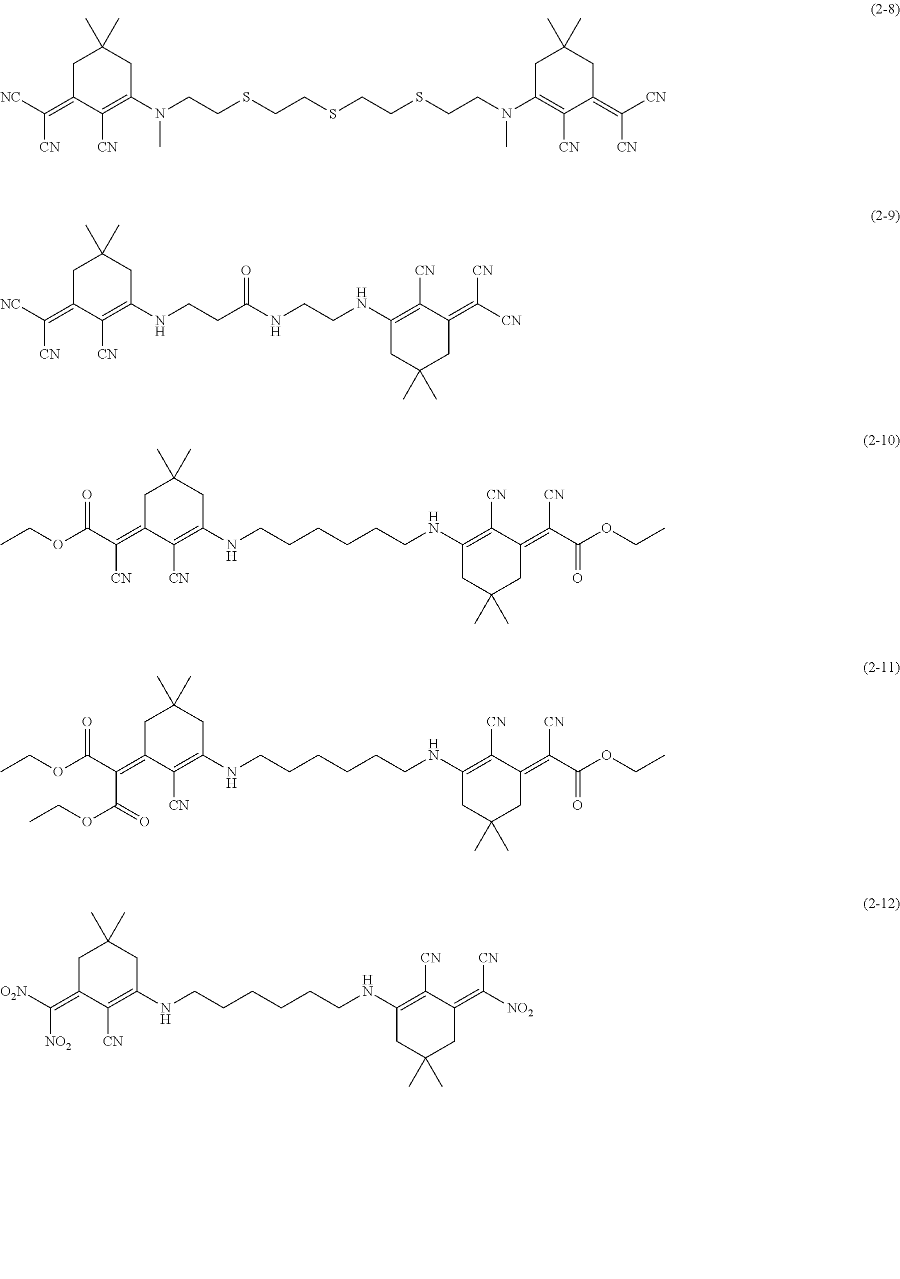

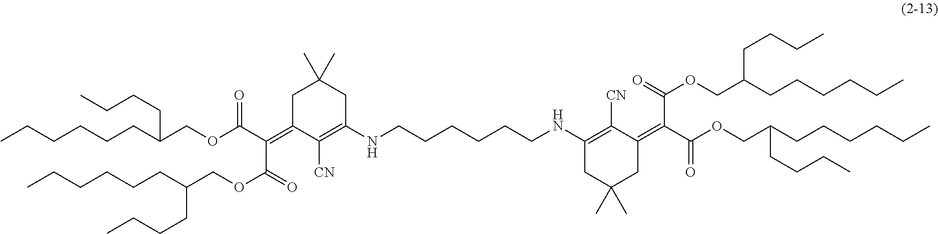 [SMILES: CCCCCCC(CCCC)COC(=O)C(C(=O)OCC(CCCC)CCCCCC)=C1CC(C)(C)CC(NCCCCCCNC2=C(C#N)C(=C(C(=O)OCC(CCCC)CCCCCC)C(=O)OCC(CCCC)CCCCCC)CC(C)(C)C2)=C1C#N]

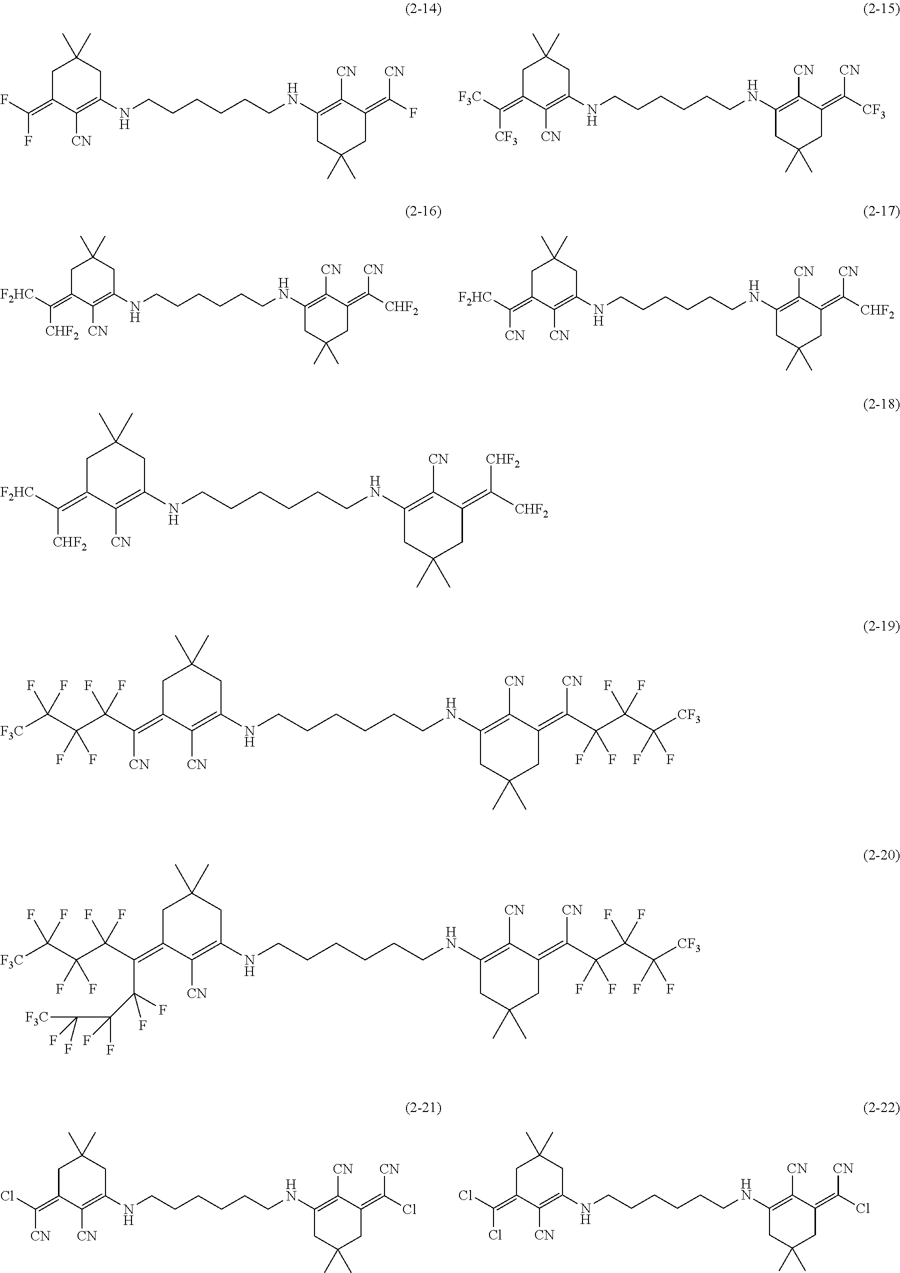

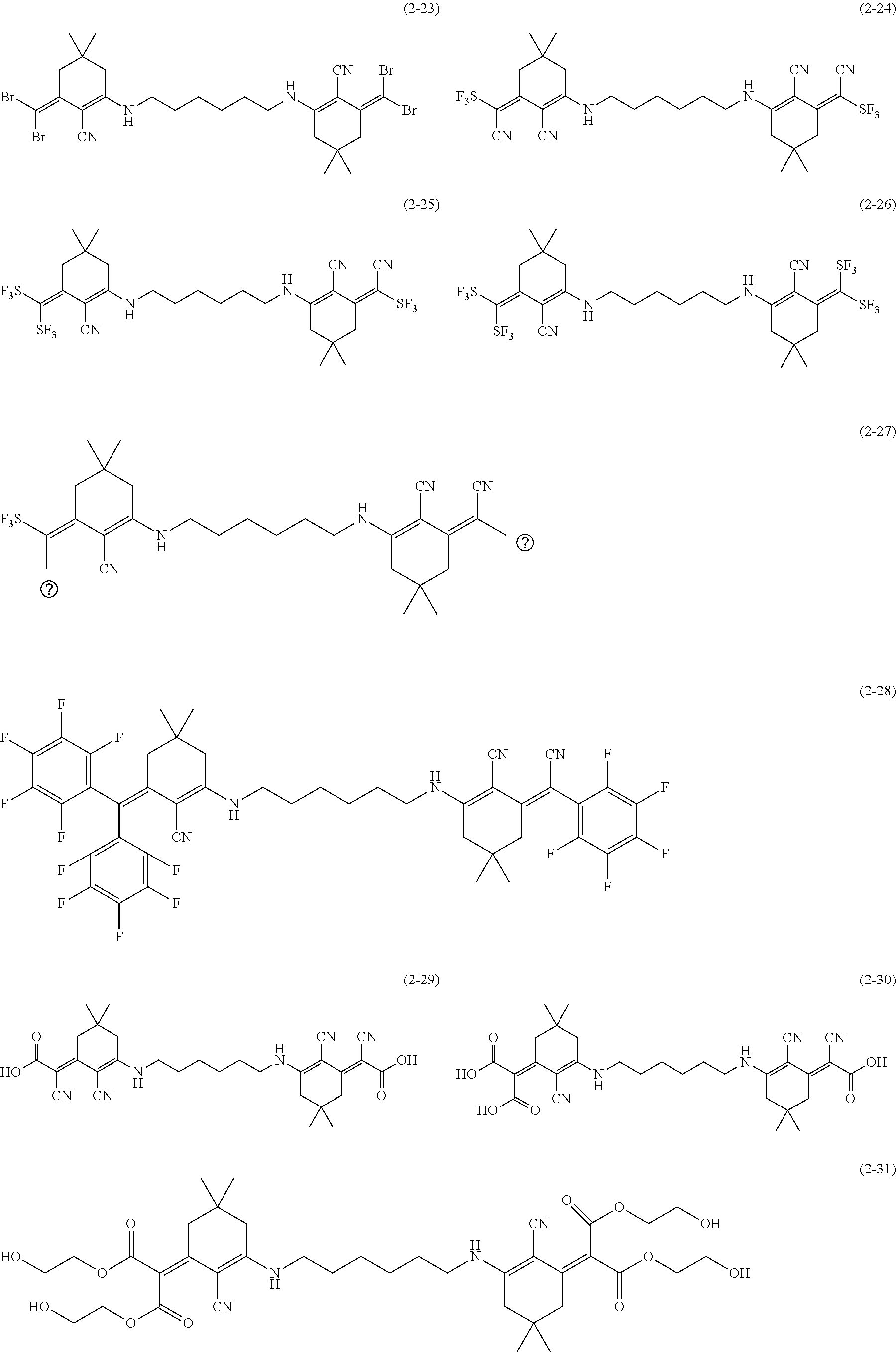

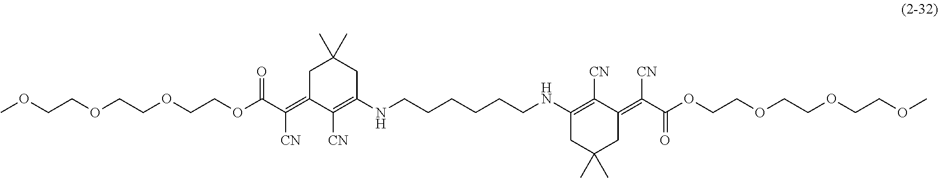

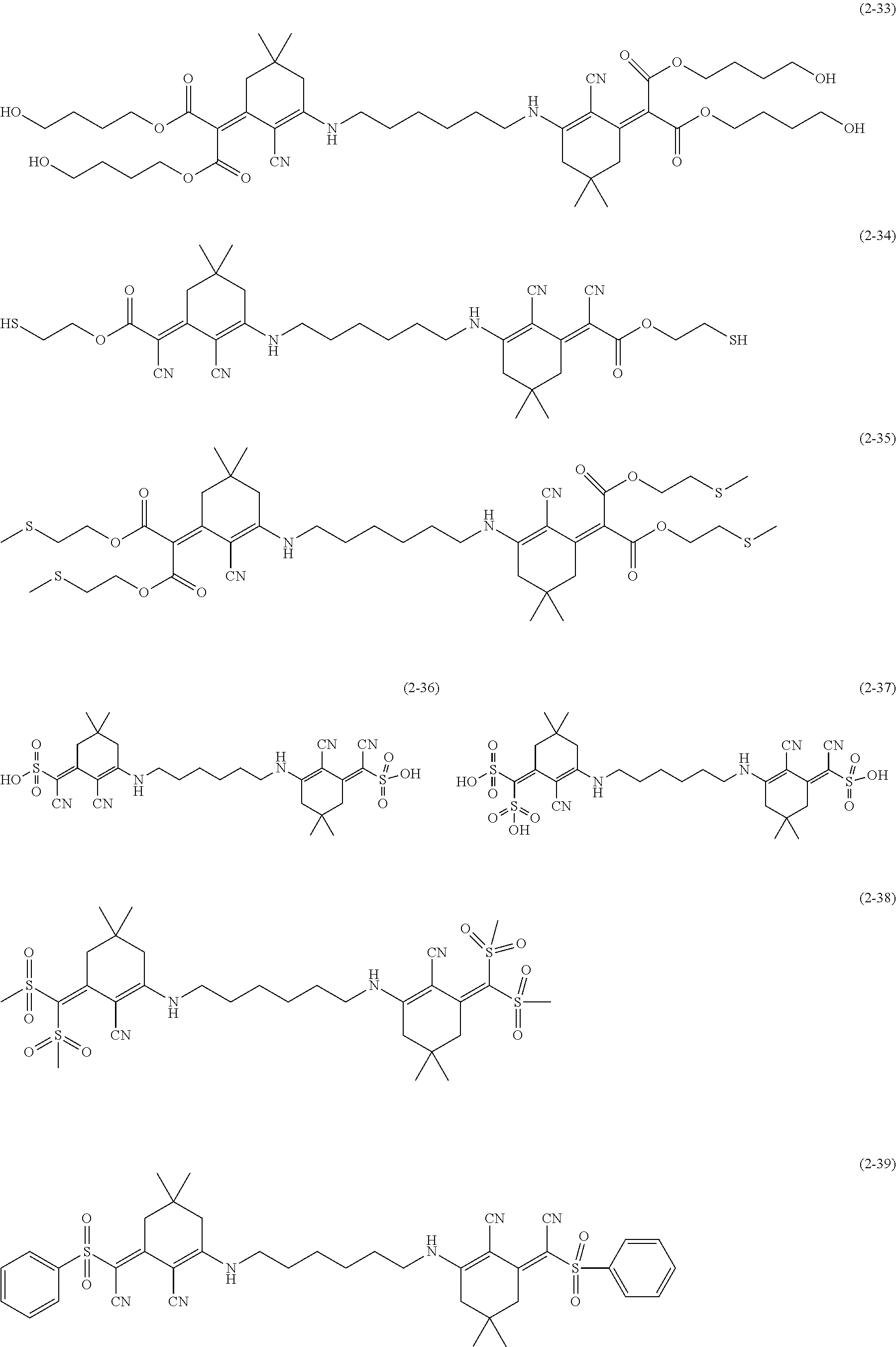

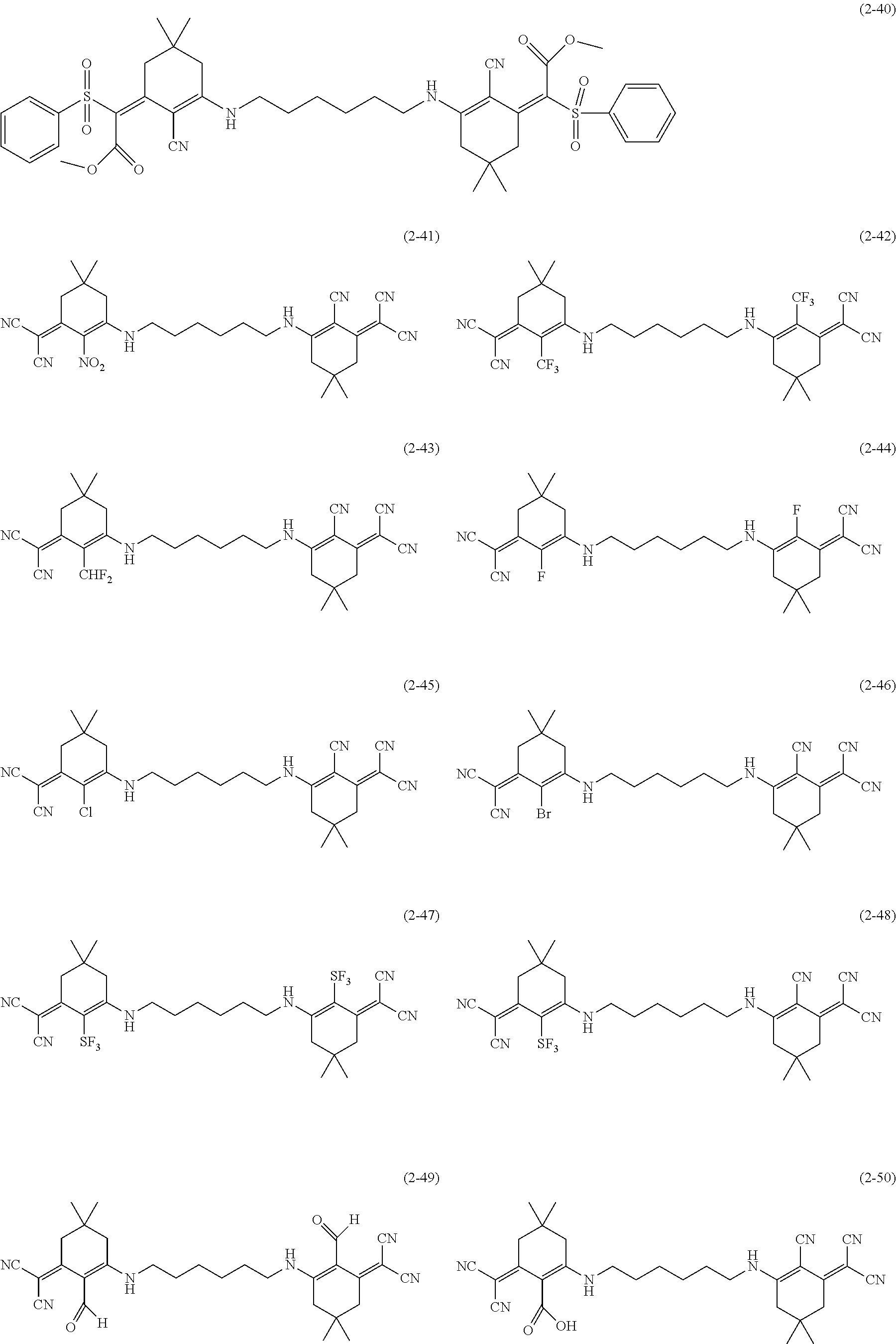

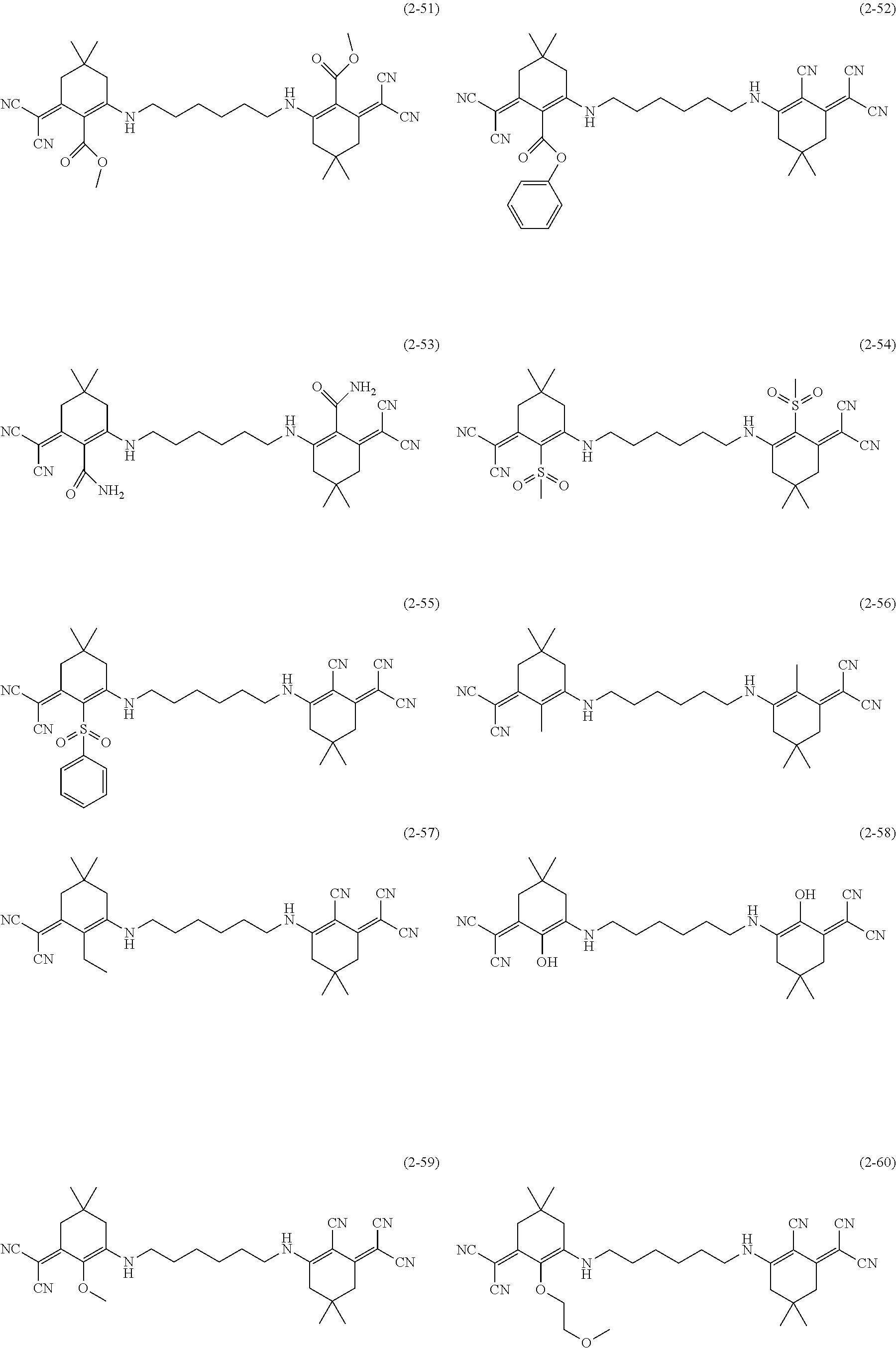

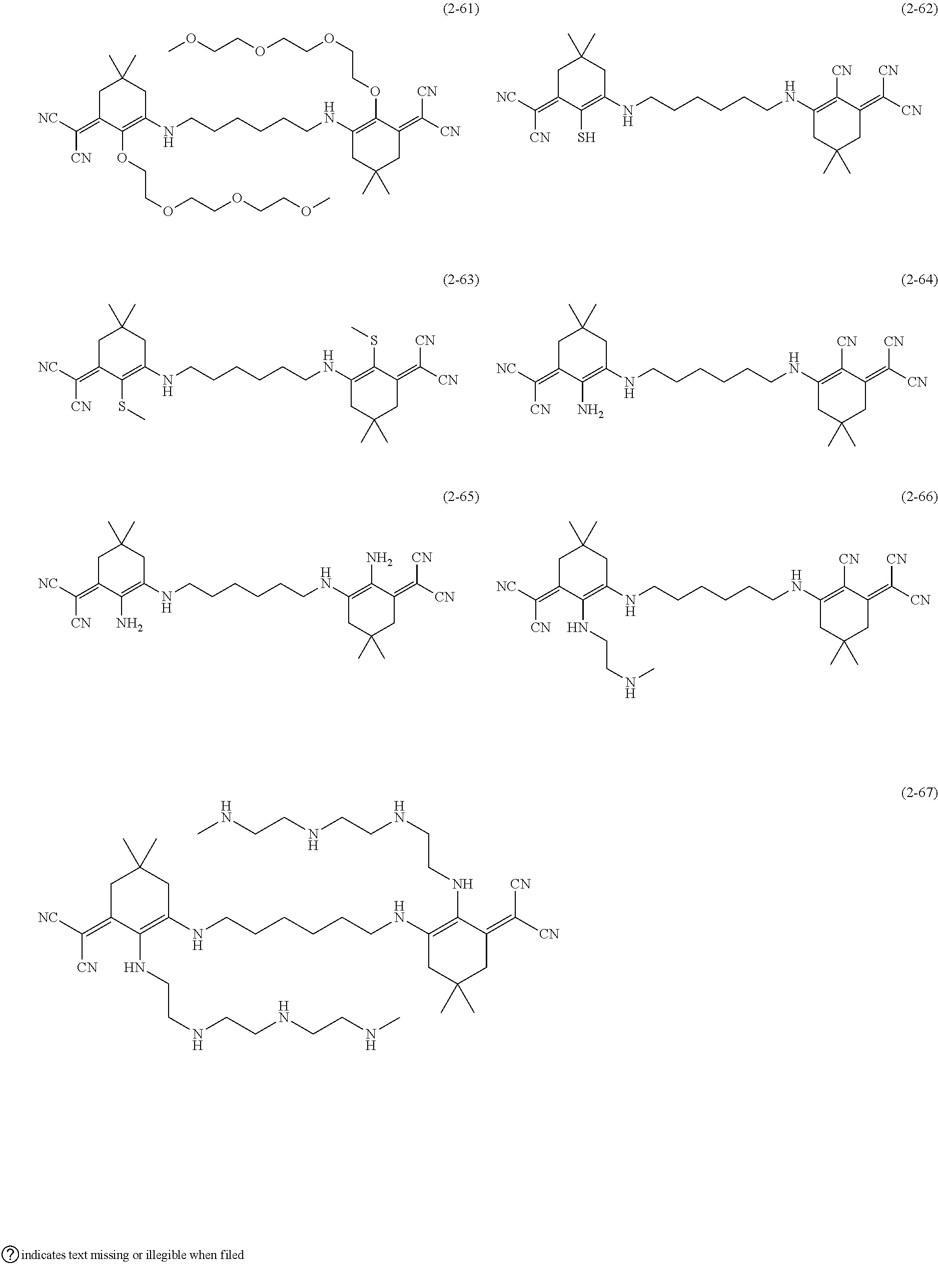

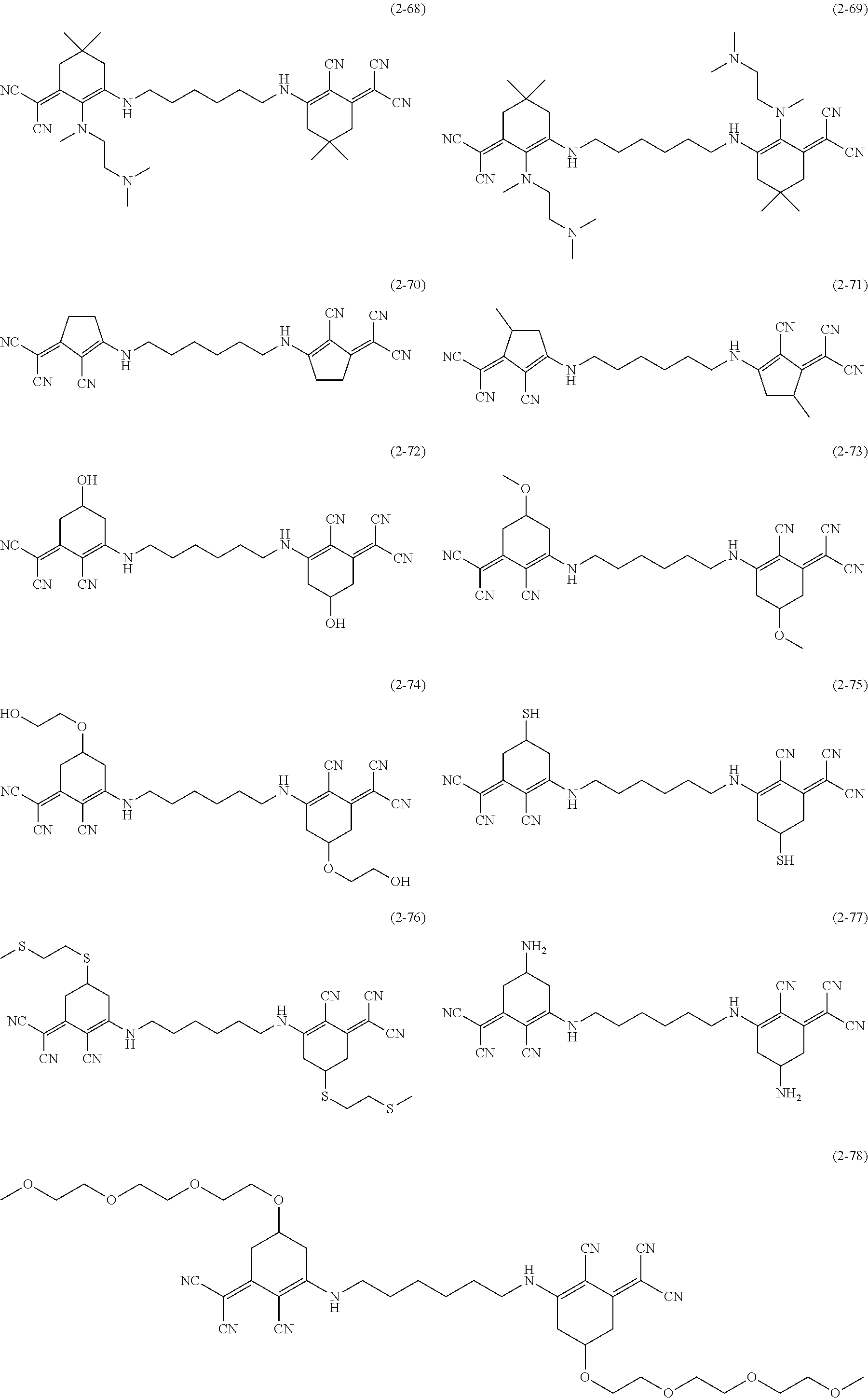

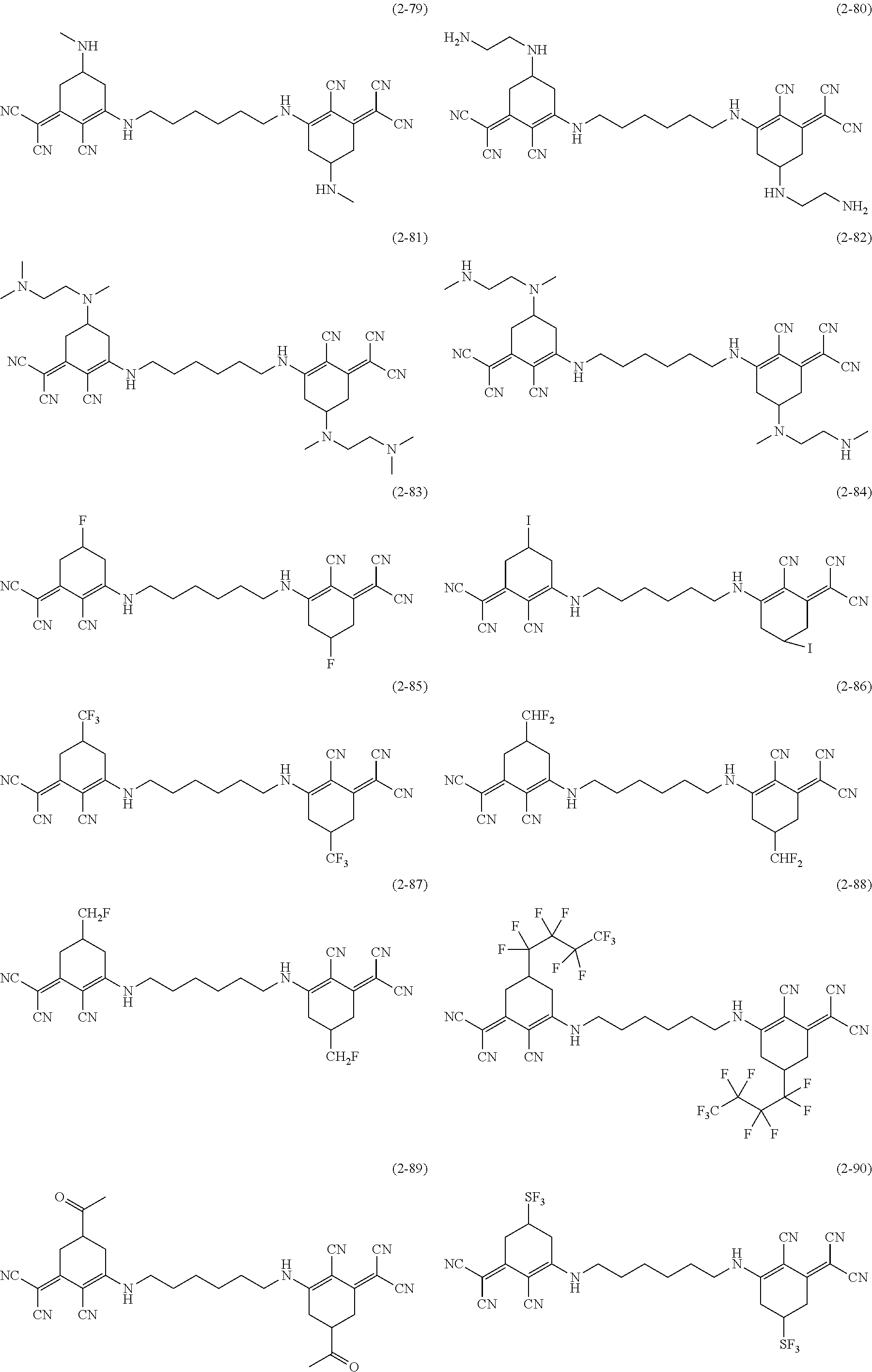

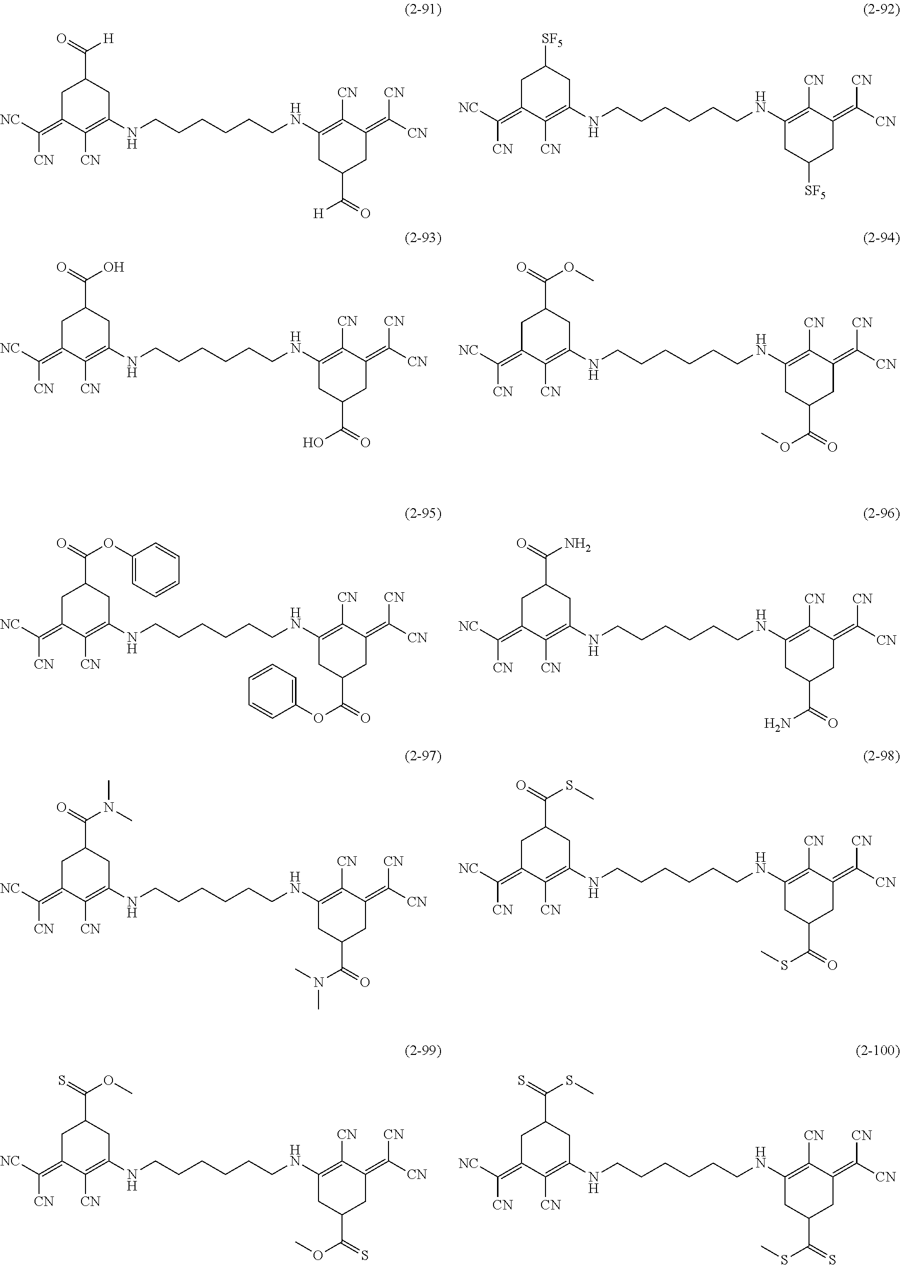

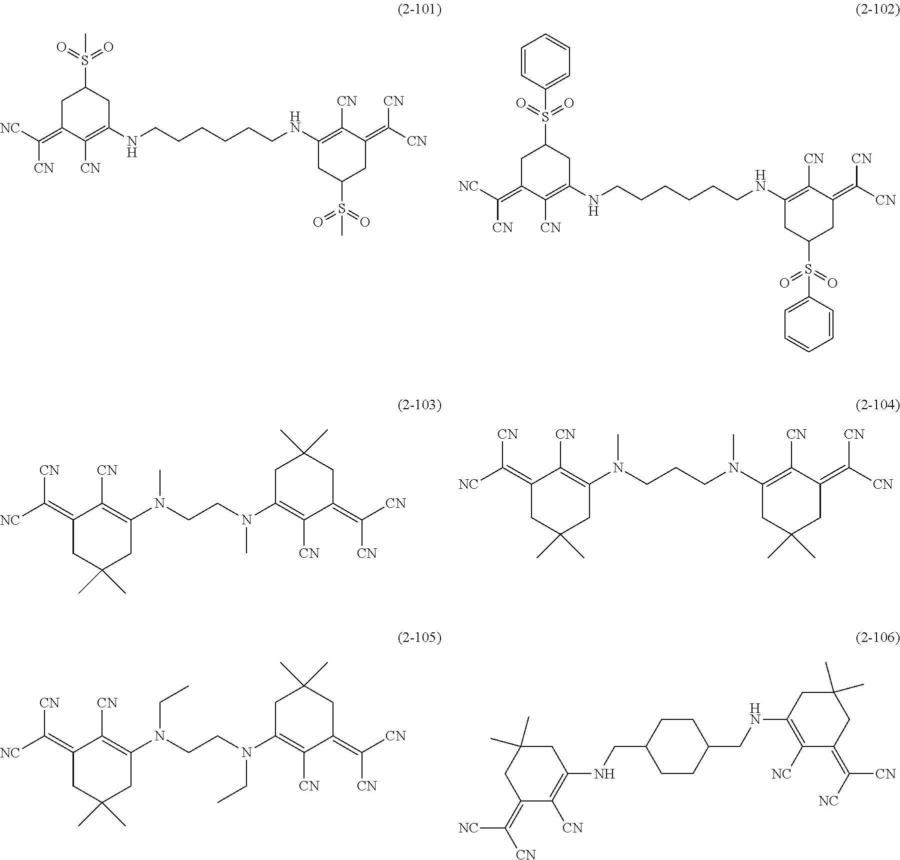 [SMILES: CS(=O)(=O)C1CC(NCCCCCCNC2=C(C#N)C(=C(C#N)C#N)CC(S(C)(=O)=O)C2)=C(C#N)C(=C(C#N)C#N)C1 ; N#CC(C#N)=C1CC(S(=O)(=O)c2ccccc2)CC(NCCCCCCNC2=C(C#N)C(=C(C#N)C#N)CC(S(=O)(=O)c3ccccc3)C2)=C1C#N ; CN(CCN(C)C1=C(C#N)C(=C(C#N)C#N)CC(C)(C)C1)C1=C(C#N)C(=C(C#N)C#N)CC(C)(C)C1 ; CN(CCCN(C)C1=C(C#N)C(=C(C#N)C#N)CC(C)(C)C1)C1=C(C#N)C(=C(C#N)C#N)CC(C)(C)C1 ; CCN(CCN(CC)C1=C(C#N)C(=C(C#N)C#N)CC(C)(C)C1)C1=C(C#N)C(=C(C#N)C#N)CC(C)(C)C1 ; CC1(C)CC(NCC2CCC(CNC3=C(C#N)C(=C(C#N)C#N)CC(C)(C)C3)CC2)=C(C#N)C(=C(C#N)C#N)C1]

Examples of the compound represented by Formula (III) (hereinafter, may be referred to as a compound (III)) include the following compounds.

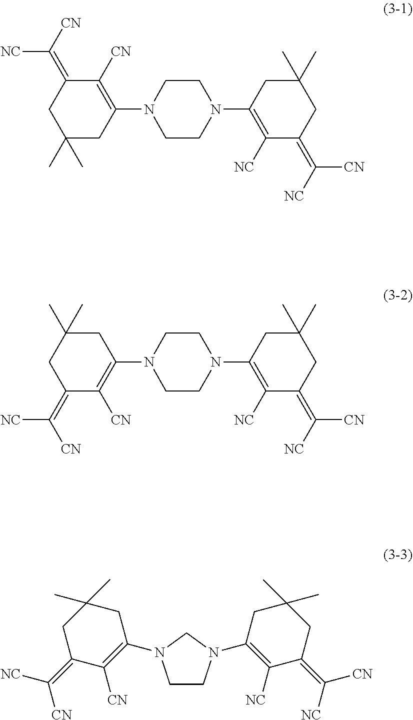 [SMILES: CC1(C)CC(=C(C#N)C#N)C(C#N)=C(N2CCN(C3=C(C#N)C(=C(C#N)C#N)CC(C)(C)C3)CC2)C1 ; CC1(C)CC(=C(C#N)C#N)C(C#N)=C(N2CCN(C3=C(C#N)C(=C(C#N)C#N)CC(C)(C)C3)CC2)C1 ; CC1(C)CC(=C(C#N)C#N)C(C#N)=C(N2CCN(C3=C(C#N)C(=C(C#N)C#N)CC(C)(C)C3)C2)C1]

Examples of the compound represented by Formula (IV) (hereinafter, may be referred to as a compound (IV)) include the following compounds.

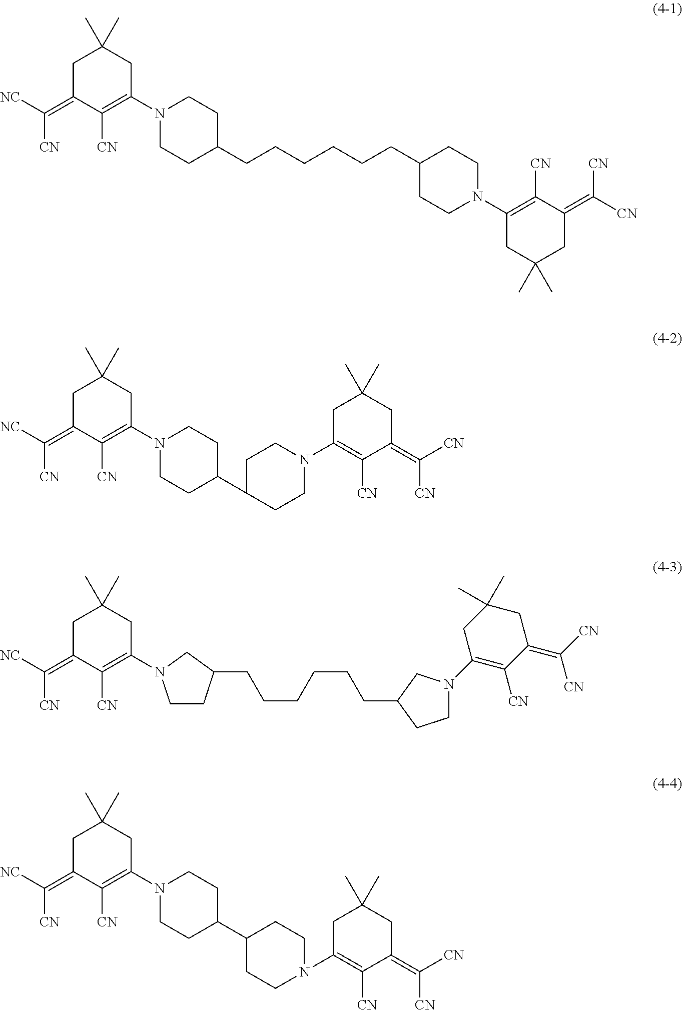 [SMILES: CC1(C)CC(=C(C#N)C#N)C(C#N)=C(N2CCC(CCCCCCC3CCN(C4=C(C#N)C(=C(C#N)C#N)CC(C)(C)C4)CC3)CC2)C1 ; CC1(C)CC(=C(C#N)C#N)C(C#N)=C(N2CCC(C3CCN(C4=C(C#N)C(=C(C#N)C#N)CC(C)(C)C4)CC3)CC2)C1 ; CC1(C)CC(=C(C#N)C#N)C(C#N)=C(N2CCC(CCCCCCC3CCN(C4=C(C#N)C(=C(C#N)C#N)CC(C)(C)C4)C3)C2)C1 ; CC1(C)CC(=C(C#N)C#N)C(C#N)=C(N2CCC(C3CCN(C4=C(C#N)C(=C(C#N)C#N)CC(C)(C)C4)CC3)CC2)C1]

Examples of the compound represented by Formula (V) (hereinafter, may be referred to as a compound (V)) include the following compounds.

The compound (V) is preferably compounds represented by Formula (5-1) to Formula (5-3), Formula (5-6), Formula (5-7), Formula (5-9), Formula (5-15), Formula (5-21), Formula (5-23), Formula (5-25), Formula (5-26), Formula (5-32), Formula (5-36), and Formula (5-38), and more preferably compounds represented by Formula (5-1) to Formula (5-3), Formula (5-21), Formula (5-25), and Formula (5-36).

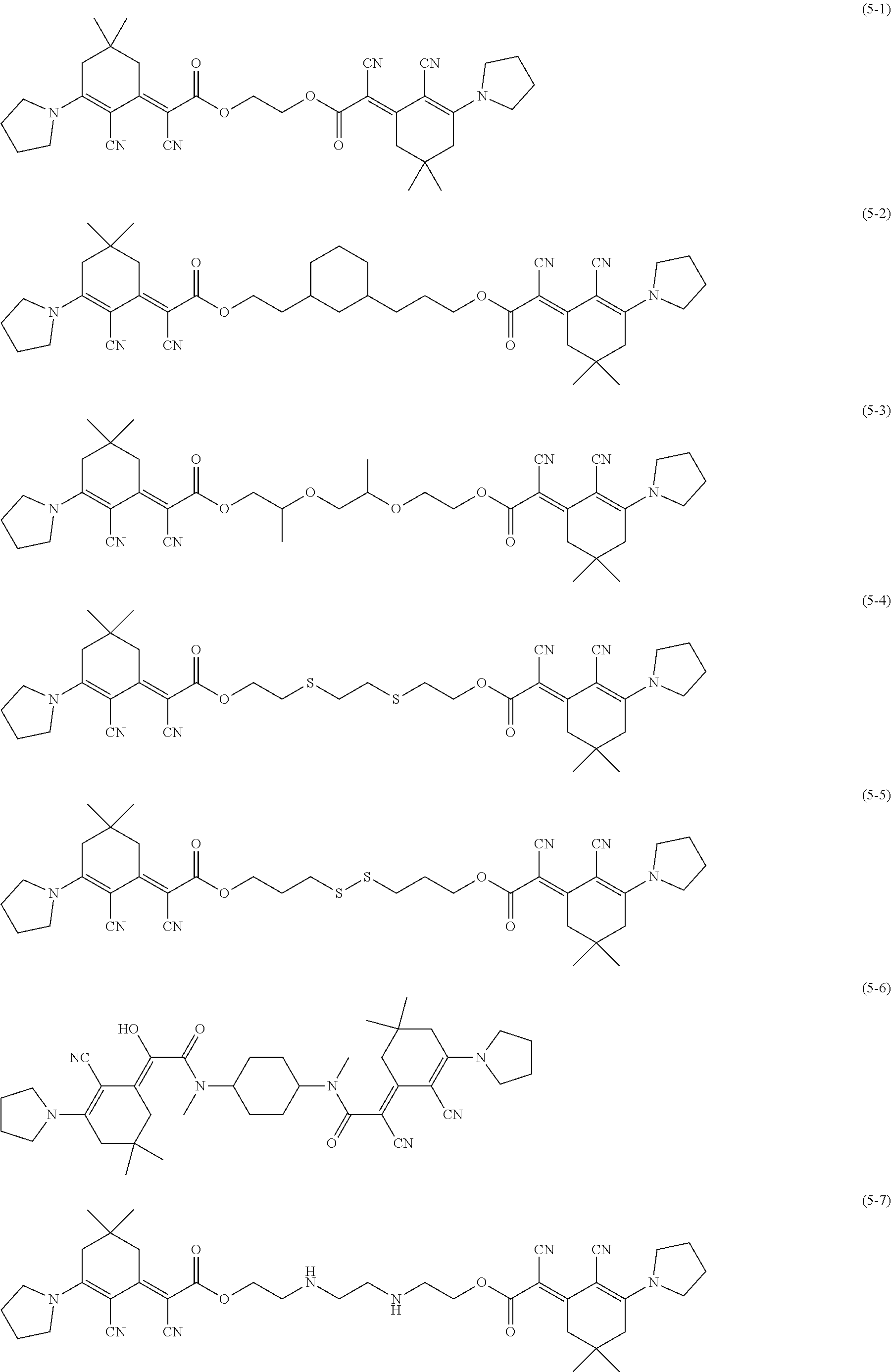

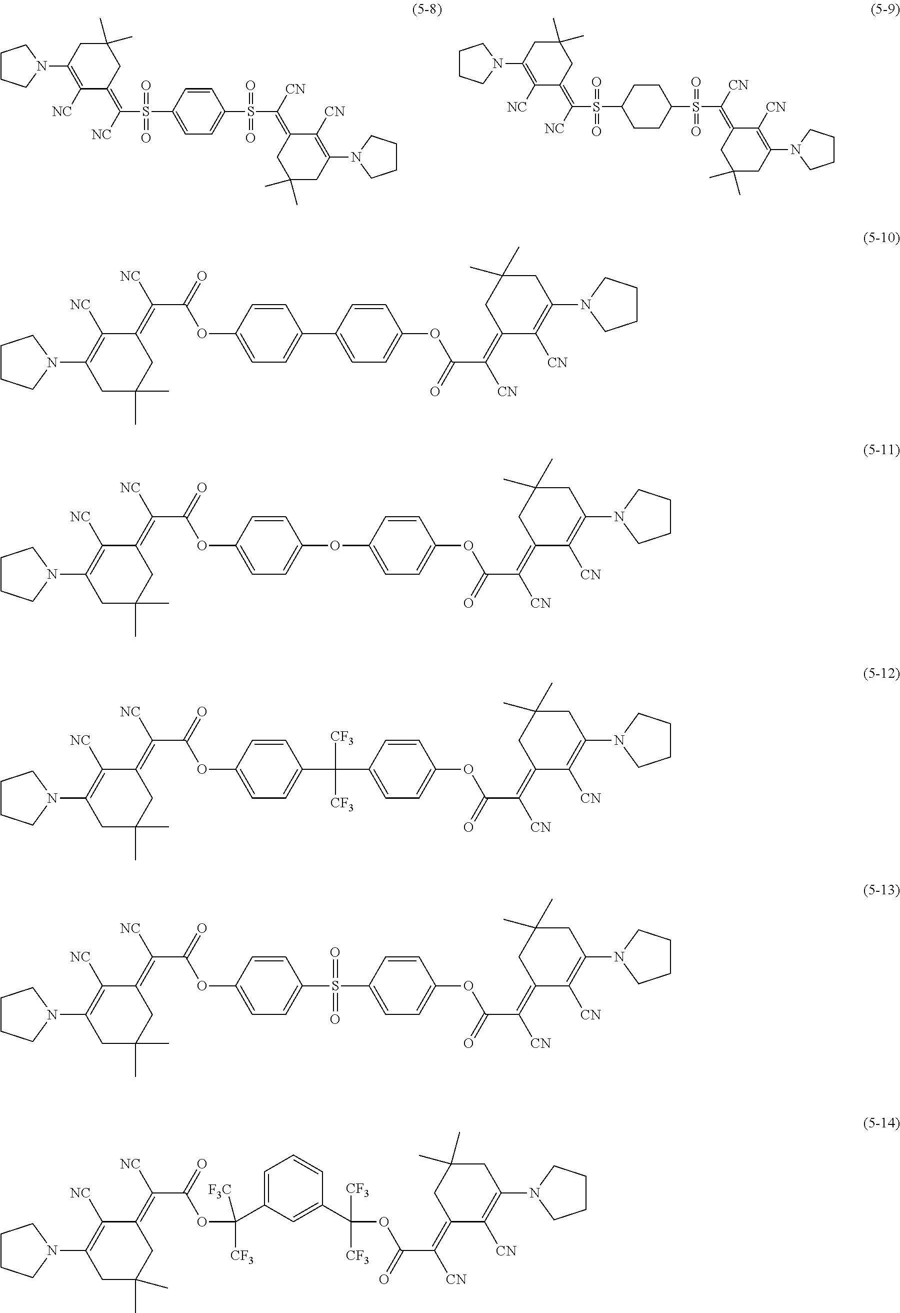

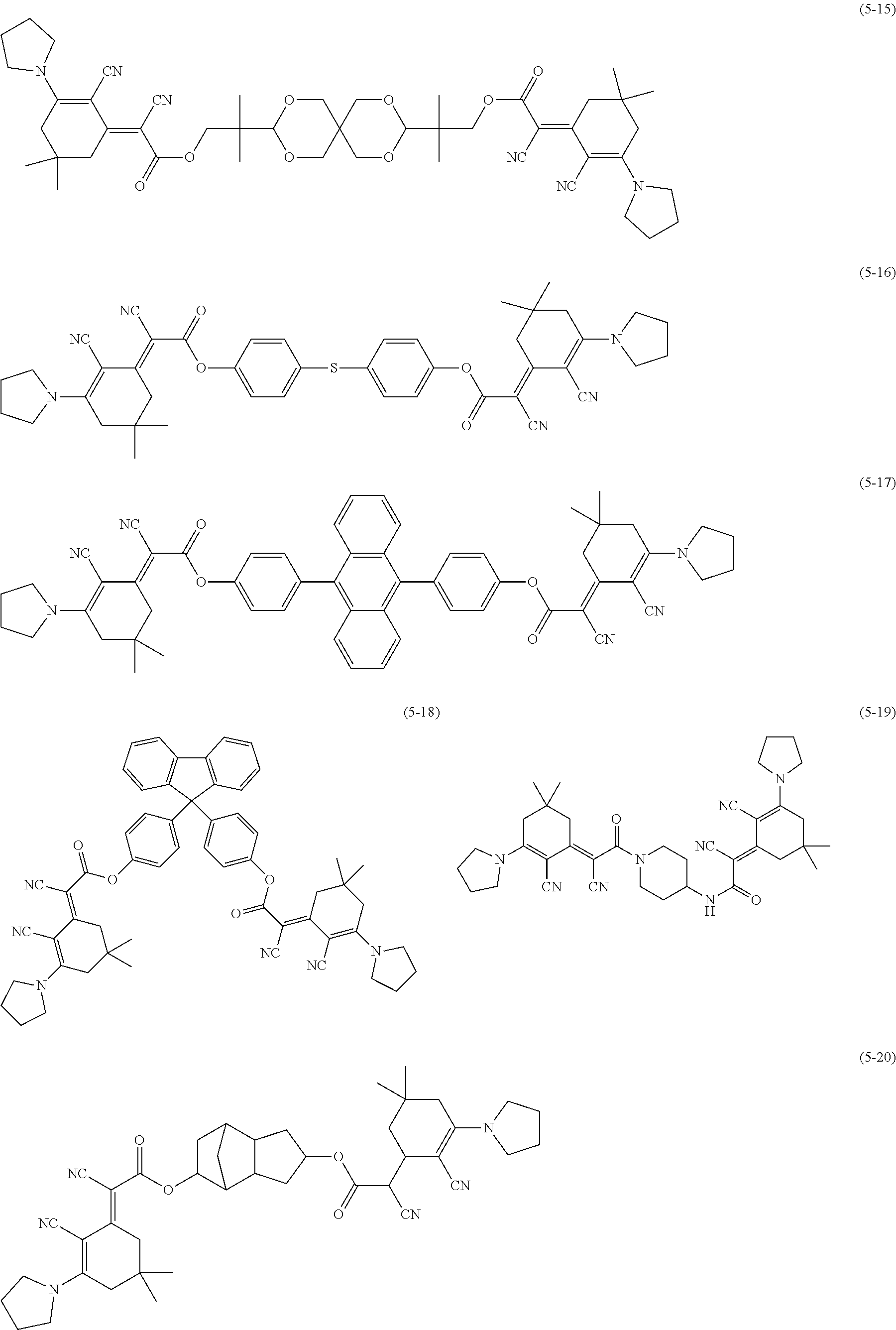

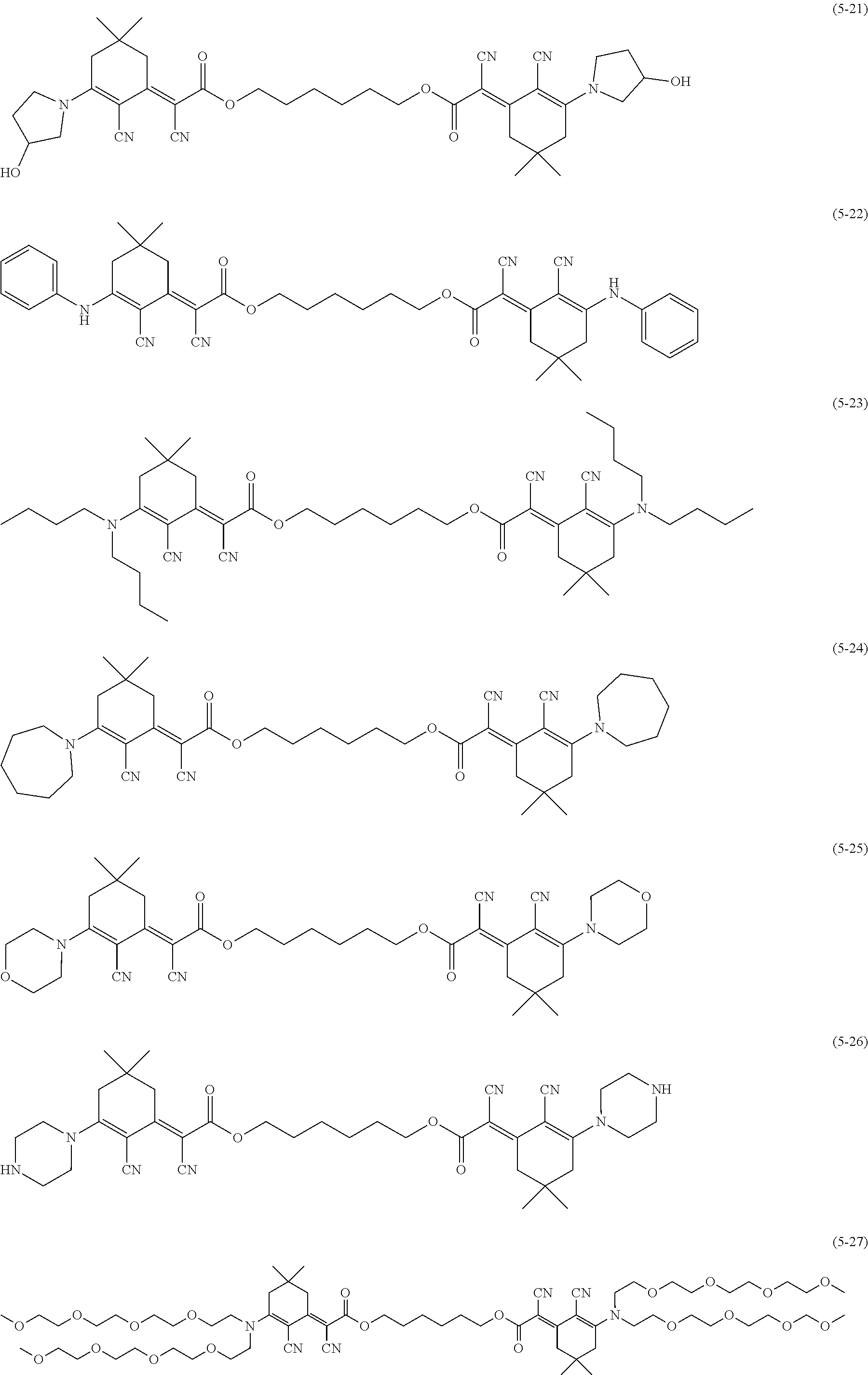

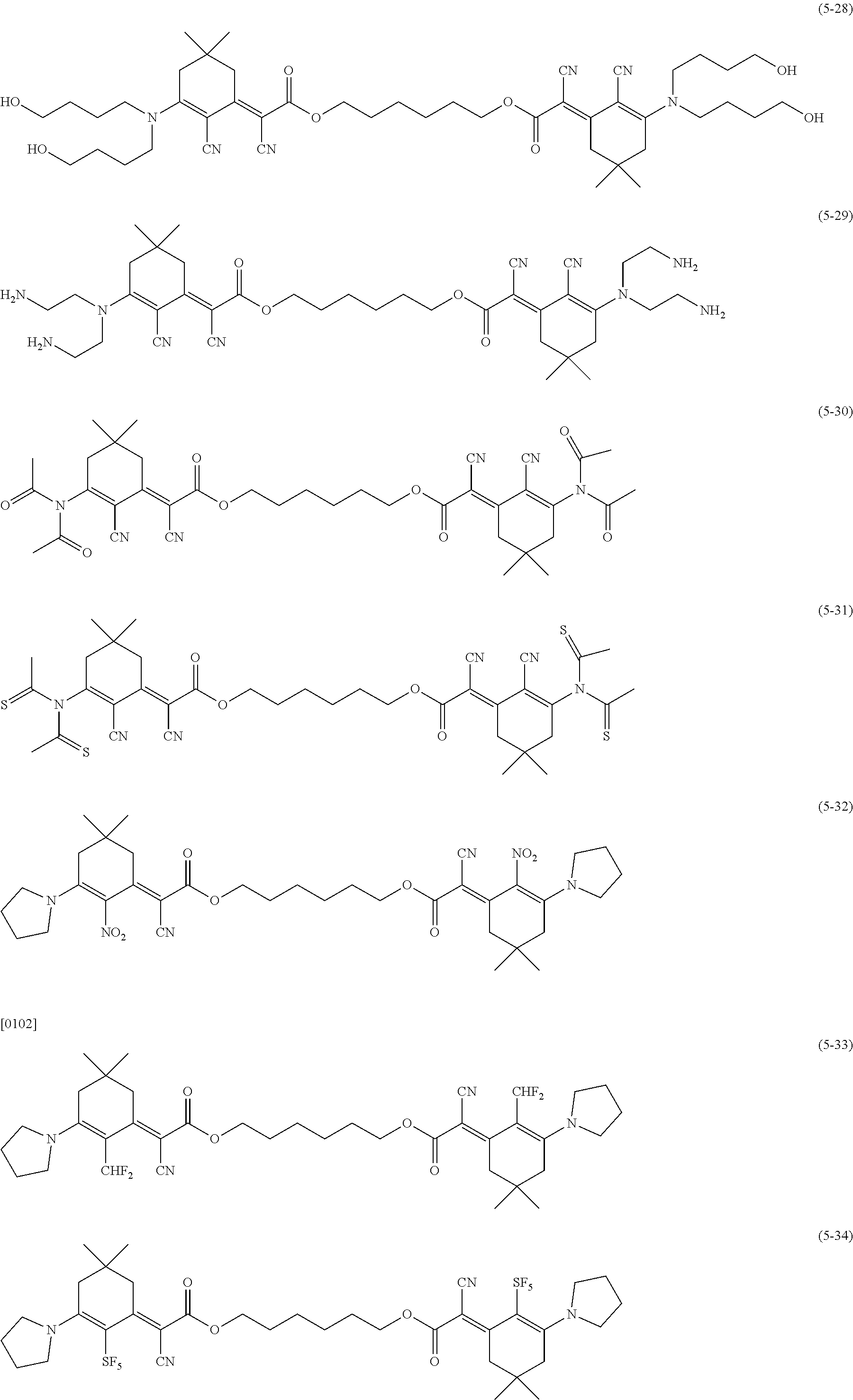

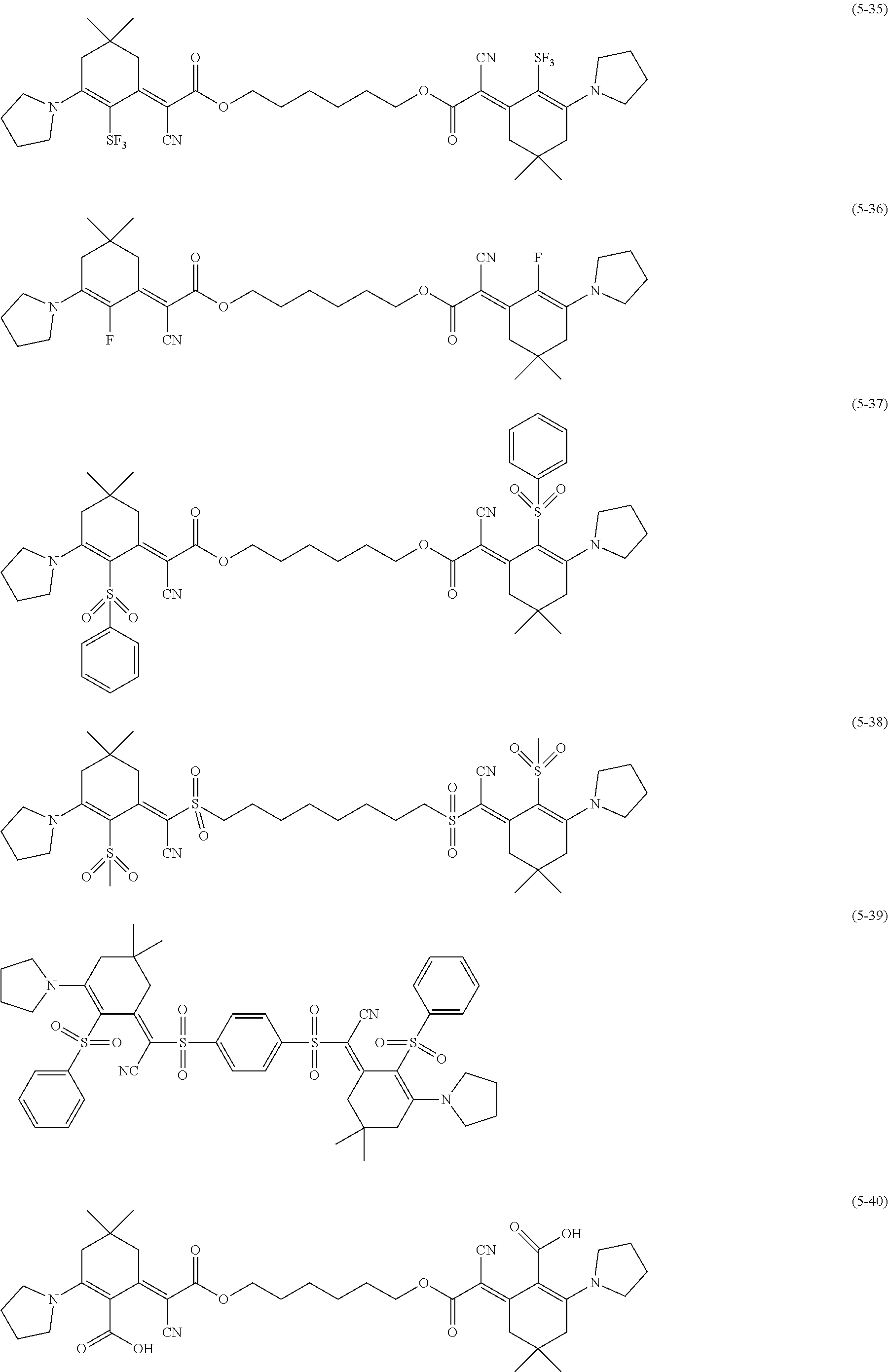

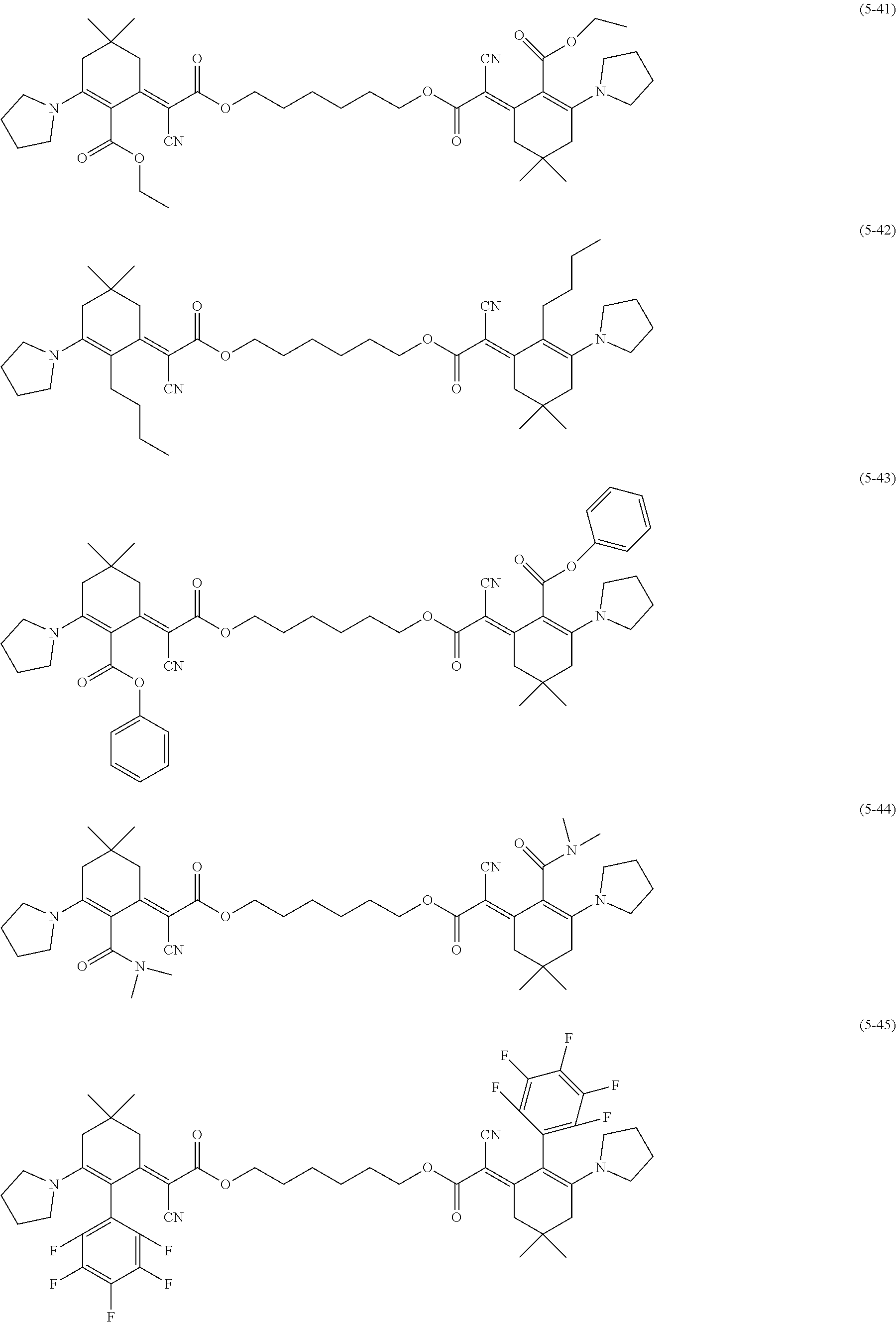

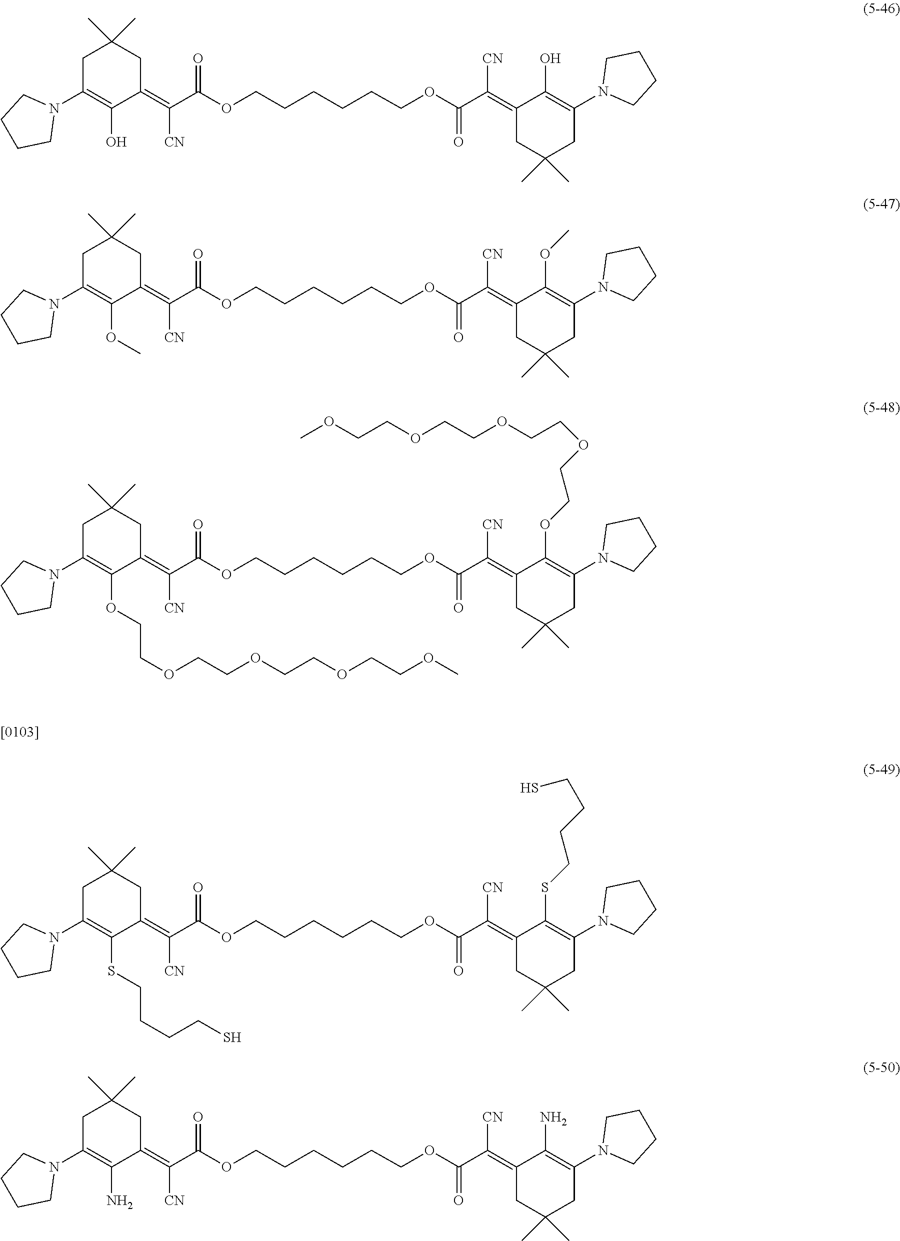

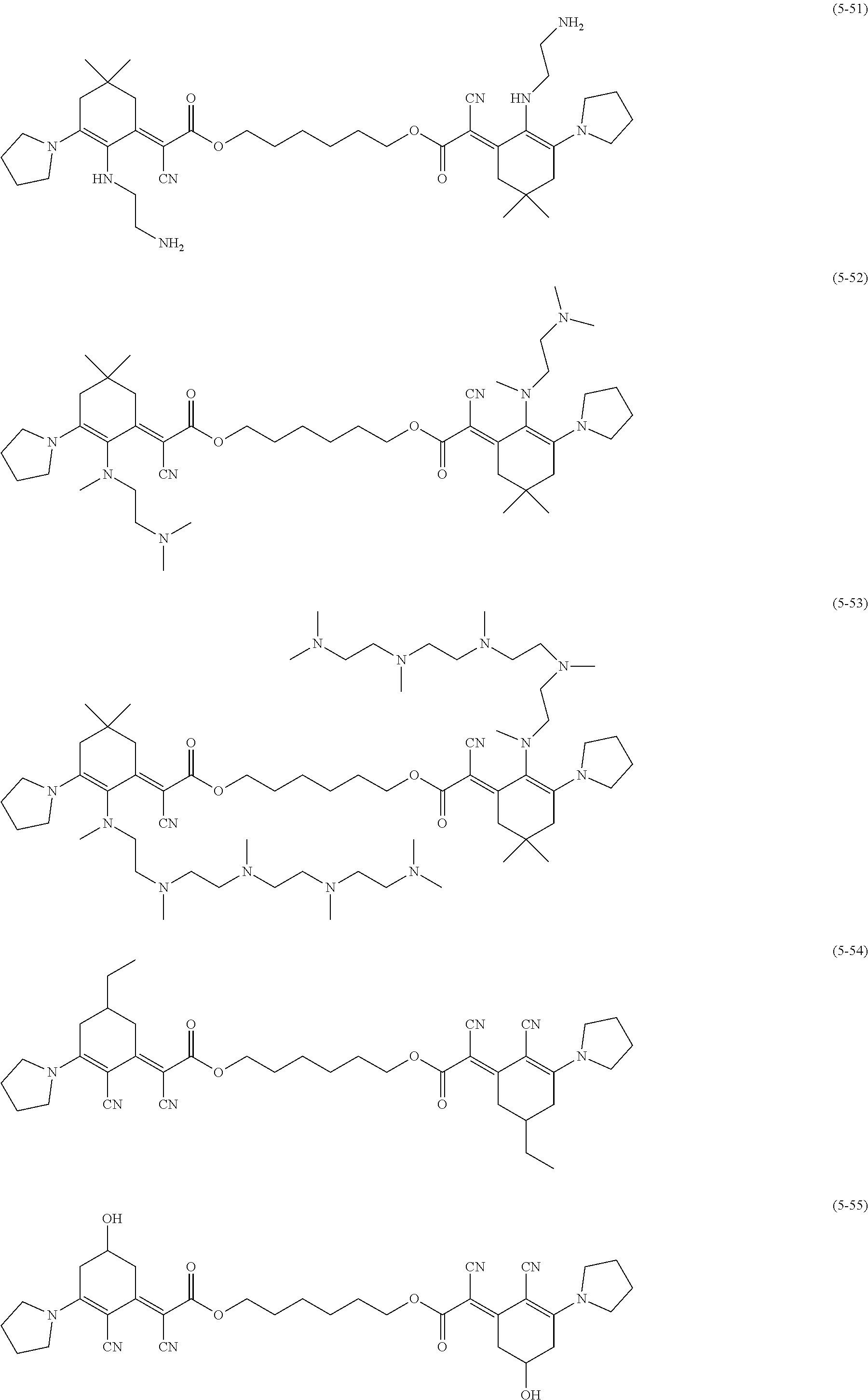

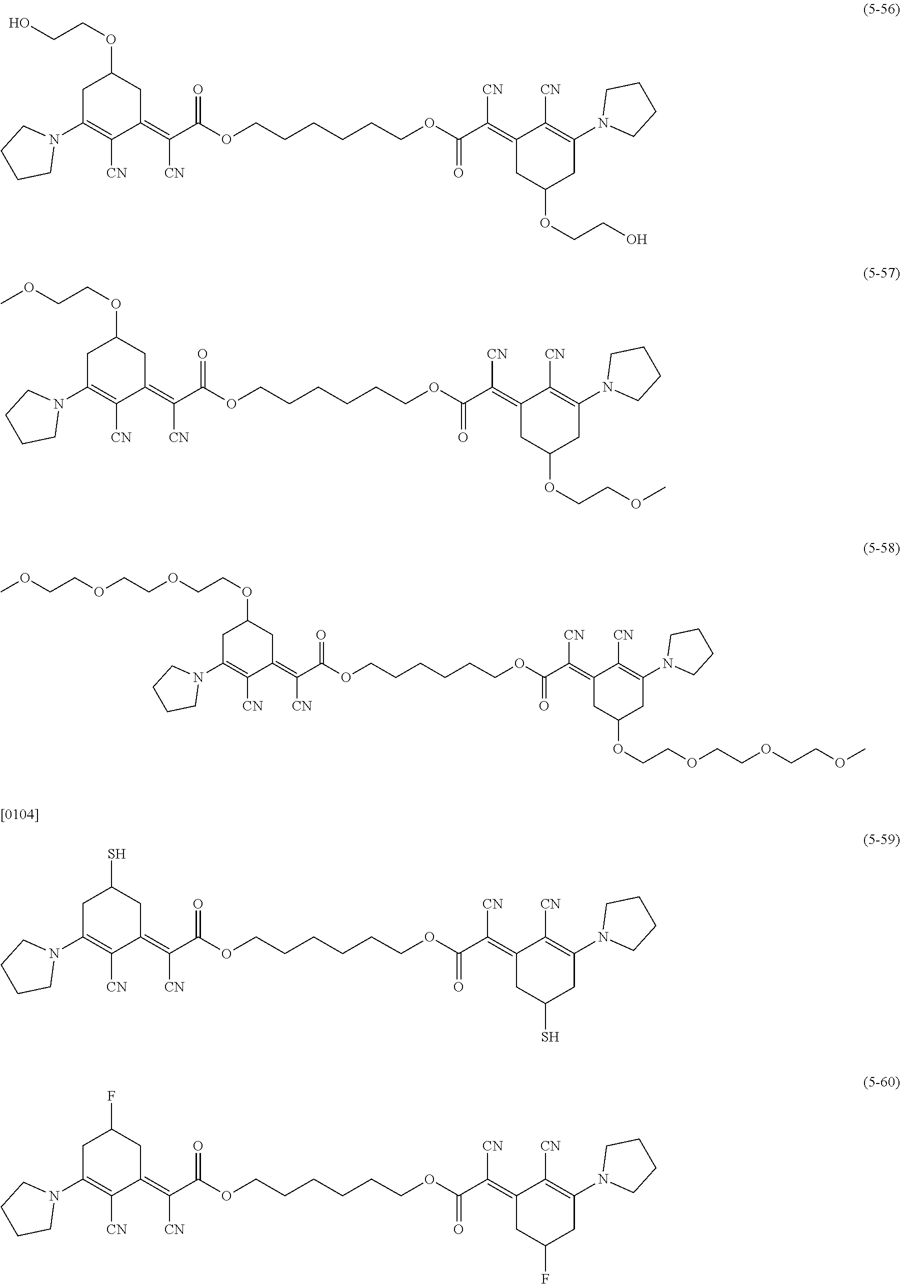

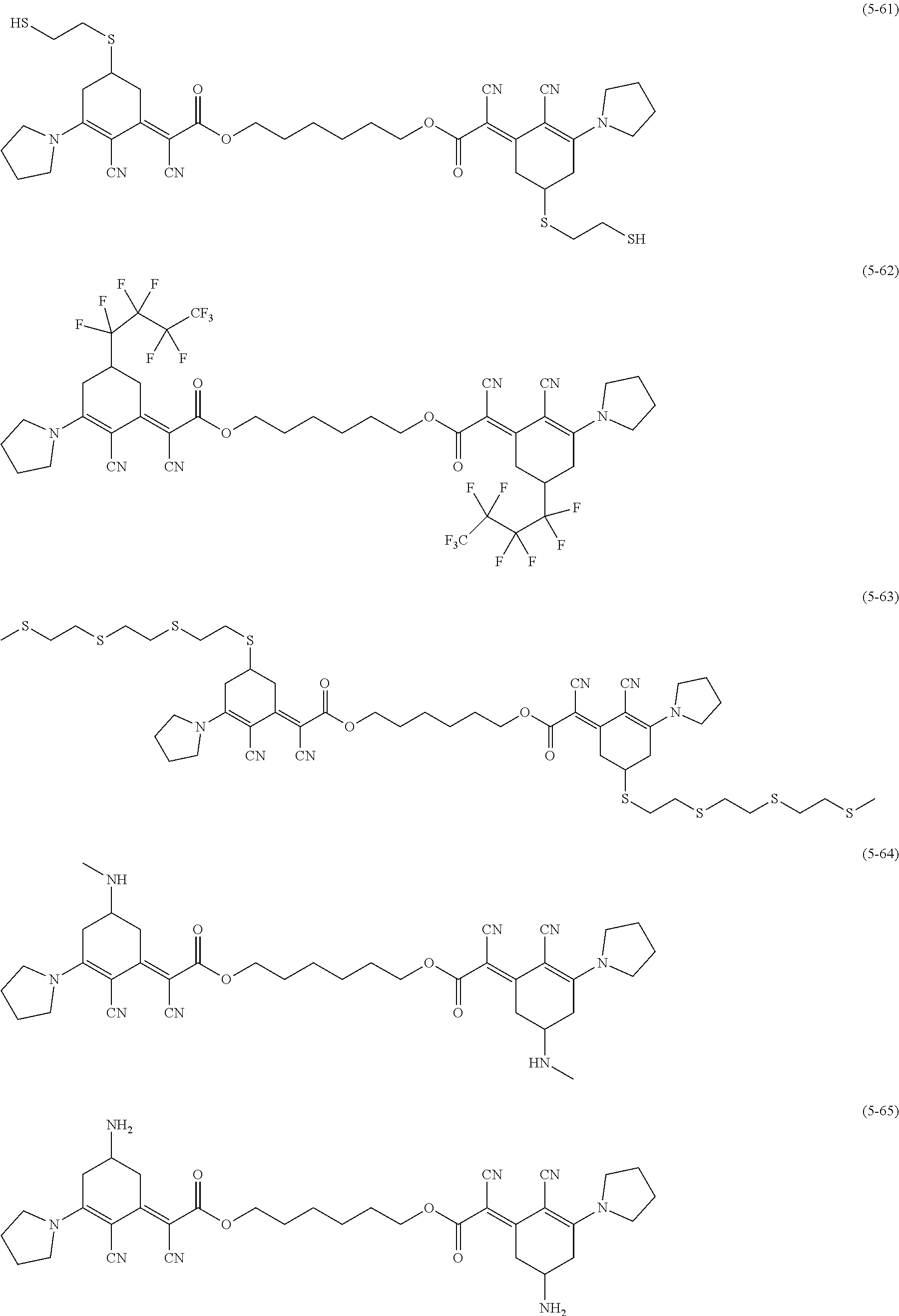

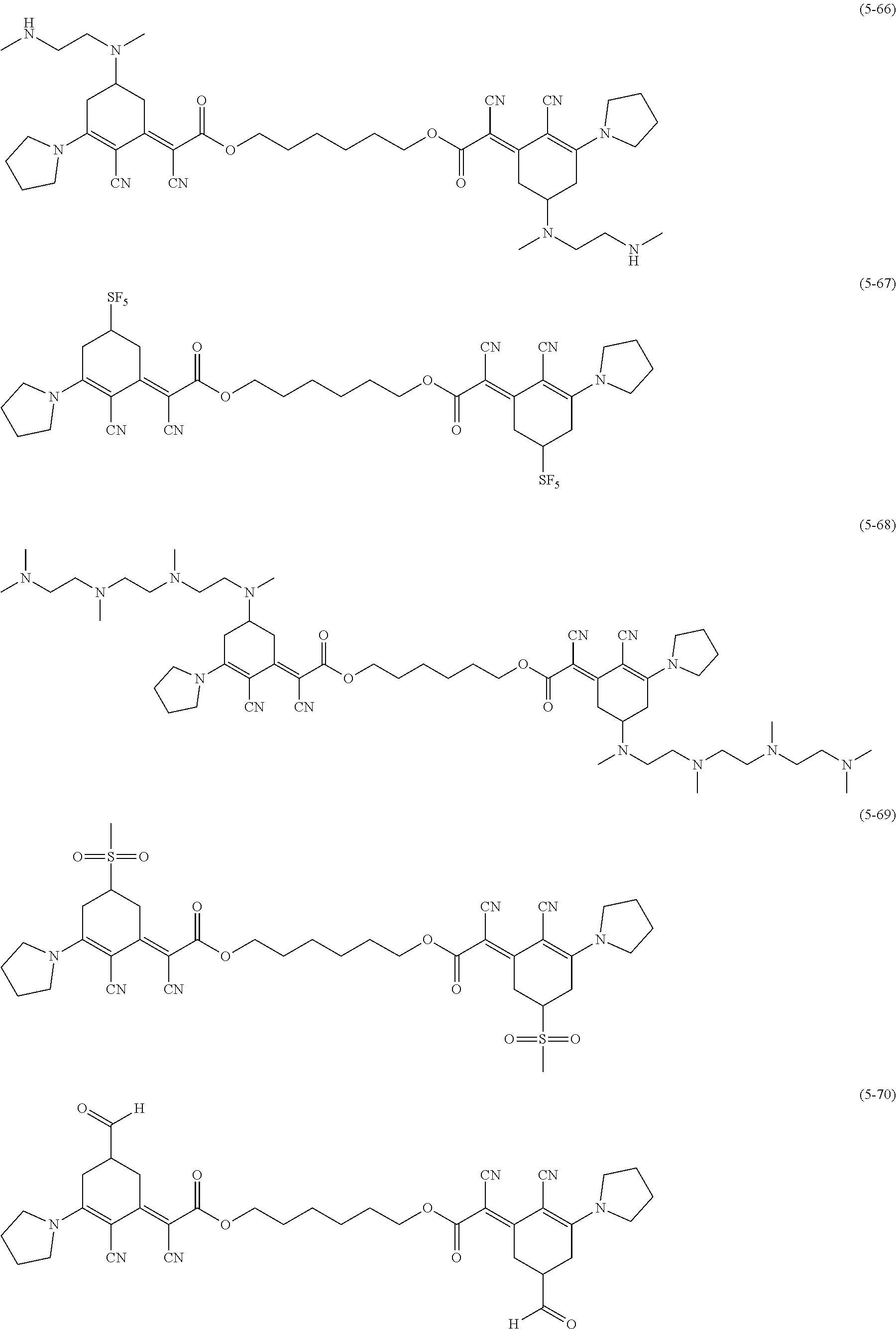

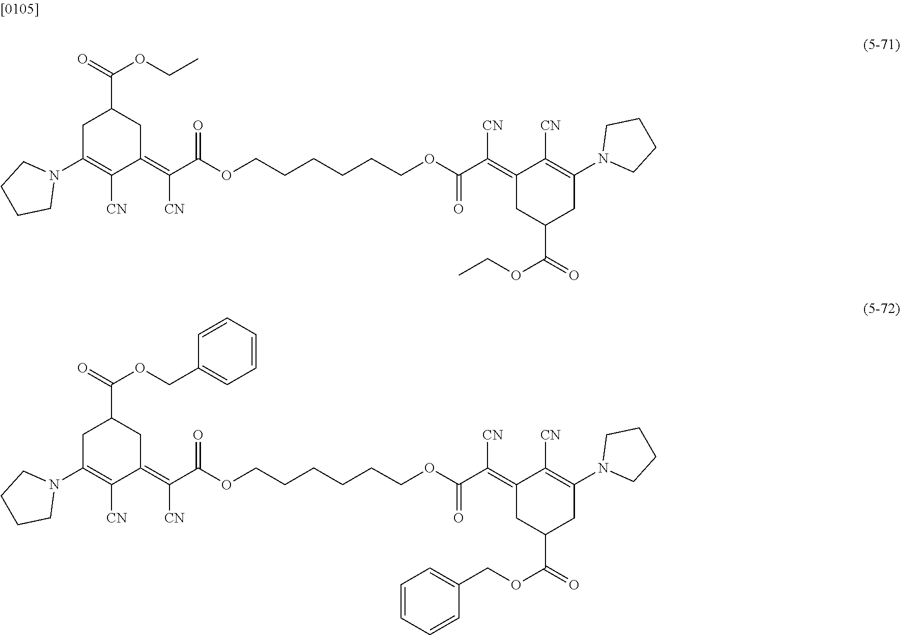 [SMILES: CCOC(=O)C1CC(N2CCCC2)=C(C#N)/C(=C(\C#N)C(=O)OCCCCCCOC(=O)/C(C#N)=C2\CC(C(=O)OCC)CC(N3CCCC3)=C2C#N)C1 ; N#CC1=C(N2CCCC2)CC(C(=O)OCc2ccccc2)C/C1=C(/C#N)C(=O)OCCCCCCOC(=O)/C(C#N)=C1\CC(C(=O)OCc2ccccc2)CC(N2CCCC2)=C1C#N]

Examples of the compound represented by Formula (VI) (hereinafter, may be referred to as a compound (VI)) include the following compounds.

The compound (VI) is preferably compounds represented by Formula (6-1), Formula (6-2), Formula (6-4), Formula (6-5), Formula (6-7), Formula (6-8), Formula (6-9), Formula (6-12), Formula (6-15), Formula (6-18), Formula (6-19), Formula (6-22), Formula (6-23), Formula (6-50), Formula (6-57), Formula (6-69), Formula (6-80), Formula (6-85), or Formula (6-94), and more preferably compounds represented by Formula (6-1), Formula (6-2), Formula (6-4), Formula (6-8), Formula (6-15), Formula (6-22), and Formula (6-80).

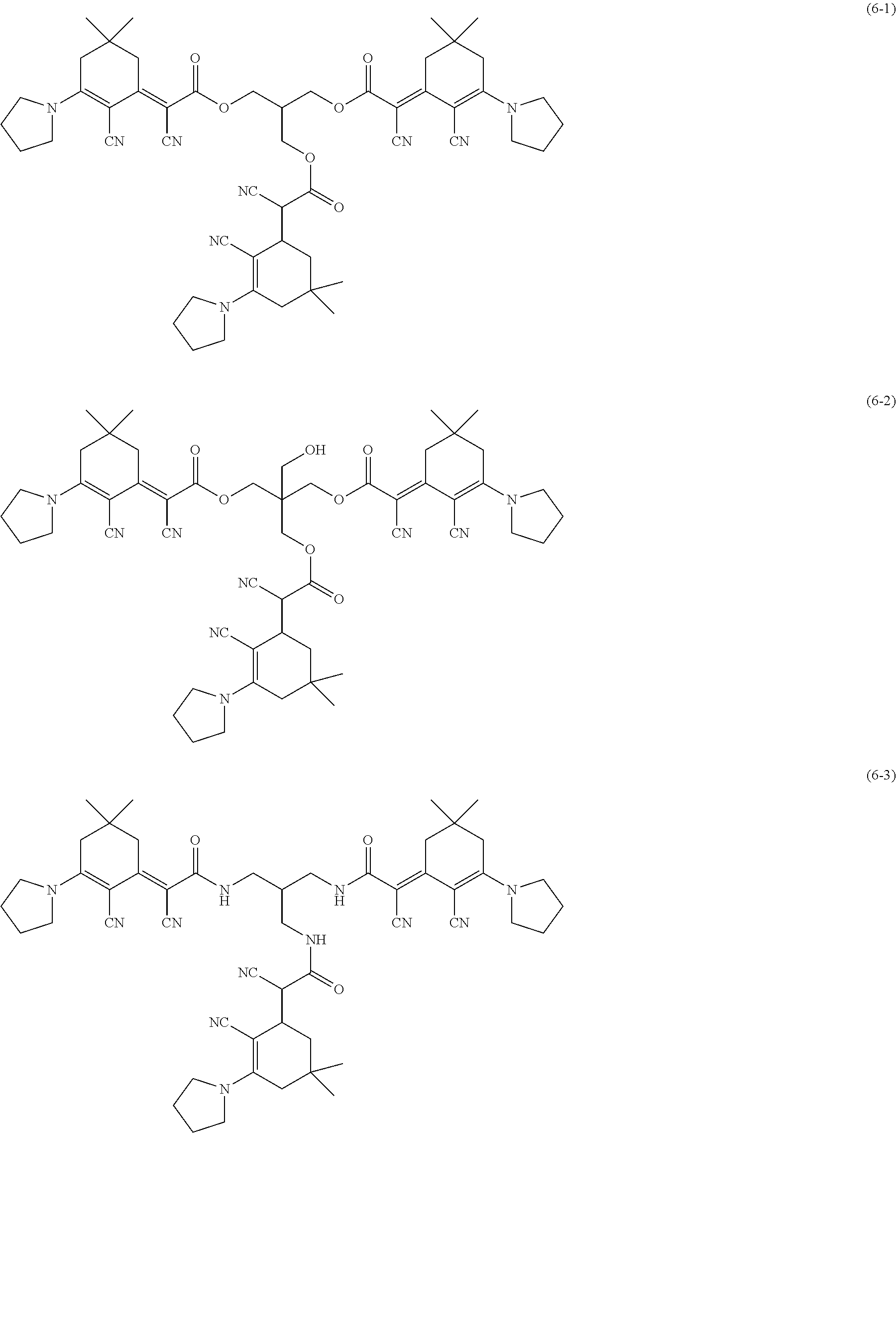 [SMILES: CC1(C)CC(N2CCCC2)=C(C#N)/C(=C(\C#N)C(=O)OCC(COC(=O)/C(C#N)=C2\CC(C)(C)CC(N3CCCC3)=C2C#N)COC(=O)C(C#N)C2CC(C)(C)CC(N3CCCC3)=C2C#N)C1 ; CC1(C)CC(N2CCCC2)=C(C#N)/C(=C(\C#N)C(=O)OCC(CO)(COC(=O)/C(C#N)=C2\CC(C)(C)CC(N3CCCC3)=C2C#N)COC(=O)C(C#N)C2CC(C)(C)CC(N3CCCC3)=C2C#N)C1 ; CC1(C)CC(N2CCCC2)=C(C#N)/C(=C(\C#N)C(=O)NCC(CNC(=O)/C(C#N)=C2\CC(C)(C)CC(N3CCCC3)=C2C#N)CNC(=O)C(C#N)C2CC(C)(C)CC(N3CCCC3)=C2C#N)C1]

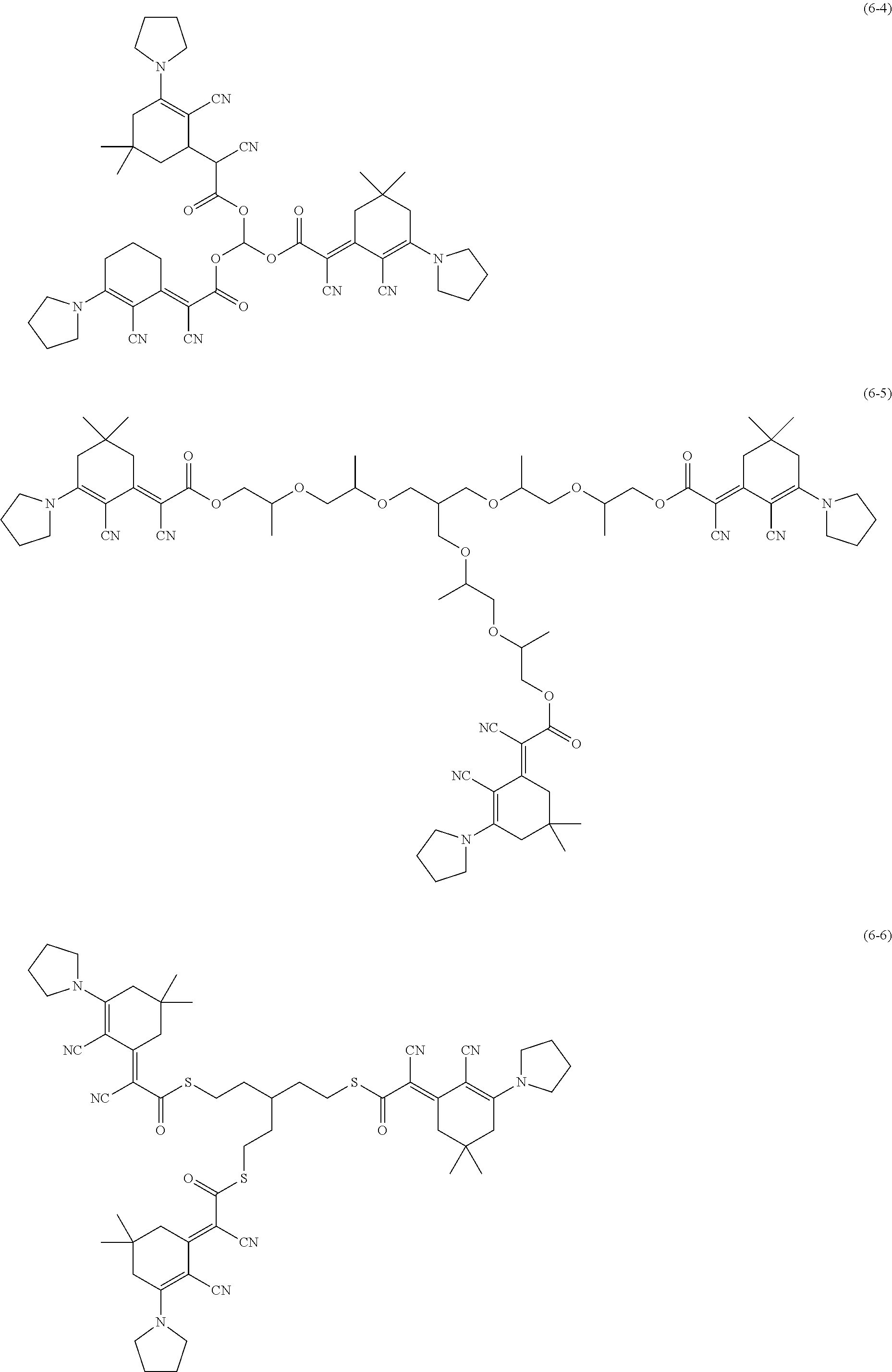 [SMILES: CC1(C)CC(N2CCCC2)=C(C#N)/C(=C(\C#N)C(=O)OC(OC(=O)/C(C#N)=C2\CCCC(N3CCCC3)=C2C#N)OC(=O)C(C#N)C2CC(C)(C)CC(N3CCCC3)=C2C#N)C1 ; CC(COC(=O)/C(C#N)=C1\CC(C)(C)CC(N2CCCC2)=C1C#N)OCC(C)OCC(COC(C)COC(C)COC(=O)/C(C#N)=C1\CC(C)(C)CC(N2CCCC2)=C1C#N)COC(C)COC(C)COC(=O)/C(C#N)=C1\CC(C)(C)CC(N2CCCC2)=C1C#N ; CC1(C)CC(N2CCCC2)=C(C#N)/C(=C(\C#N)C(=O)SCCC(CCSC(=O)/C(C#N)=C2\CC(C)(C)CC(N3CCCC3)=C2C#N)CCSC(=O)/C(C#N)=C2\CC(C)(C)CC(N3CCCC3)=C2C#N)C1]

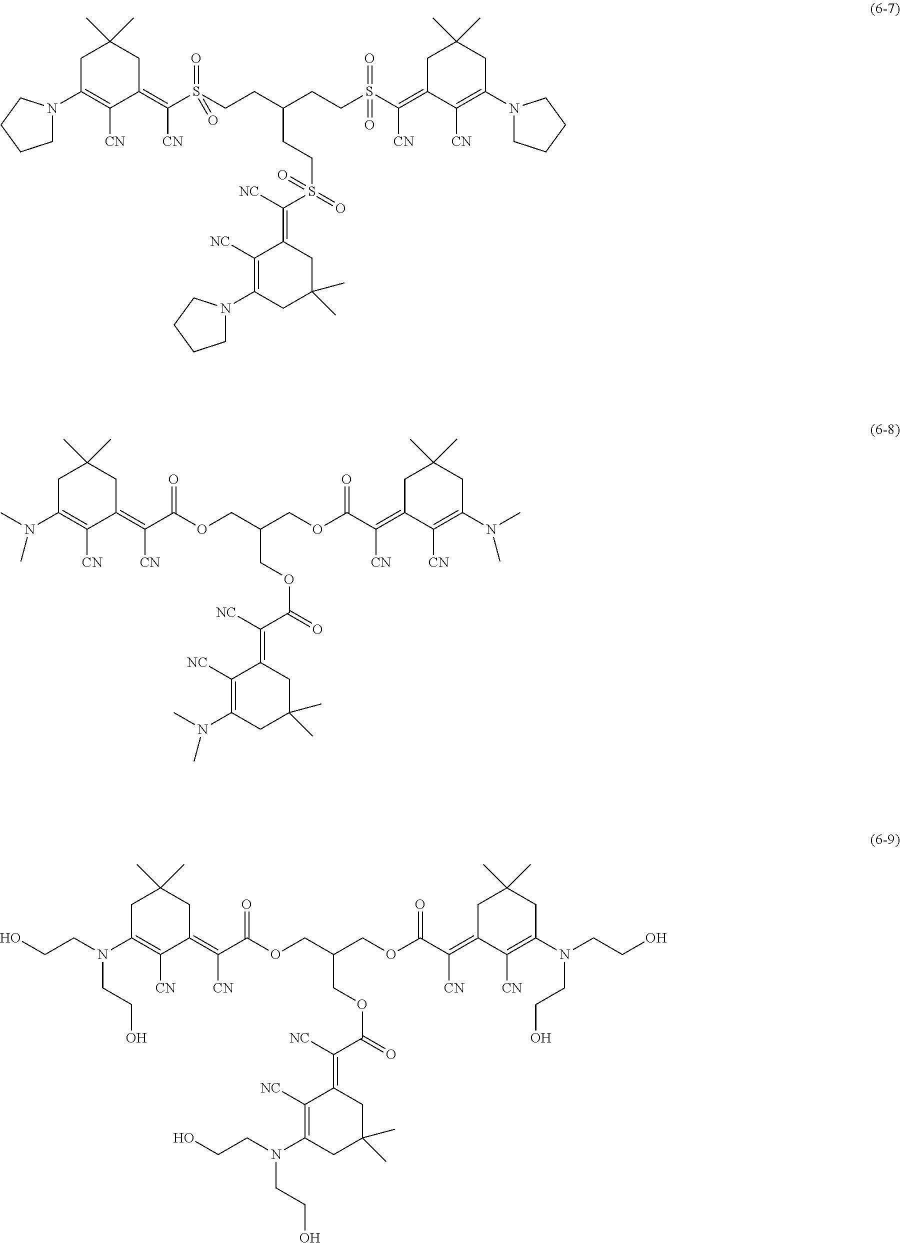

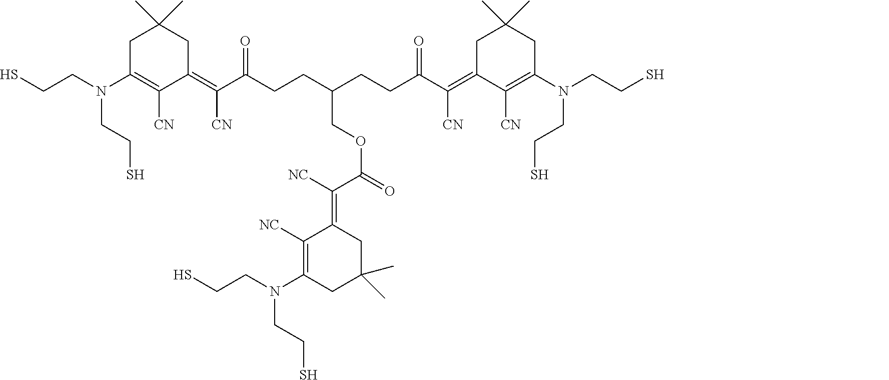 [SMILES: CC1(C)CC(N(CCS)CCS)=C(C#N)/C(=C(\C#N)C(=O)CCC(CCC(=O)/C(C#N)=C2\CC(C)(C)CC(N(CCS)CCS)=C2C#N)COC(=O)/C(C#N)=C2\CC(C)(C)CC(N(CCS)CCS)=C2C#N)C1]

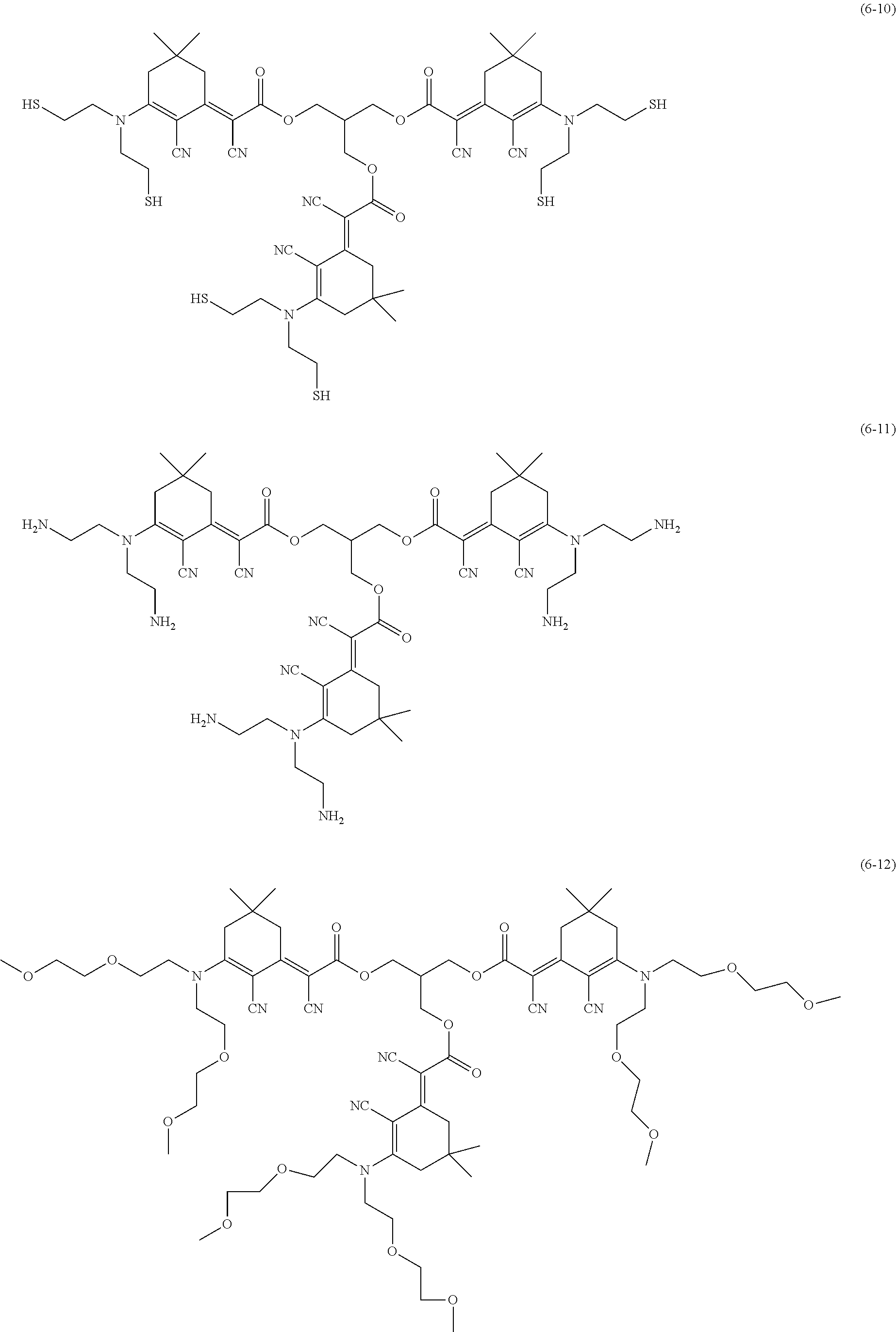

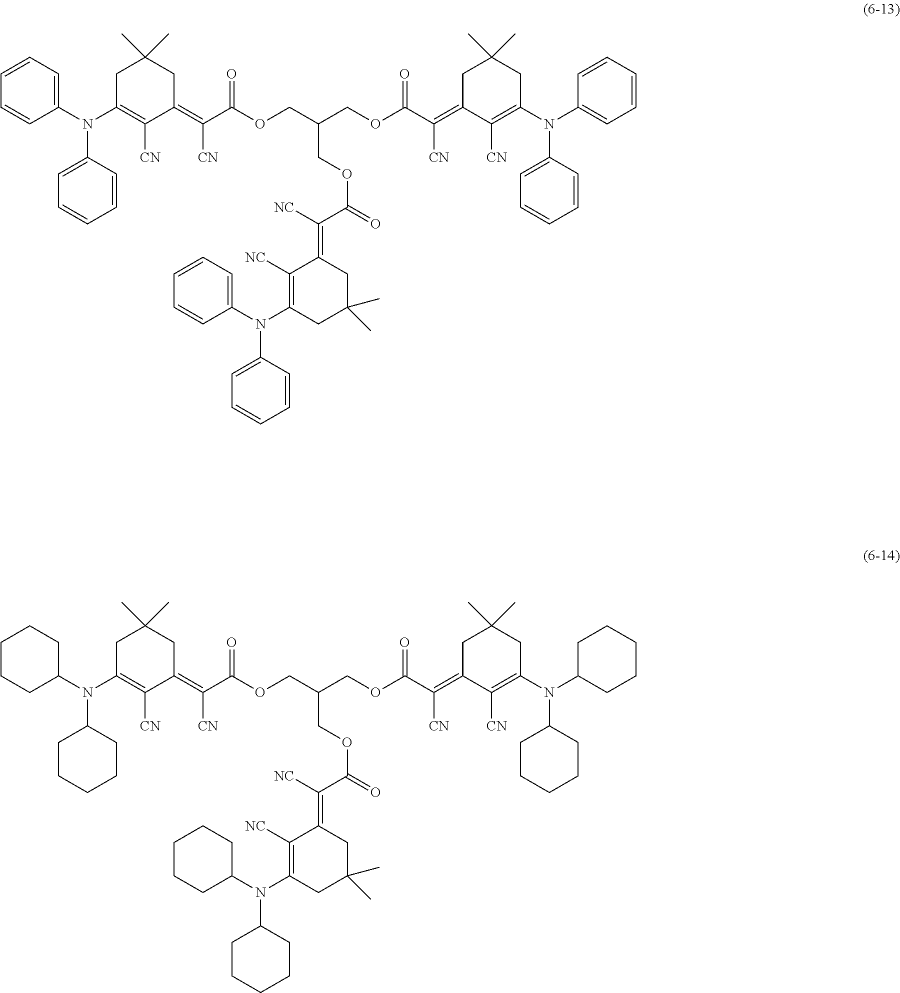 [SMILES: CC1(C)CC(N(c2ccccc2)c2ccccc2)=C(C#N)/C(=C(\C#N)C(=O)OCC(COC(=O)/C(C#N)=C2\CC(C)(C)CC(N(c3ccccc3)c3ccccc3)=C2C#N)COC(=O)/C(C#N)=C2\CC(C)(C)CC(N(c3ccccc3)c3ccccc3)=C2C#N)C1 ; CC1(C)CC(N(C2CCCCC2)C2CCCCC2)=C(C#N)/C(=C(\C#N)C(=O)OCC(COC(=O)/C(C#N)=C2\CC(C)(C)CC(N(C3CCCCC3)C3CCCCC3)=C2C#N)COC(=O)/C(C#N)=C2\CC(C)(C)CC(N(C3CCCCC3)C3CCCCC3)=C2C#N)C1]

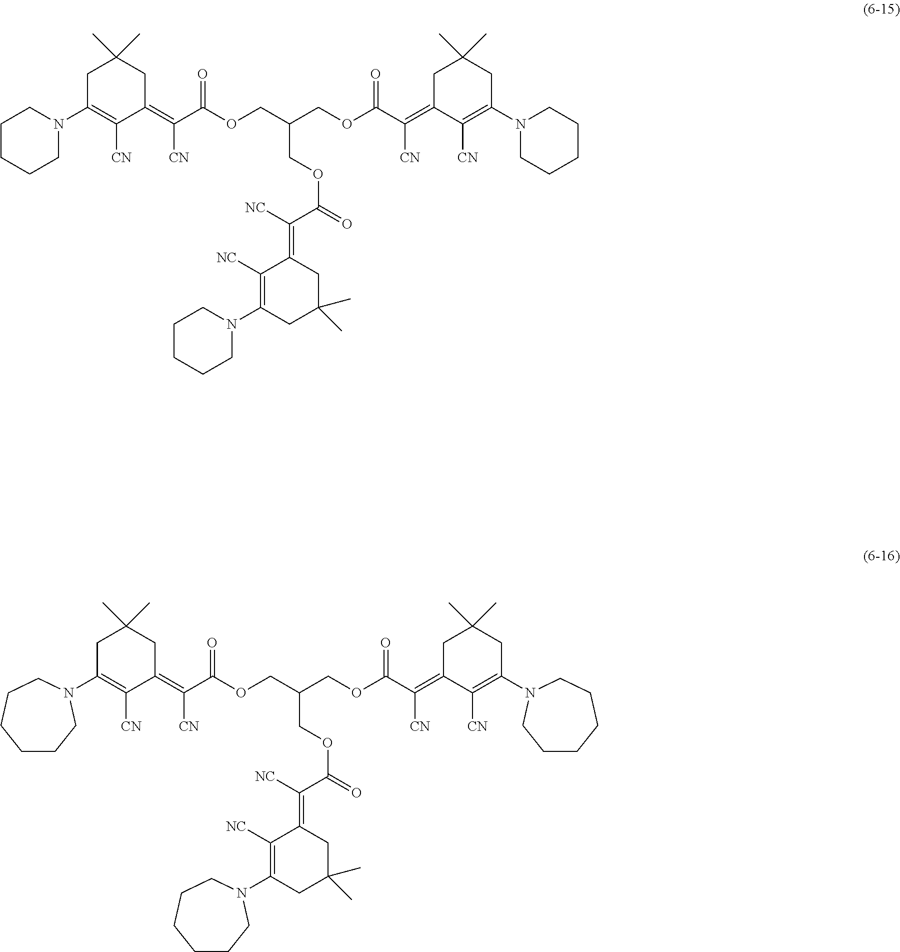 [SMILES: CC1(C)CC(N2CCCCC2)=C(C#N)/C(=C(\C#N)C(=O)OCC(COC(=O)/C(C#N)=C2\CC(C)(C)CC(N3CCCCC3)=C2C#N)COC(=O)/C(C#N)=C2\CC(C)(C)CC(N3CCCCC3)=C2C#N)C1 ; CC1(C)CC(N2CCCCCC2)=C(C#N)/C(=C(\C#N)C(=O)OCC(COC(=O)/C(C#N)=C2\CC(C)(C)CC(N3CCCCCC3)=C2C#N)COC(=O)/C(C#N)=C2\CC(C)(C)CC(N3CCCCCC3)=C2C#N)C1]

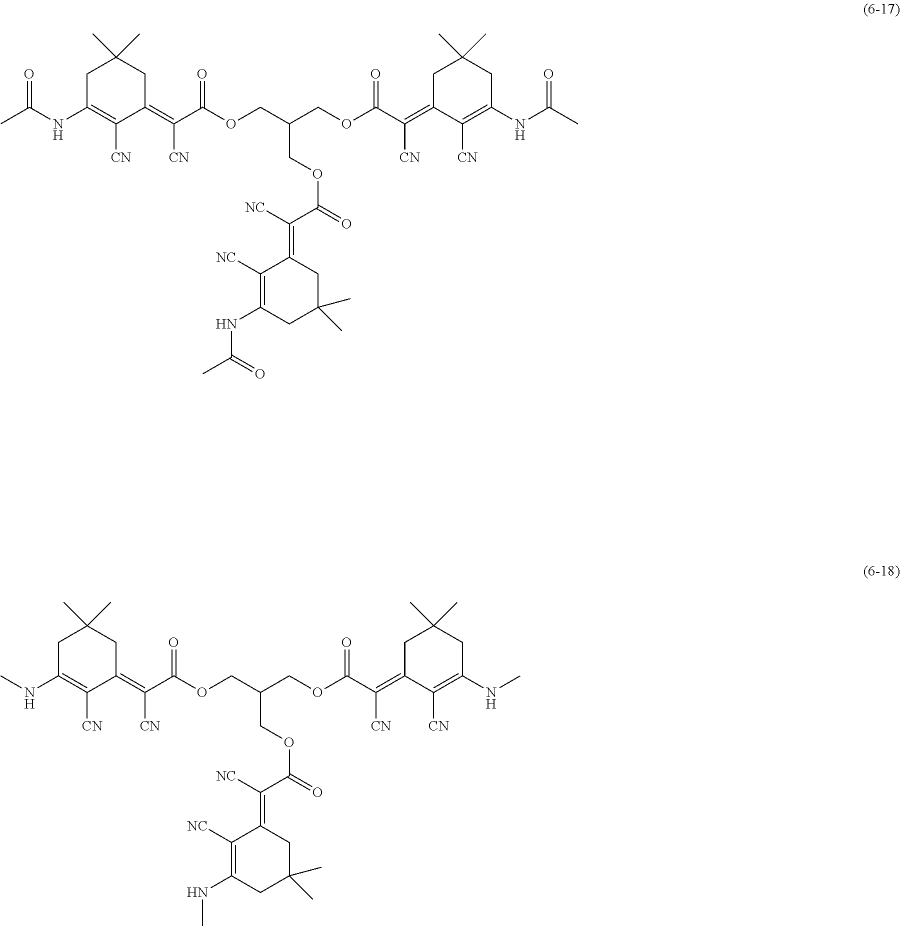

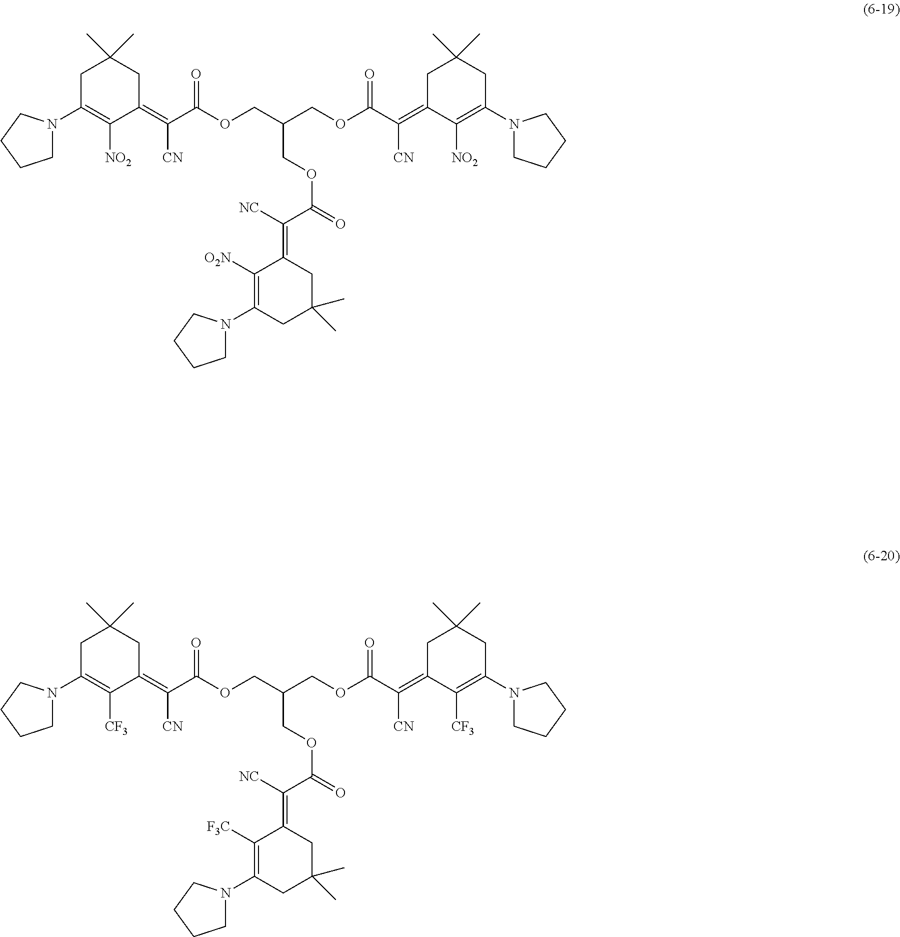 [SMILES: CC1(C)CC(N2CCCC2)=C([N+](=O)[O-])/C(=C(\C#N)C(=O)OCC(COC(=O)/C(C#N)=C2\CC(C)(C)CC(N3CCCC3)=C2[N+](=O)[O-])COC(=O)/C(C#N)=C2\CC(C)(C)CC(N3CCCC3)=C2[N+](=O)[O-])C1 ; CC1(C)CC(N2CCCC2)=C(C(F)(F)F)/C(=C(\C#N)C(=O)OCC(COC(=O)/C(C#N)=C2\CC(C)(C)CC(N3CCCC3)=C2C(F)(F)F)COC(=O)/C(C#N)=C2\CC(C)(C)CC(N3CCCC3)=C2C(F)(F)F)C1]

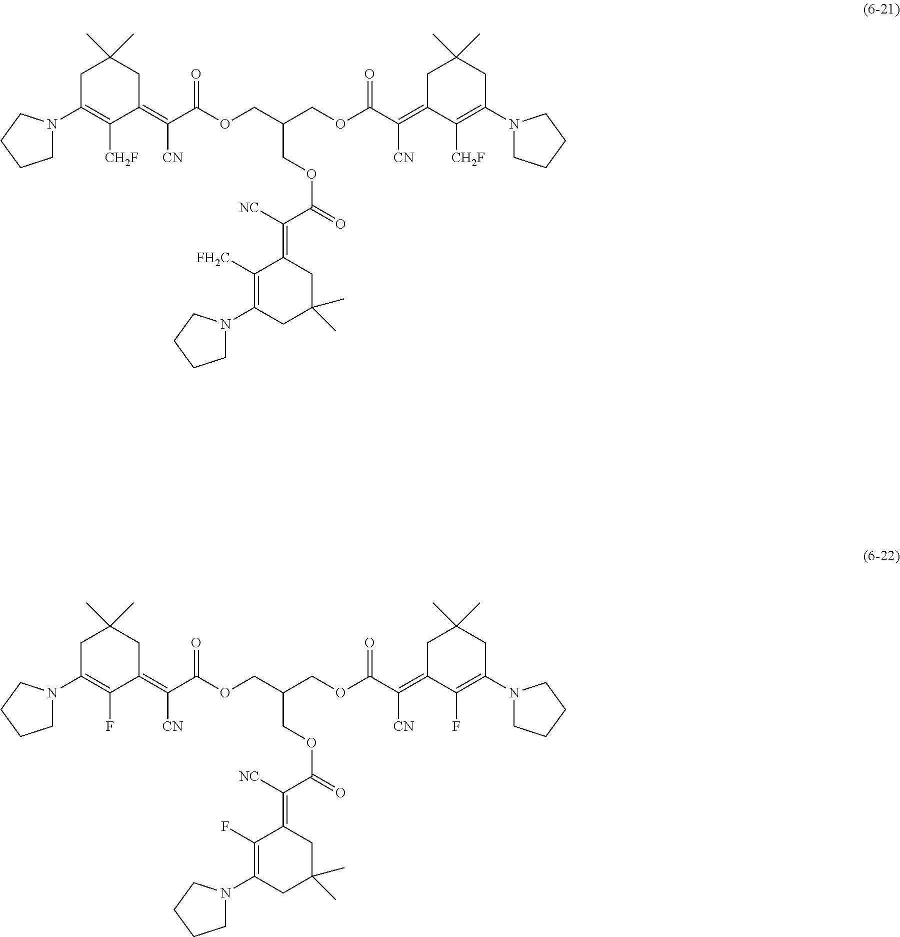

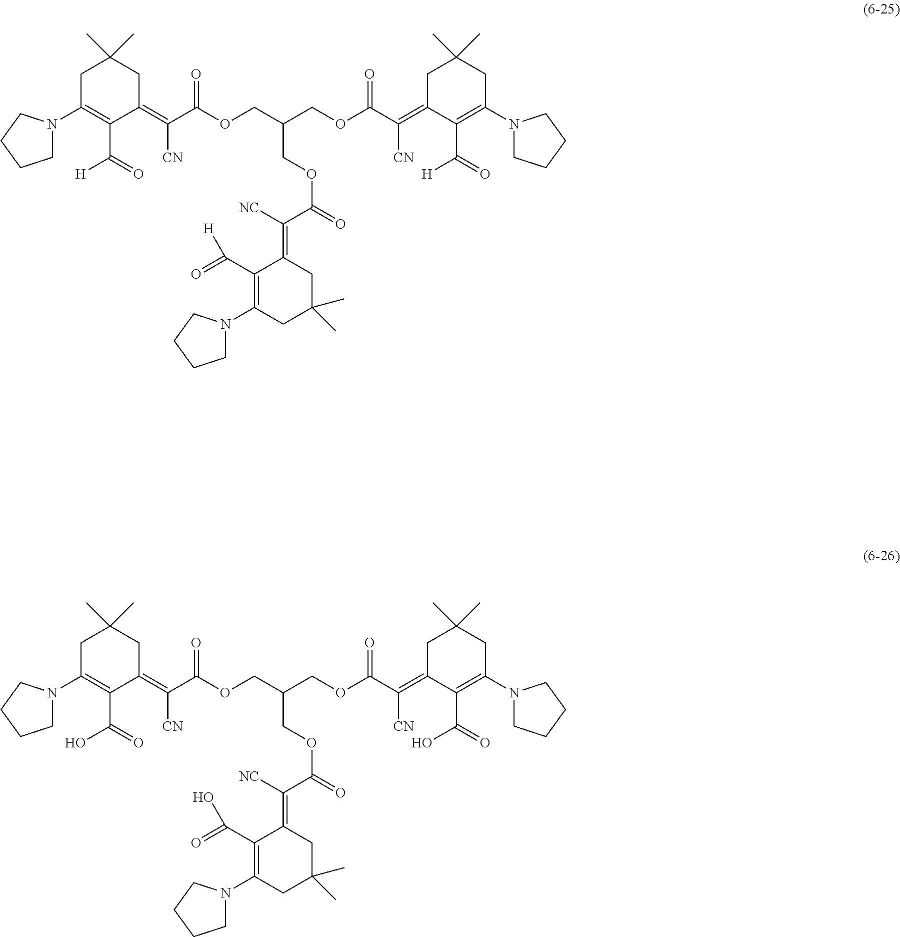 [SMILES: CC1(C)CC(N2CCCC2)=C(C=O)/C(=C(\C#N)C(=O)OCC(COC(=O)/C(C#N)=C2\CC(C)(C)CC(N3CCCC3)=C2C=O)COC(=O)/C(C#N)=C2\CC(C)(C)CC(N3CCCC3)=C2C=O)C1 ; CC1(C)CC(N2CCCC2)=C(C(=O)O)/C(=C(\C#N)C(=O)OCC(COC(=O)/C(C#N)=C2\CC(C)(C)CC(N3CCCC3)=C2C(=O)O)COC(=O)/C(C#N)=C2\CC(C)(C)CC(N3CCCC3)=C2C(=O)O)C1]

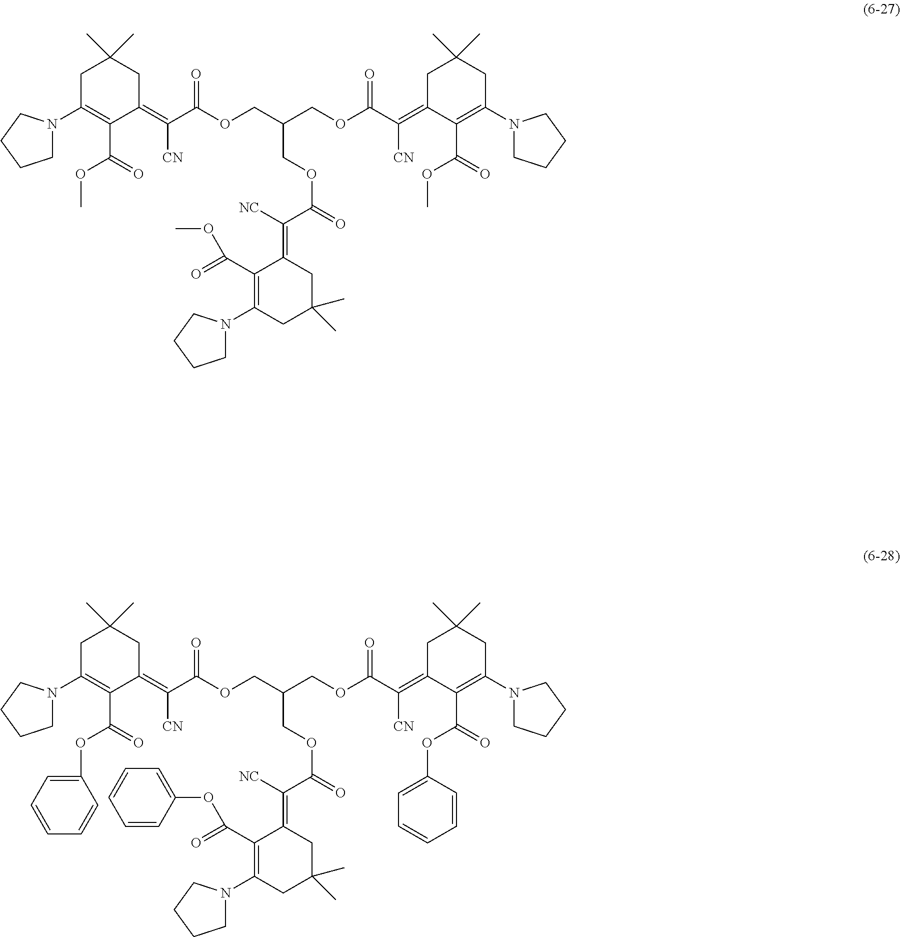 [SMILES: COC(=O)C1=C(N2CCCC2)CC(C)(C)C/C1=C(/C#N)C(=O)OCC(COC(=O)/C(C#N)=C1\CC(C)(C)CC(N2CCCC2)=C1C(=O)OC)COC(=O)/C(C#N)=C1\CC(C)(C)CC(N2CCCC2)=C1C(=O)OC ; CC1(C)CC(N2CCCC2)=C(C(=O)Oc2ccccc2)/C(=C(\C#N)C(=O)OCC(COC(=O)/C(C#N)=C2\CC(C)(C)CC(N3CCCC3)=C2C(=O)Oc2ccccc2)COC(=O)/C(C#N)=C2\CC(C)(C)CC(N3CCCC3)=C2C(=O)Oc2ccccc2)C1]

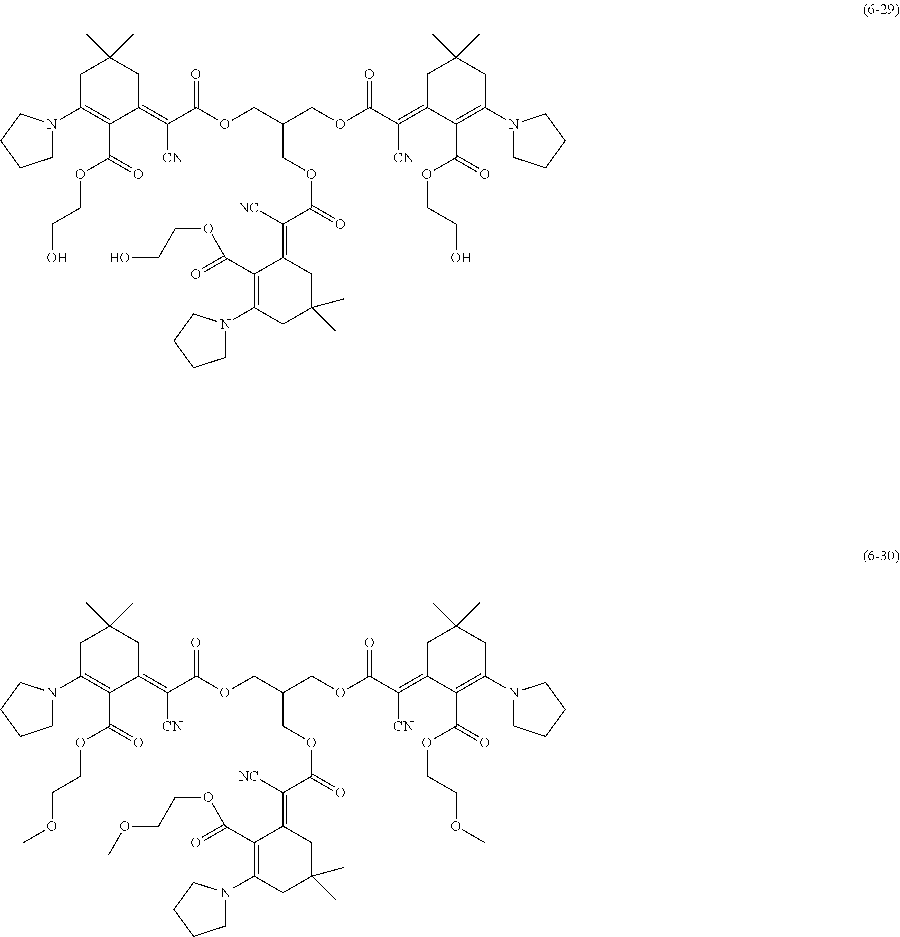 [SMILES: CC1(C)CC(N2CCCC2)=C(C(=O)OCCO)/C(=C(\C#N)C(=O)OCC(COC(=O)/C(C#N)=C2\CC(C)(C)CC(N3CCCC3)=C2C(=O)OCCO)COC(=O)/C(C#N)=C2\CC(C)(C)CC(N3CCCC3)=C2C(=O)OCCO)C1 ; COCCOC(=O)C1=C(N2CCCC2)CC(C)(C)C/C1=C(/C#N)C(=O)OCC(COC(=O)/C(C#N)=C1\CC(C)(C)CC(N2CCCC2)=C1C(=O)OCCOC)COC(=O)/C(C#N)=C1\CC(C)(C)CC(N2CCCC2)=C1C(=O)OCCOC]

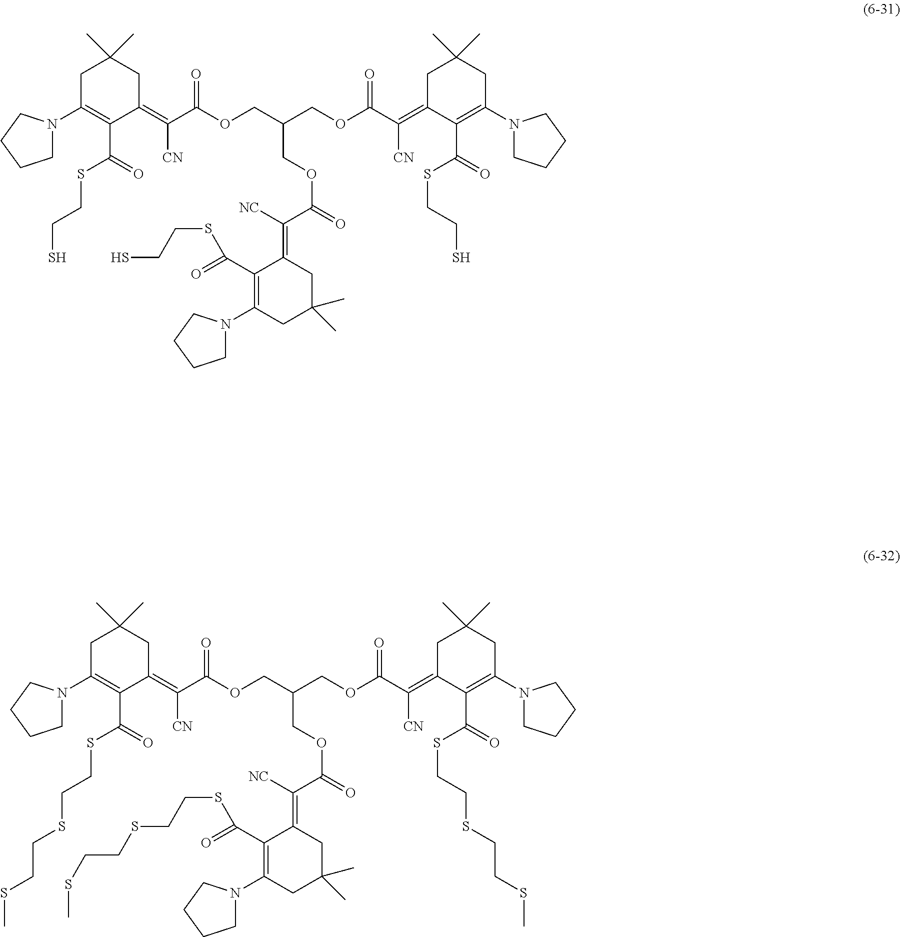 [SMILES: CC1(C)CC(N2CCCC2)=C(C(=O)SCCS)/C(=C(\C#N)C(=O)OCC(COC(=O)/C(C#N)=C2\CC(C)(C)CC(N3CCCC3)=C2C(=O)SCCS)COC(=O)/C(C#N)=C2\CC(C)(C)CC(N3CCCC3)=C2C(=O)SCCS)C1 ; CSCCSCCSC(=O)C1=C(N2CCCC2)CC(C)(C)C/C1=C(/C#N)C(=O)OCC(COC(=O)/C(C#N)=C1\CC(C)(C)CC(N2CCCC2)=C1C(=O)SCCSCCSC)COC(=O)/C(C#N)=C1\CC(C)(C)CC(N2CCCC2)=C1C(=O)SCCSCCSC]

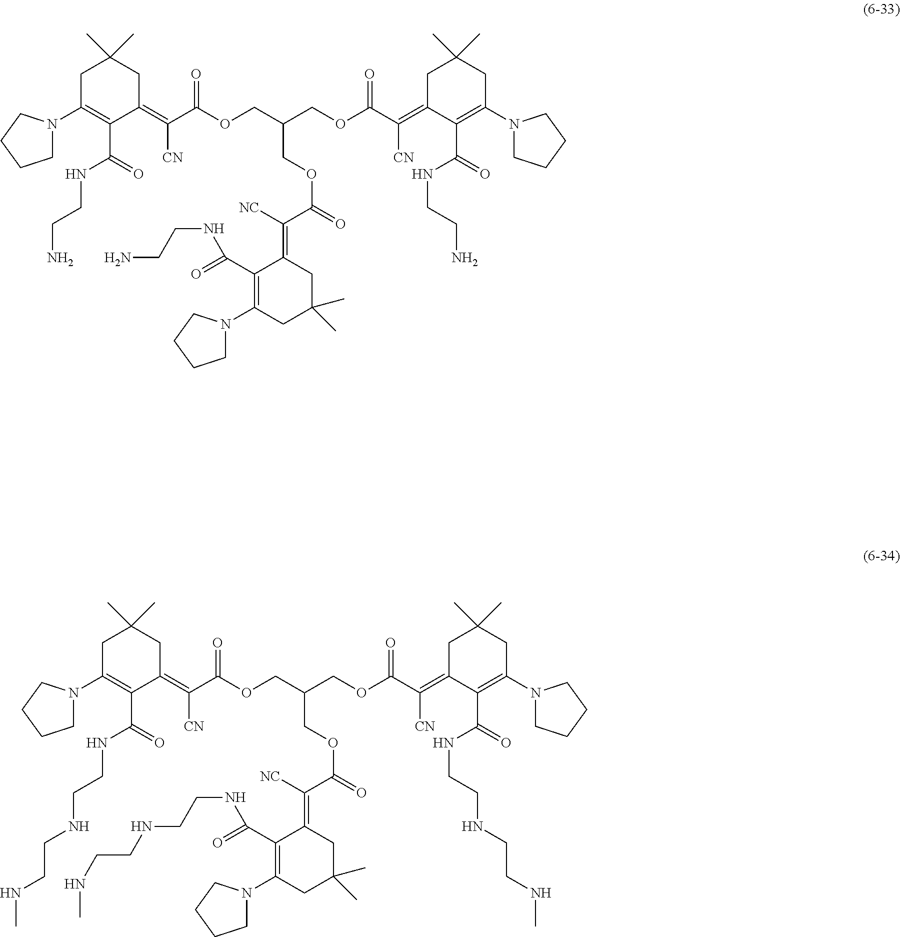 [SMILES: CC1(C)CC(N2CCCC2)=C(C(=O)NCCN)/C(=C(\C#N)C(=O)OCC(COC(=O)/C(C#N)=C2\CC(C)(C)CC(N3CCCC3)=C2C(=O)NCCN)COC(=O)/C(C#N)=C2\CC(C)(C)CC(N3CCCC3)=C2C(=O)NCCN)C1 ; CNCCNCCNC(=O)C1=C(N2CCCC2)CC(C)(C)C/C1=C(/C#N)C(=O)OCC(COC(=O)/C(C#N)=C1\CC(C)(C)CC(N2CCCC2)=C1C(=O)NCCNCCNC)COC(=O)/C(C#N)=C1\CC(C)(C)CC(N2CCCC2)=C1C(=O)NCCNCCNC]

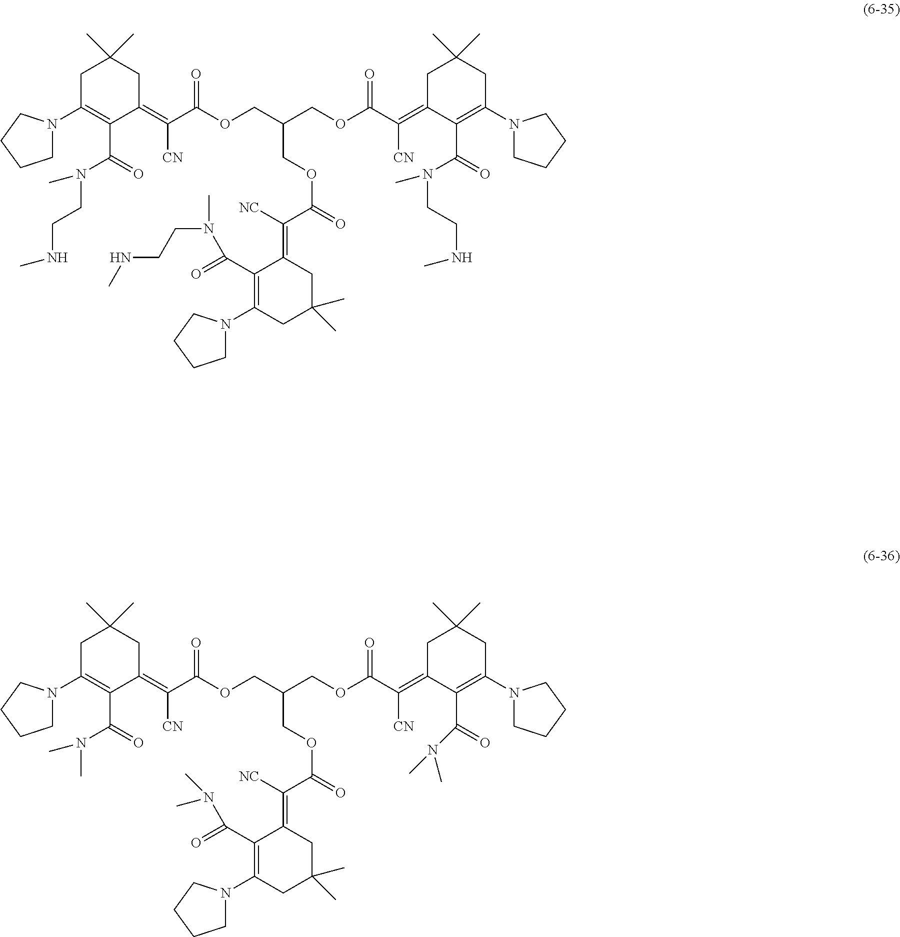 [SMILES: CNCCN(C)C(=O)C1=C(N2CCCC2)CC(C)(C)C/C1=C(/C#N)C(=O)OCC(COC(=O)/C(C#N)=C1\CC(C)(C)CC(N2CCCC2)=C1C(=O)N(C)CCNC)COC(=O)/C(C#N)=C1\CC(C)(C)CC(N2CCCC2)=C1C(=O)N(C)CCNC ; CN(C)C(=O)C1=C(N2CCCC2)CC(C)(C)C/C1=C(/C#N)C(=O)OCC(COC(=O)/C(C#N)=C1\CC(C)(C)CC(N2CCCC2)=C1C(=O)N(C)C)COC(=O)/C(C#N)=C1\CC(C)(C)CC(N2CCCC2)=C1C(=O)N(C)C]

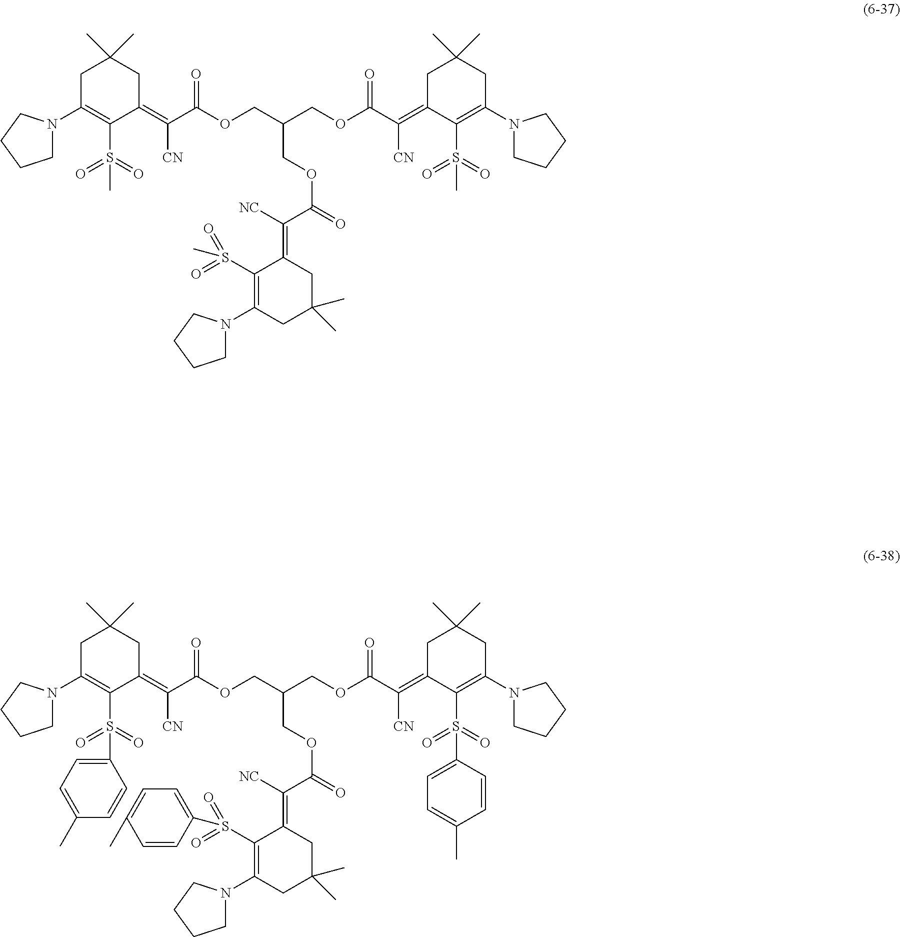 [SMILES: CC1(C)CC(N2CCCC2)=C(S(C)(=O)=O)/C(=C(\C#N)C(=O)OCC(COC(=O)/C(C#N)=C2\CC(C)(C)CC(N3CCCC3)=C2S(C)(=O)=O)COC(=O)/C(C#N)=C2\CC(C)(C)CC(N3CCCC3)=C2S(C)(=O)=O)C1 ; Cc1ccc(S(=O)(=O)C2=C(N3CCCC3)CC(C)(C)C/C2=C(/C#N)C(=O)OCC(COC(=O)/C(C#N)=C2\CC(C)(C)CC(N3CCCC3)=C2S(=O)(=O)c2ccc(C)cc2)COC(=O)/C(C#N)=C2\CC(C)(C)CC(N3CCCC3)=C2S(=O)(=O)c2ccc(C)cc2)cc1]

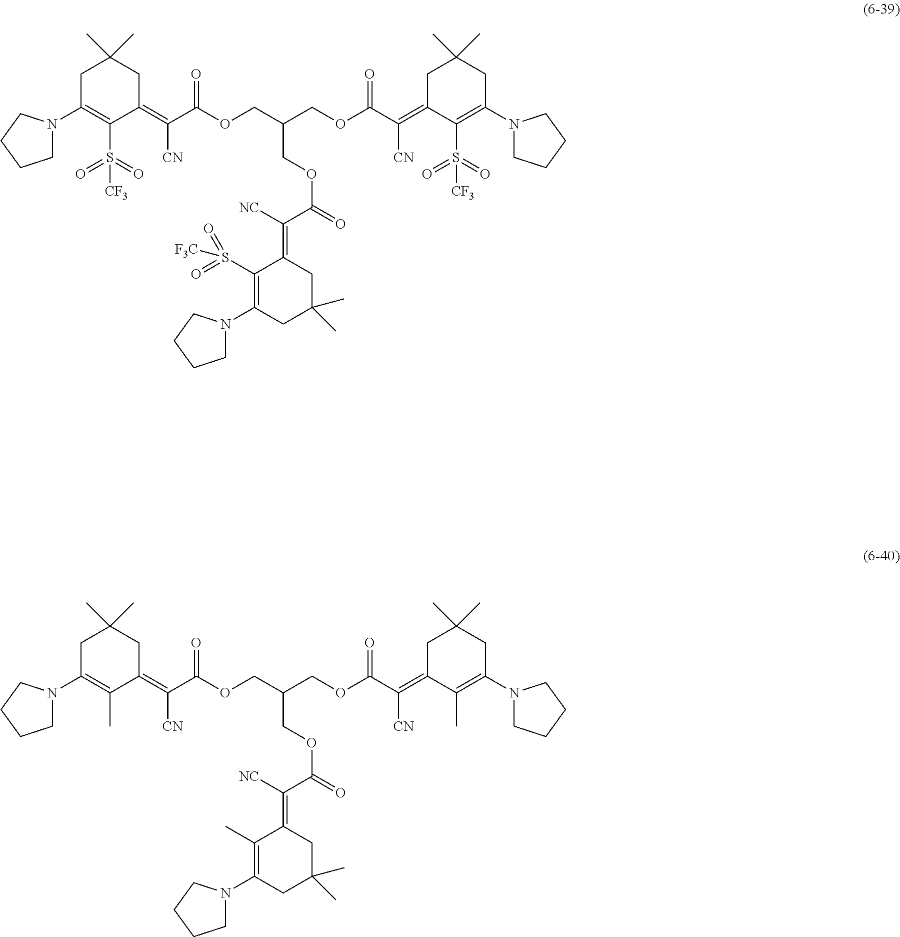 [SMILES: CC1(C)CC(N2CCCC2)=C(S(=O)(=O)C(F)(F)F)/C(=C(\C#N)C(=O)OCC(COC(=O)/C(C#N)=C2\CC(C)(C)CC(N3CCCC3)=C2S(=O)(=O)C(F)(F)F)COC(=O)/C(C#N)=C2\CC(C)(C)CC(N3CCCC3)=C2S(=O)(=O)C(F)(F)F)C1 ; CC1=C(N2CCCC2)CC(C)(C)C/C1=C(/C#N)C(=O)OCC(COC(=O)/C(C#N)=C1\CC(C)(C)CC(N2CCCC2)=C1C)COC(=O)/C(C#N)=C1\CC(C)(C)CC(N2CCCC2)=C1C]

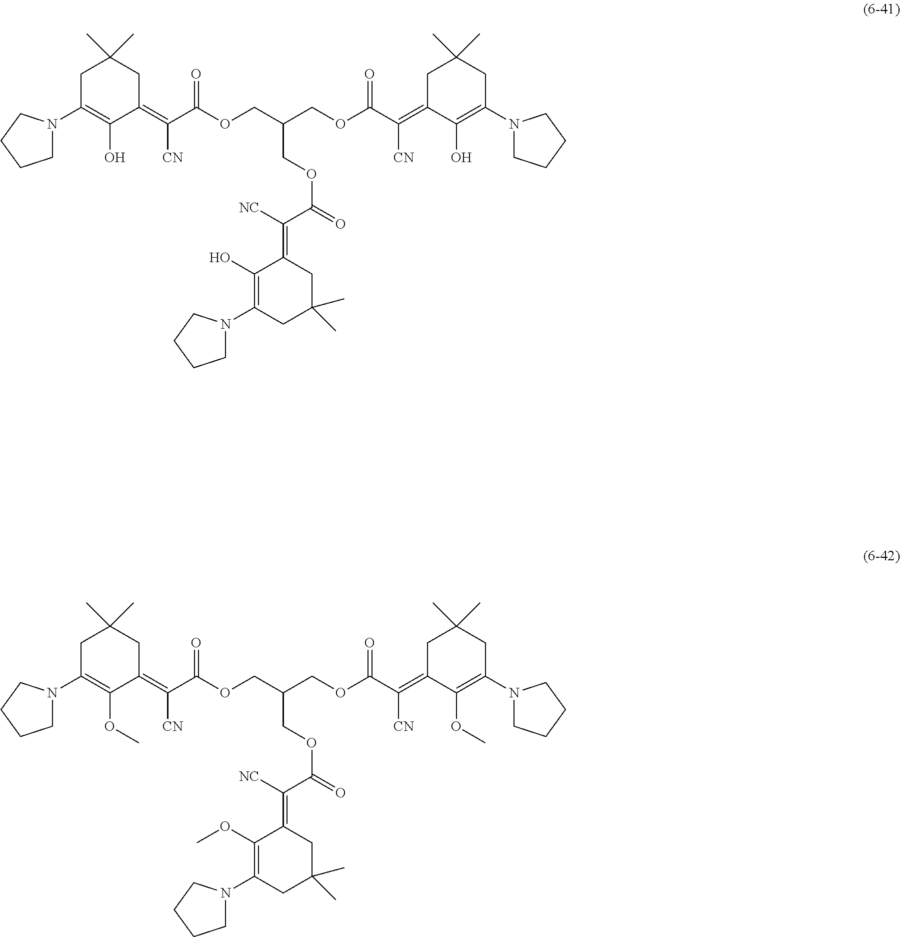 [SMILES: CC1(C)CC(N2CCCC2)=C(O)/C(=C(\C#N)C(=O)OCC(COC(=O)/C(C#N)=C2\CC(C)(C)CC(N3CCCC3)=C2O)COC(=O)/C(C#N)=C2\CC(C)(C)CC(N3CCCC3)=C2O)C1 ; COC1=C(N2CCCC2)CC(C)(C)C/C1=C(/C#N)C(=O)OCC(COC(=O)/C(C#N)=C1\CC(C)(C)CC(N2CCCC2)=C1OC)COC(=O)/C(C#N)=C1\CC(C)(C)CC(N2CCCC2)=C1OC]

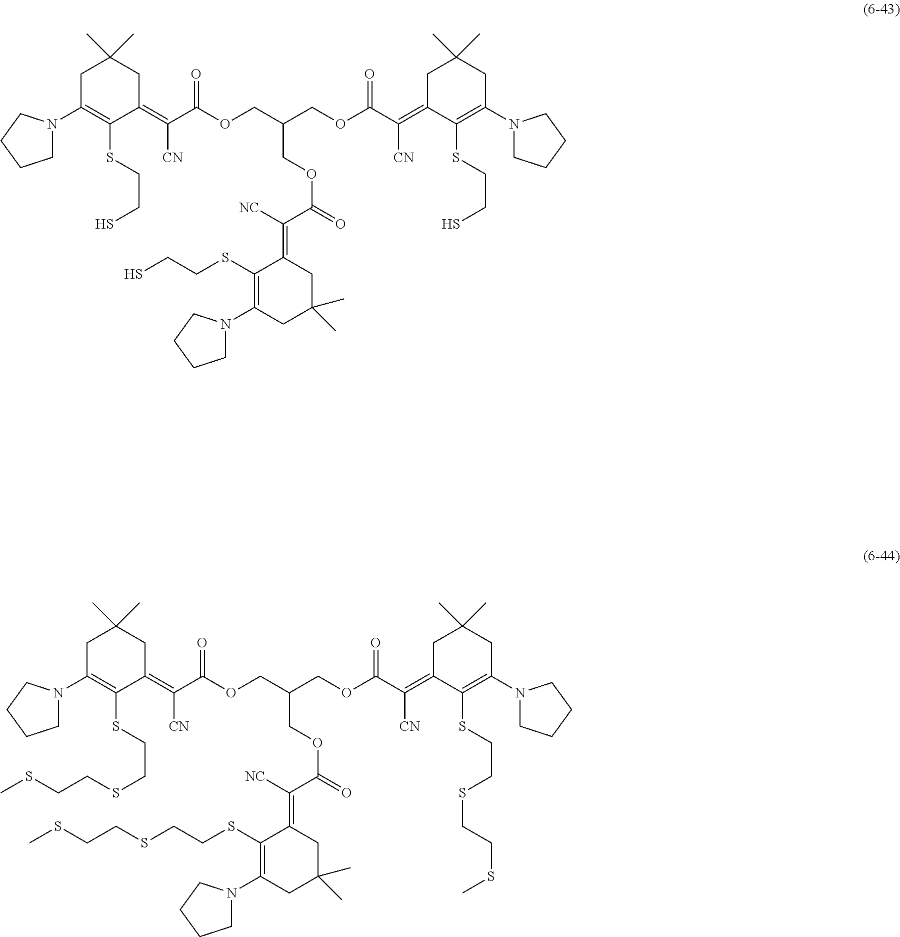 [SMILES: CC1(C)CC(N2CCCC2)=C(SCCS)/C(=C(\C#N)C(=O)OCC(COC(=O)/C(C#N)=C2\CC(C)(C)CC(N3CCCC3)=C2SCCS)COC(=O)/C(C#N)=C2\CC(C)(C)CC(N3CCCC3)=C2SCCS)C1 ; CSCCSCCSC1=C(N2CCCC2)CC(C)(C)C/C1=C(/C#N)C(=O)OCC(COC(=O)/C(C#N)=C1\CC(C)(C)CC(N2CCCC2)=C1SCCSCCSC)COC(=O)/C(C#N)=C1\CC(C)(C)CC(N2CCCC2)=C1SCCSCCSC]

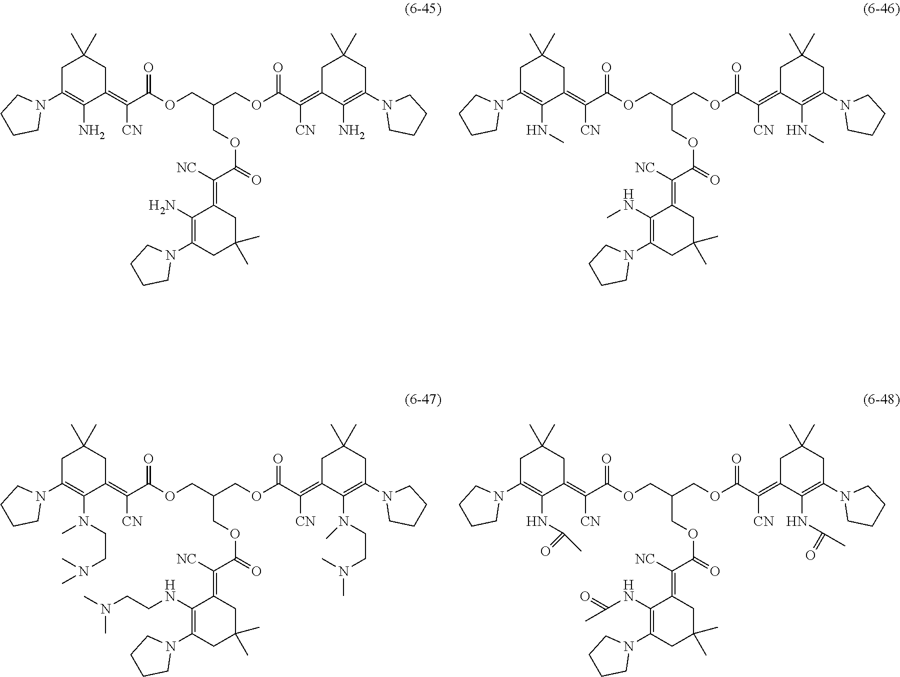

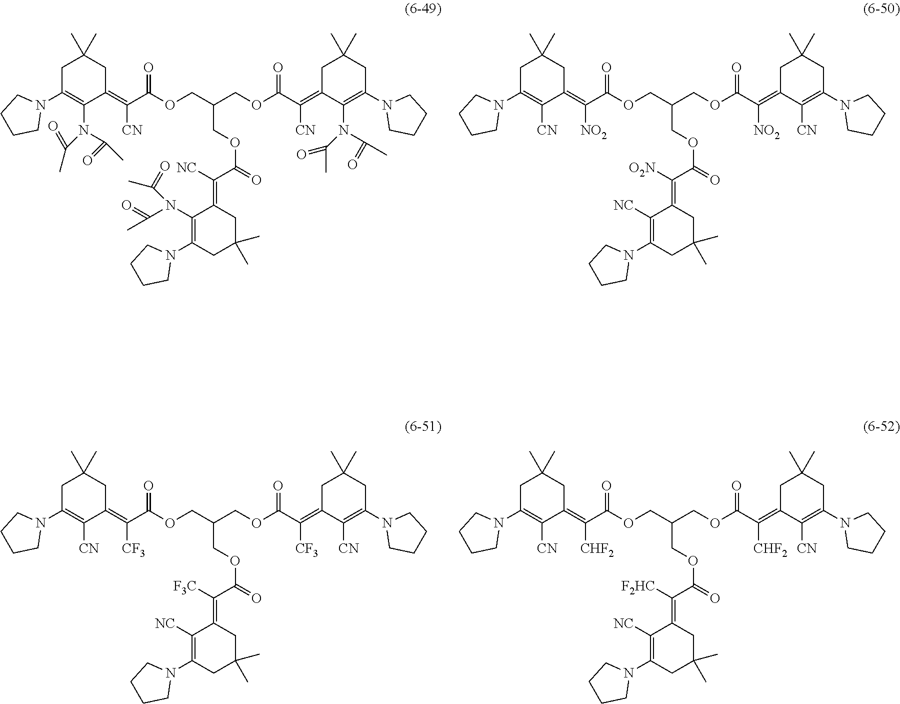

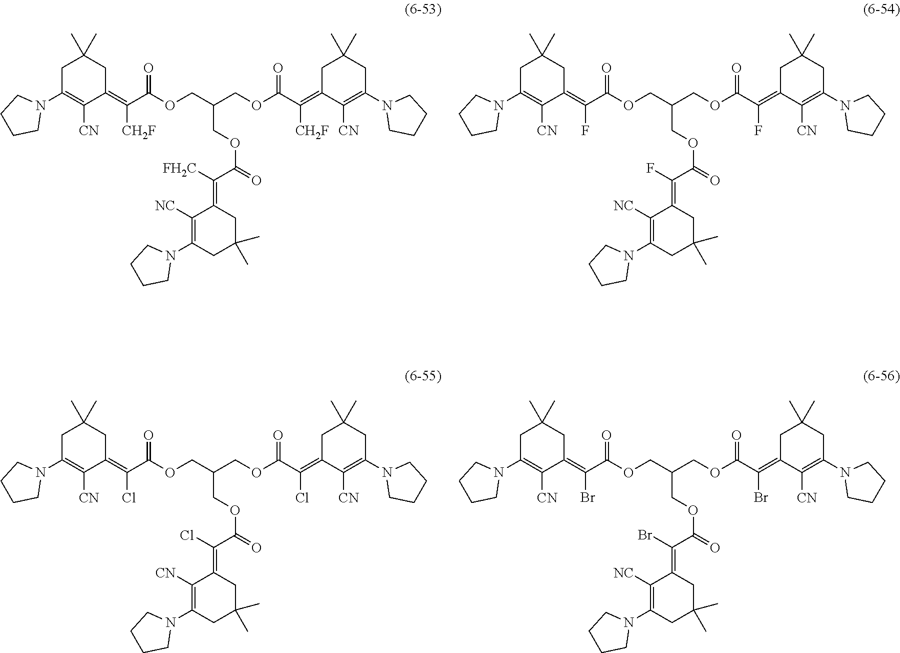

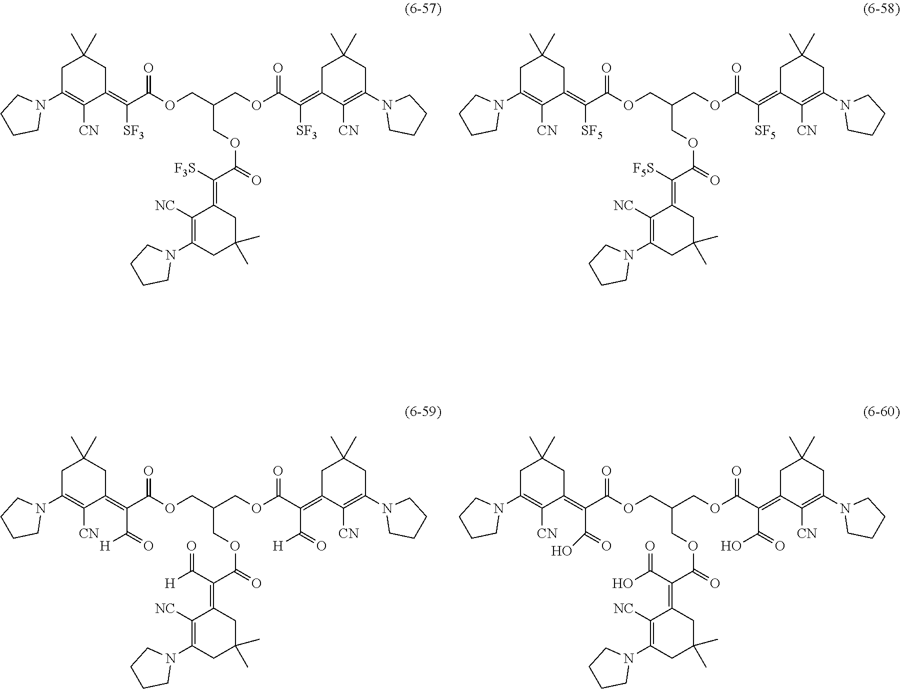

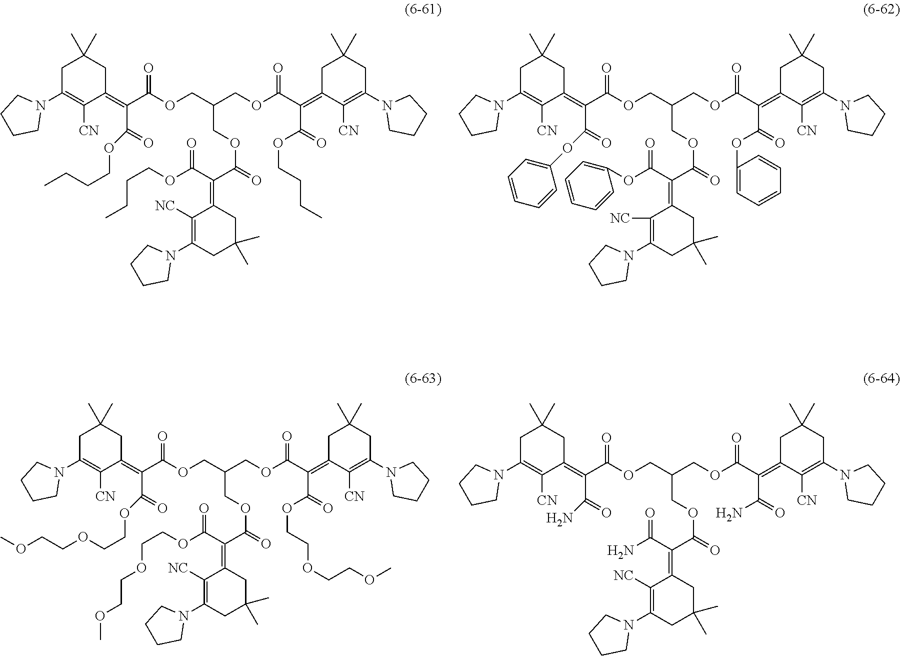

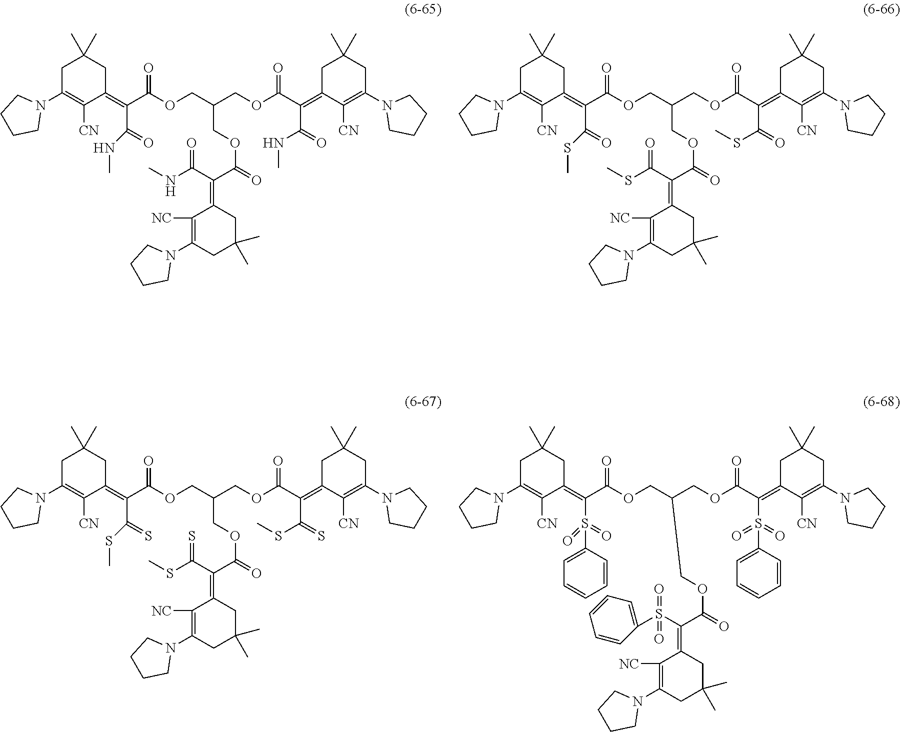

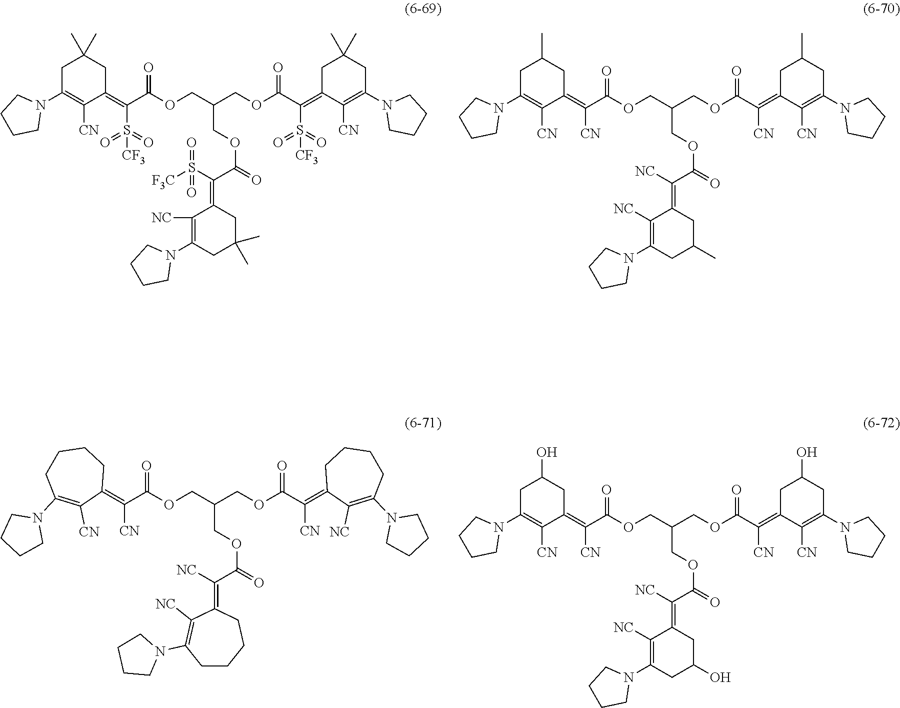

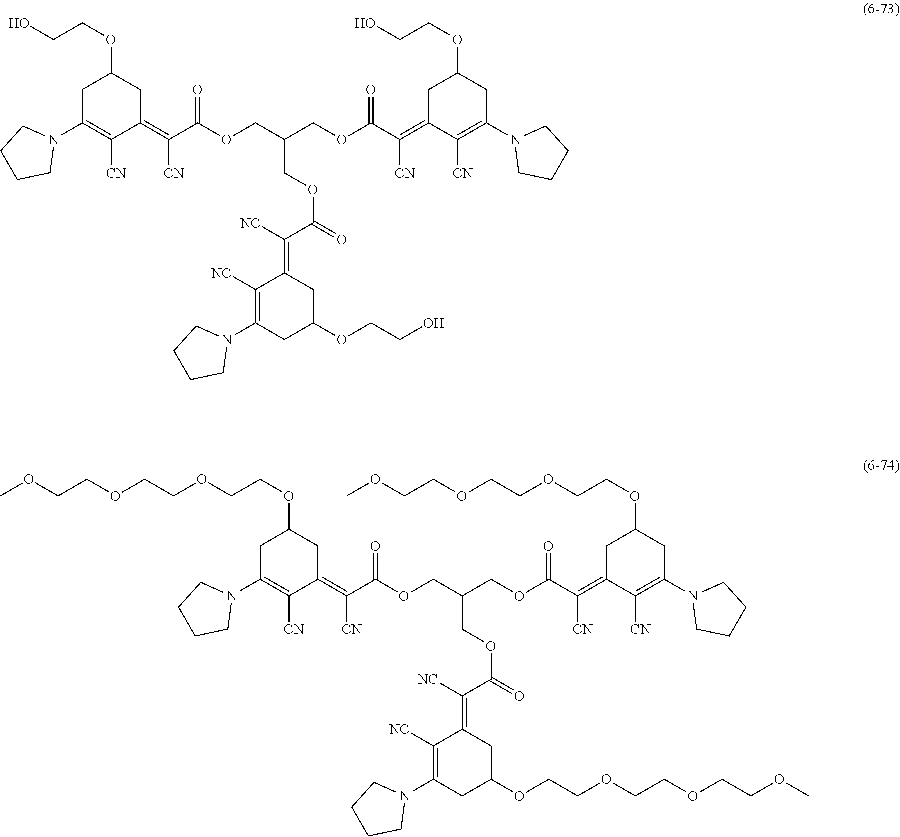

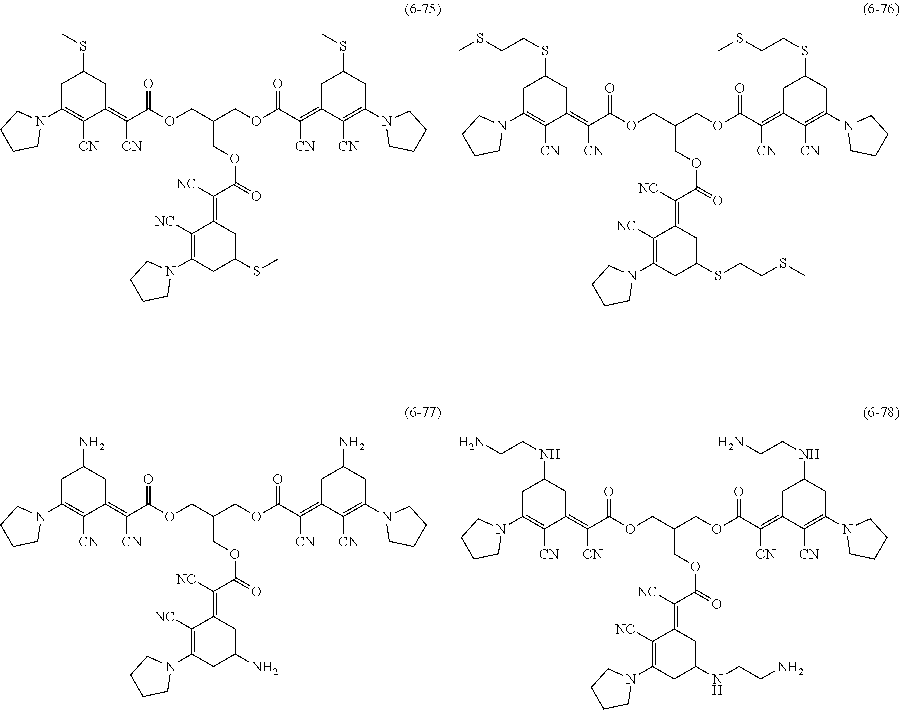

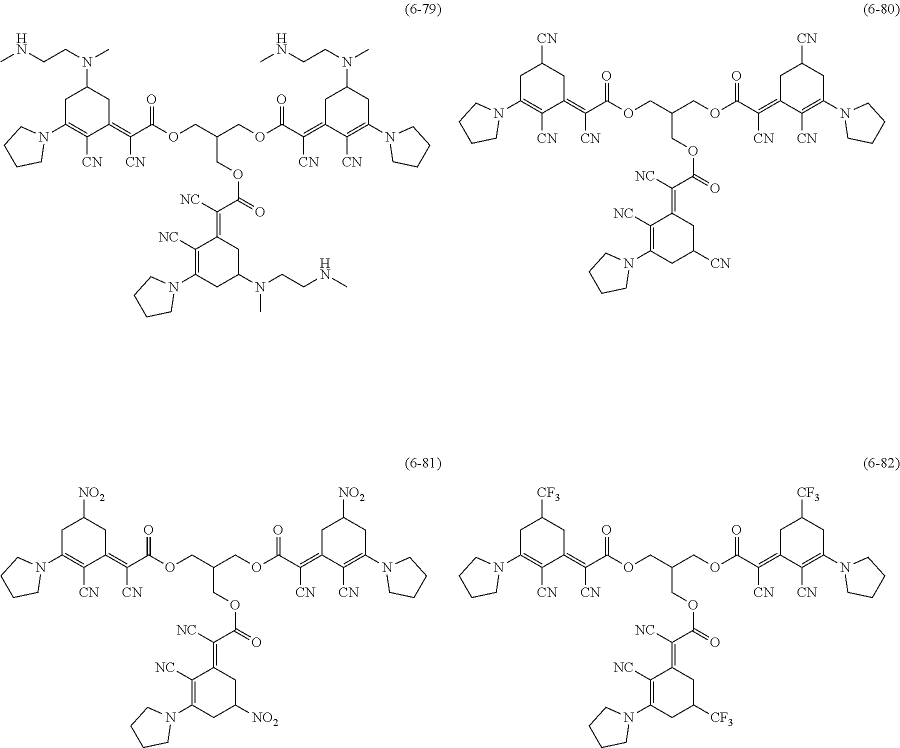

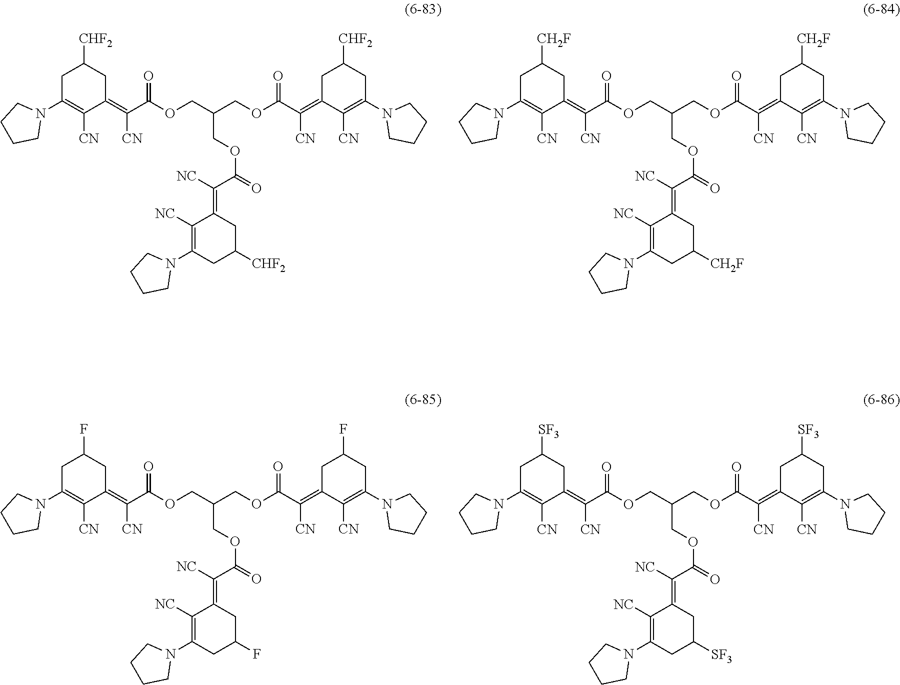

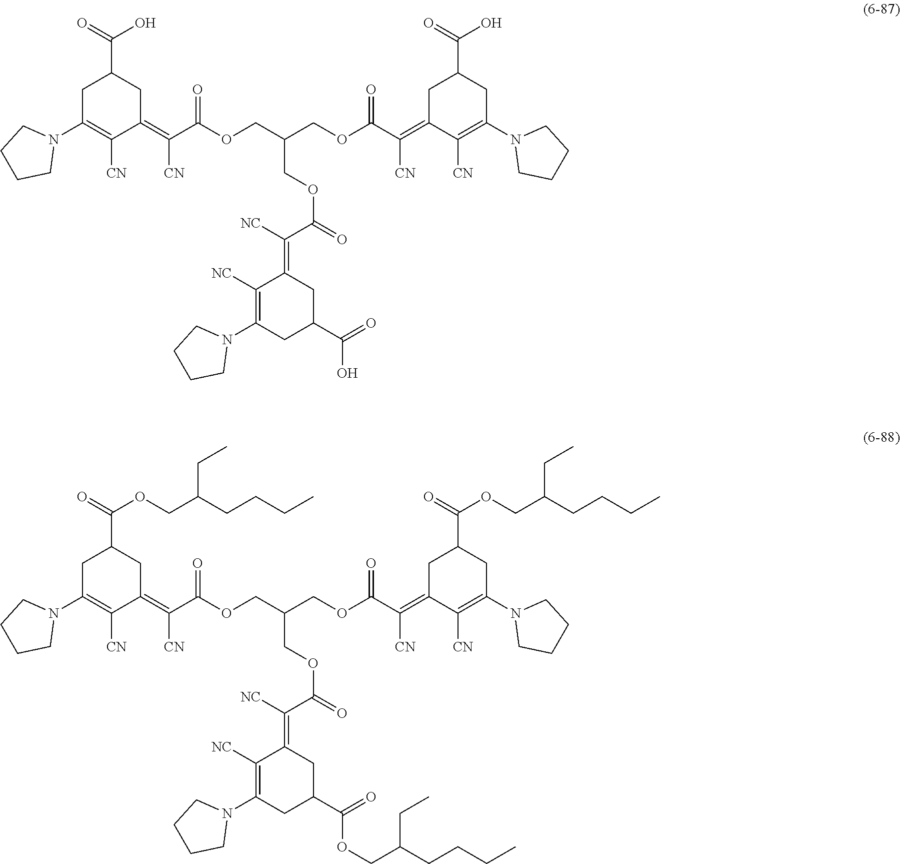 [SMILES: N#CC1=C(N2CCCC2)CC(C(=O)O)C/C1=C(/C#N)C(=O)OCC(COC(=O)/C(C#N)=C1\CC(C(=O)O)CC(N2CCCC2)=C1C#N)COC(=O)/C(C#N)=C1\CC(C(=O)O)CC(N2CCCC2)=C1C#N ; CCCCC(CC)COC(=O)C1CC(N2CCCC2)=C(C#N)/C(=C(\C#N)C(=O)OCC(COC(=O)/C(C#N)=C2\CC(C(=O)OCC(CC)CCCC)CC(N3CCCC3)=C2C#N)COC(=O)/C(C#N)=C2\CC(C(=O)OCC(CC)CCCC)CC(N3CCCC3)=C2C#N)C1]

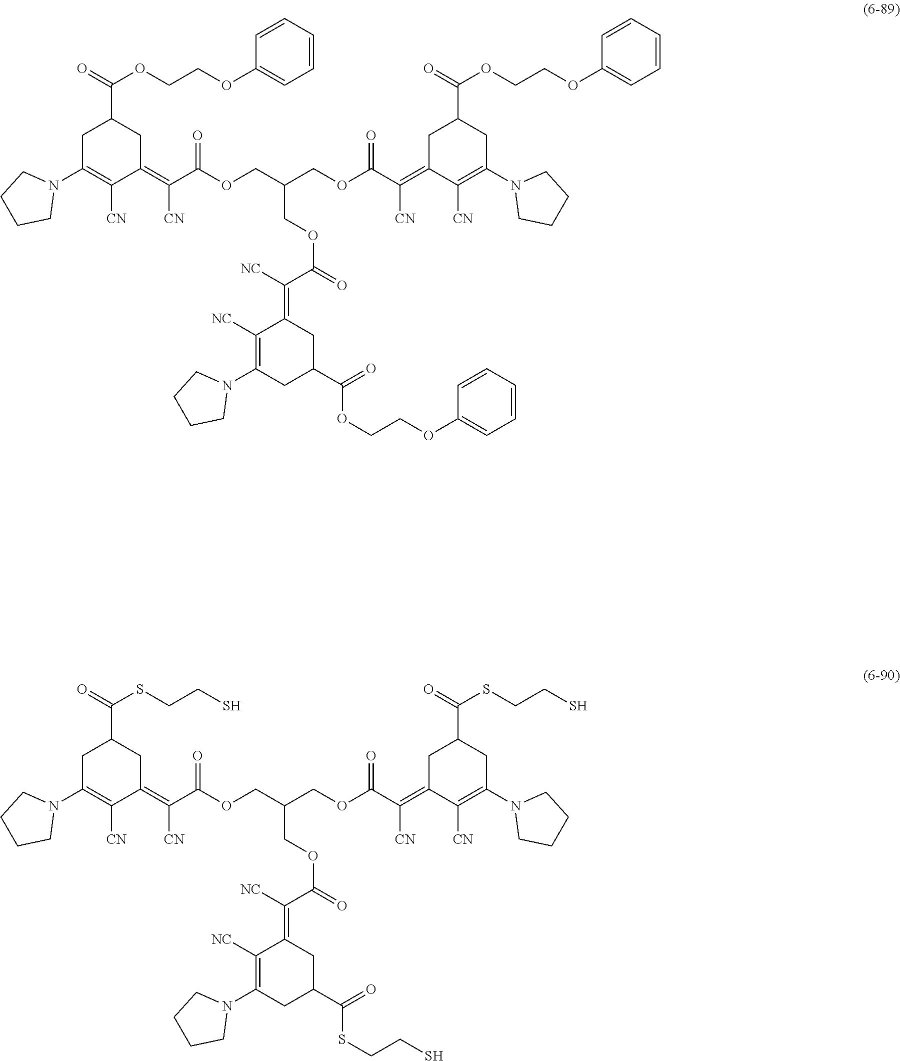 [SMILES: N#CC1=C(N2CCCC2)CC(C(=O)OCCOc2ccccc2)C/C1=C(/C#N)C(=O)OCC(COC(=O)/C(C#N)=C1\CC(C(=O)OCCOc2ccccc2)CC(N2CCCC2)=C1C#N)COC(=O)/C(C#N)=C1\CC(C(=O)OCCOc2ccccc2)CC(N2CCCC2)=C1C#N ; N#CC1=C(N2CCCC2)CC(C(=O)SCCS)C/C1=C(/C#N)C(=O)OCC(COC(=O)/C(C#N)=C1\CC(C(=O)SCCS)CC(N2CCCC2)=C1C#N)COC(=O)/C(C#N)=C1\CC(C(=O)SCCS)CC(N2CCCC2)=C1C#N]

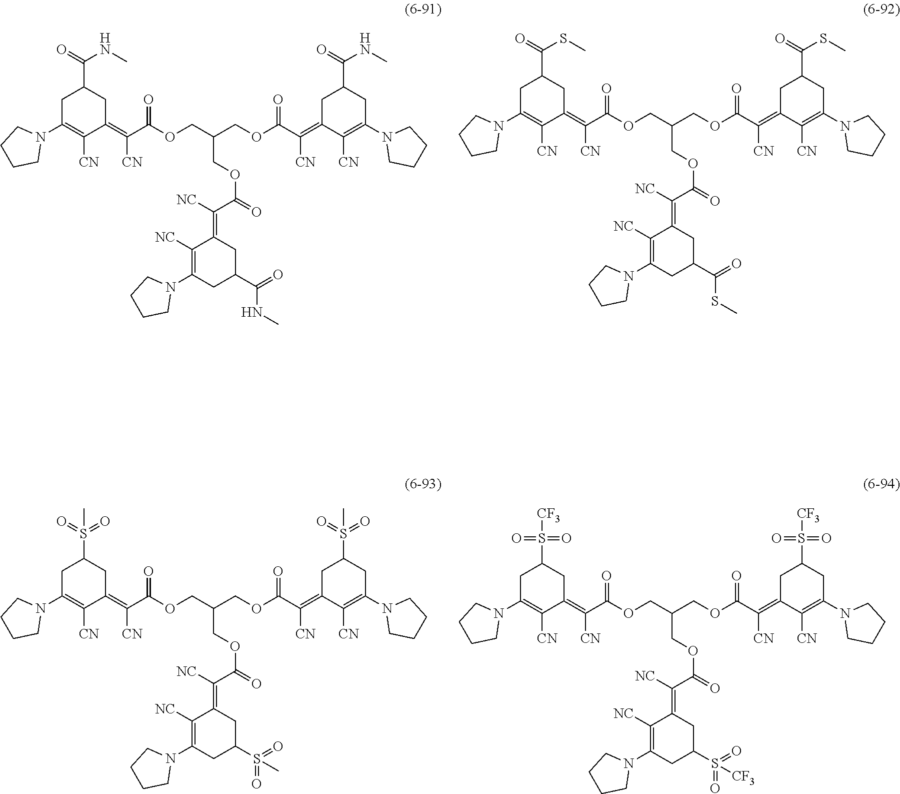

Examples of the compound represented by Formula (VII) (hereinafter, may be referred to as a compound (VII)) include the following compounds.

The compound (VII) is preferably compounds represented by Formula (7-1) to Formula (7-9), Formula (7-12), Formula (7-14), Formula (7-17), Formula (7-42) to Formula (7-44), or Formula (7-57), and more preferably a compound represented by Formula (7-1) to Formula (7-8).

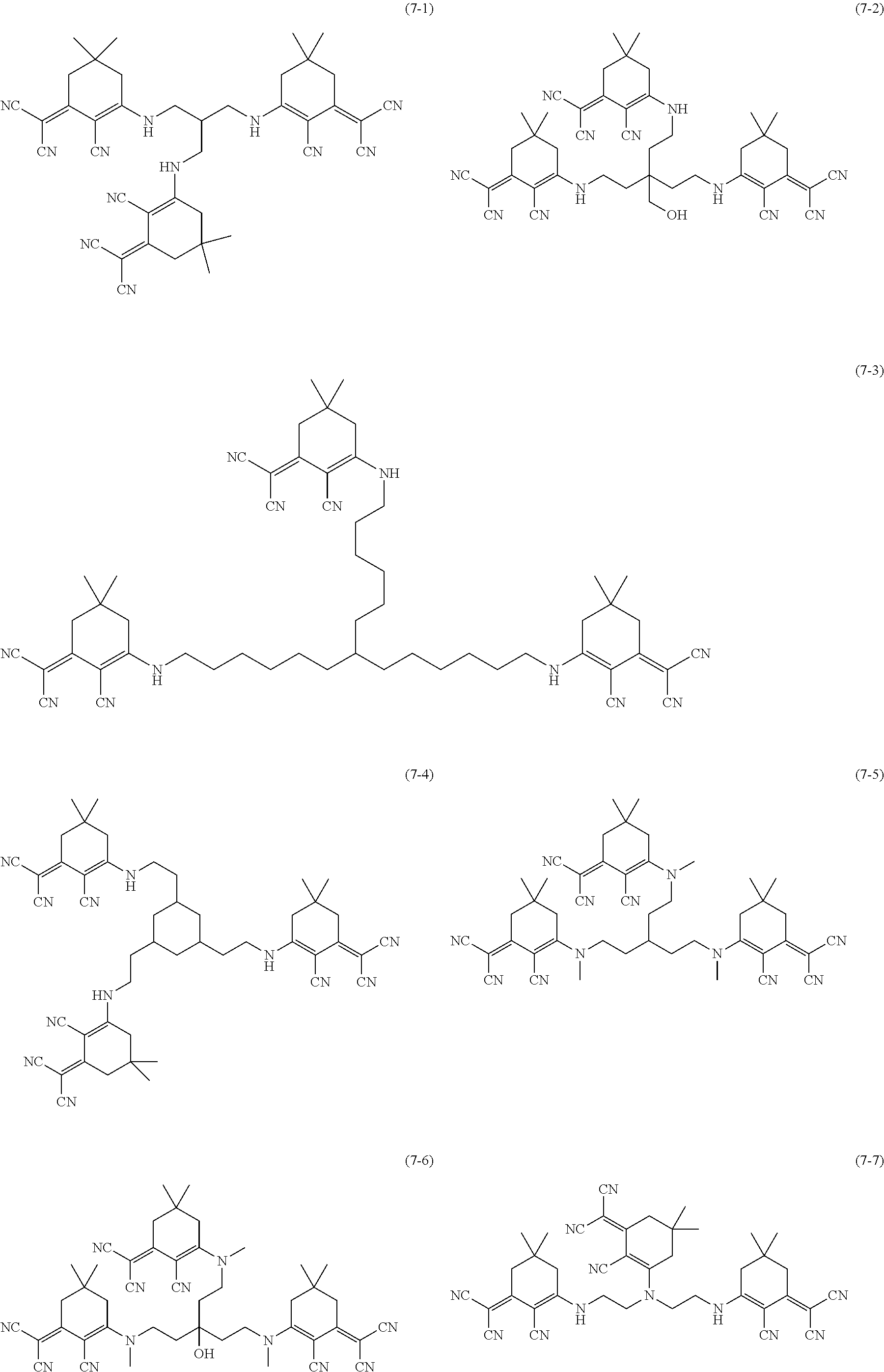

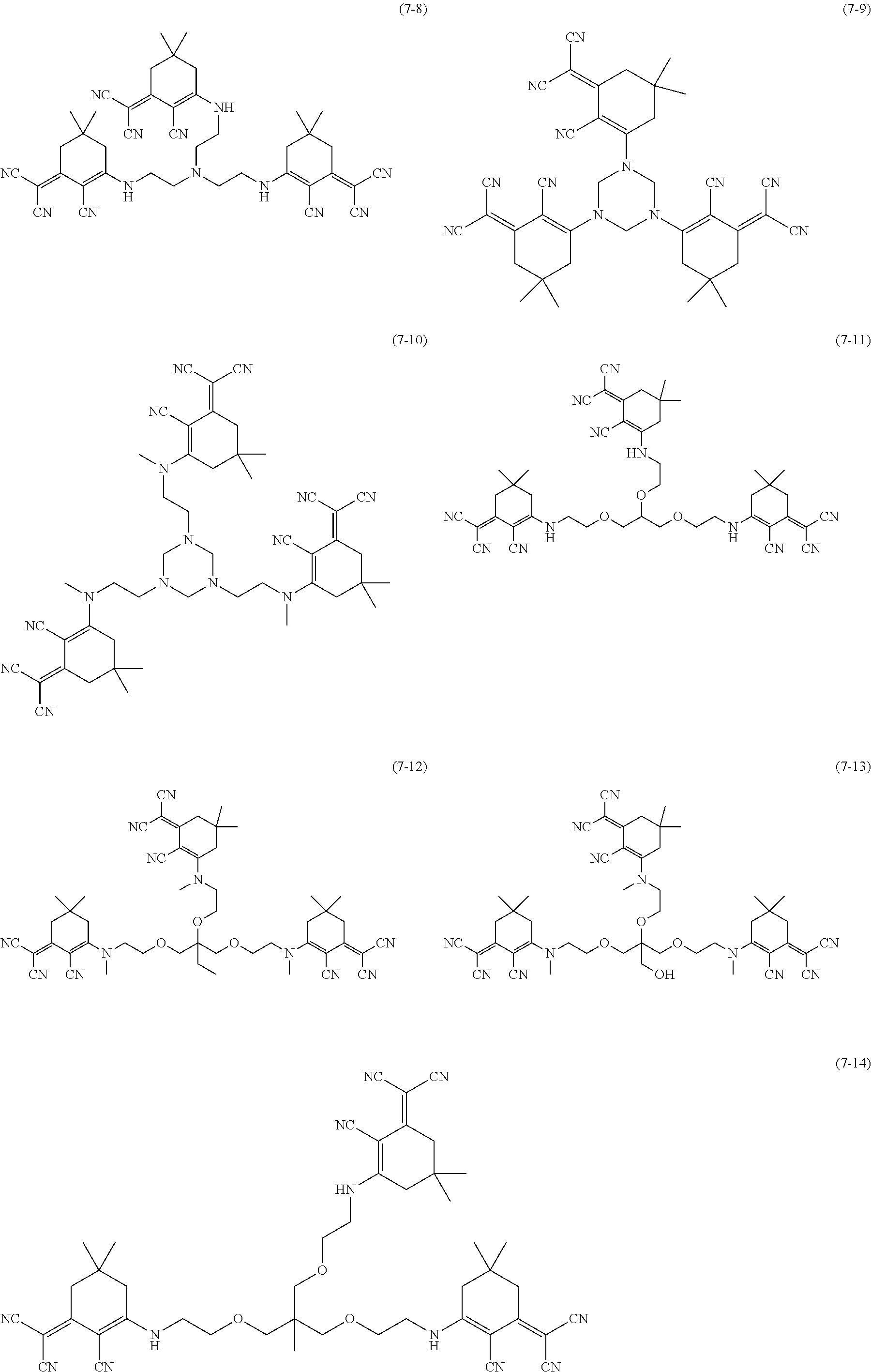

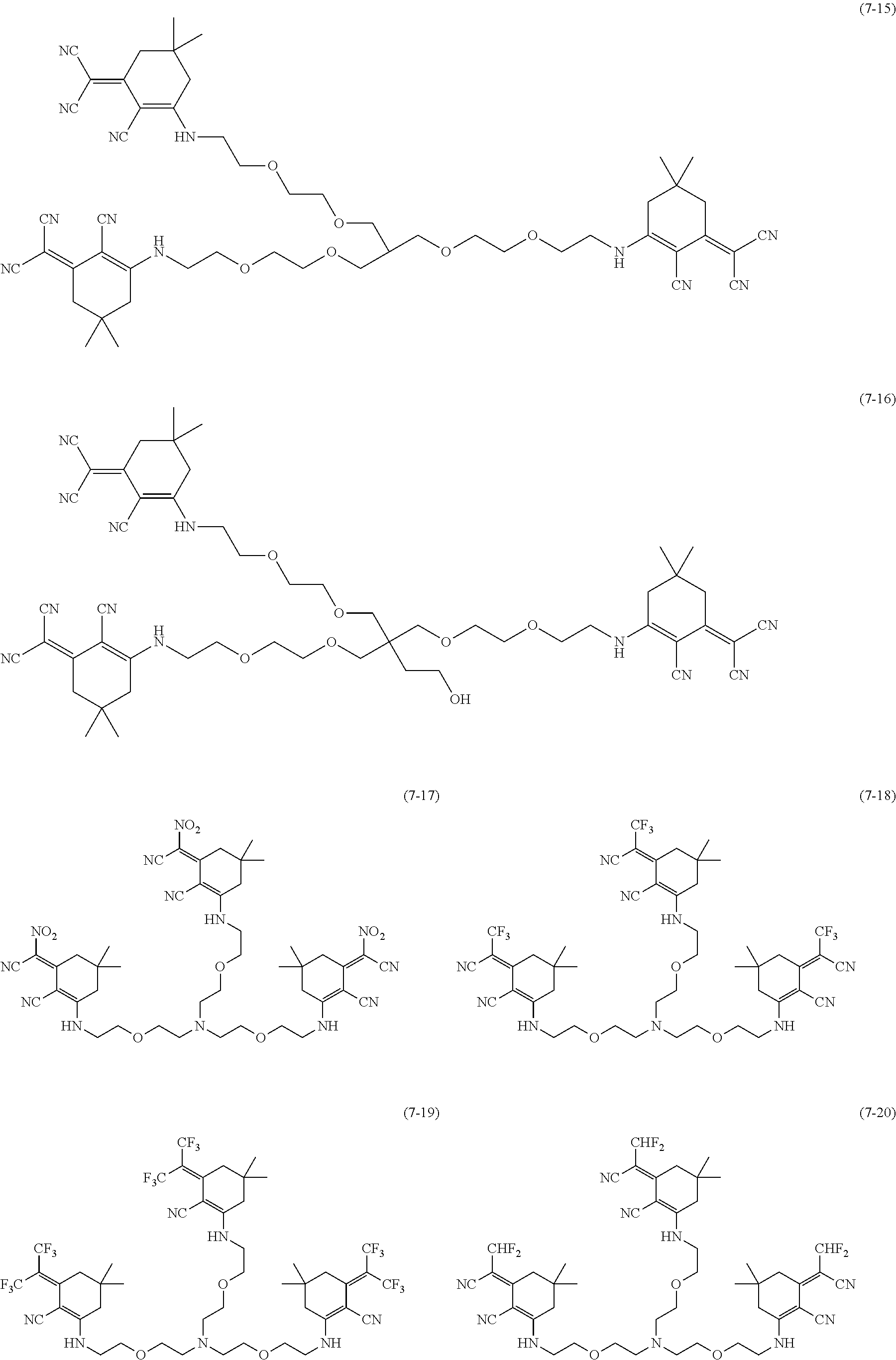

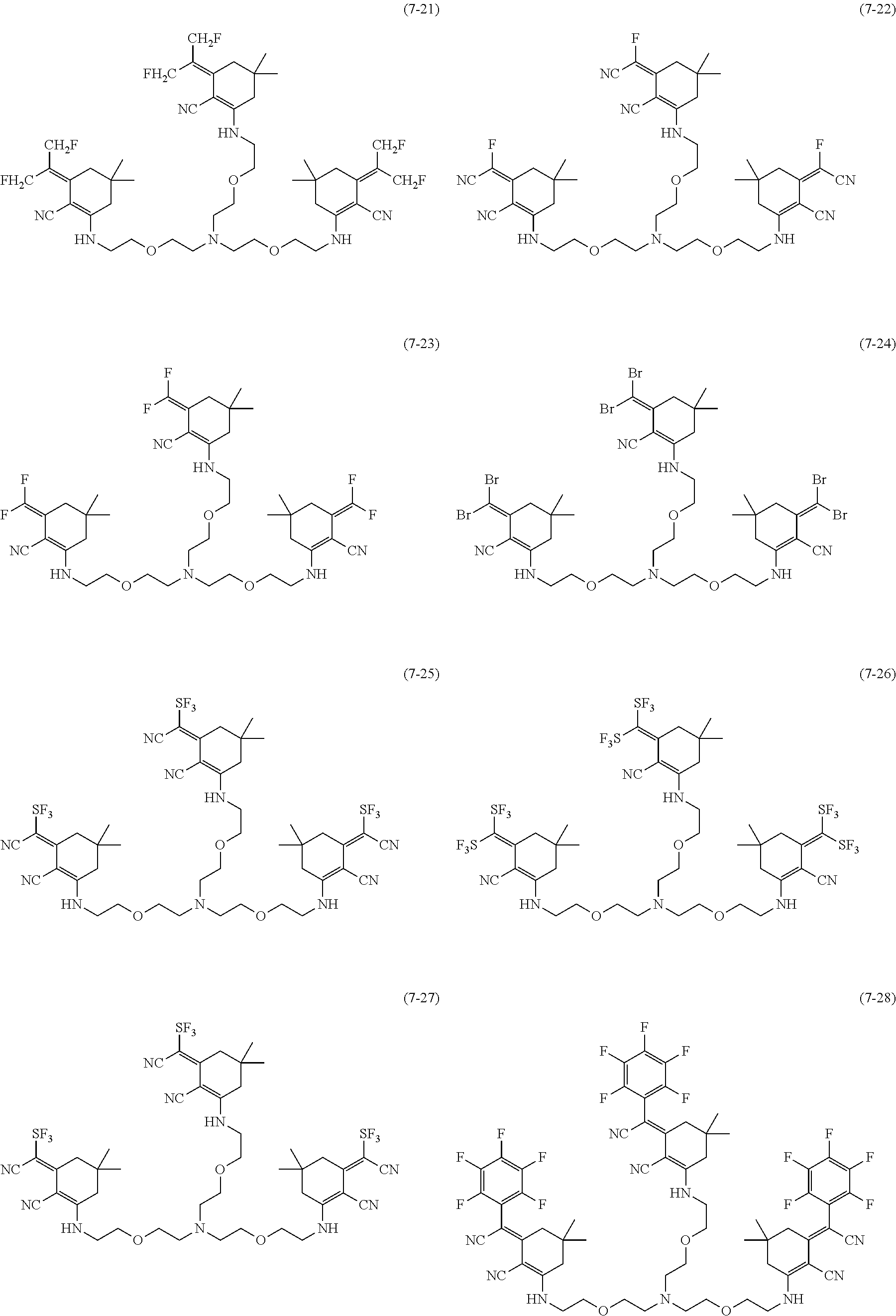

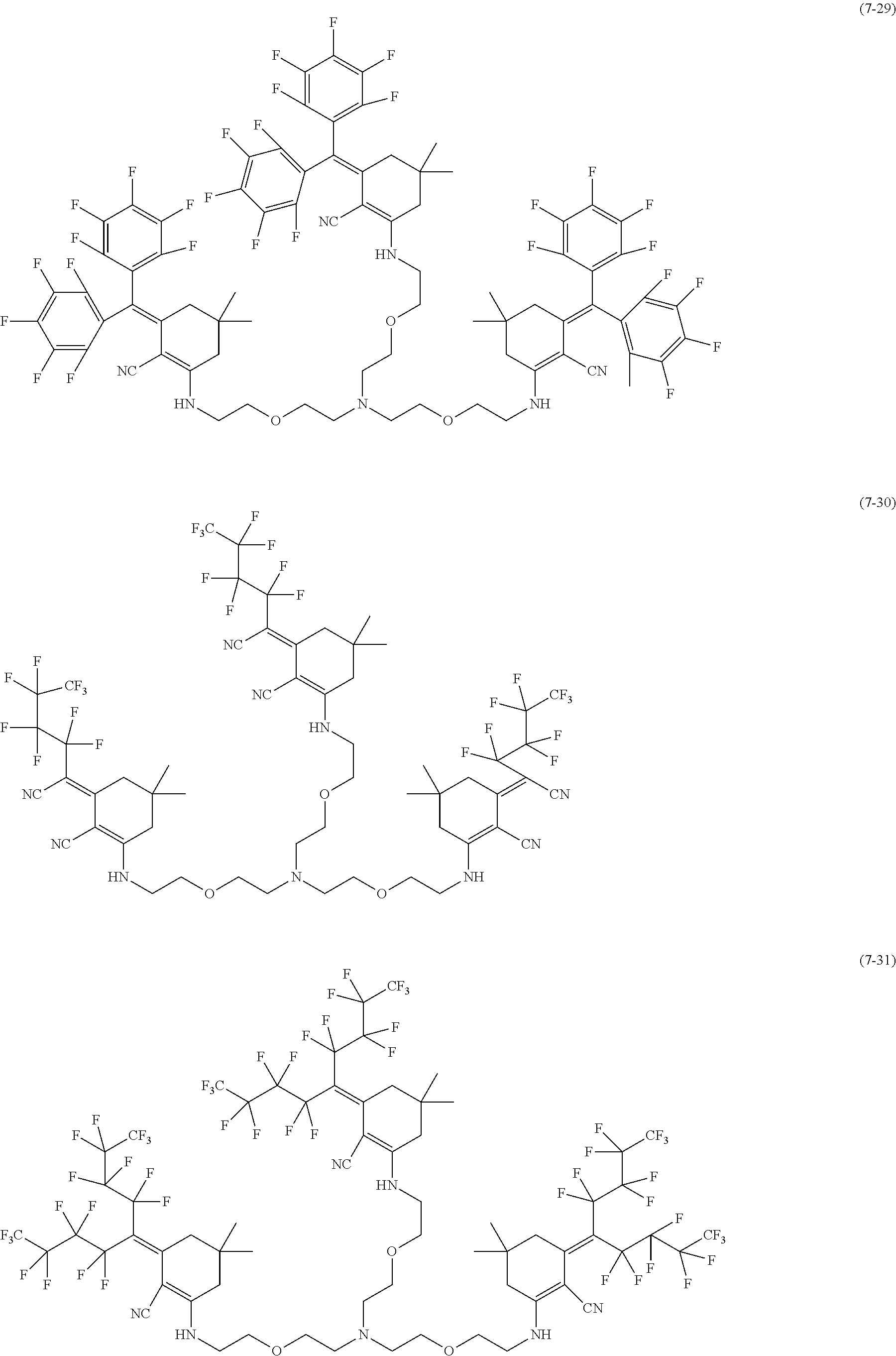

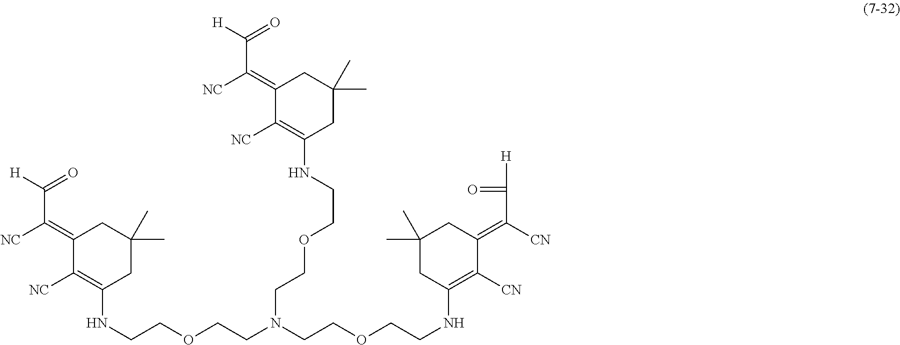 [SMILES: CC1(C)CC(NCCOCCN(CCOCCNC2=C(C#N)/C(=C(\C#N)C=O)CC(C)(C)C2)CCOCCNC2=C(C#N)/C(=C(\C#N)C=O)CC(C)(C)C2)=C(C#N)/C(=C(\C#N)C=O)C1]

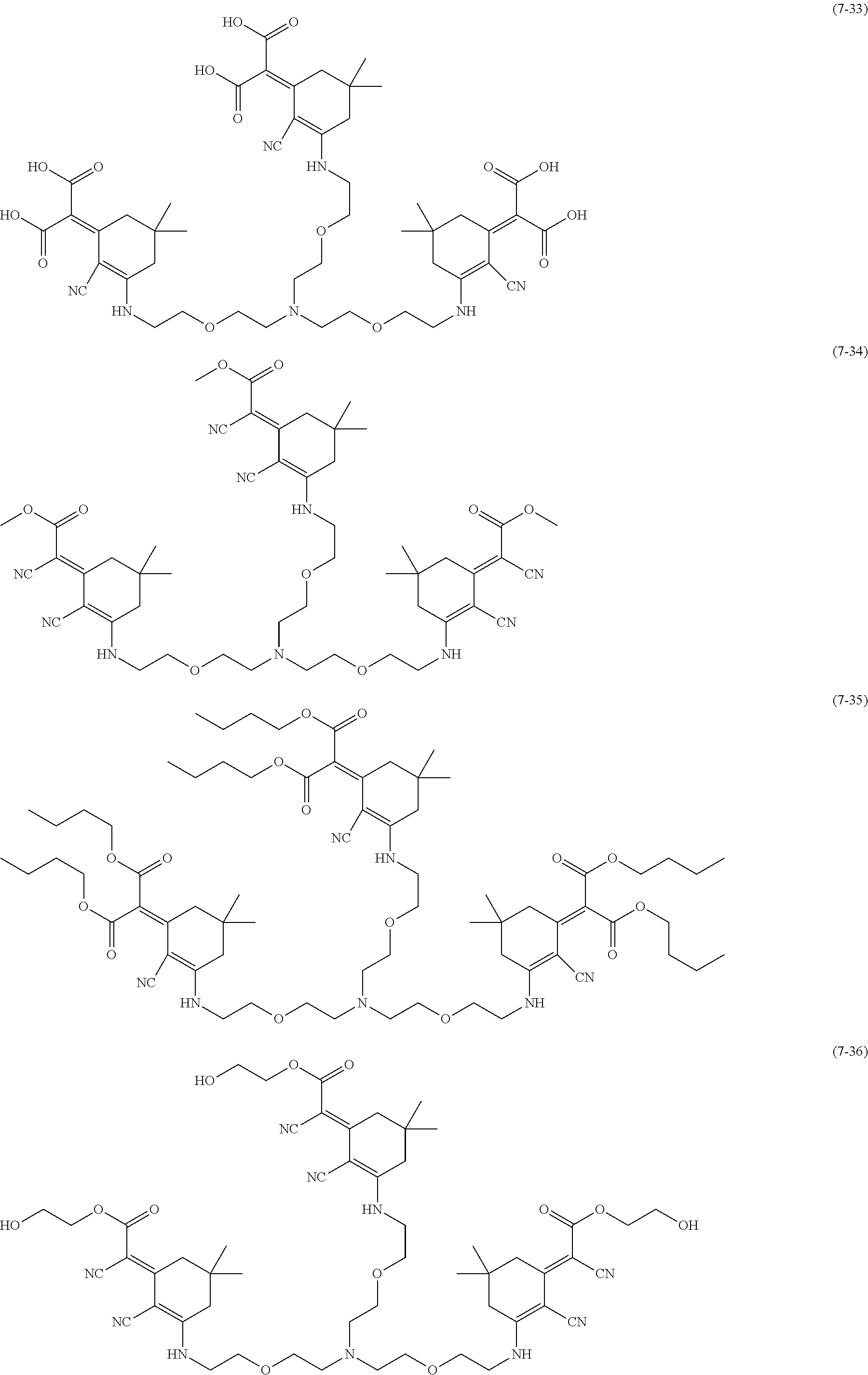

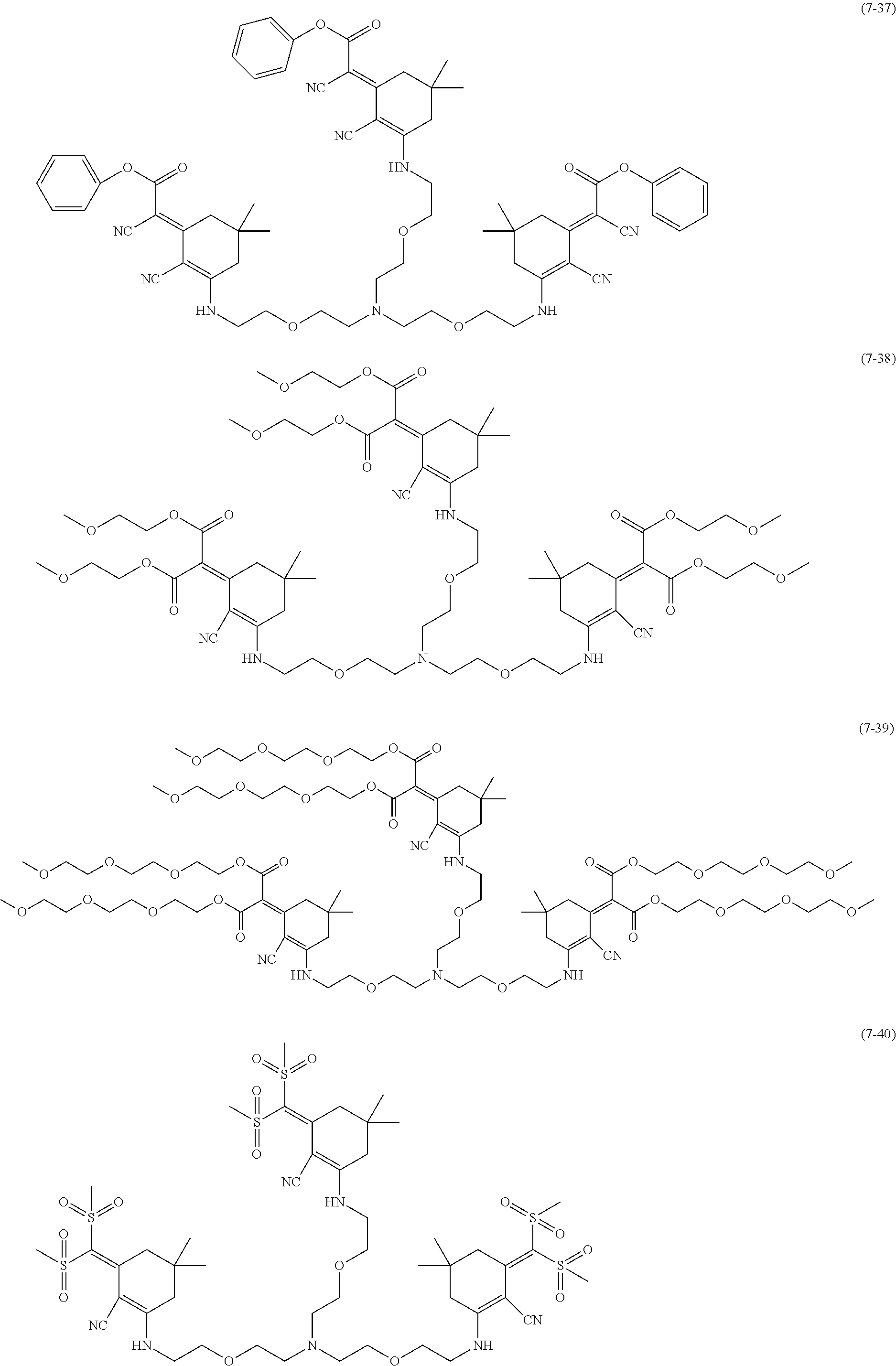

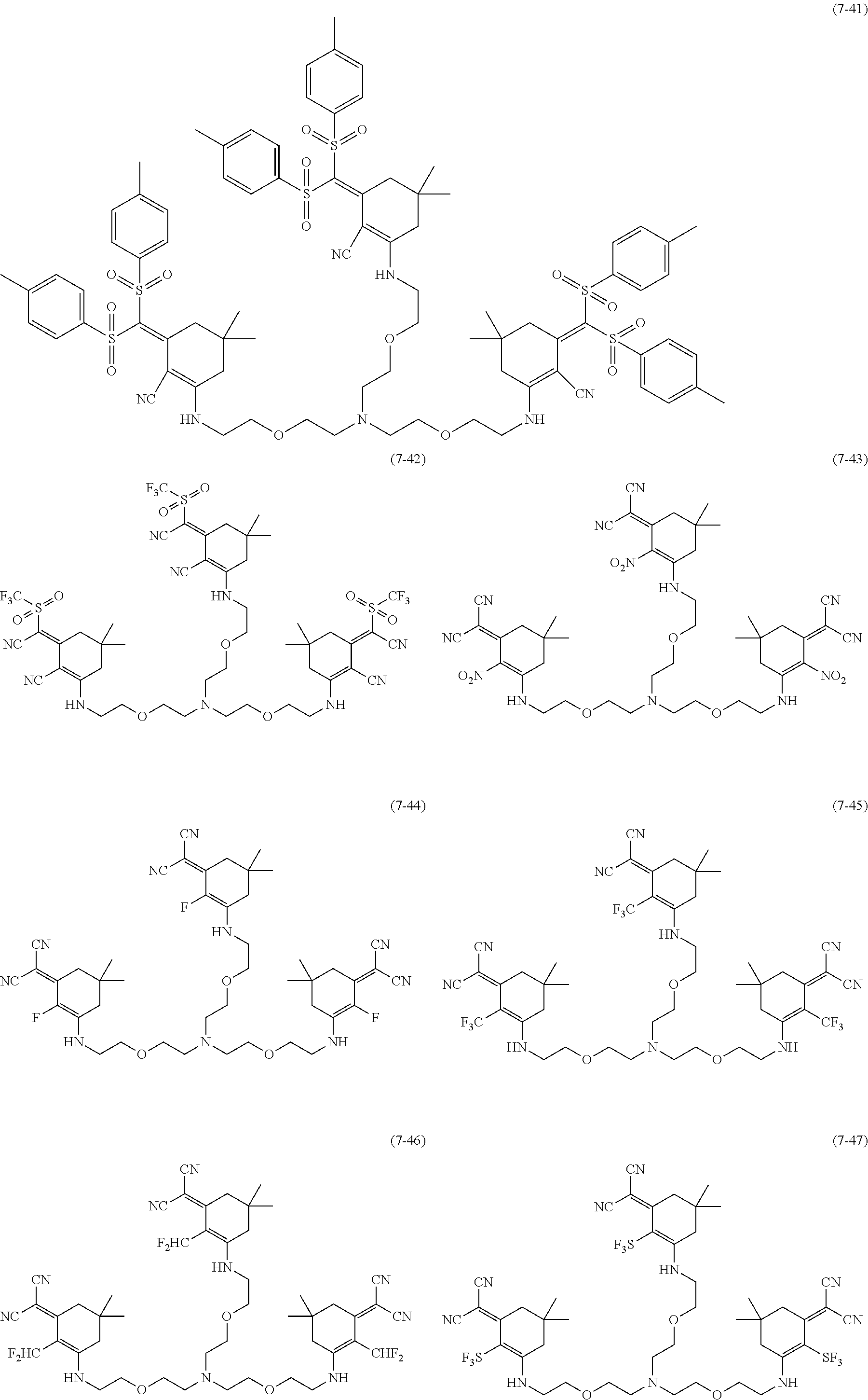

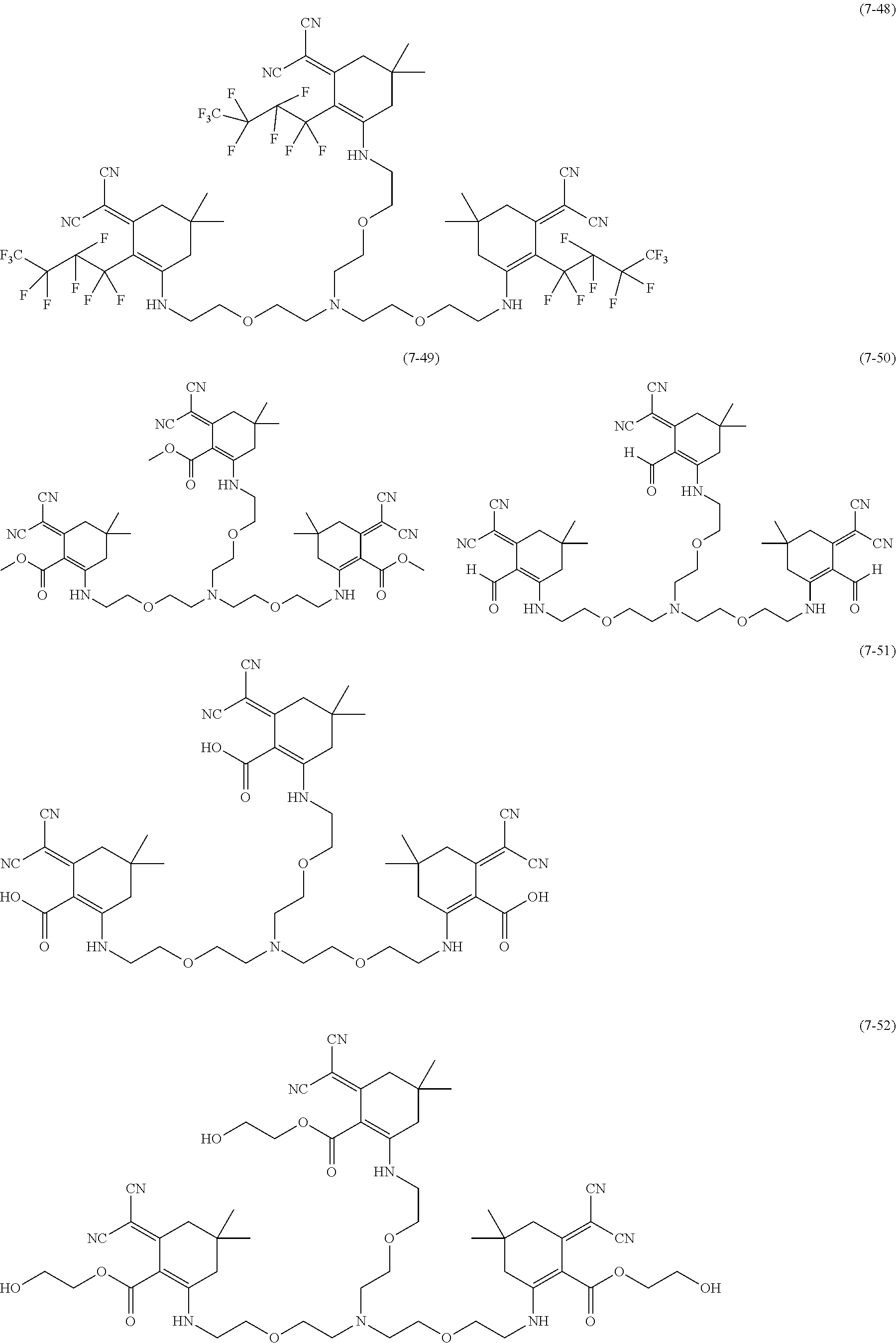

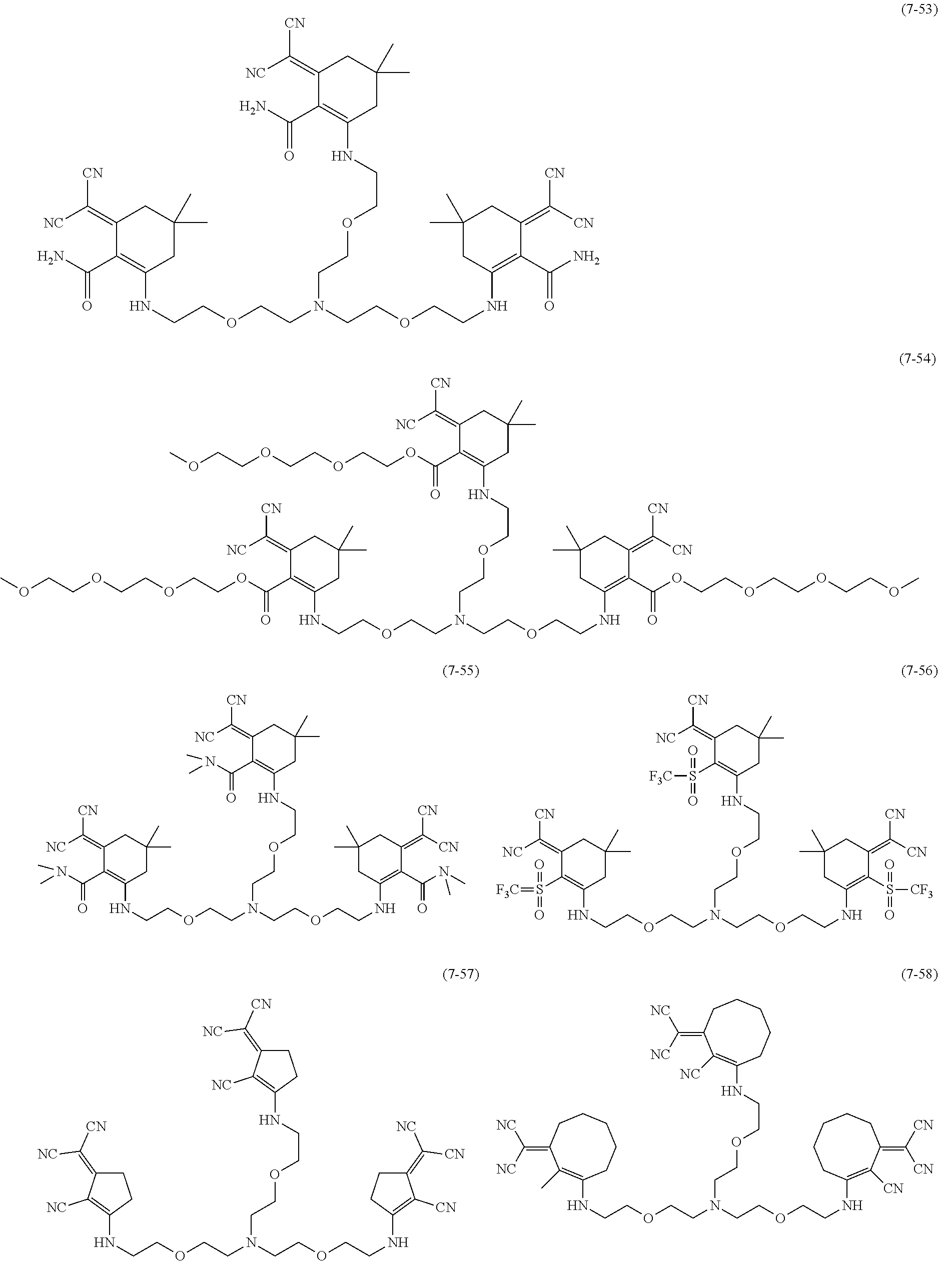

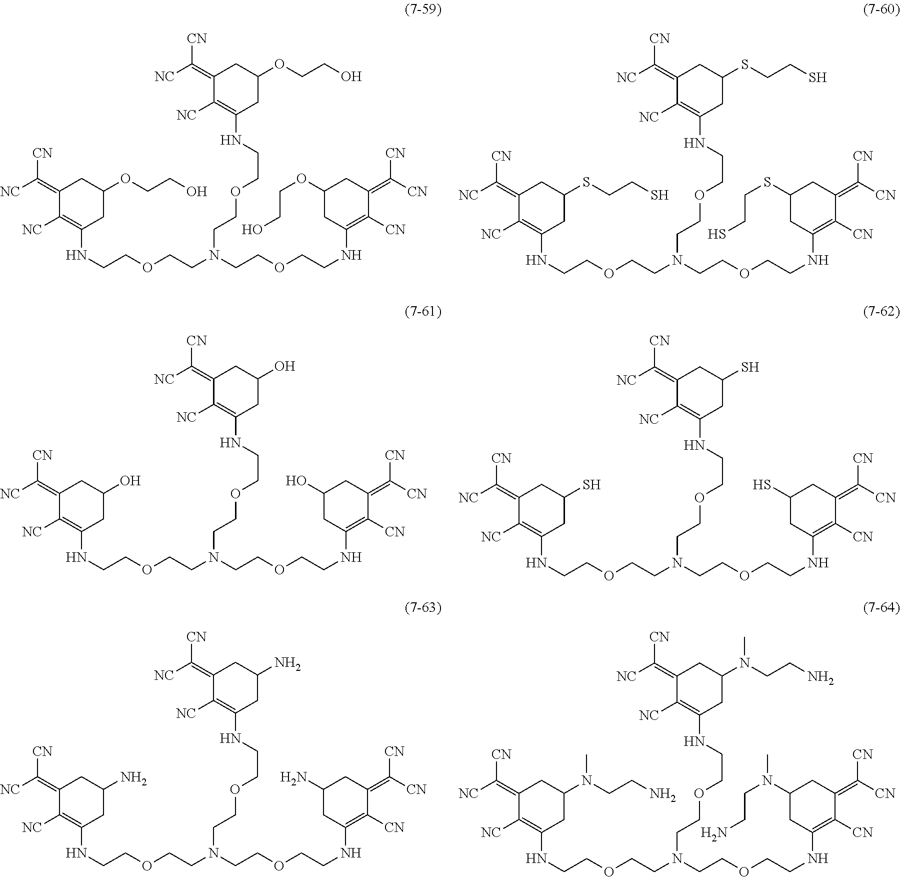

Examples of the compound represented by Formula (VIII) (hereinafter, may be referred to as a compound (VIII)) include the following compounds.

The compound (VIII) is preferably compounds represented by Formula (8-1), Formula (8-2), Formula (8-4), Formula (8-5), Formula (8-11), Formula (8-13) to Formula (8-17), Formula (8-25), Formula (8-26), Formula (8-47), and Formula (8-48), and more preferably a compound represented by Formula (8-1), Formula (8-4), Formula (8-5), Formula (8-15), Formula (8-17), and Formula (8-25).

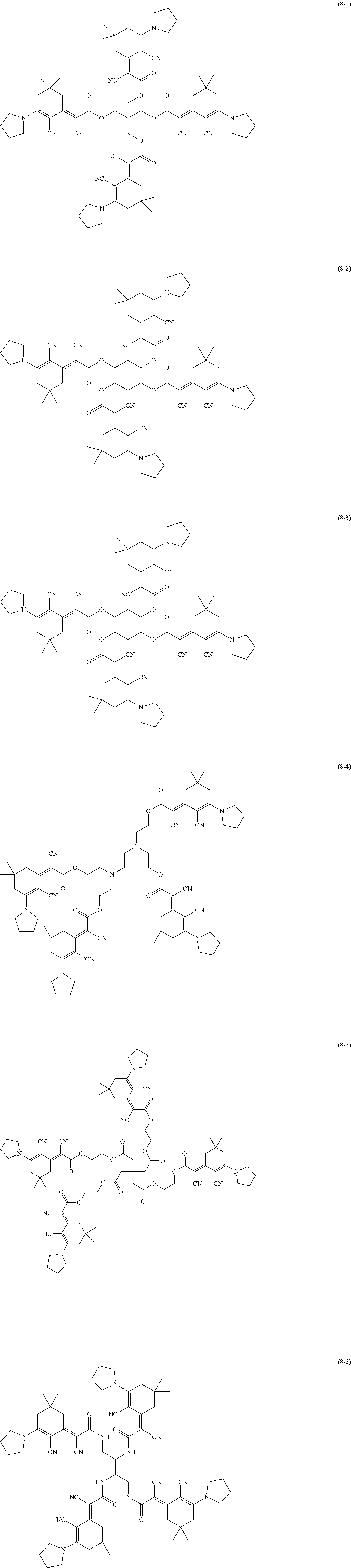

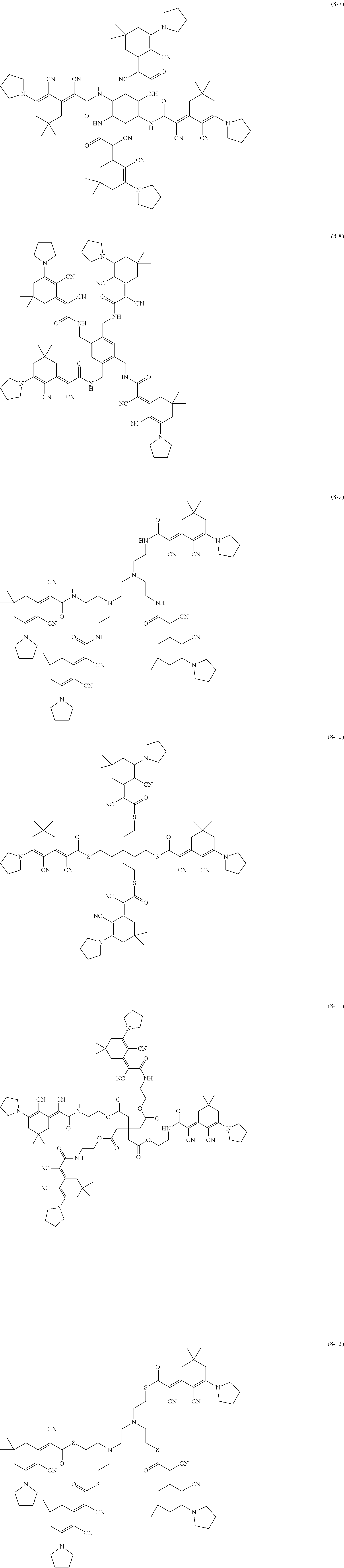

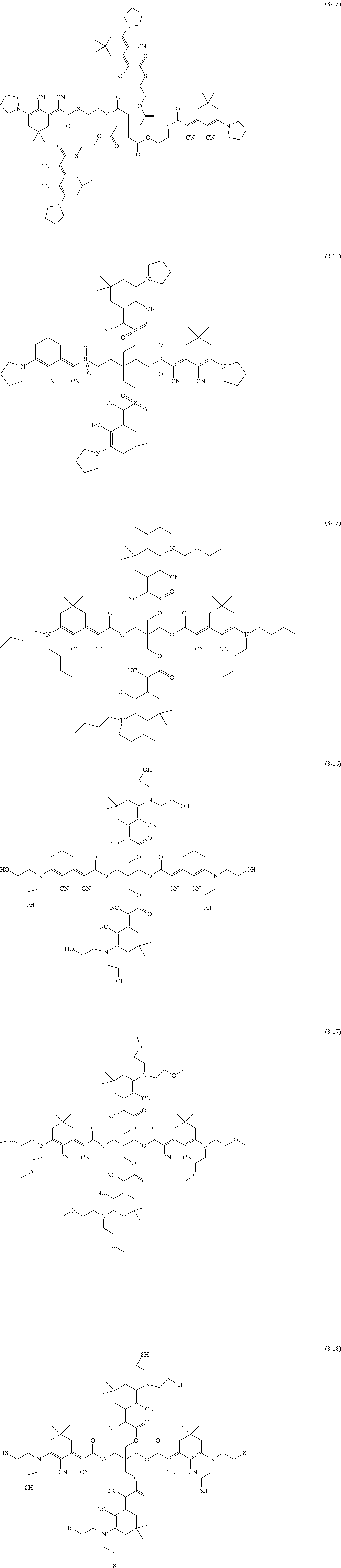

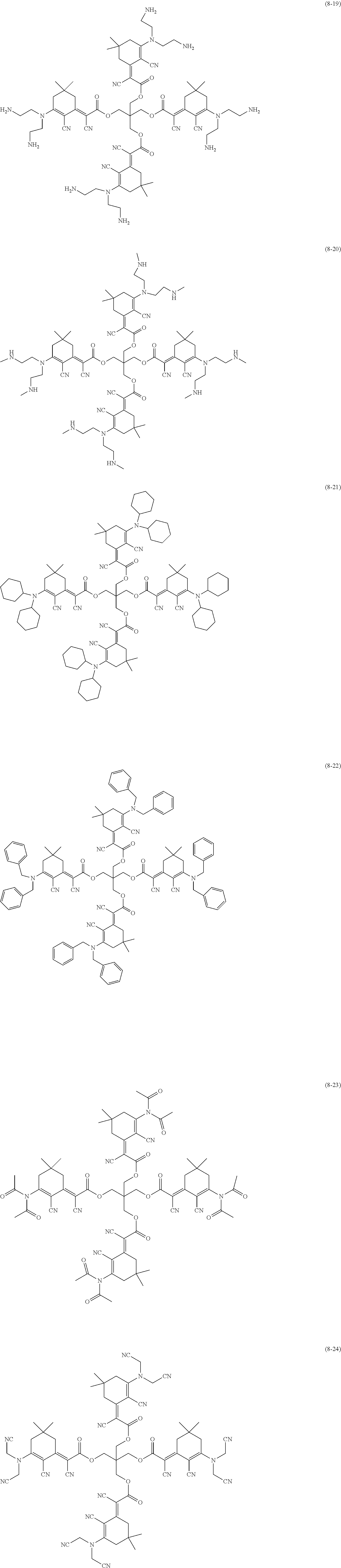

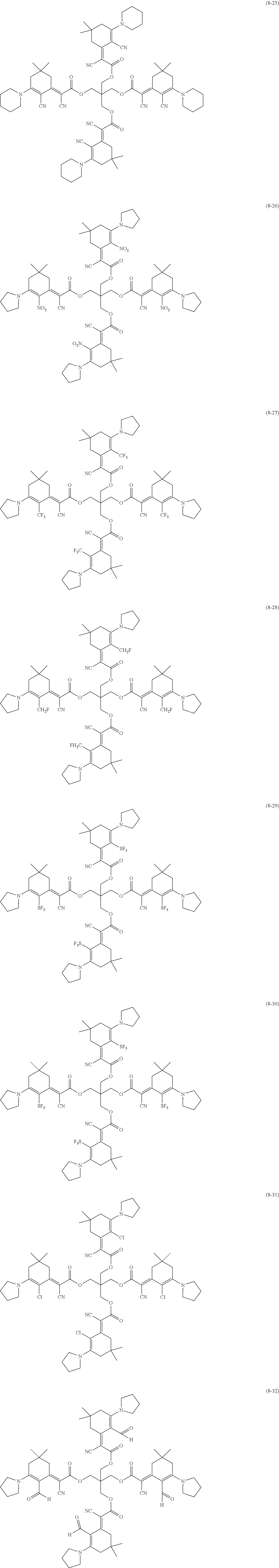

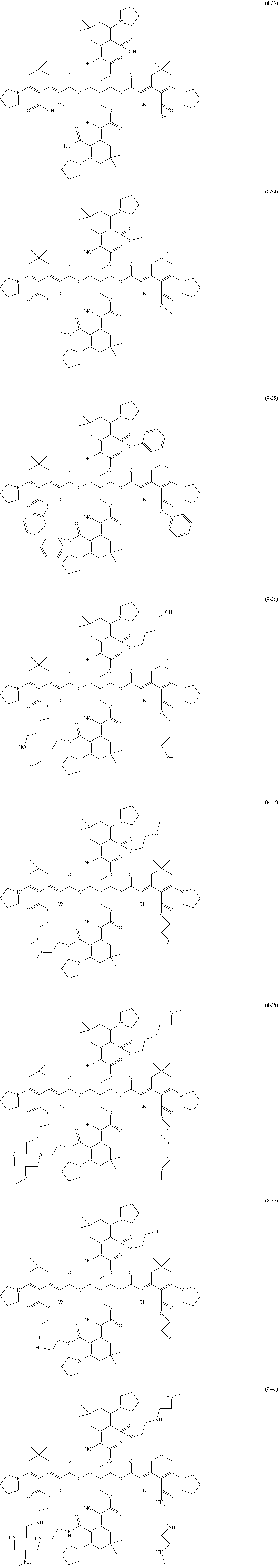

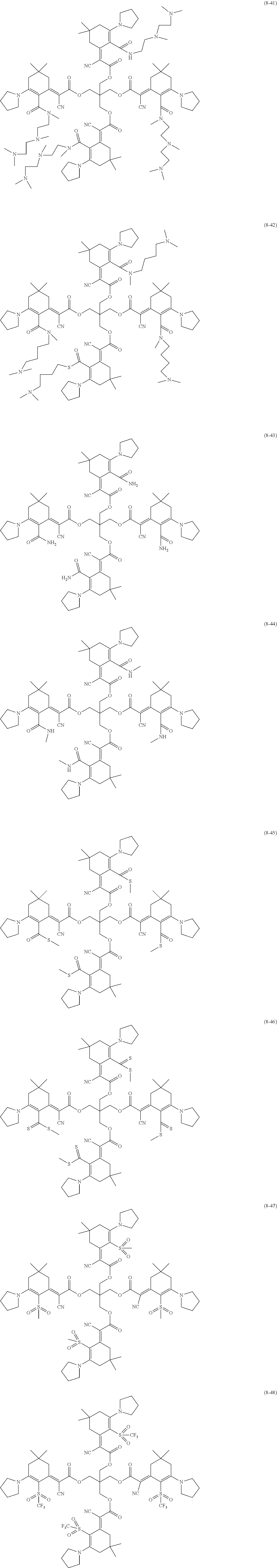

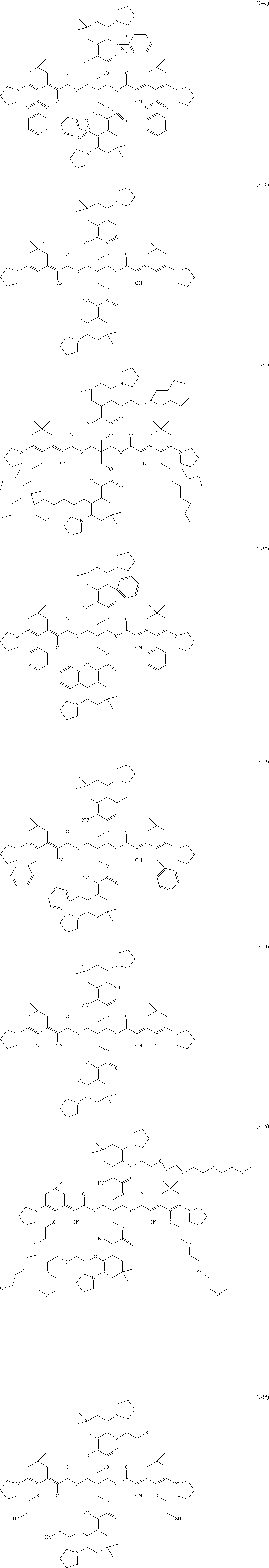

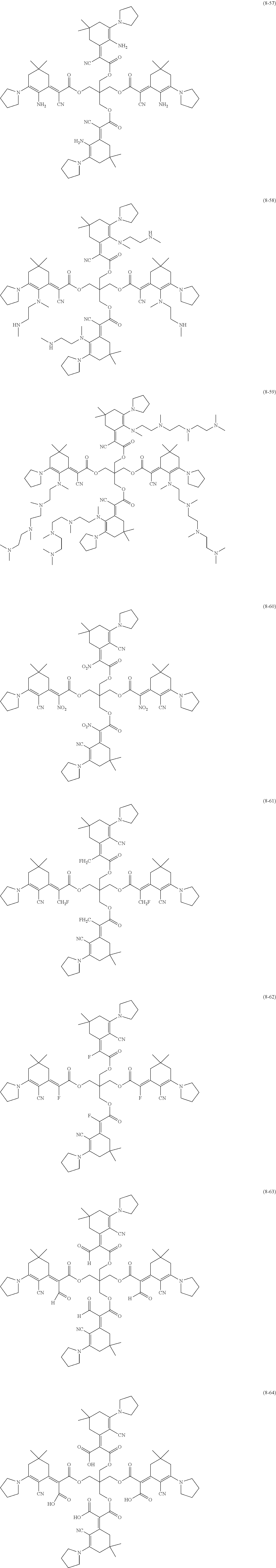

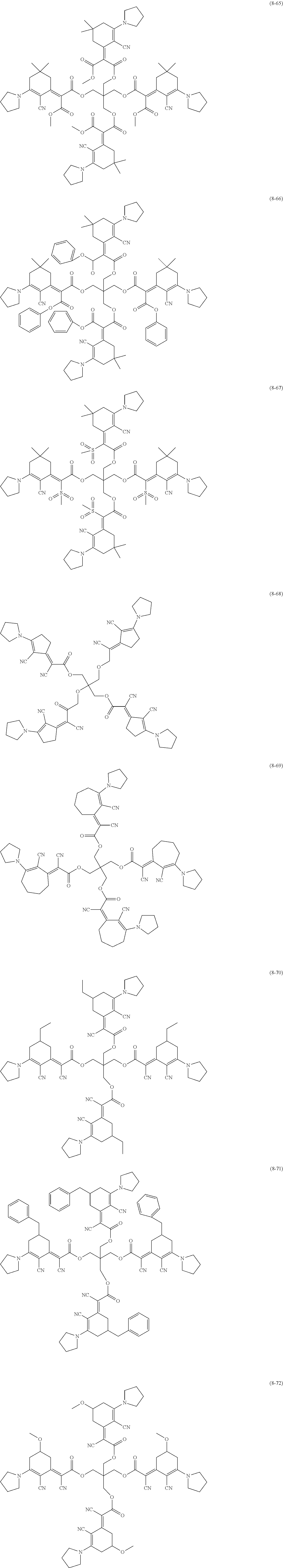

<Method for producing compound (I)>

The compound (I) can be obtained, for example, by reacting a compound represented by Formula (I-1) (hereinafter, may be referred to as a compound (I-1)) with a compound represented by Formula (I-2) (hereinafter, may be referred to as a compound (I-2)).

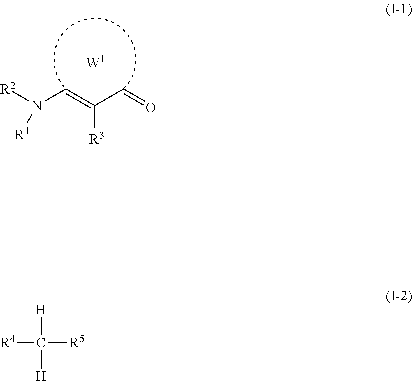

[in the formula, the ring W¹ and R¹ to R⁵ have the same meaning as described above].

The reaction between the compound (I-1) and the compound (I-2) is usually performed by mixing the compound (I-1) and the compound (I-2), and it is preferable to add the compound (I-2) to the compound (I-1).

In addition, as for the reaction between the compound (I-1) and the compound (I-2), it is preferable to mix the compound (I-1) and the compound (I-2) in the presence of a base and a methylating agent,

it is preferable to mix the compound (1-1), the compound (I-2), a base, and a methylating agent,

it is more preferable to mix the compound (I-2) and a base in a mixture of the compound (1-1) and a methylating agent, and

it is still more preferable to add a mixture of the compound (I-2) and a base to the mixture of the compound (1-1) and the methylating agent.

Examples of the base include metal hydroxides (preferably alkali metal hydroxides) such as sodium hydroxide, lithium hydroxide, potassium hydroxide, cesium hydroxide, rubidium hydroxide, calcium hydroxide, barium hydroxide, and magnesium hydroxide; metal alkoxides (preferably alkali metal alkoxides) such as sodium methoxide, potassium methoxide, lithium methoxide, sodium ethoxide, sodium isopropoxide, sodium tertiary butoxide, and potassium tertiary butoxide; metal hydrides such as lithium hydride, sodium hydride, potassium hydride, lithium aluminum hydride, sodium borohydride, aluminum hydride, and sodium aluminum hydride; metal oxides such as calcium oxide and magnesium oxide; metal carbonates (preferably alkaline earth metal carbonates) such as sodium hydrogen carbonate, sodium carbonate, and potassium carbonate; organic alkyl metal compounds such as n-butyl lithium, tertiary butyl lithium, methyl lithium, and Grignard reagent; amine compounds (preferably tertiary amines such as triethylamine and diisopropylethylamine) such as ammonia, triethylamine, diisopropylethylamine, ethanolamine, pyrrolidine, piperidine, diazabicycloundecene, diazabicyclononene, guanidine, tetramethylammonium hydroxide, tetraethylammonium hydroxide, pyridine, aniline, dimethoxyaniline, ammonium acetate, and R-alanine; metal amide compounds (preferably alkali metal amides) such as lithium diisopropylamide, sodium amide, and potassium hexamethyldisilazide; sulfonium compounds such as trimethylsulfonium hydroxide; iodonium compounds such as diphenyliodonium hydroxide; and phosphazene bases.

The use amount of the base is usually 0.1 to 5 mol and preferably 0.5 to 2 mol, with respect to 1 mol of the compound (I-1).

Examples of the methylating agent include iodomethane, dimethyl sulfate, methyl methanesulfonate, methyl fluorosulfonate, methyl p-toluenesulfonate, methyl trifluoromethanesulfonate, and trimethyloxonium tetrafluoroborate.

The use amount of the methylating agent is usually 0.1 to 5 mol and preferably 0.5 to 2 mol, with respect to 1 mol of the compound (I-1).

The reaction between the compound (I-1) and the compound (I-2) may be performed in the presence of a solvent. Examples of the solvent include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Acetonitrile, tetrahydrofuran, chloroform, dichloromethane, and diethyl ether are preferable, acetonitrile, tetrahydrofuran, and chloroform are more preferable, and acetonitrile is still more preferable.

The solvent is preferably a dehydrated solvent.

The reaction time between the compound (I-1) and the compound (I-2) is usually 0.1 to 10 hours, and preferably 0.2 to 3 hours.

The reaction temperature between the compound (I-1) and the compound (I-2) is usually −50 to 150° C., and preferably −20 to 100° C.

The use amount of the compound (I-2) is usually 0.1 to 10 mol, and preferably 0.5 to 5 mol, with respect to 1 mol of the compound (I-1).

Examples of the compound (I-1) include the compounds described below.

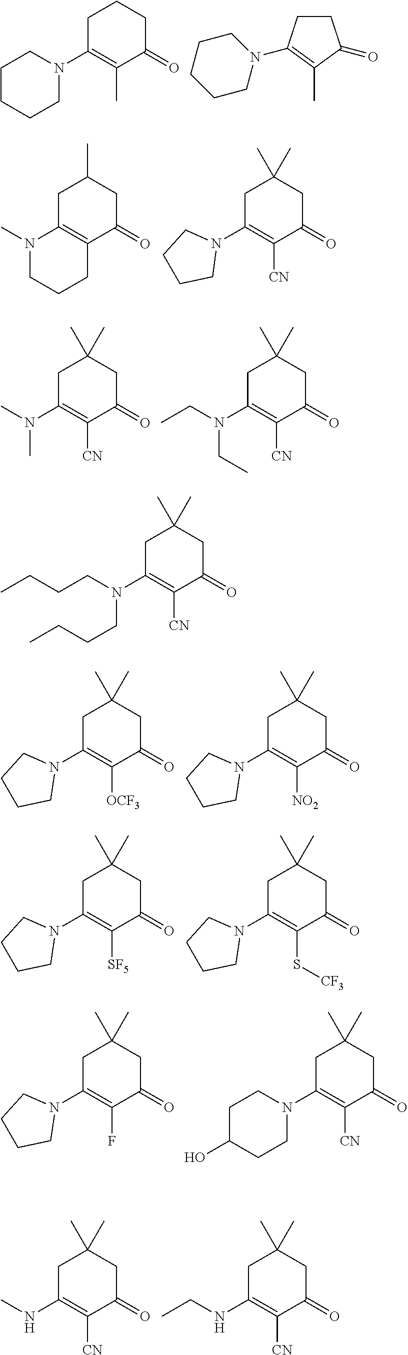

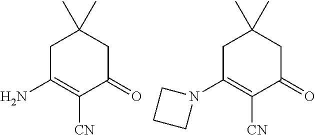

As the compound (I-2), a commercially available product may be used, and examples thereof include the compounds described below.

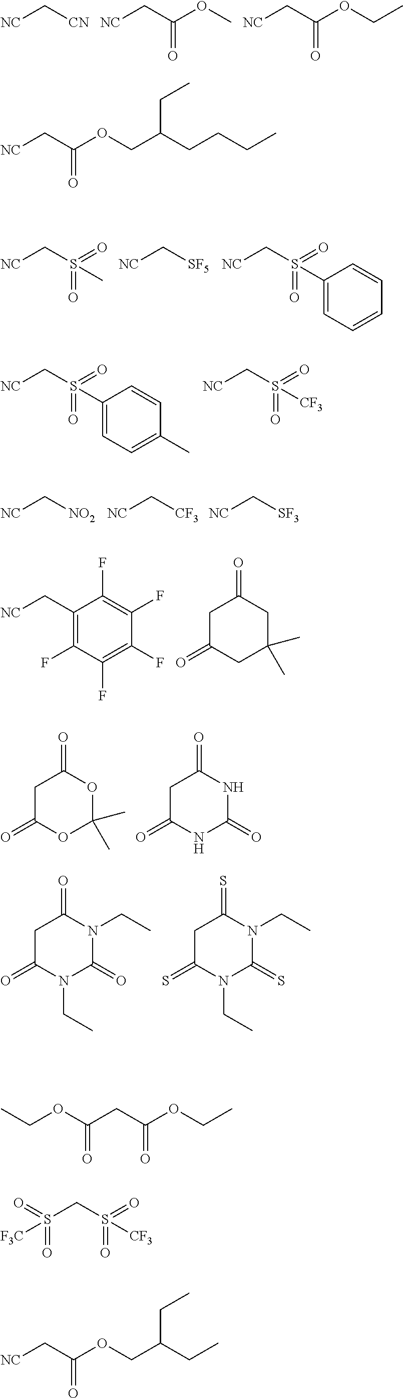

The compound (I-1) can be obtained, for example, by reacting a compound represented by Formula (I-3) (hereinafter, may be referred to as a compound (I-3)) with a compound represented by Formula (I-4) (hereinafter, may be referred to as a compound (I-4)).

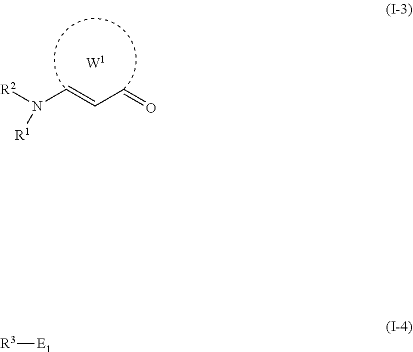

[in Formula (I-3), the ring W¹, R¹, R², and R³ have the same meaning as described above; and E₁ represents a leaving group].

Examples of the leaving group represented by E₁ include a halogen atom, a p-toluenesulfonyl group, and a trifluoromethylsulfonyl group.

The reaction between the compound (I-3) and the compound (I-4) is performed by mixing the compound (I-3) and the compound (I-4).

The use amount of the compound (I-4) is usually 0.1 to 5 mol, and preferably 0.5 to 2 mol, with respect to 1 mol of the compound (I-3).

The reaction between the compound (I-3) and the compound (I-4) may be performed in the presence of a solvent. Examples of the solvent include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Acetonitrile, tetrahydrofuran, chloroform, dichloromethane, and diethyl ether are preferable, acetonitrile, tetrahydrofuran, and chloroform are more preferable, and methanol, ethanol, isopropanol, and acetonitrile are still more preferable.

The reaction time between the compound (I-3) and the compound (I-4) is usually 0.1 to 10 hours.

The reaction temperature between the compound (I-3) and the compound (I-4) is usually −50 to 150° C.

Examples of the compound (I-3) include the compounds described below.

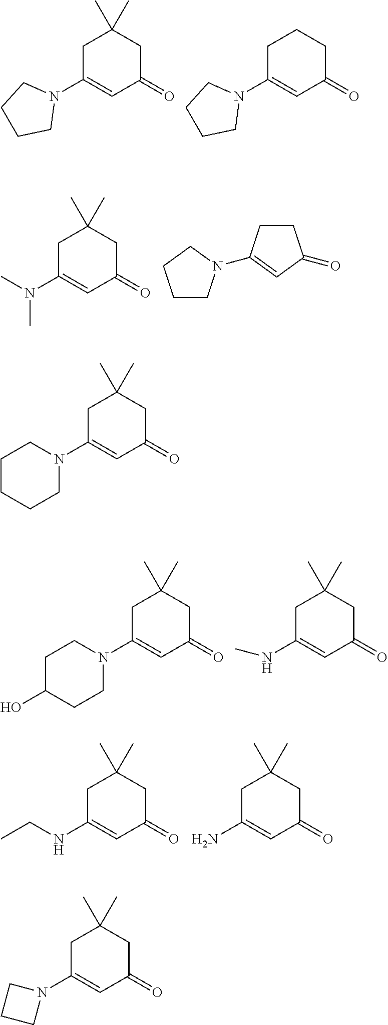

As the compound (I-4), a commercially available product may be used. Examples thereof include chlorocyanine, bromocyanine, p-toluenesulfonylcyanide, trifluoromethanesulfonylcyanide, 1-chloromethyl-4 fluoro-1,4-diazoniabicyclo [2.2.2] octane bis(tetrafluoroborate (also referred to as selected fluoro (registered trademark of Air Products and Chemicals)), benzoyl (phenyliodonio) (trifluoromethanesulfonyl) methanide, 2,8-difluoro-5-(trifluoromethyl)-5H-dibenzo [b,d] thiophene-5-ium trifluoromethanesulfonate, N-bromosuccinimide, N-chlorosuccinimide, and N-iodosuccinimide.

The compound (I-3) can be obtained, for example, by reacting a compound represented by Formula (I-5) (hereinafter, may be referred to as a compound (I-5)) with a compound represented by Formula (I-6) (hereinafter, may be referred to as a compound (I-6)).

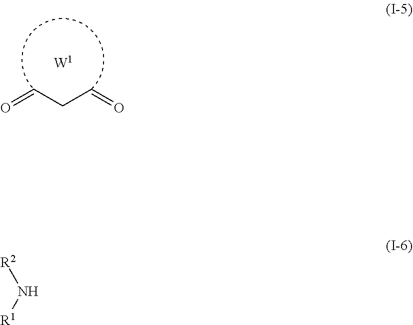

[in the formula, the ring W¹, R¹, and R² have the same meaning as described above].

The reaction between the compound (I-5) and the compound (I-6) is performed by mixing the compound (I-5) and the compound (I-6).

The use amount of the compound (I-6) is usually 0.1 to 5 mol, and preferably 0.5 to 2 mol, with respect to 1 mol of the compound (I-5).

The reaction between the compound (I-5) and the compound (I-6) may be performed in the presence of a solvent. Examples of thereof include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Benzene, toluene, ethanol, and acetonitrile are preferable.

The reaction time between the compound (I-5) and the compound (I-6) is usually 0.1 to 10 hours.

The reaction temperature between the compound (I-5) and the compound (I-6) is usually −50 to 150° C.

Examples of the compound (I-5) include the compounds described below.

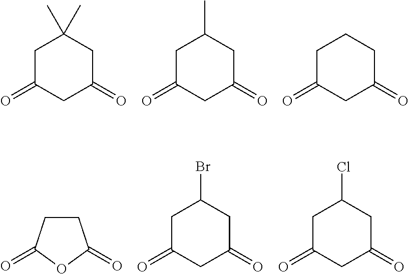

Examples of the compound (I-6) include ammonia; primary amines such as methylamine, ethylamine, ethanolamine, and 4-hydroxybutylamine; secondary amines such as dimethylamine, diethylamine, dibutylamine, pyrrolidine, piperidine, 3-hydroxypyrrolidine, 4-hydroxypiperidine, and azetidine.

The compound (I-1) can also be obtained by reacting compound (hereinafter, may be referred to as a compound (I-5-1)) represented by Formula (I-5-1) with a compound (I-6).

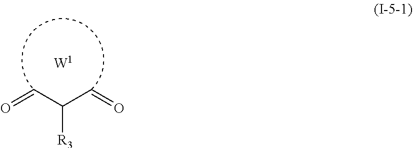

[in Formula (I-5-1), the ring W¹ and R³ have the same meaning as described above].

The reaction between the compound (I-5-1) and the compound (I-6) is performed by mixing the compound (I-5-1) and the compound (I-6).

The use amount of the compound (I-6) is usually 0.1 to 5 mol, and preferably 0.5 to 2 mol, with respect to 1 mol of the compound (I-5-1).

The reaction between the compound (I-5-1) and the compound (I-6) may be performed in the presence of a solvent. Examples of thereof include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Benzene, toluene, ethanol, and acetonitrile are preferable.

The reaction time between the compound (I-5-1) and the compound (I-6) is usually 0.1 to 10 hours.

The reaction temperature between the compound (I-5-1) and the compound (I-6) is usually −50 to 150° C.

Examples of the compound represented by Formula (I-5-1) include the compounds described below.

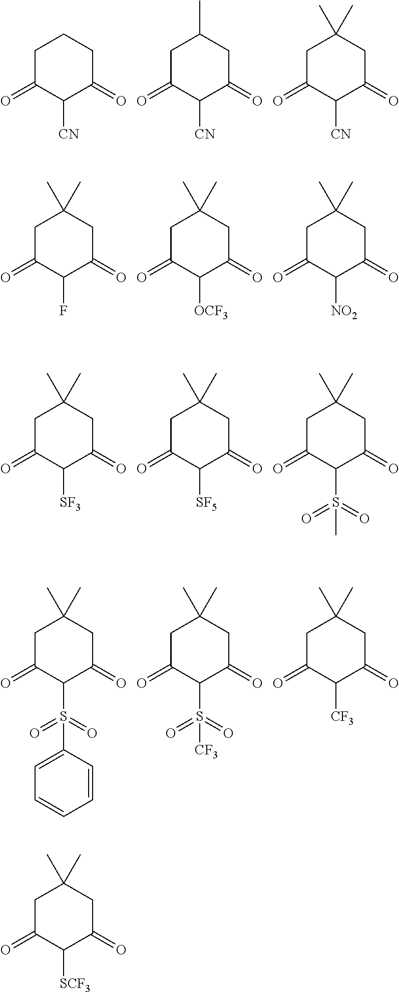

The compound (I) can also be obtained by reacting compound (hereinafter, may be referred to as a compound (I-7)) represented by Formula (I-7) with a compound (I-6).

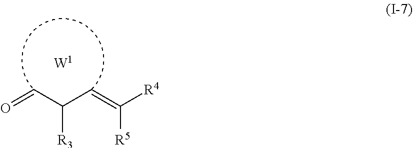

[in Formula (I-7), the ring W¹, R³, R⁴, and R⁵ have the same meaning as described above].

The reaction between the compound (I-7) and the compound (I-6) is usually performed by mixing the compound (I-7) and the compound (I-6), and it is preferable to add the compound (I-7) to the compound (I-6).

The reaction between the compound (I-7) and the compound (I-6) is preferably performed by mixing the compound (I-7) and the compound (I-6) in the presence of a base and a methylating agent,

it is more preferable to mix the compound (I-7), the compound (I-6), a base, and a methylating agent, and

it is more preferable to mix the compound (I-7) with a mixture of the compound (I-6), a methylating agent, and a base.

Examples of the base include the same bases as those used for the reaction between the compound (I-1) and the compound (I-2).

The use amount of the base is usually 0.1 to 5 mol and preferably 0.5 to 2 mol, with respect to 1 mol of the compound (1-7).

Examples of the methylating agent include the same methylating agents as those used for the reaction between the compound (I-1) and the compound (I-2).

The use amount of the methylating agent is usually 0.1 to 5 mol and preferably 0.5 to 2 mol, with respect to 1 mol of the compound (I-7).

The use amount of the compound (I-6) is usually 0.1 to 10 mol, and preferably 0.5 to 5 mol, with respect to 1 mol of the compound (I-7).

The reaction between the compound (I-7) and the compound (I-6) may be performed in the presence of a solvent. Examples of the solvent include the same solvents as those used for the reaction between the compound (I-1) and the compound (I-2). Methanol, ethanol, isopropanol, toluene, and acetonitrile are preferable.

The reaction time between the compound (I-7) and the compound (I-6) is usually 0.1 to 10 hours.

The reaction temperature between the compound (I-7) and the compound (I-6) is usually −50 to 150° C.

Examples of the compound (I-7) include the compounds described below.

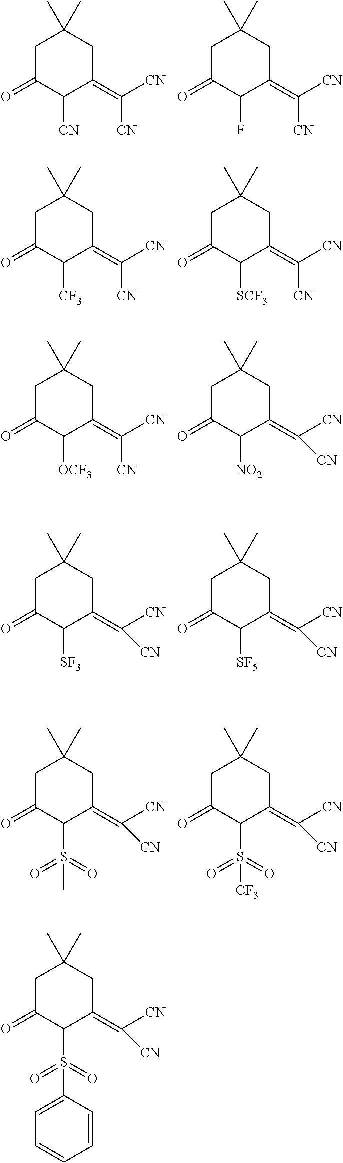

The compound (I-7) can also be obtained by reacting compound represented by Formula (I-8) with a compound (I-4).

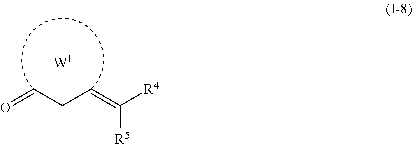

[in Formula (I-8), the ring W₁, R⁴, and R⁵ have the same meaning as described above].

The reaction between the compound (I-8) and the compound (I-4) can be performed by mixing the compound (I-8) and the compound (I-4).

The reaction between the compound (I-8) and the compound (I-4) is preferably performed in the presence of a base. Examples of the base include the same bases as those used for the reaction between the compound (I-1) and the compound (I-2). Metal hydroxides (more preferably alkali metal hydroxides), metal alkoxides (more preferably alkali metal alkoxides), amine compounds, and metal amide compounds (more preferably alkali metal amides) are more preferable.

The use amount of the base is usually 0.1 to 10 mol and preferably 0.5 to 2 mol, with respect to 1 mol of the compound (I-8).

The reaction between the compound (I-8) and the compound (I-4) may be performed in the presence of a solvent. Examples of the solvent include the same solvents as those used for the reaction between the compound (I-1) and the compound (I-2). Toluene, acetonitrile, methanol, ethanol, and isopropanol are preferable.

The reaction time between the compound (I-8) and the compound (I-4) is usually 0.1 to 10 hours.

The reaction temperature between the compound (I-8) and the compound (I-4) is usually −50 to 150° C.

Examples of the compound (I-8) include the compounds described below.

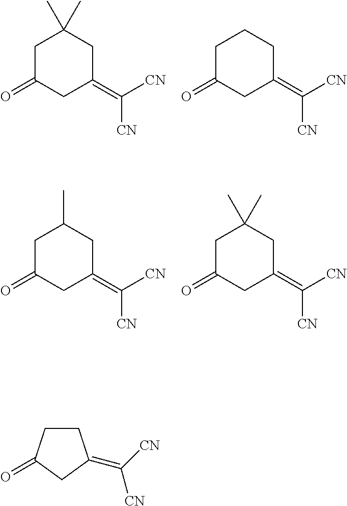

The compound (I-8) can also be obtained by reacting the compound (I-5) with the compound (I-2). The reaction between the compound (I-5) and the compound (I-2) can be performed by mixing the compound (I-5) and the compound (I-2).

The reaction between the compound (I-5) and the compound (I-2) is preferably performed in the presence of a base. Examples of the base include the same bases as those used for the reaction between the compound (I-1) and the compound (I-2). The use amount of the base is usually 0.1 to 5 mol and preferably 0.5 to 2 mol, with respect to 1 mol of the compound (I-5).

The reaction between the compound (I-5) and the compound (I-2) may be performed in the presence of a solvent. Examples of the solvent include the same solvents as those used for the reaction between the compound (I-1) and the compound (I-2). Methanol, ethanol, isopropanol, toluene, and acetonitrile are preferable.

The reaction time between the compound (I-5) and the compound (I-2) is usually 0.1 to 10 hours.

The reaction temperature between the compound (I-5) and the compound (I-2) is usually −50 to 150° C.

The use amount of the compound (I-2) is usually 0.1 to 10 mol, and preferably 0.5 to 2 mol, with respect to 1 mol of the compound (I-5).

In addition, the compound (I-7) can also be obtained by reacting the compound (I-5-1) with the compound (I-2).

The reaction between the compound (I-5-1) and the compound (I-2) is performed by mixing the compound (I-5-1) and the compound (I-2).

The use amount of the compound (I-2) is usually 0.1 to 5 mol, and preferably 0.5 to 2 mol, with respect to 1 mol of the compound (I-5-1).

The reaction between the compound (I-5-1) and the compound (I-2) may be performed in the presence of a solvent. Examples of thereof include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Benzene, toluene, ethanol, and acetonitrile are preferable.

The reaction time between the compound (I-5-1) and the compound (I-2) is usually 0.1 to 10 hours.

The reaction temperature between the compound (I-5-1) and the compound (I-2) is usually −50 to 150° C.

<Method for Producing Compounds (II) to (VIII)>

The compound (II) can be obtained, for example, by reacting 2 molar equivalents of the compound (I-7) with 1 molar equivalent of the compound represented by Formula (II-1).

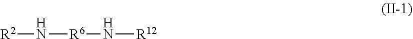

[in the formula, R², R¹², and R¹ have the same meaning as described above].

Examples of the compound represented by Formula (II-1) include the compounds described below.

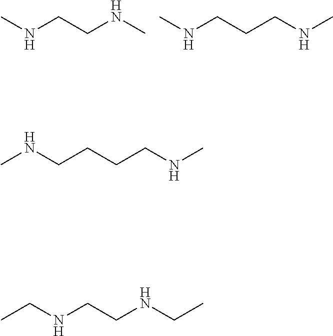

The compound (III) can be obtained, for example, by reacting 2 molar equivalents of the compound (I-7) with 1 molar equivalent of the compound represented by Formula (III-1)

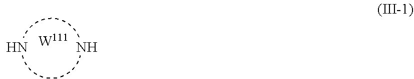

[in the formula, the ring W¹¹¹ has the same meaning as described above].

Examples of the compound represented by Formula (III-1) include the compounds described below.

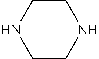

The compound (IV) can be obtained, for example, by reacting 2 molar equivalents of the compound (I-7) with 1 molar equivalent of the compound represented by Formula (IV-1).

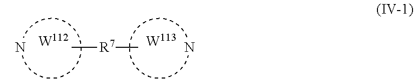

[in the formula, the rings W¹¹², W¹¹³, and R⁷ have the same meanings as described above].

Examples of the compound represented by Formula (IV-1) include the compounds described below.

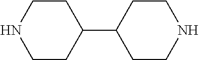 [SMILES: C1CC(C2CCNCC2)CCN1]

The compound (V) can be obtained, for example, by reacting 2 molar equivalents of the compound (I-1) with 1 molar equivalent of the compound represented by Formula (V-1).

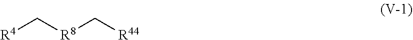

[in the formula, R⁴, R⁸, and R⁴⁴ have the same meaning as described above].

Examples of the compound represented by Formula (V-1) include the compounds described below.

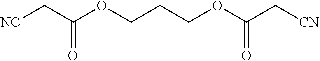 [SMILES: N#CCC(=O)OCCCOC(=O)CC#N]

The compound (VI) can be obtained, for example, by reacting 3 molar equivalents of the compound (I-1) with 1 molar equivalent of the compound represented by Formula

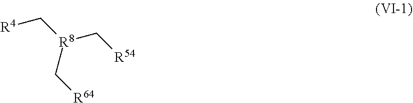

[in the formula, R⁴, R⁸, R⁵⁴, and R⁶⁴ have the same meaning as described above].

Examples of the compound represented by Formula (VI-1) include the compounds described below.

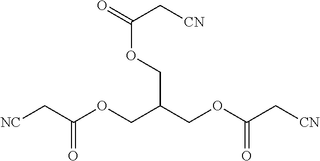 [SMILES: N#CCC(=O)OCC(COC(=O)CC#N)COC(=O)CC#N]

The compound (VII) can be obtained, for example, by reacting 3 molar equivalents of the compound (I-7) with 1 molar equivalent of the compound represented by Formula (VII-1).

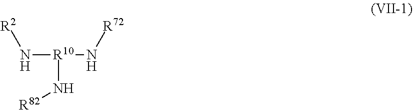

[in the formula, R², R¹⁰, R⁷², and R³² have the same meaning as described above].

Examples of the compound represented by Formula (VII-1) include the compounds described below.

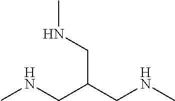

The compound (VIII) can be obtained, for example, by reacting 4 mol equivalents of the compound (I-7) with 1 mol equivalent of the compound represented by Formula (VIII-1).

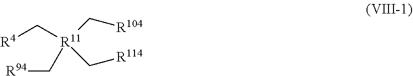

[in the formula, R⁴, R¹¹, R⁹⁴, R¹⁰⁴, and R¹¹⁴ have the same meaning as described above].

Examples of the compound represented by Formula (VIII-1) include the compounds described below.

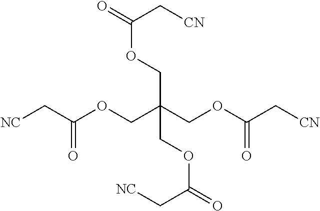 [SMILES: N#CCC(=O)OCC(COC(=O)CC#N)(COC(=O)CC#N)COC(=O)CC#N]

<Composition Containing Compound (X)>

The invention also includes compositions containing compound (X), (preferably any of compounds (I) to (VIII)).

The composition containing the compound (X) of the present invention (preferably any of the compounds (I) to (VIII)) is preferably a resin composition containing the compound (X) (preferably any of the compounds (I) to (VIII)) and a resin.

The composition can be used for all applications, and among them, the composition can be particularly suitably used for applications that may be exposed to sunlight or light including ultraviolet rays. Specific examples thereof include glass substitutes and surface coating materials thereof; coating materials for window glass, lighting glass, and light source protective glass for housing, facility, transport equipment and the like; window films for housing, facility, transport equipment and the like; interior/exterior materials and interior/exterior paints for housing, facility, transport equipment and the like, and coating films formed by the paint; alkyd resin lacquer paints and coating films formed by the paint; acrylic lacquer paints and coating films formed by the paint; light source members that emit ultraviolet rays, such as a fluorescent lamp and a mercury lamp; materials for blocking electromagnetic waves generated from precision machinery, electronic and electrical equipment members, and various displays; containers or packaging materials for foods, chemicals, chemicals, and the like; bottles, boxes, blisters, cups, special packaging, compact disc coats, agricultural and industrial sheets, or film materials; anti-fading agents for a printed matter, a dyed matter, a dyeing pigment, and the like; protective films for polymer supports (for example, for plastic parts such as machine and automotive parts); printed matter overcoat; inkjet medium coating; laminated mattes; optical light films; safety glass/windshield intermediate layers; electrochromic/photochromic applications; overlaminated films; solar heat control films; cosmetics such as sunscreen, shampoo, conditioner, and hair styling products; textile products and textiles for clothing such as sportswear, stockings and hats; household interiors such as curtains, rugs, and wallpaper; medical instruments such as plastic lenses, contact lenses, and artificial eyes; optical supplies such as optical filters, backlight display films, prisms, mirrors, and photographic materials; stationery such as mold films, transfer stickers, graffiti prevention films, tapes, and inks; and marking boards, signing device, and the like, and surface coating materials thereof.

A shape of a polymer molded article formed from the resin composition may be any shape such as a flat film shape, a powder shape, a spherical particle shape, a crushed particle shape, a massive continuous body, a fiber shape, a tubular shape, a hollow fiber shape, a granular shape, a plate shape, and a porous shape.

Examples of the resin used in the resin composition include a thermoplastic resin and a thermosetting resin conventionally used in the production of known various molded bodies, sheets, films and the like.

Examples of the thermoplastic resin include olefin resins such as a polyethylene resin, a polypropylene resin, and a polycycloolefin resin, polyester resins such as a poly (meth)acrylic acid ester resin, a polystyrene resin, a styrene-acrylonitrile resin, an acrylonitrile-butadiene-styrene resin, a polyvinyl chloride resin, a polyvinylidene chloride resin, a polyvinyl acetate resin, a polyvinyl butyral resin, an ethylene-vinyl acetate copolymer, an ethylene-vinyl alcohol resin, a polyethylene terephthalate resin, a polybutylene terephthalate resin, and a liquid crystal polyester resin, a polyacetal resin, a polyamide resin, a polycarbonate resin, a polyurethane resin, and a polyphenylene sulfide resin. These resins may be used as one or more polymer blends or polymer alloys.

Examples of the thermosetting resin include an epoxy resin, a melamine resin, an unsaturated polyester resin, a phenol resin, a urea resin, an alkyd resin, and a thermosetting polyimide resin.

When the resin composition is used as an ultraviolet absorbing filter or an ultraviolet absorbing film, the resin is preferably a transparent resin.

The resin composition can be obtained by mixing a compound (X) and a resin. The compound (X) only needs to be contained in an amount necessary for imparting desired performance, and can be contained, for example, in an amount of 0.01 to 20 parts by mass or the like with respect to 100 parts by mass of the resin.

The composition of the present invention may contain other additives such as a solvent, a crosslinking catalyst, a tackifier, a plasticizer, a softener, a dye, a pigment, and an inorganic filler as necessary.

The composition and the resin composition may be a spectacle lens composition. A spectacle lens can be formed by molding or the like using the spectacle lens composition. The method for molding the spectacle lens composition may be injection molding or cast polymerization molding. The cast polymerization molding is a method in which a spectacle lens composition mainly composed of a monomer or oligomer resin is injected into a lens mold, and the spectacle lens composition is cured by heat or light to be molded into a lens.

The spectacle lens composition may have a composition suitable for the molding method. For example, when the spectacle lens is formed by injection molding, a resin composition for a spectacle lens containing the resin and the compound (X) may be used. In addition, when the spectacle lens is formed by cast polymerization molding, a spectacle lens composition containing a curable monomer that cures by heat or light and the compound (X) may be used.

Examples of the resin contained in the spectacle lens composition include the resins described above, and a transparent resin is preferable. The resin contained in the spectacle lens composition is preferably used as a polymer blend or a polymer alloy of one or more of a poly (meth)acrylic acid ester resin, a polycarbonate resin, a polyamide resin, a polyurethane resin, and a polythiourethane resin. In addition, not only the polymer but also a monomer component may be contained.

The spectacle lens composition may be a composition containing the curable monomer and the compound (X). Two or more curable monomers may be contained. Specifically, it may be a mixture of a polyol compound and an isocyanate compound or a mixture of a thiol compound and an isocyanate compound, and is preferably a mixture of a thiol compound and an isocyanate, and more preferably a mixture of a polyfunctional thiol compound and a polyfunctional isocyanate compound.

The thiol compound is not particularly limited as long as it is a compound having at least one thiol group in the molecule. It may be chain or cyclic. In addition, a sulfide bond, a polysulfide bond, and other functional groups may be present in the molecule. Specific examples of the thiol compound include thiol group-containing organic compounds having one or more thiol groups in one molecule described in JP-A-2004-315556, such as an aliphatic polythiol compound, an aromatic polythiol compound, a thiol group-containing cyclic compound, and a thiol group-containing sulfide compound. Among them, from the viewpoint of improving the refractive index and the glass transition temperature of the optical material, a polyfunctional thiol compound having two or more thiol groups is preferable, an aliphatic polythiol compound having two or more thiol groups and a sulfide compound containing two or more thiol groups are more preferable, and bis(mercaptomethyl) sulfide, 1,2-bis[(2-mercaptoethyl) thio]-3 mercaptopropane, pentaerythritol tetrakisthiopropionate, and 4,8-dimercaptomethyl-1,11 mercapto-3,6,9-trithiaundecane are still more preferable. The thiol compounds may be used alone or in combination of two or more thereof.

The isocyanate compound is preferably a polyfunctional isocyanate compound having at least two isocyanato groups (—NCO) in the molecule, and examples thereof include an aliphatic isocyanate compound (for example, hexamethylene diisocyanate), an alicyclic isocyanate compound (for example, isophorone diisocyanate, hydrogenated diphenylmethane diisocyanate, and hydrogenated xylylene diisocyanate), and an aromatic isocyanate compound (for example, tolylene diisocyanate, xylylene diisocyanate, diphenylmethane diisocyanate, naphthalene diisocyanate, and triphenylmethane triisocyanate). In addition, the isocyanate compound may be an adduct of the isocyanate compound with a polyhydric alcohol compound [adducts with, for example, glycerol, and trimethylolpropane], a derivative of an isocyanurate compound, a biuret type compound, and a urethane prepolymer type isocyanate compound obtained by addition reaction with polyether polyol, polyester polyol, acrylic polyol, polybutadiene polyol, polyisoprene polyol, or the like.

When the spectacle lens composition contains a curable monomer, a curing catalyst may be contained in order to improve curability. Examples of the curing catalyst include a tin compound such as dibutyltin chloride, amines disclosed in JP-A-2004-315556, phosphines, quaternary ammonium salts, quaternary phosphonium salts, tertiary sulfonium salts, secondary iodonium salts, mineral acids, Lewis acids, organic acids, silicic acids, tetrafluoroboric acids, peroxides, an azo based compound, a condensate of aldehyde and an ammonia compound, guanidines, thioureas, thiazoles, sulfenamides, thiurams, dithiocarbamates, xanthogenates, and acidic phosphoric esters. These curing catalysts may be used alone or in combination of two or more thereof.

When the spectacle lens composition is a resin composition, the content of the compound (X) in the spectacle lens composition can be, for example, 0.01 to 20 parts by mass based on 100 parts by mass of the resin. When the spectacle lens composition is a curable composition, for example, the content of the compound (X) can be 0.00001 to 20 parts by mass based on 100 parts by mass of the curable component. The content of the compound (X) is preferably 0.0001 to 15 parts by mass, more preferably 0.001 to 10 parts by mass, still more preferably 0.01 to 5 parts by mass, and particularly preferably 0.1 to 3 parts by mass, based on 100 parts by mass of the resin or the curable component.

The addition amount of the curing catalyst is preferably 0.0001 to 10.0% by mass, and more preferably 0.001 to 5.0% by mass based on 100% by mass of the spectacle lens composition.

The spectacle lens composition may contain the above-described additives.

### EXAMPLES

Hereinafter, the present invention will be described more specifically with reference to examples and comparative examples, but the present invention is not limited by these examples. In the examples, % and part representing the content or use amount are on a mass basis unless otherwise specified.

### (Example 1) Synthesis of Compound Represented by Formula (UVA-1)

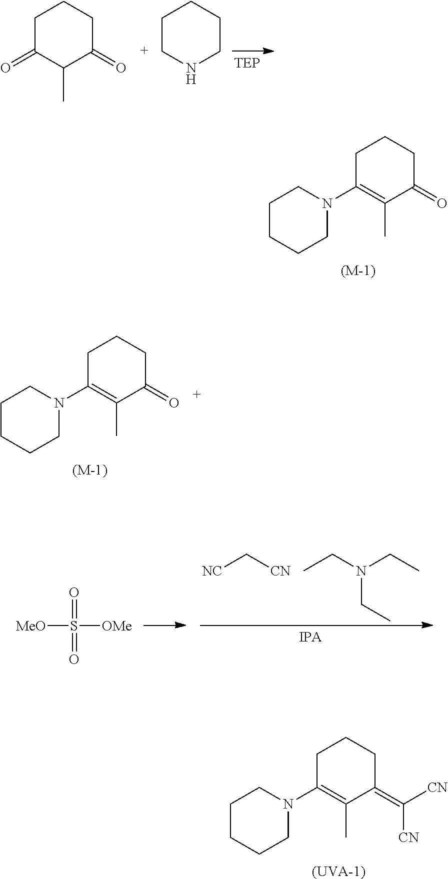

The inside of a 300 mL-four-necked flask equipped with a Dimroth cooling tube and a thermometer was made into a nitrogen atmosphere, and 5 parts of 2-methyl 1,3-cyclohexanedione, 3.7 parts of piperidine, and 50 parts of toluene were added thereto, and the mixture was stirred under reflux for 5 hours. The solvent was distilled off from the obtained mixture and purification was performed to obtain 6.8 parts of a compound represented by Formula (M-1).

Under a nitrogen atmosphere, the obtained compound represented by Formula (M-1), 1.3 parts of dimethyl sulfuric acid, and 4 parts of acetonitrile were mixed, and the mixture was stirred at 20 to 30° C. for 3 hours. 0.75 parts of malononitrile, 1.2 parts of triethylamine, and 4 parts of isopropanol were added to the obtained mixture, and the mixture was stirred at 20 to 30° C. for 3 hours. The solvent was distilled off from the obtained mixture and purified to obtain 0.3 parts of a compound represented by Formula (UVA-1).

LC-MS measurement and ¹H-NMR analysis were performed, and it was confirmed that a compound represented by Formula (UVA-1) was produced.

¹H-NMR (deuterated dimethyl sulfoxide (hereinafter, may be referred to as deuterated DMSO) δ: 1.68 to 1.75 (m, 8H), 2.16 (s, 3H), 2.50 to 2.62 (dt, 4H) 3.40 to 3.43 (t, 4H)

LC-MS; [M+H]⁺=242.5

<Measurement of Maximum Absorption Wavelength and Gram Absorption Coefficient ε>

A 2-butanone solution (0.006 g/L) of the obtained compound represented by Formula (UVA-1) was charged into a 1 cm quartz cell, the quartz cell was set in a spectrophotometer UV-2450 (manufactured by Shimadzu Corporation), and absorbance in a wavelength range of 300 to 800 nm was measured every 1 nm step by a double beam method. From the obtained absorbance value, the concentration of the compound represented by Formula (UVA-1) in the solution, and an optical path length of the quartz cell, a gram absorption coefficient for each wavelength was calculated.

ε(λ)=A(λ)/CL

[in the equation, ε(X) represents a gram absorption coefficient (L/(g·cm)) of the compound represented by Formula (UVA-1) at a wavelength of λ nm, A(λ) represents absorbance at a wavelength of λ nm, C represents concentration (g/L), and L represents an optical path length (cm) of the quartz cell].

The maximum absorption wavelength of the obtained compound represented by Formula (UVA-1) was 412.9 nm. The obtained compound represented by Formula (UVA-1) had ε(λmax) of 1.946 L/(g·cm), ε(λmax+30 nm) of 0.138 L/(g·cm), and ε(λmax)/ε(λmax+30 nm) of 14.1.

### (Example 2) Synthesis of Compound Represented by Formula (UVA-2)

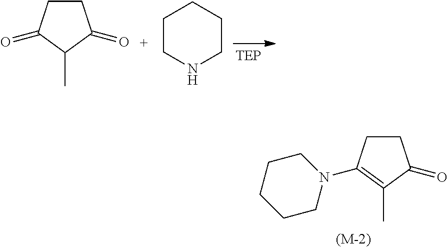

The inside of a 300 mL-four-necked flask equipped with a Dimroth cooling tube and a thermometer was made into a nitrogen atmosphere, and 5 parts of 2-methyl 1,3-cyclopentanedione, 4.2 parts of piperidine, and 50 parts of toluene were added thereto, and the mixture was stirred under reflux for 5 hours. The solvent was distilled off from the obtained mixture and purification was performed to obtain 4 parts of a compound represented by Formula (M-2).

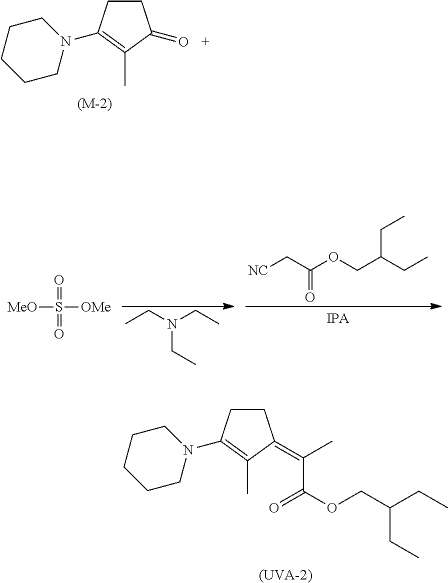

Under a nitrogen atmosphere, the obtained compound represented by Formula (M-2), 1.7 parts of dimethyl sulfuric acid, and 4.5 parts of acetonitrile were mixed, and the mixture was stirred at 20 to 30° C. for 3 hours. 2.4 parts of (2-ethylbutyl) cyanoacetate, 1.4 parts of triethylamine, and 4.5 parts of isopropanol were added to the obtained mixture, and the mixture was stirred at 20 to 30° C. for 3 hours. The solvent was distilled off from the obtained mixture and purified to obtain 1.5 parts of a compound represented by Formula (UVA-2).

LC-MS measurement and ¹H-NMR analysis were performed, and it was confirmed that a compound represented by Formula (UVA-2) was produced.

¹H-NMR (deuterated DMSO) δ: 0.89-0.93 (t, 6H), 1.36-1.48 (m, 4H), 1.52-1.62 (m, 2H) 1.69-1.71 (m, 6H), 2.22 (s, 3H), 2.57-2.60 (t, 2H), 3.15-3.18 (t, 2H), 3.53-3.55 (t, 4H), 4.05-4.06 (d, 2H)

LC-MS; [M+H]⁺=331.5

In addition, the maximum absorption wavelength and the gram absorption coefficient were measured in the same manner as described above. The maximum absorption wavelength of the obtained compound represented by Formula (UVA-2) was 382.6 nm. The obtained compound represented by Formula (UVA-2) had ε(λmax) of 1.9 L/(g·cm), ε(λmax+30 nm) of 0.057 L/(g·cm), and ε(λmax)/ε(λmax+30 nm) of 33.3.

### (Example 3) Synthesis of Compound Represented by Formula (UVA-3)

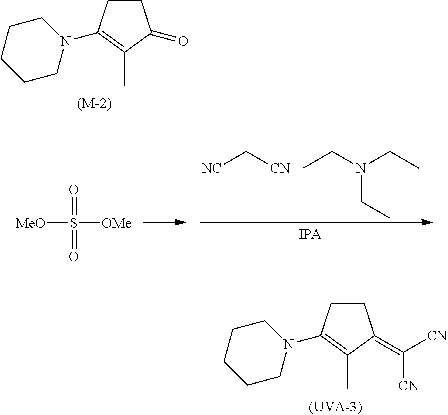

Under a nitrogen atmosphere, 2 parts of the compound represented by Formula (M-2), 1.5 parts of dimethyl sulfuric acid, and 4 parts of acetonitrile were mixed, and the mixture was stirred at 20 to 30° C. for 3 hours. Further, 0.8 parts of malononitrile, 1.2 parts of triethylamine, and 4 parts of isopropanol were added to the obtained mixture, and the mixture was stirred at 20 to 30° C. for 3 hours. The solvent was distilled off from the obtained mixture and purified to obtain 1.7 parts of a compound represented by Formula (UVA-3).

LC-MS measurement and ¹H-NMR analysis were performed, and it was confirmed that a compound represented by Formula (UVA-3) was produced.

¹H-NMR (deuterated DMSO) δ: 1.69-1.74 (m, 6H), 2.19 (s, 3H), 2.65-2.81 (dt, 4H) 3.57-3.59 (t, 4H) LC-MS; [M+H]⁺=228.5(+H)

In addition, the maximum absorption wavelength and the gram absorption coefficient were measured in the same manner as described above. The maximum absorption wavelength of the obtained compound represented by Formula (UVA-3) was 376.8 nm. The obtained compound represented by Formula (UVA-3) had ε(λmax) of 2.81 L/(g·cm), ε(λmax+30 nm) of 0.058 L/(g·cm), and ε(λmax)/ε(λmax+30 nm) of 48.4.

### (Example 4) Synthesis of Compound Represented by Formula (UVA-4)

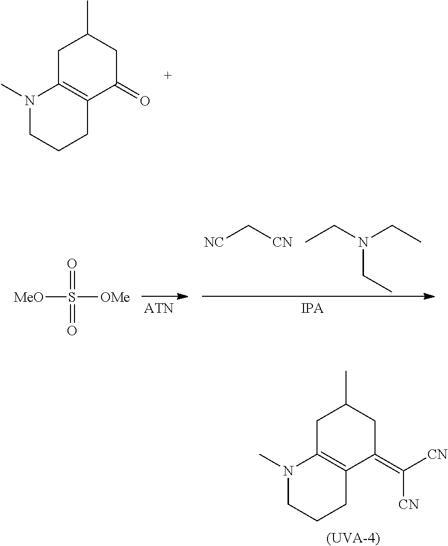

In a nitrogen atmosphere, 1.5 parts of 1,7-dimethyl-1-2,3,4,6,7,8-hexahydroquinoline-5 (1H)-one, 1.1 parts of dimethyl sulfuric acid, and 9 parts of acetonitrile were added, and the mixture was stirred at 20 to 30° C. for 3 hours. 0.6 parts of malononitrile, 0.9 parts of triethylamine, and 9 parts of isopropanol were added to the obtained mixture, and the mixture was stirred at 20 to 30° C. for 3 hours. The solvent was distilled off from the obtained mixture and purified to obtain 1.2 parts of a compound represented by Formula (UVA-4).

LC-MS measurement and ¹H-NMR analysis were performed, and it was confirmed that a compound represented by Formula (UVA-4) was produced.

¹H-NMR (deuterated DMSO) δ: 1.08-1 09 (d, 3H), 1.76-2.13 (m, 5H), 2.55-2.59 (dd, 1H), 2.66-2.74 (m, 1H), 2.81-2.93 (m, 2H), 3.12 (s, 3H), 3.28-3.37 (m, 2H)

LC-MS; [M+H]⁺=228.2

In addition, the maximum absorption wavelength and the gram absorption coefficient were measured in the same manner as described above. The maximum absorption wavelength of the obtained compound represented by Formula (UVA-4) was 401.8 nm. The obtained compound represented by Formula (UVA-4) had ε(λmax) of 2.76 L/(g·cm), ε(λmax+30 nm) of 0.055 L/(g·cm), and ε(λmax)/ε(λmax+30 nm) of 50.1.

### (Example 5) Synthesis of Compound Represented by Formula (UVA-5)

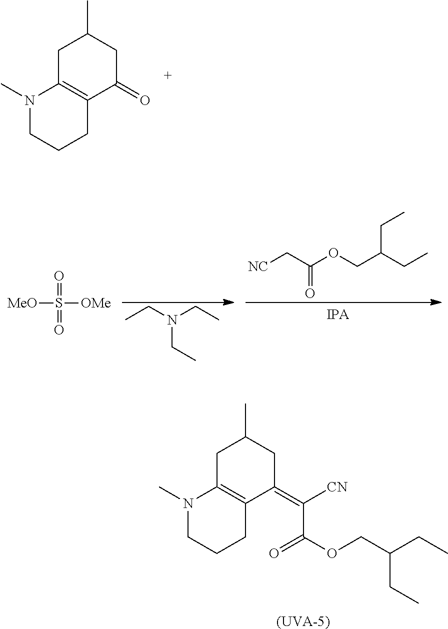

In a nitrogen atmosphere, 1.5 parts of 1,7-dimethyl-1-2,3,4,6,7,8-hexahydroquinoline-5 (1H)-one, 1.1 parts of dimethyl sulfuric acid, and 9 parts of acetonitrile were mixed, and the mixture was stirred at 20 to 30° C. for 3 hours. 1.6 parts of (2-ethylbutyl) cyanoacetate, 0.9 parts of triethylamine, and 9 parts of isopropanol were added to the obtained mixture, and the mixture was stirred at 20 to 30° C. for 3 hours. The solvent was distilled off from the obtained mixture and purified to obtain 1 part of a compound represented by Formula (UVA-5).

LC-MS measurement and ¹H-NMR analysis were performed, and it was confirmed that a compound represented by Formula (UVA-5) was produced.

¹H-NMR (deuterated DMSO) δ: 0.89-0.93 (t, 6H), 1.07-1.08 (d, 3H), 1.36-1.48 (m, 4H), 1.57-1.62 (m, 3H), 1.82-2.04 (m, 4H), 2.04-2.21 (dd, 1H), 2.52-2.57 (dd, 1H), 2.73 (m, 1H), 3.09 (s, 3H), 3.30-3.33 (t, 2H), 4.04-4.06 (dd, 2H)

LC-MS; [M+H]⁺=:331.2

In addition, the maximum absorption wavelength and the gram absorption coefficient were measured in the same manner as described above. The maximum absorption wavelength of the obtained compound represented by Formula (UVA-5) was 412.7 nm. The obtained compound represented by Formula (UVA-5) had ε(λmax) of 1.36 L/(g·cm), ε(λmax+30 nm) of 0.202 L/(g·cm), and ε(λmax)/ε(λmax+30 nm) of 6.74.

### (Example 6) Synthesis of Compound Represented by Formula (UVA-6)

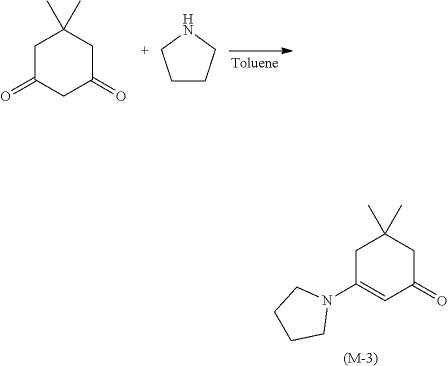

The inside of a 500 mL-four-necked flask equipped with a Dimroth cooling tube and a thermometer was made into a nitrogen atmosphere, and 20 parts of dimedone, 11.2 parts of pyrrolidine, and 200 parts of toluene were added thereto, and the mixture was stirred under reflux for 5 hours. The solvent was distilled off from the obtained mixture and purification was performed to obtain 27.4 parts of a compound represented by Formula (M-3).

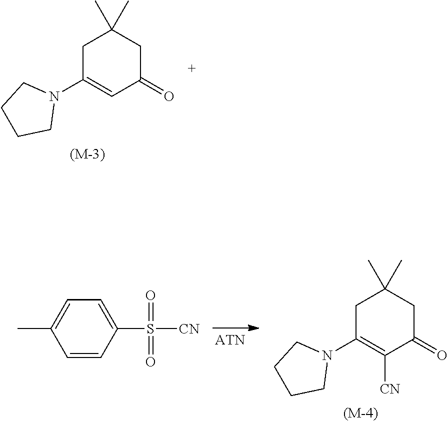

Under a nitrogen atmosphere, 1.0 part of the obtained compound represented by Formula (M-3), 2.8 parts of p-toluenesulfonyl cyanide, and 10 parts of acetonitrile were mixed. The obtained mixture was allowed to stir at 0 to 5° C. for 5 hours. The solvent was distilled off from the obtained mixture and purified to obtain 0.6 parts of a compound represented by Formula (M-4).

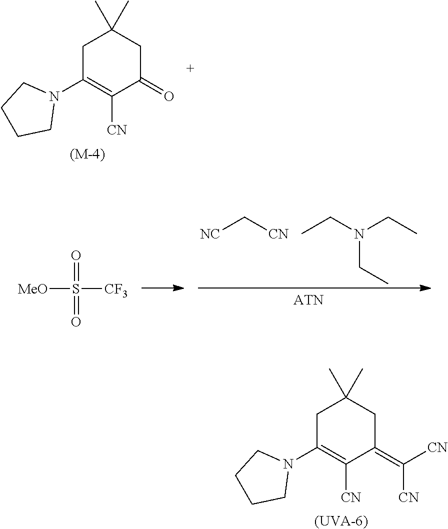

Under a nitrogen atmosphere, 4.8 parts of a compound represented by Formula (M-4), 4.6 parts of methyl triflate, and 24 parts of acetonitrile were mixed, and the mixture was stirred at 20 to 30° C. for 3 hours. 1.9 parts of malononitrile, 3 parts of triethylamine, and 24 parts of acetonitrile were added to the obtained mixture, and the mixture was stirred at 20 to 30° C. for 3 hours. The solvent was distilled off from the obtained mixture and purified to obtain 2.9 parts of a compound represented by Formula (UVA-6).

LC-MS measurement and ¹H-NMR analysis were performed, and it was confirmed that a compound represented by Formula (UVA-6) was produced.

¹H-NMR (CDCl₃) δ: 0.99 (s, 6H), 1.90-1.96 (m, 4H), 2.48-2.51 (m, 4H), 3.70-3.88 (dt, 4H)

LC-MS; [M+H]⁺=284.5

In addition, the maximum absorption wavelength and the gram absorption coefficient were measured in the same manner as described above. The maximum absorption wavelength of the obtained compound represented by Formula (UVA-6) was 380 nm. The obtained compound represented by Formula (UVA-6) had ε(λmax) of 1.75 L/(g·cm), ε(λmax+30 nm) of 0.032 L/(g·cm), and ε(λmax)/ε(λmax+30 nm) of 54.53.

### (Example 7) Synthesis of Compound Represented by Formula (UVA-7)

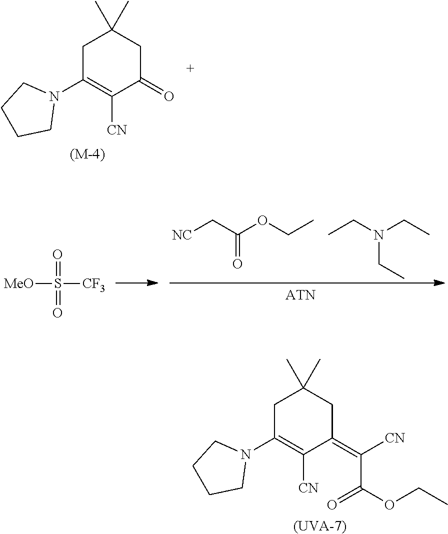

Under a nitrogen atmosphere, 1 part of a compound represented by Formula (M-4), 0.6 parts of methyl triflate, and 10 parts of acetonitrile were mixed, and the mixture was stirred at 20 to 30° C. for 3 hours. 5.2 parts of ethyl cyanoacetate, 4.6 parts of triethylamine, and 10 parts of acetonitrile were added to the obtained mixture, and the mixture was stirred at 20 to 30° C. for 3 hours. The solvent was distilled off from the obtained mixture and purified to obtain 0.5 parts of a compound represented by Formula (UVA-7).

In the same manner as described above, LC-MS measurement and ¹H-NMR analysis were performed, and it was confirmed that a compound represented by Formula (UVA-7) was produced.

¹H-NMR (deuterated DMSO) δ: 0.960-0.994 (d, 6H), 1.20-1.26 (m, 3H), 1.93 (m, 4H), 2.53-2.91 (m, 4H), 3.77-3.81 (m, 4H), 4.10-4.19 (m, 2H)

LC-MS; [M+H]⁺=314.5(+H)

In addition, the maximum absorption wavelength and the gram absorption coefficient were measured in the same manner as described above. The maximum absorption wavelength of the obtained compound represented by Formula (UVA-7) was 382.7 nm. The obtained compound represented by Formula (UVA-7) had ε(λmax) of 1.08 L/(g·cm), ε(λmax+30 nm) of 0.153 L/(g·cm), and ε(λmax)/ε(λmax+30 nm) of 7.04.

### (Example 8) Synthesis of Compound Represented by Formula (UVA-8)

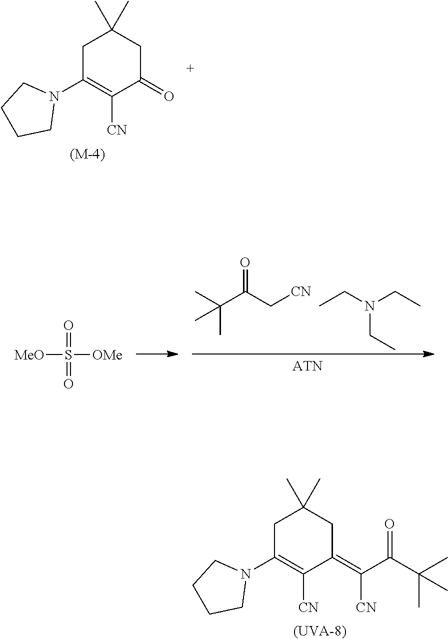

Under a nitrogen atmosphere, 0.5 parts of the compound represented by Formula (M-4), 0.5 parts of dimethyl sulfuric acid, and 5 parts of acetonitrile were mixed, and the mixture was stirred and reacted at 20 to 30° C. for 3 hours. Further, 0.4 parts of pivaloylacetonitrile, 0.5 parts of triethylamine, and 5.0 parts of acetonitrile were added, and the mixture was stirred and reacted at 20 to 30° C. for 3 hours. After completion of the reaction, the solvent was distilled off and purified to obtain 0.07 parts of a compound represented by Formula (UVA-8).

LC-MS measurement and ¹H-NMR analysis were performed, and it was confirmed that a compound represented by Formula (UVA-8) was produced.

¹H-NMR (deuterated DMSO) δ: 0.92 (s, 6H), 1.26 (s, 9H), 1.90 (s, 4H), 2.55 (m, 4H), 3.64-3.71 (m, 4H)

LC-MS; [M+H]⁺=326.5

In addition, the maximum absorption wavelength and the gram absorption coefficient were measured in the same manner as described above. The maximum absorption wavelength of the obtained compound represented by Formula (UVA-8) was 377.4 nm. The obtained compound represented by Formula (UVA-8) had ε(λmax) of 0.66 L/(g·cm), ε(λmax+30 nm) of 0.395 L/(g·cm), and ε(λmax)/ε(λmax+30 nm) of 1.68.

### (Example 9) Synthesis of Compound Represented by Formula (UVA-9)

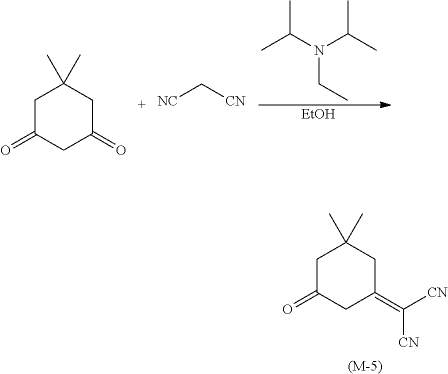

The inside of a 300 mL-four-necked flask equipped with a Dimroth cooling tube and a thermometer was made into a nitrogen atmosphere, and 70.0 parts of dimedone, 10.4 parts of malononitrile, 40.6 parts of diisopropylethylamine, and 100.0 parts of ethanol were charged thereto, and the mixture was heated and stirred under reflux for 3 hours. After completion of the reaction, the solvent was distilled off and purified to obtain 15.1 parts of a compound represented by Formula (M-5).

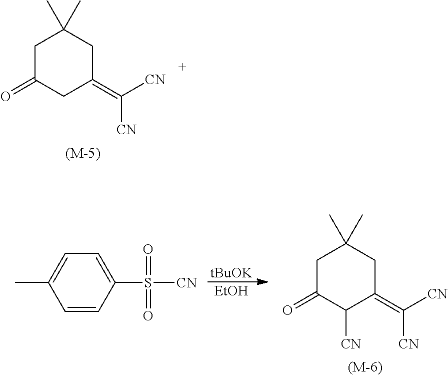

Under a nitrogen atmosphere, 5 parts of the compound represented by Formula (M-5), 5.8 parts of p-toluenesulfonyl cyanide, 3 parts of potassium tert-butoxide, and 50 parts of ethanol were mixed. The obtained mixture was allowed to stir at 0 to 5° C. for 3 hours. The solvent was distilled off from the obtained mixture and purified to obtain 3.3 parts of a compound represented by Formula (M-6).

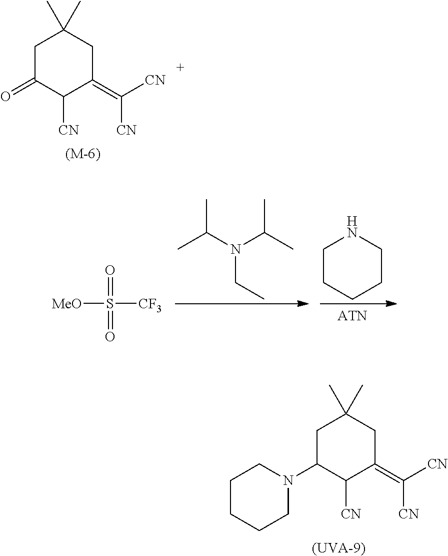

Under a nitrogen atmosphere, 1 part of a compound represented by Formula (M-6), 1 part of methyl triflate, 0.8 parts of diisopropylethylamine and 20 parts of acetonitrile were mixed, and the mixture was stirred at 20 to 30° C. for 3 hours. 1.4 parts of piperidine and 20 parts of acetonitrile were added to the obtained mixture, and the mixture was stirred at 20 to 30° C. for 3 hours. The solvent was distilled off from the obtained mixture and purified to obtain 0.5 parts of a compound represented by Formula (UVA-9).

LC-MS measurement and ¹H-NMR analysis were performed, and it was confirmed that a compound represented by Formula (UVA-9) was produced.

¹H-NMR (deuterated DMSO) δ: 0.99 (s, 6H), 1.60 (m, 6H), 2.71 (s, 2H), 3.80 (m, 4H)

LC-MS; [M+H]⁺=281.5

In addition, the maximum absorption wavelength and the gram absorption coefficient were measured in the same manner as described above. The maximum absorption wavelength of the obtained compound represented by Formula (UVA-9) was 385.6 nm. The obtained compound represented by Formula (UVA-9) had ε(λmax) of 1.65 L/(g·cm), ε(λmax+30 nm) of 0.088 L/(g·cm), and ε(λmax)/ε(λmax+30 nm) of 18.8.

### (Synthesis Example 1) Synthesis of Compound Represented by Formula (UVA-A1)

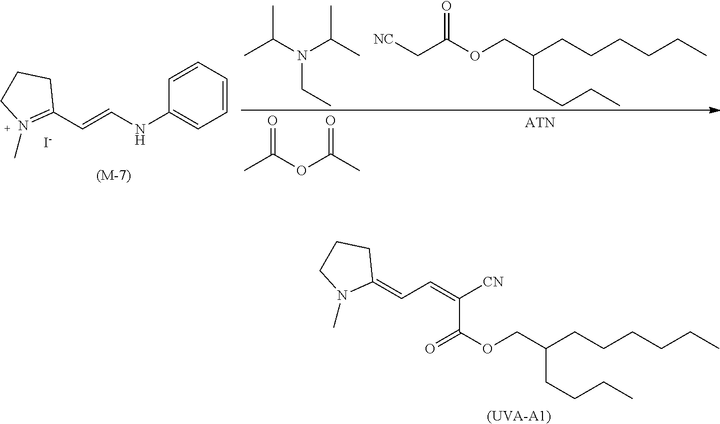

The inside of a 200 mL-four-necked flask equipped with a Dimroth cooling tube and a thermometer was made into a nitrogen atmosphere, and 10 parts of a compound represented by Formula (M-7) synthesized with reference to JP-A-2014-194508, 3.6 parts of acetic anhydride, 6.9 parts of (2-butyloctyl) cyanoacetate, and 60 parts of acetonitrile were charged, and the obtained mixture was stirred at 20 to 30° C. 4.5 parts of diisopropylethylamine was added dropwise to the obtained mixture over 1 hour, and the mixture was stirred for 2 hours. The solvent was distilled off from the obtained mixture and purified to obtain 4.6 parts of a compound represented by Formula (UVA-A1).

### (Synthesis Example 2) Synthesis of Compound Represented by Formula (UVA-A2)

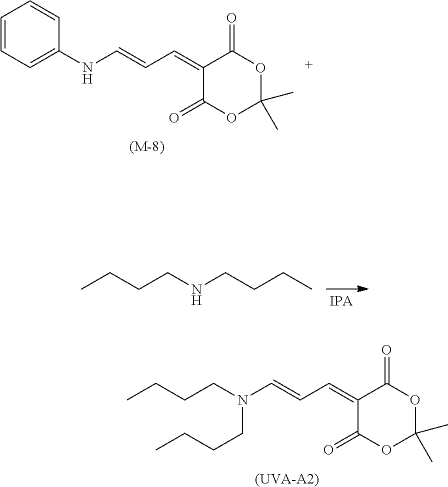

The inside of a 100 mL-four-necked flask equipped with a Dimroth cooling tube and a thermometer was made into a nitrogen atmosphere, and 6 parts of the compound represented by Formula (M-8), 14.2 parts of dibutylamine, and 31.3 parts of isopropanol were mixed, heated and refluxed, and then stirred for 3 hours. The solvent was distilled off from the obtained mixture and purified to obtain 4.6 parts of a compound represented by Formula (UVA-A2).

### (Synthesis Example 3) Synthesis of Compound Represented by Formula (UVA-A3)

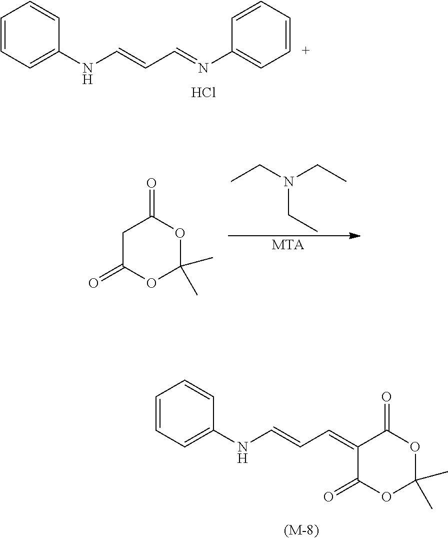

The inside of a 300 mL-four-necked flask equipped with a Dimroth cooling tube and a thermometer was made into a nitrogen atmosphere, and 30 parts of malonaldehyde dianilide hydrochloride, 18.4 parts of meldrum acid, 12.9 parts of triethylamine, and 90 parts of methanol were charged, and the mixture was stirred and reacted at 20 to 30° C. for 3 hours. After completion of the reaction, the solvent was distilled off and purified to obtain 24.4 parts of a compound represented by Formula (M-8).

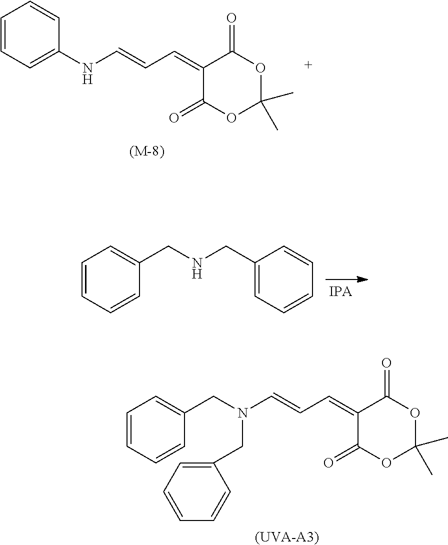

6 parts of the compound represented by Formula (M-8), 21.7 parts of dibenzylamine, and 31.3 parts of isopropanol were mixed, heated and refluxed, and the mixture was stirred and reacted for 3 hours. The solvent was distilled off from the obtained mixture and purified to obtain 3.5 parts of a compound represented by Formula (UVA-A3).

### (Synthesis Example 4) Synthesis of Compound Represented by Formula (UVA-A4)

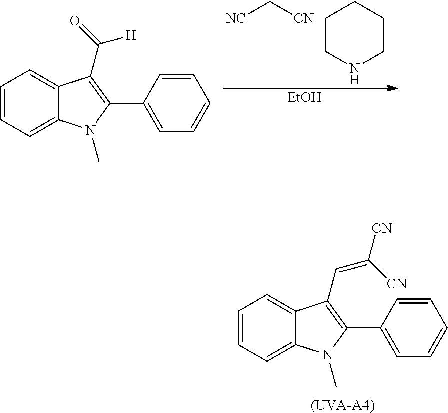

The inside of a 100 mL-four-necked flask equipped with a Dimroth cooling tube and a thermometer was made into a nitrogen atmosphere, and 5 parts of 2-phenyl-1 methylindole-3-carboxyaldehyde, 1.8 parts of piperidine, 1.5 parts of malononitrile, and 20 parts of ethanol were mixed, heated and refluxed, and the mixture was stirred for 18 hours. The obtained mixture was heated to 80° C. and kept at 80° C. for 18 hours. The solvent was distilled off from the obtained mixture and purified to obtain 4.9 parts of a compound represented by Formula (UVA-A4).

### (Example 10) Preparation of Light-Selective Absorption Composition (1)

The respective components were mixed in the following proportions to prepare a light-selective absorption composition (active energy ray-curable resin composition) (1).

Polyfunctional acrylate (“A-DPH-12E”: produced by Shin-Nakamura Chemical Co., Ltd.) 70 parts

Urethane acrylate (“UV-7650B”: produced by Nippon Chemical Industrial Co., Ltd.) 30 parts

Polymerization initiator (“NCI-730”: produced by ADEKA Corporation) 3 parts

Compound Represented by Formula (UVA-1) Synthesized in Example 1 2 parts

Methyl ethyl ketone 34 parts

### (Example 11) Preparation of Light-Selective Absorption Composition (2)

A light-selective absorption composition (2) was prepared in the same manner as in Example 10 except that a compound represented by Formula (UVA-1) was changed to a compound represented by Formula (UVA-2).

### (Example 12) Preparation of Light-Selective Absorption Composition (3)

A light-selective absorption composition (3) was prepared in the same manner as in Example 10 except that a compound represented by Formula (UVA-1) was changed to a compound represented by Formula (UVA-3).

### (Example 13) Preparation of Light-Selective Absorption Composition (4)

A light-selective absorption composition (4) was prepared in the same manner as in Example 10 except that a compound represented by Formula (UVA-1) was changed to a compound represented by Formula (UVA-4).

### (Example 14) Preparation of Light-Selective Absorption Composition (5)

A light-selective absorption composition (5) was prepared in the same manner as in Example 10 except that a compound represented by Formula (UVA-1) was changed to a compound represented by Formula (UVA-5).

### (Example 15) Preparation of Light-Selective Absorption Composition (6)

A light-selective absorption composition (6) was prepared in the same manner as in Example 10 except that a compound represented by Formula (UVA-1) was changed to a compound represented by Formula (UVA-6).

### (Example 16) Preparation of Light-Selective Absorption Composition (7)

A light-selective absorption composition (7) was prepared in the same manner as in Example 10 except that a compound represented by Formula (UVA-1) was changed to a compound represented by Formula (UVA-7).

### (Example 17) Preparation of Light-Selective Absorption Composition (8)

A light-selective absorption composition (8) was prepared in the same manner as in Example 10 except that a compound represented by Formula (UVA-1) was changed to a compound represented by Formula (UVA-8).

### (Example 18) Preparation of Light-Selective Absorption Composition (9)

A light-selective absorption composition (9) was prepared in the same manner as in Example 10 except that a compound represented by Formula (UVA-1) was changed to a compound represented by Formula (UVA-9).

### (Preparation Example 1) Preparation of Light-Selective Absorption Composition (A1)

A light-selective absorption composition (A1) was prepared in the same manner as in Example 10 except that a compound represented by Formula (UVA-1) was changed to a compound represented by Formula (UVA-A1).

### (Preparation Example 2) Preparation of Light-Selective Absorption Composition (A2)

A light-selective absorption composition (A2) was prepared in the same manner as in Example 10 except that a compound represented by Formula (UVA-1) was changed to a compound represented by Formula (UVA-A2).

### (Preparation Example 3) Preparation of Light-Selective Absorption Composition (A3)

A light-selective absorption composition (A3) was prepared in the same manner as in Example 10 except that a compound represented by Formula (UVA-1) was changed to a compound represented by Formula (UVA-A4).

### (Example 19) Preparation of Film with Cured Layer (1)

A surface of a resin film [Trade name: “ZEONOR”, produced by Zeon Corporation] made of a cyclic polyolefin resin and having a thickness of 23 μm was subjected to a corona discharge treatment, and the corona discharge-treated surface was coated with the light-selective absorption composition (6) using a bar coater. The coated film was charged into a drying oven and dried at 100° C. for 2 minutes. The dried coating film was charged into a nitrogen replacement box, nitrogen was sealed in the box for 1 minute, and then ultraviolet irradiation was performed from the coated surface side to obtain a film with a cured layer (6). The thickness of the cured layer was about 6.0 μm.

As an ultraviolet irradiation device, an ultraviolet irradiation device with a belt conveyor [a lamp was “H valve” manufactured by Fusion UV Systems, Inc.] was used, and ultraviolet rays were irradiated so that the integrated amount of light was 400 mJ/cm² (UVB).

### (Comparative Example 1) Preparation of Film with Cured Layer (A1)

A film with a cured layer (A1) was obtained in the same manner as in Example 19 except that the light-selective absorption composition (6) was replaced with the light-selective absorption composition (A1).

### (Comparative Example 2) Preparation of Film with Cured Layer (A2)

A film with a cured layer (A2) was obtained in the same manner as in Example 19 except that the light-selective absorption composition (6) was replaced with the light-selective absorption composition (A2).

### (Comparative Example 3) Preparation of Film with Cured Layer (A3)

A film with a cured layer (A3) was obtained in the same manner as in Example 19 except that the light-selective absorption composition (6) was replaced with the light-selective absorption composition (A3).

<Absorbance Measurement of Film with Cured Layer>

The film with a cured layer (1) obtained in Example 19 was cut into a size of 30 mm×30 mm to give a sample (1). The obtained sample (1) and alkali-free glass [trade name “EAGLE XG” produced by Corning Incorporated] were bonded to each other with an acrylic pressure-sensitive adhesive interposed therebetween to obtain a sample (2). The absorbance of the prepared sample (2) in the wavelength range of 300 to 800 nm was measured every 1 nm step using a spectrophotometer (UV-2450: manufactured by Shimadzu Corporation). The measured absorbances at a wavelength of 395 nm and a wavelength of 430 nm were taken as the absorbances at a wavelength of 395 nm and a wavelength of 430 nm of the film with a cured layer (1). The results are shown in Table 1. The absorbance of the alkali-free glass at a wavelength of 395 nm and a wavelength of 430 nm is almost 0, the absorbance of the resin film made of the cyclic polyolefin resin at a wavelength of 395 nm and a wavelength of 430 nm is almost 0, and the absorbance of the acrylic pressure-sensitive adhesive at a wavelength of 395 nm and a wavelength of 430 nm is almost 0.

<Measurement of Absorbance Retention of Film with Cured Layer>

The sample (2) after the absorbance measurement was charged into a Sunshine weathermeter (manufactured by Suga Test Instruments Co., Ltd.) for 48 hours under the conditions of a temperature of 63° C. and a relative humidity of 50% RH to perform a weather resistance test. The absorbance of the sample (2) after the weather resistance test was measured in the same manner as described above. From the measured absorbance, the absorbance retention of the sample (2) at a wavelength of 395 nm was determined based on the following formula. The results are shown in Table 1. A value of the absorbance retention closer to 100 indicates that the light selective absorption function is not deteriorated and good weather resistance is obtained. A (395) represents absorbance at a wavelength of 395 nm.

Absorbance retention (%)=(A(395) after durability test/A(395) before durability test)×100

A film with a cured layer (A1), a film with a cured layer (A2), and a film with a cured layer (A3) were each used instead of the film with a cured layer (1), and evaluation was performed in the same manner as in the film with a cured layer (1). The results are shown in Table 1.

TABLE 1

| | | | | A (395)/ | Absorbance |
|---|---|---|---|---|---|
| | Compounds | A (395) | A (430) | A (430) | retention |
| Example 19 | Formula (UVA-6) | 1.24 | 0.03 | 40.0 | 63.9 |
| Comparative | Formula (UVA-A1) | 2.08 | 0.05 | 40.8 | 4.9 |
| Example 1 | | | | | |
| Comparative | Formula (UVA-A2) | 2.51 | 0.04 | 58.3 | 6.6 |
| Example 2 | | | | | |
| Comparative | Formula (UVA-A4) | 1.72 | 0.26 | 6.7 | 35.1 |
| Example 3 | | | | | |

### (Example 20) Preparation of Optical Film (1)

A resin solution (solid concentration: 25% by mass) containing 70 parts of a polymethyl methacrylate resin (SUMIPEX MH produced by Sumitomo Chemical Co., Ltd.), 30 parts of rubber particles having a particle diameter of 250 nm and having a core-shell structure of polymethyl methacrylate resin (PMMA)/polybutyl acrylate resin (PBA), 2 parts of a compound represented by Formula (UVA-6), and 2-butanone was charged into a mixing tank, and stirred to dissolve the respective components.

The obtained solution was uniformly cast on a glass support using an applicator, dried in an oven at 40° C. for 10 minutes, and then further dried in an oven at 80° C. for 10 minutes. After drying, an optical film (1) was peeled off from the glass support to obtain the optical film (1) having a light-selective absorption capacity. The film thickness of the optical film (1) after drying was 30 μm.

### (Example 21) Preparation of Optical Film (2)

A resin solution (solid concentration: 7% by mass) containing 100 parts of cellulose triacetate (degree of acetyl substitution: 2.87), 2 parts of a compound represented by Formula (UVA-6), and a mixed solution of chloroform and ethanol (mass ratio, chloroform:ethanol=90:10) was charged into a mixing tank, and stirred to dissolve the respective components.

The obtained solution was uniformly cast on a glass support using an applicator, dried in an oven at 40° C. for 10 minutes, and then further dried in an oven at 80° C. for 10 minutes. After drying, an optical film (2) was peeled off from the glass support to obtain the optical film (2) having a light-selective absorption capacity. The film thickness of the optical film (2) after drying was 30 μm.

### (Example 22) Preparation of Optical Film (3)

A resin solution (solid concentration: 20% by mass) containing 100 parts of a cycloolefin polymer resin (ARTON F 4520 produced by JSRCorporation), 2 parts of a compound represented by Formula (UVA-6), and a mixed solution of dichloromethane and toluene (mass ratio, dichloromethane:toluene=50:50) was charged into a mixing tank, and stirred to dissolve each component.

The obtained solution was uniformly cast on a glass support using an applicator, dried in an oven at 40° C. for 10 minutes, and then further dried in an oven at 80° C. for 10 minutes. After drying, an optical film (3) was peeled off from the glass support to obtain the optical film (3) having a light-selective absorption capacity. The film thickness of the optical film (3) after drying was 30 μm.

### (Comparative Example 4) Preparation of Optical Film (4)

An optical film (4) was produced in the same manner as in Example 20 except that the compound represented by Formula (UVA-1) was changed to a compound represented by Formula (UVA-A1).

### (Comparative Example 5) Preparation of Optical Film (5)

An optical film (5) was produced in the same manner as in Example 21 except that the compound represented by Formula (UVA-1) was changed to a compound represented by Formula (UVA-A1).

### (Comparative Example 6) Preparation of Optical Film (6)

An optical film (6) was produced in the same manner as in Example 20 except that the compound represented by Formula (UVA-1) was changed to a compound represented by Formula (UVA-A4).

### (Comparative Example 7) Preparation of Optical Film (7)

An optical film (7) was produced in the same manner as in Example 21 except that the compound represented by Formula (UVA-1) was changed to a compound represented by Formula (UVA-A4).

<Absorbance Measurement of Optical Film>

One surface of the optical film (1) obtained in Example 20 was subjected to a corona discharge treatment, and then an acrylic pressure-sensitive adhesive was bonded thereto by a laminator, and cured for 7 days under conditions of a temperature of 23° C. and a relative humidity of 65% RH to obtain an optical film with a pressure-sensitive adhesive (1). Next, the optical film with a pressure-sensitive adhesive (1) was cut into a size of 30 mm×30 mm and bonded to alkali-free glass [trade name “EAGLE XG” produced by Corning Incorporated] to prepare a sample (3). The absorbance of the prepared sample (3) in the wavelength range of 300 to 800 nm was measured every 1 nm step using a spectrophotometer (UV-2450: manufactured by Shimadzu Corporation). The measured absorbances at a wavelength of 395 nm and a wavelength of 430 nm were taken as the absorbances at a wavelength of 395 nm and a wavelength of 430 nm of the optical film (1). The results are shown in Table 2. The absorbance of the alkali-free glass at a wavelength of 395 nm and a wavelength of 430 nm is almost 0, and the absorbance of the acrylic pressure-sensitive adhesive at a wavelength of 395 nm and a wavelength of 430 nm is almost 0.

The sample (3) after the absorbance measurement was charged into a Sunshine weathermeter (manufactured by Suga Test Instruments Co., Ltd.) for 200 hours under the conditions of a temperature of 63° C. and a relative humidity of 50% RH to perform a weather resistance test. The absorbance of the sample (3) after the weather resistance test was measured in the same manner as described above. From the measured absorbance, the absorbance retention of the sample at a wavelength of 395 nm was determined based on the following formula. The results are shown in Table 2. A value of the absorbance retention closer to 100 indicates that the light selective absorption function is not deteriorated and good weather resistance is obtained.

Absorbance retention (%)=(A(395) after durability test/A(395) before durability test)×100

Each of the optical films (2) to (7) was used instead of the optical film (1), and evaluation was performed in the same manner as in the optical film (1). The results are shown in Table 2.

TABLE 2

| | | | | | A (395)/ | Absorbance |
|---|---|---|---|---|---|---|
| | Compounds | Resin | A (395) | A (430) | A (430) | retention |
| Example 20 | Formula | Polymethyl | 3.01 | 0.03 | 103.8 | 100.0 |
| | (UVA-6) | methacrylate resin | | | | |
| Example 21 | Formula | Cellulose acetate | 3.42 | 0.03 | 126.6 | 95.6 |
| | (UVA-6) | resin | | | | |
| Example 22 | Formula | Cycloolefin resin | 3.26 | 0.02 | 163.1 | 76.3 |
| | (UVA-6) | | | | | |
| Comparative | Formula | Polymethyl | 3.56 | 0.02 | 161.9 | 11.3 |
| Example 4 | (UVA-A1) | methacrylate resin | | | | |
| Comparative | Formula | Cellulose acetate | 3.64 | 0.04 | 93.4 | 3.4 |
| Example 5 | (UVA-A1) | resin | | | | |
| Comparative | Formula | Polymethyl | 4.04 | 0.56 | 7.2 | 53.8 |
| Example 6 | (UVA-A4) | methacrylate resin | | | | |
| Comparative | Formula | Cellulose acetate | 4.22 | 0.74 | 5.7 | 46.6 |
| Example 7 | (UVA-A4) | resin | | | | |

(Example 23) Preparation of Pressure-Sensitive Adhesive Composition (1)

<Preparation of Acrylic Resin (A)>

In a reaction container equipped with a cooling tube, a nitrogen introduction tube, a thermometer, and a stirrer, a mixed solution of 81.8 parts of ethyl acetate as a solvent, 70.4 parts of butyl acrylate, 20.0 parts of methyl acrylate, 8.0 parts of 2-phenoxyethyl acrylate, 1.0 part of 2-hydroxyethyl acrylate, and 0.6 parts of acrylic acid as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 1.42 million and Mw/Mn of 5.2 as measured by GPC. This is referred to as an acrylic resin (A).

<Preparation of Pressure-Sensitive Adhesive Composition (1)>

To 100 parts of solid content of the ethyl acetate solution (1) (resin concentration: 20%) of the acrylic resin (A) synthesized above, 0.5 parts of a crosslinking agent (ethyl acetate solution of trimethylolpropane adduct of tolylene diisocyanate (solid concentration: 75%), produced by Tosoh Corporation, trade name “Coronate L”), 0.5 parts of a silane compound (3-glycidoxypropyltrimethoxysilane, produced by Shin-Etsu Chemical Co., Ltd., trade name “KBM 403”), and 2.0 parts of a compound represented by Formula (UVA-1) were mixed, and ethyl acetate was further added so that the solid content concentration was 14% to obtain a pressure-sensitive adhesive composition (1). The blending amount of the crosslinking agent is the number of parts by mass as an active component.

### (Example 24) Preparation of Pressure-Sensitive Adhesive Composition (2)

A pressure-sensitive adhesive composition (2) was obtained in the same manner as that in Example 23 except that the compound represented by Formula (UVA-1) was changed to the compound represented by Formula (UVA-2).

### (Example 25) Preparation of Pressure-Sensitive Adhesive Composition (3)

A pressure-sensitive adhesive composition (3) was obtained in the same manner as that in Example 23 except that the compound represented by Formula (UVA-1) was changed to the compound represented by Formula (UVA-3).

### (Example 26) Preparation of Pressure-Sensitive Adhesive Composition (4)

A pressure-sensitive adhesive composition (4) was obtained in the same manner as that in Example 23 except that the compound represented by Formula (UVA-1) was changed to the compound represented by Formula (UVA-4).

### (Example 27) Preparation of Pressure-Sensitive Adhesive Composition (5)

A pressure-sensitive adhesive composition (5) was obtained in the same manner as that in Example 23 except that the compound represented by Formula (UVA-1) was changed to the compound represented by Formula (UVA-5).

### (Example 28) Preparation of Pressure-Sensitive Adhesive Composition (6)

A pressure-sensitive adhesive composition (6) was obtained in the same manner as that in Example 23 except that the compound represented by Formula (UVA-1) was changed to the compound represented by Formula (UVA-6).

### (Example 29) Preparation of Pressure-Sensitive Adhesive Composition (7)

A pressure-sensitive adhesive composition (7) was obtained in the same manner as that in Example 23 except that the compound represented by Formula (UVA-1) was changed to the compound represented by Formula (UVA-7).

### (Example 30) Preparation of Pressure-Sensitive Adhesive Composition (8)

A pressure-sensitive adhesive composition (8) was obtained in the same manner as that in Example 23 except that the compound represented by Formula (UVA-1) was changed to the compound represented by Formula (UVA-8).

### (Example 31) Preparation of Pressure-Sensitive Adhesive Composition (9)

A pressure-sensitive adhesive composition (9) was obtained in the same manner as that in Example 23 except that the compound represented by Formula (UVA-1) was changed to the compound represented by Formula (UVA-9).

### (Comparative Example 8) Preparation of Pressure-Sensitive Adhesive Composition (10)

A pressure-sensitive adhesive composition (10) was obtained in the same manner as that in Example 23 except that the compound represented by Formula (UVA-1) was changed to the compound represented by Formula (UVA-A1).

### (Example 32) Preparation of Pressure-Sensitive Adhesive Layer (1) and Pressure-Sensitive Adhesive Sheet (1)

The obtained pressure-sensitive adhesive composition (6) was applied to a release-treated surface of a separator film [trade name “PLR-382190” available from Lintec Corporation] made of a release-treated polyethylene terephthalate film using an applicator, and dried at 100° C. for 1 minute to prepare a pressure-sensitive adhesive layer (1). The thickness of the obtained pressure-sensitive adhesive layer was 15 μm.

The obtained pressure-sensitive adhesive layer (1) was bonded to a 23 μm ultraviolet absorber-containing cycloolefin film [trade name “ZEONOR” available from Zeon Corporation] by a laminator, and then aged for 7 days under conditions of a temperature of 23° C. and a relative humidity of 65% to obtain a pressure-sensitive adhesive sheet (1).

### (Example 33) Preparation of Pressure-Sensitive Adhesive Layer (2) and Pressure-Sensitive Adhesive Sheet (2)

A pressure-sensitive adhesive layer (2) and a pressure-sensitive adhesive sheet (2) were prepared in the same manner as in Example 32 except that the pressure-sensitive adhesive composition (6) was changed to the pressure-sensitive adhesive composition (7).

### (Comparative Example 9) Preparation of Pressure-Sensitive Adhesive Layer (3) and Pressure-Sensitive Adhesive Sheet (3)

A pressure-sensitive adhesive layer (3) and a pressure-sensitive adhesive sheet (3) were prepared in the same manner as in Example 32 except that the pressure-sensitive adhesive composition (6) was changed to the pressure-sensitive adhesive composition (10).

<Absorbance Measurement of Pressure-Sensitive Adhesive Sheet>

The obtained pressure-sensitive adhesive sheet (1) was cut into a size of 30 mm×30 mm, the separate film was peeled off, and the pressure-sensitive adhesive layer (1) and alkali-free glass [trade name “EAGLE XG” produced by Corning Incorporated] were bonded to each other to prepare a sample (4). The absorbance of the prepared sample (4) in the wavelength range of 300 to 800 nm was measured every 1 nm step using a spectrophotometer (UV-2450: manufactured by Shimadzu Corporation). The measured absorbances at a wavelength of 395 nm and a wavelength of 430 nm were taken as the absorbances at a wavelength of 395 nm and a wavelength of 430 nm of the pressure-sensitive adhesive sheet (1). The results are shown in Table 3. Both the cycloolefin film alone and the non-alkali glass alone have zero absorbance at a wavelength of 395 nm and a wavelength of 430 nm.

<Measurement of Absorbance Retention of Pressure-Sensitive Adhesive Sheet>

The sample (4) after the absorbance measurement was charged into a Sunshine weathermeter (manufactured by Suga Test Instruments Co., Ltd.) for 200 hours under the conditions of a temperature of 63° C. and a relative humidity of 50% RH to perform a weather resistance test. The absorbance of the sample (4) taken out was measured in the same manner as described above. From the measured absorbance, the absorbance retention of the sample at 395 nm was determined based on the following formula. The results are shown in Table 3. A value of the absorbance retention closer to 100 indicates that the light selective absorption function is not deteriorated and good weather resistance is obtained.

Absorbance retention (%)=(A(395) after durability test/A(395) before durability test)×100

Evaluation was performed in the same manner as in the case of the pressure-sensitive adhesive sheet (1) using each of the pressure-sensitive adhesive sheet (2) and the pressure-sensitive adhesive sheet (3) instead of the pressure-sensitive adhesive sheet (1). The results are shown in Table 3.

TABLE 3

| | | | | A (395)/ | Absorbance |
|---|---|---|---|---|---|
| | Compounds | A (395) | A (430) | A (430) | retention |
| Example 32 | Formula (UVA-6) | 1.45 | 0.01 | 111.4 | 100 |
| Example 33 | Formula (UVA-7) | 1.26 | 0.03 | 43.3 | 99.4 |
| Comparative | Formula (UVA-A1) | 2.82 | 0.01 | 216.7 | 6.8 |
| Example 9 | | | | | |

### (Example 34) Synthesis of Compound Represented by Formula (UVA-10)

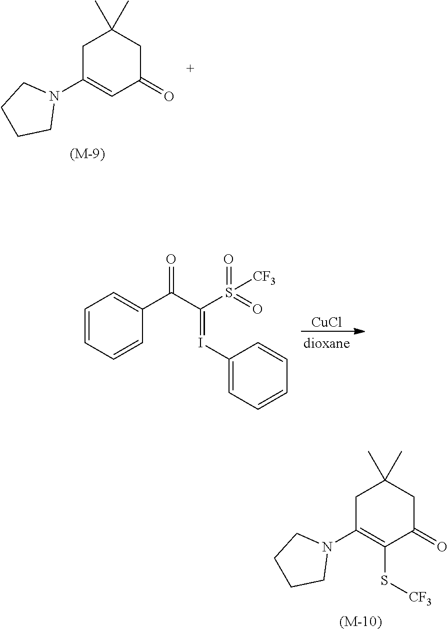

Under a nitrogen atmosphere, 2.5 parts of a compound represented by Formula (M-9), 15.1 parts of benzoyl (phenyliodonium) (trifluoromethanesulfonyl) methanide, 0.4 parts of copper (I) chloride, and 100 parts of dioxane were mixed. The obtained mixture was allowed to stir at 30° C. for 3 hours. The solvent was distilled off from the obtained mixture and purified to obtain 1.7 parts of a compound represented by Formula (M-10).

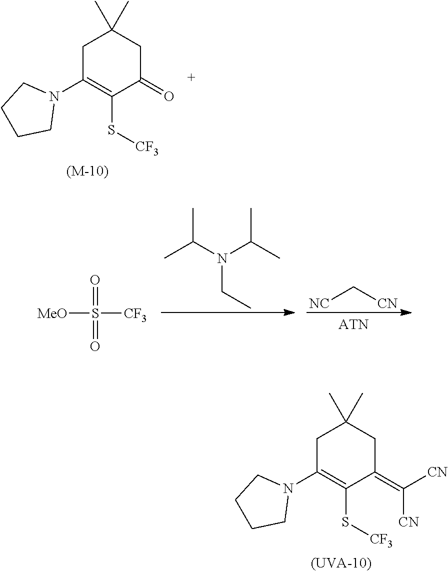

Under a nitrogen atmosphere, 1.5 parts of a compound represented by Formula (M-10), 1.4 parts of methyl triflate, and 10 parts of acetonitrile were mixed, and the mixture was stirred at 20 to 30° C. for 3 hours. 1.3 parts of diisopropylethylamine and 0.7 parts of malononitrile were added to the obtained mixture, and the mixture was stirred at 20 to 30° C. for 3 hours. The solvent was distilled off from the obtained mixture and purified to obtain 1.0 part of a compound represented by Formula (UVA-10).

In the same manner as described above, LC-MS measurement and ¹H-NMR analysis were performed, and it was confirmed that a compound represented by Formula (UVA-10) was produced.

¹H-NMR (deuterated DMSO) δ: 1.00 (s, 3H), 1.15 (s, 3H), 1.86 (m, 2H), 2.18 (m, 2H), 2.32-2.91 (m, 4H), 3.50-4.20 (m, 4H)

LC-MS; [M+H]⁺=343.5

In addition, the maximum absorption wavelength and the gram absorption coefficient were measured in the same manner as described above. The maximum absorption wavelength of the obtained compound represented by Formula (UVA-10) was 384.2 nm. The obtained compound represented by Formula (UVA-10) had ε(λmax) of 1.29 L/(g·cm), ε(λmax+30 nm) of 0.075 L/(g·cm), and ε(λmax)/ε(λmax+30 nm) of 17.2.

### (Example 35) Synthesis of Compound Represented by Formula (UVA-11)

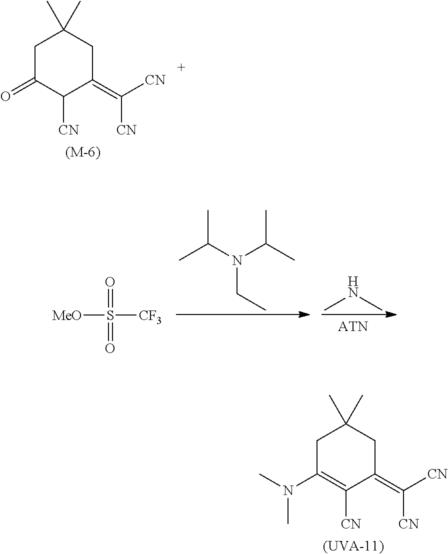

Under a nitrogen atmosphere, 5 parts of a compound represented by Formula (M-6), 4.9 parts of methyl triflate, 3.8 parts of diisopropylethylamine and 10 parts of acetonitrile were mixed, and the mixture was stirred at 20 to 30° C. for 3 hours. 5 parts of dimethylamine was added to the obtained mixture, and the mixture was stirred at 20 to 30° C. for 3 hours. The solvent was distilled off from the obtained mixture and purified to obtain 3.1 parts of a compound represented by Formula (UVA-11).

In the same manner as described above, LC-MS measurement and ¹H-NMR analysis were performed, and it was confirmed that a compound represented by Formula (UVA-11) was produced.

¹H-NMR (deuterated DMSO) δ: 1.08 (s, 6H), 2.42 (s, 2H), 2.55 (s, 2H), 3.40 (m, 6H)

LC-MS; [M+H]⁺=241.5

In addition, the maximum absorption wavelength and the gram absorption coefficient were measured in the same manner as described above. The maximum absorption wavelength of the obtained compound represented by Formula (UVA-11) was 379.4 nm. The obtained compound represented by Formula (UVA-11) had ε(λmax) of 1.93 L/(g·cm), ε(λmax+30 nm) of 0.063 L/(g·cm), and ε(λmax)/ε(λmax+30 nm) of 30.6.

### (Example 36) Synthesis of Compound Represented by Formula (UVA-12)

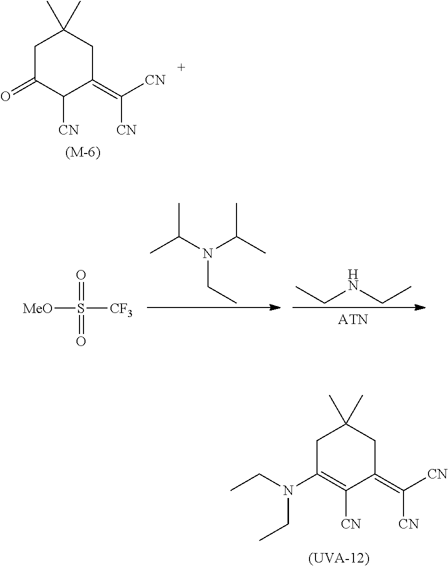

Under a nitrogen atmosphere, 5 parts of a compound represented by Formula (M-6), 4.9 parts of methyl triflate, 3.8 parts of diisopropylethylamine and 10 parts of acetonitrile were mixed, and the mixture was stirred at 20 to 30° C. for 3 hours. 8.4 parts of diethylamine was added to the obtained mixture, and the mixture was stirred at 20 to 30° C. for 3 hours. The solvent was distilled off from the obtained mixture and purified to obtain 2.9 parts of a compound represented by Formula (UVA-12).

In the same manner as described above, LC-MS measurement and ¹H-NMR analysis were performed, and it was confirmed that a compound represented by Formula (UVA-12) was produced.

¹H-NMR (deuterated DMSO) δ: 1.08 (s, 6H), 1.39 (t, 6H), 2.44 (s, 2H), 2.58 (s, 2H), 3.74 (m, 4H)

LC-MS; [M+H]⁺=269.5

In addition, the maximum absorption wavelength and the gram absorption coefficient were measured in the same manner as described above. The maximum absorption wavelength of the obtained compound represented by Formula (UVA-12) was 380.5 nm. The obtained compound represented by Formula (UVA-12) had ε(λmax) of 1.75 L/(g·cm), ε(λmax+30 nm) of 0.098 L/(g·cm), and ε(λmax)/ε(λmax+30 nm) of 17.6.

### (Example 37) Synthesis of Compound Represented by Formula (UVA-13)

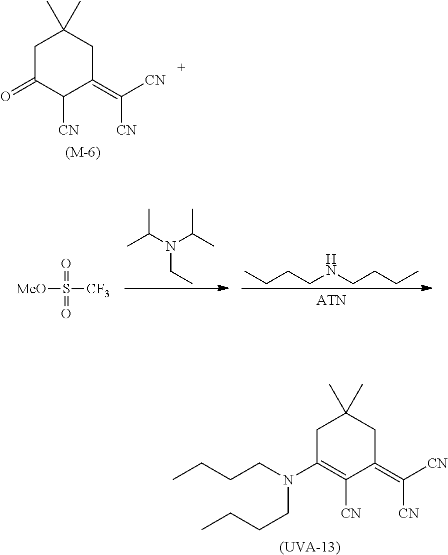

Under a nitrogen atmosphere, 5 parts of a compound represented by Formula (M-6), 4.9 parts of methyl triflate, 3.8 parts of diisopropylethylamine and 10 parts of acetonitrile were mixed, and the mixture was stirred at 20 to 30° C. for 3 hours. 14.8 parts of dibutylamine was added to the obtained mixture, and the mixture was stirred at 20 to 30° C. for 3 hours. The solvent was distilled off from the obtained mixture and purified to obtain 2.5 parts of a compound represented by Formula (UVA-13).

In the same manner as described above, LC-MS measurement and ¹H-NMR analysis were performed, and it was confirmed that a compound represented by Formula (UVA-13) was produced.

¹H-NMR (deuterated DMSO) δ: 0.99 (t, 6H), 1.07 (s, 6H), 1.32 to 1.46 (m, 4H), 1.70 (m, 4H), 2.40 (s, 2H), 2.57 (s, 2H), 3.32 to 3.85 (m, 4H).

LC-MS; [M+H]⁺=325.5

In addition, the maximum absorption wavelength and the gram absorption coefficient were measured in the same manner as described above. The maximum absorption wavelength of the obtained compound represented by Formula (UVA-13) was 382.8 nm. The obtained compound represented by Formula (UVA-13) had ε(λmax) of 1.42 L/(g·cm), ε(λmax+30 nm) of 0.095 L/(g·cm), and ε(λmax)/ε(λmax+30 nm) of 14.9.

### (Example 38) Synthesis of Compound Represented by Formula (UVA-14)

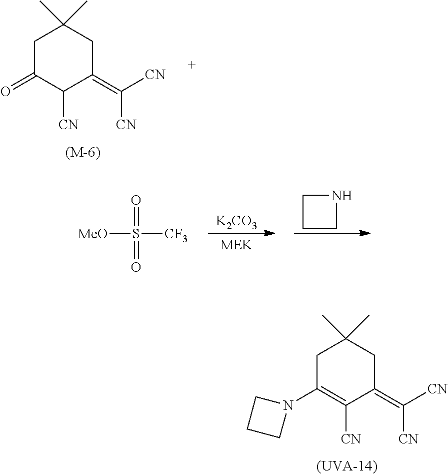

Under a nitrogen atmosphere, 5 parts of a compound represented by Formula (M-6), 3.6 parts of potassium carbonate, 7.7 parts of methyl triflate and 40 parts of methyl ethyl ketone were mixed, and the mixture was stirred at 0 to 5° C. for 4 hours. 2 parts of azetidine was added to the obtained mixture, and the mixture was stirred at 0 to 5° C. for 10 minutes. The solvent was distilled off from the obtained mixture and purified to obtain 2.6 parts of a compound represented by Formula (UVA-14).

LC-MS measurement and ¹H-NMR analysis were performed, and it was confirmed that a compound represented by Formula (UVA-14) was produced.

¹H-NMR (deuterated DMSO) δ: 1.05 (s, 6H), 2.14 (s, 2H), 2.45-2.53 (m, 4H), 4.36 (t, 2H), 4.91 (t, 2H)

LC-MS; [M+H]⁺=253.3

In addition, the maximum absorption wavelength and the gram absorption coefficient were measured in the same manner as described above. The maximum absorption wavelength of the obtained compound represented by Formula (UVA-14) was 377.2 nm. The obtained compound represented by Formula (UVA-14) had ε(λmax) of 1.93 L/(g·cm), ε(λmax+30 nm) of 0.028 L/(g·cm), and ε(λmax)/ε(λmax+30 nm) of 68.9.

### (Example 39) Synthesis of Compound Represented by Formula (UVA-15)

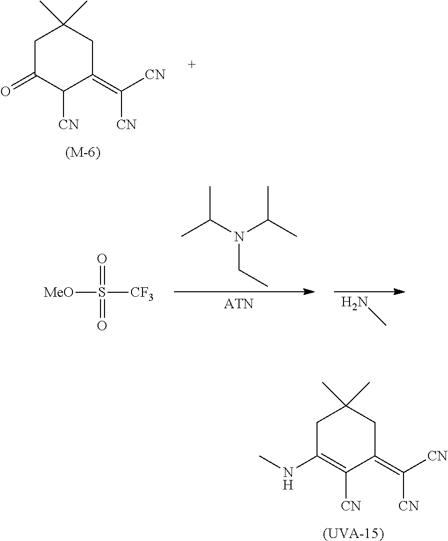

Under a nitrogen atmosphere, 4.0 parts of a compound represented by Formula (M-6), 3.7 parts of methyl triflate, and 40 parts of acetonitrile were mixed, and the mixture was stirred at 20 to 30° C. for 3 hours. To the obtained mixture, 2.9 parts of diisopropylethylamine and 40 parts of a solution obtained by dissolving methylamine in tetrahydrofuran (concentration of methylamine; 7% by mass) was added, and the mixture was stirred at 20 to 30° C. for 3 hours. The solvent was distilled off from the obtained mixture and purified to obtain 1.9 parts of a compound represented by Formula (UVA-15).

LC-MS measurement and ¹H-NMR analysis were performed, and it was confirmed that a compound represented by Formula (UVA-15) was produced.

¹H-NMR (deuterated DMSO) δ: 0.98 (s, 6H), 2.48 to 2.58 (m, 4H), 3.03 (s, 3H), 9.15 (s, 1H)

LC-MS; [M+H]⁺=226.5

In addition, the maximum absorption wavelength and the gram absorption coefficient were measured in the same manner as described above. The maximum absorption wavelength of the obtained compound represented by Formula (UVA-15) was 364.8 nm. The obtained compound represented by Formula (UVA-15) had ε(λmax) of 1.86 L/(g·cm), ε(λmax+30 nm) of 0.066 L/(g·cm), and ε(λmax)/ε(λmax+30 nm) of 28.2.

### (Example 40) Synthesis of Compound Represented by Formula (UVA-16)

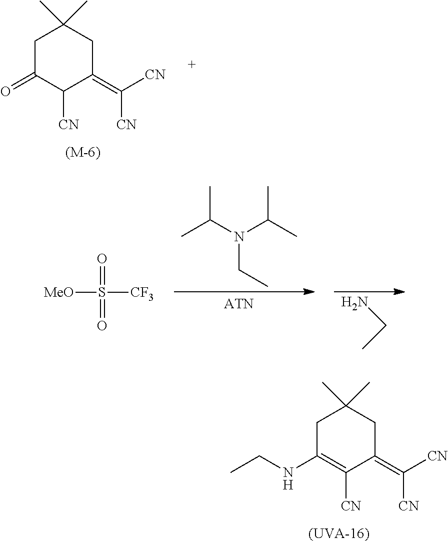

Under a nitrogen atmosphere, 4.0 parts of a compound represented by Formula (M-6), 3.7 parts of methyl triflate, and 40 parts of acetonitrile were mixed, and the mixture was stirred at 20 to 30° C. for 3 hours. To the obtained mixture, 2.9 parts of diisopropylethylamine and 40 parts of a solution obtained by dissolving ethylamine in tetrahydrofuran (concentration of ethylamine; 10% by mass) was added, and the mixture was stirred at 20 to 30° C. for 3 hours. The solvent was distilled off from the obtained mixture and purified to obtain 1.5 parts of a compound represented by Formula (UVA-16).

LC-MS measurement and ¹H-NMR analysis were performed, and it was confirmed that a compound represented by Formula (UVA-16) was produced.

¹H-NMR (deuterated DMSO) δ: 0.98 (s, 6H), 2.48-2.58 (m, 4H), 3.03 (t, 3H), 4.21 (m, 2H), 9.15 (s, 1H)

LC-MS; [M+H]⁺=240.5

In addition, the maximum absorption wavelength and the gram absorption coefficient were measured in the same manner as described above. The maximum absorption wavelength of the obtained compound represented by Formula (UVA-16) was 364.8 nm. The obtained compound represented by Formula (UVA-16) had ε(λmax) of 1.80 L/(g·cm), ε(λmax+30 nm) of 0.074 L/(g·cm), and ε(λmax)/ε(λmax+30 nm) of 24.4.

### (Example 41) Synthesis of Compound Represented by Formula (UVA-17)

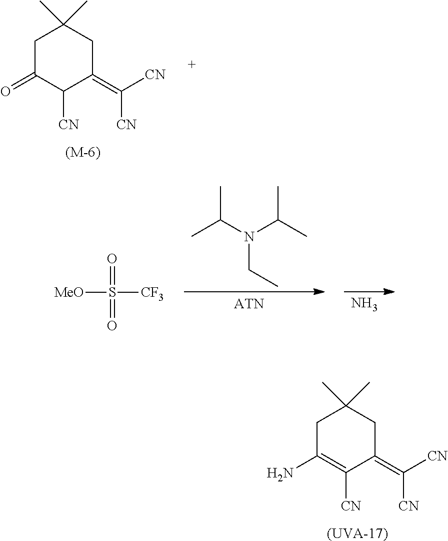

Under a nitrogen atmosphere, 1.7 parts of a compound represented by Formula (M-6), 1.6 parts of methyl triflate, and 17 parts of acetonitrile were mixed, and the mixture was stirred at 20 to 30° C. for 3 hours. To the obtained mixture, 1.2 parts of diisopropylethylamine and 100 parts of a solution obtained by dissolving ammonia in tetrahydrofuran (molar concentration of ammonia; 0.4 mol %) and stirred at 20 to 30° C. for 3 hours. The solvent was distilled off from the obtained mixture and purified to obtain 0.7 parts of a compound represented by Formula (UVA-17).

LC-MS measurement and ¹H-NMR analysis were performed, and it was confirmed that a compound represented by Formula (UVA-17) was produced.

¹H-NMR (deuterated DMSO) δ: 0.98 (s, 6H), 2.48-2.58 (m, 4H), 9.15 (m, 2H)

LC-MS; [M+H]⁺=213.5

In addition, the maximum absorption wavelength and the gram absorption coefficient were measured in the same manner as described above. The maximum absorption wavelength of the obtained compound represented by Formula (UVA-17) was 352.6 nm. The obtained compound represented by Formula (UVA-17) had ε(λmax) of 1.75 L/(g·cm), ε(λmax+30 nm) of 0.11 L/(g·cm), and ε(λmax)/ε(λmax+30 nm) of 15.9.

### (Example 42) Synthesis of Compound Represented by Formula (UVA-18)

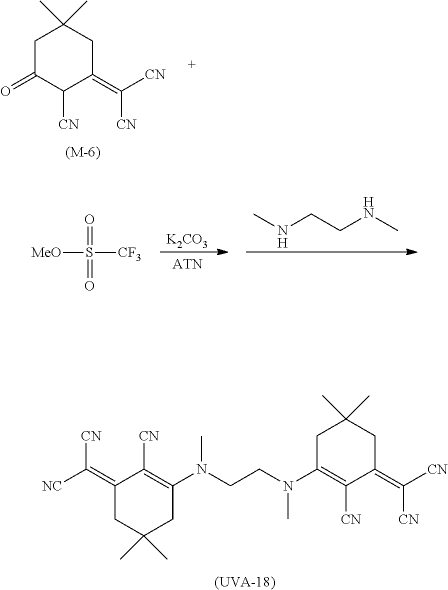

Under a nitrogen atmosphere, 3.5 parts of a compound represented by Formula (M-6), 3.2 parts of methyl triflate, and 35 parts of acetonitrile were mixed, and the mixture was stirred at 20 to 30° C. for 3 hours. 2.2 parts of potassium carbonate and 0.8 parts of N,N′-dimethylethylenediamine were added to the obtained mixture, and the mixture was stirred at 20 to 30° C. for 3 hours. The solvent was distilled off from the obtained mixture and purified to obtain 0.4 parts of a compound represented by Formula (UVA-18).

LC-MS measurement and ¹H-NMR analysis were performed, and it was confirmed that a compound represented by Formula (UVA-18) was produced.

¹H-NMR (deuterated DMSO) δ: 0.98 (s, 12H), 2.67 (m, 4H), 3.44 (m, 8H), 4.05 (m, 6H)

LC-MS; [M+H]⁺=479.7

In addition, the maximum absorption wavelength and the gram absorption coefficient were measured in the same manner as described above. The maximum absorption wavelength of the obtained compound represented by Formula (UVA-18) was 391.4 nm. The obtained compound represented by Formula (UVA-18) had ε(λmax) of 1.52 L/(g·cm), ε(λmax+30 nm) of 0.036 L/(g·cm), and ε(λmax)/ε(λmax+30 nm) of 42.2.

### (Example 43) Synthesis of Compound Represented by Formula (UVA-19)

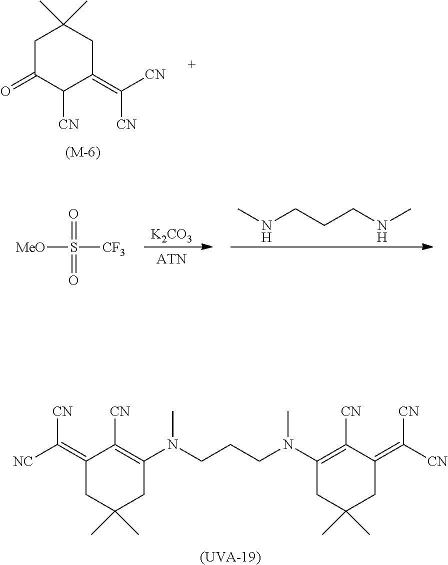

Under a nitrogen atmosphere, 3.5 parts of a compound represented by Formula (M-6), 3.2 parts of methyl triflate, and 35 parts of acetonitrile were mixed, and the mixture was stirred at 20 to 30° C. for 3 hours. 2.2 parts of potassium carbonate and 1.0 part of N,N′-dimethyltrimethylenediamine were added to the obtained mixture, and the mixture was stirred at 20 to 30° C. for 3 hours. The solvent was distilled off from the obtained mixture and purified to obtain 0.2 parts of a compound represented by Formula (UVA-19).

LC-MS measurement and ¹H-NMR analysis were performed, and it was confirmed that a compound represented by Formula (UVA-19) was produced.

¹H-NMR (deuterated DMSO) δ: 0.99 (s, 12H), 2.50 (m, 8H), 2.66 (m, 6H), 3.32 (m, 6H)

LC-MS; [M+H]⁺=493.7

In addition, the maximum absorption wavelength and the gram absorption coefficient were measured in the same manner as described above. The maximum absorption wavelength of the obtained compound represented by Formula (UVA-19) was 384.9 nm. The obtained compound represented by Formula (UVA-19) had ε(λmax) of 1.63 L/(g·cm), ε(λmax+30 nm) of 0.036 L/(g·cm), and ε(λmax)/ε(λmax+30 nm) of 45.3.

### (Example 44) Preparation of Light-Selective Absorption Composition (10)

A light-selective absorption composition (10) was prepared in the same manner as in Example 10 except that a compound represented by Formula (UVA-1) was changed to a compound represented by Formula (UVA-10).

### (Example 45) Preparation of Light-Selective Absorption Composition (11)

A light-selective absorption composition (11) was prepared in the same manner as in Example 10 except that a compound represented by Formula (UVA-1) was changed to a compound represented by Formula (UVA-11).

### (Example 46) Preparation of Light-Selective Absorption Composition (12)

A light-selective absorption composition (12) was prepared in the same manner as in Example 10 except that a compound represented by Formula (UVA-1) was changed to a compound represented by Formula (UVA-12).

### (Example 47) Preparation of Light-Selective Absorption Composition (13)

A light-selective absorption composition (13) was prepared in the same manner as in Example 10 except that a compound represented by Formula (UVA-1) was changed to a compound represented by Formula (UVA-13).

### (Example 48) Preparation of Film with Cured Layer (2)

A film with a cured layer (2) was obtained in the same manner as in Example 19 except that the light-selective absorption composition (1) was replaced with the light-selective absorption composition (11).

### (Example 49) Preparation of Film with Cured Layer (3)

A film with a cured layer (3) was obtained in the same manner as in Example 19 except that the light-selective absorption composition (1) was replaced with the light-selective absorption composition (12).

<Absorbance Measurement and Absorbance Retention Measurement of Film With Cured Layer>

The absorbance was measured in the same manner as in <Absorbance Measurement of Film with Cured Layer> described above except that the film with a cured layer (2) and the film with a cured layer (3) were used instead of the film with a cured layer (1).

In addition, the absorbance retention of the film with a cured layer (1) obtained in Example 19 and the film with a cured layer (A3) obtained in Comparative Example 3 were measured in the same manner as in <Measurement of Absorbance Retention of Film with Cured Layer> described above except that the charging time to the sunshine weathermeter was set to 75 hours.

Further, the absorbance retention was measured in the same manner as in <Measurement of Absorbance Retention of Film with Cured Layer> described above except that the film with a cured layer (2) and the film with a cured layer (3) were used instead of the film with a cured layer (1), and the charging time to the sunshine weathermeter was set to 75 hours.

These results are shown in Table 4. Table 4 also shows the absorbance values of the film with a cured layer (1) obtained in Example 19 and the film with a cured layer (A3) obtained in Comparative Example 3.

TABLE 4

| | | | | A (395)/ | Absorbance |
|---|---|---|---|---|---|
| | Compounds | A (395) | A (430) | A (430) | retention |
| Example 48 | Formula (UVA-11) | 1.199 | 0.044 | 27.3 | 56 |
| Example 49 | Formula (UVA-12) | 1.163 | 0.022 | 52.9 | 54 |
| Example 19 | Formula (UVA-6) | 1.24 | 0.03 | 40.0 | 39.8 |
| Comparative | Formula (UVA-A4) | 1.72 | 0.26 | 6.7 | 7.1 |
| Example 3 | | | | | |

### (Example 50) Preparation of Pressure-Sensitive

Adhesive Composition (11)

A pressure-sensitive adhesive composition (11) was obtained in the same manner as that in Example 23 except that the compound represented by Formula (UVA-1) was changed to the compound represented by Formula (UVA-10).

### (Example 51) Preparation of Pressure-Sensitive Adhesive Composition (12)

A pressure-sensitive adhesive composition (12) was obtained in the same manner as that in Example 23 except that the compound represented by Formula (UVA-1) was changed to the compound represented by Formula (UVA-11).

### (Example 52) Preparation of Pressure-Sensitive Adhesive Composition (13)

A pressure-sensitive adhesive composition (13) was obtained in the same manner as that in Example 23 except that the compound represented by Formula (UVA-1) was changed to the compound represented by Formula (UVA-12).

### (Example 53) Preparation of Pressure-Sensitive Adhesive Composition (14)

A pressure-sensitive adhesive composition (14) was obtained in the same manner as that in Example 23 except that the compound represented by Formula (UVA-1) was changed to the compound represented by Formula (UVA-13).

### (Example 54) Preparation of Pressure-Sensitive Adhesive Layer (4) and Pressure-Sensitive Adhesive Sheet (4)

A pressure-sensitive adhesive layer (4) and a pressure-sensitive adhesive sheet (4) were prepared in the same manner as in Example 32 except that the pressure-sensitive adhesive composition (6) was changed to the pressure-sensitive adhesive composition (9).

### (Example 55) Preparation of Pressure-Sensitive Adhesive Layer (5) and Pressure-Sensitive Adhesive Sheet (5)

A pressure-sensitive adhesive layer (5) and a pressure-sensitive adhesive sheet (5) were prepared in the same manner as in Example 32 except that the pressure-sensitive adhesive composition (6) was changed to the pressure-sensitive adhesive composition (11).

### (Example 56) Preparation of Pressure-Sensitive Adhesive Layer (6) and Pressure-Sensitive Adhesive Sheet (6)

A pressure-sensitive adhesive layer (6) and a pressure-sensitive adhesive sheet (6) were prepared in the same manner as in Example 32 except that the pressure-sensitive adhesive composition (6) was changed to the pressure-sensitive adhesive composition (12).

### (Example 57) Preparation of Pressure-Sensitive Adhesive Layer (7) and Pressure-Sensitive Adhesive Sheet (7)

A pressure-sensitive adhesive layer (7) and a pressure-sensitive adhesive sheet (7) were prepared in the same manner as in Example 32 except that the pressure-sensitive adhesive composition (6) was changed to the pressure-sensitive adhesive composition (13).

### (Example 58) Preparation of Pressure-Sensitive Adhesive Layer (8) and Pressure-Sensitive Adhesive Sheet (8)

A pressure-sensitive adhesive layer (8) and a pressure-sensitive adhesive sheet (8) were prepared in the same manner as in Example 32 except that the pressure-sensitive adhesive composition (6) was changed to the pressure-sensitive adhesive composition (14).

### (Example 59) Preparation of Pressure-Sensitive Adhesive Composition (15)

A pressure-sensitive adhesive composition (15) was obtained in the same manner as that in Example 23 except that the compound represented by Formula (UVA-1) was changed to the compound represented by Formula (UVA-18), and the content of the compound was 1.0 part by mass based on 100 parts by mass of the acrylic resin (A).

### (Example 60) Preparation of Pressure-Sensitive Adhesive Layer (9) and Pressure-Sensitive Adhesive Sheet (9)

A pressure-sensitive adhesive layer (9) and a pressure-sensitive adhesive sheet (9) were prepared in the same manner as in Example 32 except that the pressure-sensitive adhesive composition (6) was changed to the pressure-sensitive adhesive composition (15).

<Absorbance Measurement and Absorbance Retention Measurement of Pressure-Sensitive Adhesive Sheet>

The absorbance and the absorbance retention were measured in the same manner as in <Measurement of Absorbance of Pressure-Sensitive Adhesive Sheet> and <Measurement of Absorbance Retention of Pressure-Sensitive Adhesive Sheet> described above except that the pressure-sensitive adhesive sheet (4) to the pressure-sensitive adhesive sheet (9) were used instead of the pressure-sensitive adhesive sheet (1). The results are shown in Table 5.

TABLE 5

| | | | | A (395)/ | Absorbance |
|---|---|---|---|---|---|
| | Compounds | A (395) | A (430) | A (430) | retention |
| Example 54 | Formula (UVA-9) | 3.18 | 0.025 | 127.4 | 96.2 |
| Example 55 | Formula (UVA-10) | 2.35 | 0.031 | 75.9 | 84.8 |
| Example 56 | Formula (UVA-11) | 2.02 | 0.009 | 224.3 | 98.9 |
| Example 57 | Formula (UVA-12) | 2.29 | 0.014 | 163.9 | 98.2 |
| Example 58 | Formula (UVA-13) | 0.97 | 0.001 | 974.0 | 85.8 |
| Example 60 | Formula (UVA-18) | 2.16 | 0.200 | 10.8 | 72.8 |

(Example 61) Synthesis of Compound Represented by Formula (UVA-20)

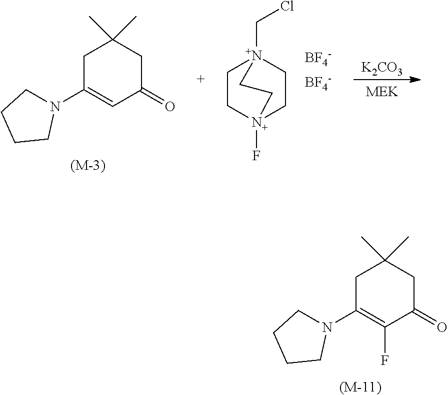

Under a nitrogen atmosphere, 17 parts of a compound represented by Formula (M-3), 12.2 parts of potassium carbonate, 15.9 parts of 1-chloromethyl-4 fluoro-1,4-diazoniabicyclo [2.2.2.] octane bis(tetrafluoroborate) (selectfluoro, registered trademark of Air Products and Chemicals), and 85 parts of methyl ethyl ketone were mixed, and the mixture was stirred in an ice bath for 3 hours. The solvent was distilled off from the obtained mixture and purified to obtain 3.7 parts of a compound represented by Formula (M-11).

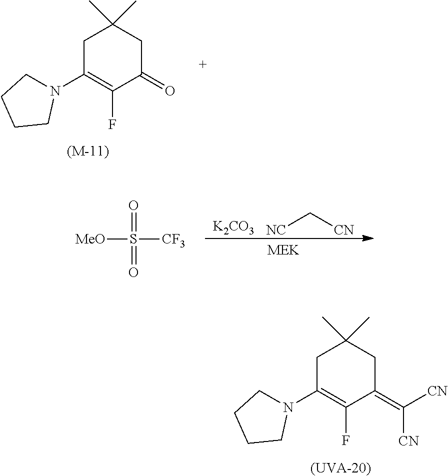

Under a nitrogen atmosphere, 18 parts of a compound represented by Formula (M-11), 28 parts of methyl triflate and 90 parts of methyl ethyl ketone were mixed, and the mixture was stirred at 20 to 30° C. for 3 hours. 13.0 parts of potassium carbonate and 8.4 parts of malononitrile were added to the obtained mixture, and the mixture was stirred at 20 to 30° C. for 3 hours. The solvent was distilled off from the obtained mixture and purified to obtain 5.8 parts of a compound represented by Formula (UVA-20).

LC-MS measurement and ¹H-NMR analysis were performed, and it was confirmed that a compound represented by Formula (UVA-20) was produced.

¹H-NMR (deuterated DMSO) δ: 1.08 (s, 6H), 1.97 (m, 4H), 2.40 (d, 2H), 2.50 (d, 2H), 3.53 (m, 2H), 3.86 (m, 2H)

LC-MS; [M+H]⁺=260.5

In addition, the maximum absorption wavelength and the gram absorption coefficient were measured in the same manner as described above. The maximum absorption wavelength of the obtained compound represented by Formula (UVA-20) was 407.5 nm. The obtained compound represented by Formula (UVA-20) had ε(λmax) of 2.30 L/(g·cm), ε(λmax+30 nm) of 0.041 L/(g·cm), and ε(λmax)/ε(λmax+30 nm) of 56.0.

### (Example 62) Synthesis of Compound Represented by Formula (UVA-21)

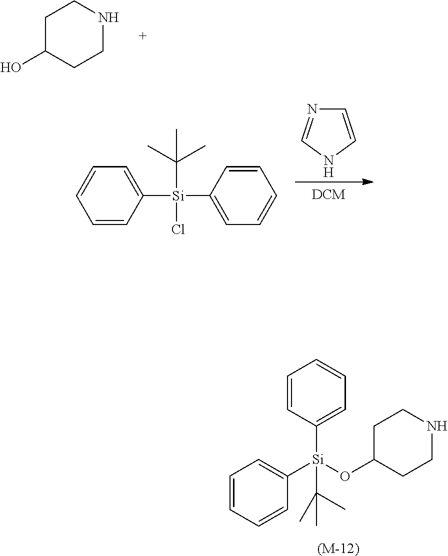

Under a nitrogen atmosphere, 5 parts of 3-hydroxypiperidine, 13.6 parts of tertiary-butyldiphenylsilyl chloride, 6.7 parts of imidazole, and 40 parts of dichloromethane were mixed, and the mixture was stirred at 20 to 30° C. for 4 hours. The solvent was distilled off from the obtained mixture and purified to obtain 10.5 parts of a compound represented by Formula (M-12).

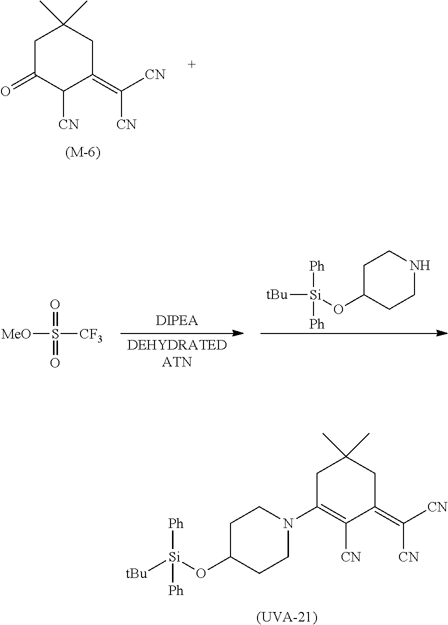

Under a nitrogen atmosphere, 4.0 parts of a compound represented by Formula (M-6), 3.2 parts of diisopropylethylamine, 4.0 parts of methyl triflate, and 80 parts of acetonitrile were mixed, and the mixture was stirred at 20 to 30° C. for 4 hours. 8.3 parts of a compound represented by Formula (M-12) was added to the obtained mixture, and the mixture was stirred at 20 to 30° C. for 3 hours. The solvent was distilled off from the obtained mixture and purified to obtain 6.5 parts of a compound represented by Formula (UVA-21).

LC-MS measurement and ¹H-NMR analysis were performed, and it was confirmed that a compound represented by Formula (UVA-21) was produced.

¹H-NMR (deuterated DMSO) δ: 0.97 (s, 6H), 1.04 (s, 9H), 1.70 (m, 2H), 1.85 (m, 2H), 2.48 (s, 2H), 2.65 (s, 2H), 3.72 (m, 2H), 3.94 (m, 2H), 4.13 (m, 1H), 7.42-7.52 (m, 6H), 7.61-7.64 (m, 4H)

LC-MS; [M+H]⁺=535.9

### (Example 63) Synthesis of Compound Represented by Formula (UVA-22)

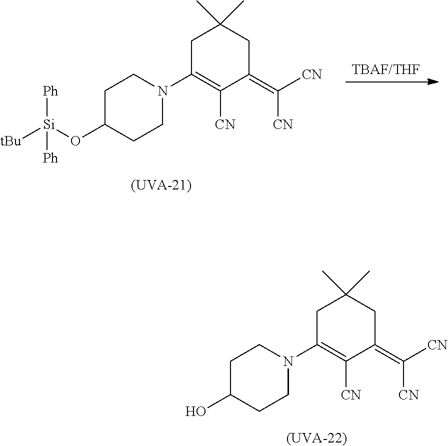

Under a nitrogen atmosphere, 4.2 parts of a compound represented by Formula (UVA-21) and 50 parts of a 1 M solution of tetrabutylammonium fluoride/tetrahydrofuran were mixed, and the mixture was stirred at 20 to 30° C. for 40 hours. The solvent was distilled off from the obtained mixture and purified to obtain 1.8 parts of a compound represented by Formula (UVA-22).

LC-MS measurement and ¹H-NMR analysis were performed, and it was confirmed that a compound represented by Formula (UVA-22) was produced.

¹H-NMR (deuterated DMSO) δ: 0.98 (s, 6H), 1.59 (m, 2H), 1.92 (m, 2H), 2.67 (s, 2H), 3.68-3.95 (m, 4H), 4.97 (m, 1H)

LC-MS; [M+H]⁺=297.5

In addition, the maximum absorption wavelength and the gram absorption coefficient were measured in the same manner as described above. The maximum absorption wavelength of the obtained compound represented by Formula (UVA-21) was 384.6 nm. The obtained compound represented by Formula (UVA-21) had ε(λmax) of 1.43 L/(g·cm), ε(λmax+30 nm) of 0.085 L/(g·cm), and ε(λmax)/ε(λmax+30 nm) of 16.8.

### (Example 64) Synthesis of Compound Represented by Formula (UVA-23)

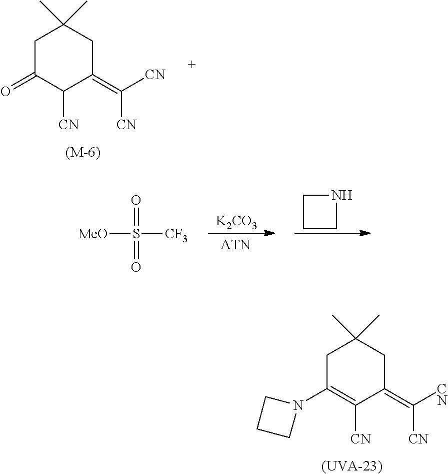

Under a nitrogen atmosphere, 5.0 parts of a compound represented by Formula (M-6), 3.6 parts of potassium carbonate, 7.7 parts of methyl triflate, and 40 parts of acetonitrile were mixed, and the mixture was stirred at 20 to 30° C. for 4 hours. 2.0 parts of azetidine was added to the obtained mixture, and the mixture was stirred at 20 to 30° C. for 4 hours. The solvent was distilled off from the obtained mixture and purified to obtain 2.3 parts of a compound represented by Formula (UVA-23).

LC-MS measurement and ¹H-NMR analysis were performed, and it was confirmed that a compound represented by Formula (UVA-23) was produced.

¹H-NMR (deuterated DMSO) δ: 1.05 (s, 6H), 2.14 (s, 2H), 2.44-2.53 (m, 4H), 4.36 (t, 2H), 4.91 (t, 2H)

LC-MS; [M+H]⁺=253.5

In addition, the maximum absorption wavelength and the gram absorption coefficient were measured in the same manner as described above. The maximum absorption wavelength of the obtained compound represented by Formula (UVA-23) was 377.2 nm. The obtained compound represented by Formula (UVA-23) had ε(λmax) of 1.93 L/(g·cm), ε(λmax+30 nm) of 0.028 L/(g·cm), and ε(λmax)/ε(λmax+30 nm) of 68.9.

### (Example 65) Synthesis of Compound Represented by Formula (UVA-24)

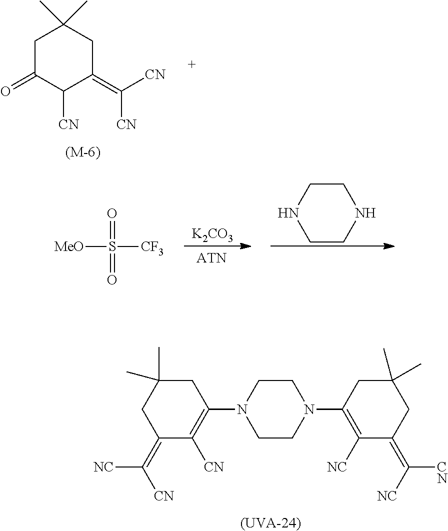

Under a nitrogen atmosphere, 2.5 parts of a compound represented by Formula (M-6), 1.6 parts of potassium carbonate, 2.3 parts of methyl triflate, and 25 parts of acetonitrile were mixed, and the mixture was stirred at 20 to 30° C. for 4 hours. 0.6 parts of piperazine was added to the obtained mixture, and the mixture was stirred at 20 to 30° C. for 4 hours. The solvent was distilled off from the obtained mixture and purified to obtain 1.0 part of a compound represented by Formula (UVA-24).

LC-MS measurement and ¹H-NMR analysis were performed, and it was confirmed that a compound represented by Formula (UVA-24) was produced.

¹H-NMR (deuterated DMSO) δ: 0.93 (s, 2H), 1.01 (s, 12H), 1.24 (s, 2H), 2.65 (s, 4H), 4.09 (m, 8H)

LC-MS; [M+H]⁺=477.5

In addition, the maximum absorption wavelength and the gram absorption coefficient were measured in the same manner as described above. The maximum absorption wavelength of the obtained compound represented by Formula (UVA-24) was 390.5 nm. The obtained compound represented by Formula (UVA-24) had ε(λmax) of 1.92 L/(g·cm), ε(λmax+30 nm) of 0.033 L/(g·cm), and ε(λmax)/ε(λmax+30 nm) of 58.2.

### (Example 66) Synthesis of Compound Represented by Formula (UVA-25)

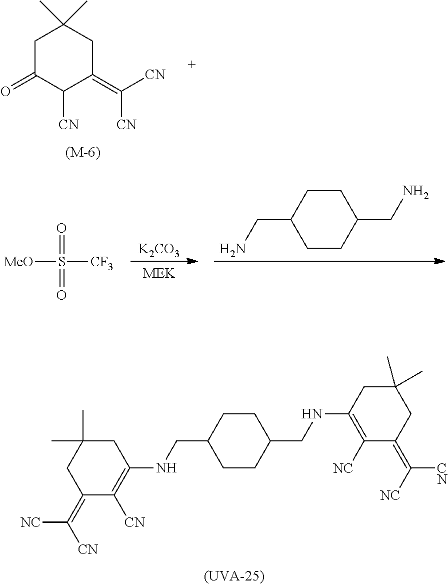

Under a nitrogen atmosphere, 2.5 parts of a compound represented by Formula (M-6), 1.6 parts of potassium carbonate, 2.3 parts of methyl triflate and 25 parts of methyl ethyl ketone were mixed, and the mixture was stirred at 20 to 30° C. for 4 hours. 1.0 part of 1,4 bisaminomethylcyclohexane was added to the obtained mixture, and the mixture was stirred at 20 to 30° C. for 4 hours. The solvent was distilled off from the obtained mixture and purified to obtain 1.0 part of a compound represented by Formula (UVA-25).

LC-MS measurement and ¹H-NMR analysis were performed, and it was confirmed that a compound represented by Formula (UVA-25) was produced.

¹H-NMR (deuterated DMSO) δ: 0.98 (m, 12H), 1.38 to 1.78 (m, 10H), 2.67 (m, 6H), 3.40 (m, 2H), 9.15 (m, 2H)

LC-MS; [M+H]⁺=533.6

In addition, the maximum absorption wavelength and the gram absorption coefficient were measured in the same manner as described above. The maximum absorption wavelength of the obtained compound represented by Formula (UVA-25) was 372.7 nm. The obtained compound represented by Formula (UVA-25) had ε(λmax) of 1.59 L/(g·cm), ε(λmax+30 nm) of 0.036 L/(g·cm), and ε(λmax)/ε(λmax+30 nm) of 44.1.

### (Example 67) Synthesis of Compound Represented by Formula (UVA-26)

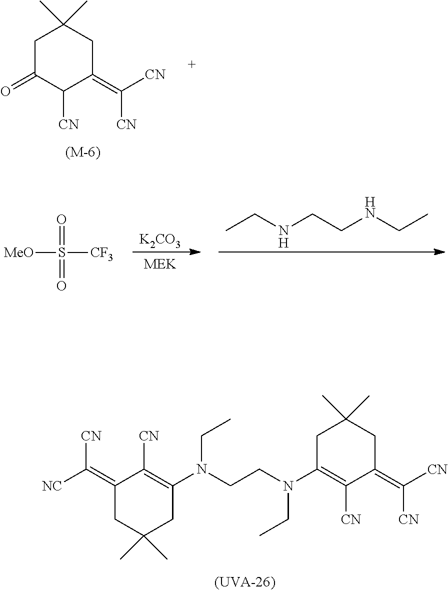

Under a nitrogen atmosphere, 2.5 parts of a compound represented by Formula (M-6), 1.6 parts of potassium carbonate, 2.3 parts of methyl triflate and 25 parts of methyl ethyl ketone were mixed, and the mixture was stirred at 20 to 30° C. for 4 hours. 0.8 parts of 1,2 bis(ethylamino) ethane was added to the obtained mixture, and the mixture was stirred at 20 to 30° C. for 4 hours. The solvent was distilled off from the obtained mixture and purified to obtain 0.9 parts of a compound represented by Formula (UVA-26).

LC-MS measurement and ¹H-NMR analysis were performed, and it was confirmed that a compound represented by Formula (UVA-26) was produced.

¹H-NMR (deuterated DMSO) δ: 1.00 (s, 12H), 1.29 (t, 6H), 2.56 (s, 4H), 2.70 (s, 4H), 3.85 (m, 4H), 4.05 (m, 4H)

LC-MS; [M+H]⁺=507.7

In addition, the maximum absorption wavelength and the gram absorption coefficient were measured in the same manner as described above. The maximum absorption wavelength of the obtained compound represented by Formula (UVA-26) was 390.7 nm. The obtained compound represented by Formula (UVA-26) had ε(λmax) of 1.30 L/(g·cm), ε(λmax+30 nm) of 0.048 L/(g·cm), and ε(λmax)/(λmax+30 nm) of 27.1.

### (Example 68) Preparation of Pressure-Sensitive Adhesive Composition (16)

A pressure-sensitive adhesive composition (16) was obtained in the same manner as that in Example 23 except that the compound represented by Formula (UVA-1) was changed to the compound represented by Formula (UVA-23), and the content of the compound was 0.5 parts based on 100 parts of the acrylic resin (A).

### (Example 69) Preparation of Pressure-Sensitive Adhesive Composition (17)

A pressure-sensitive adhesive composition (17) was obtained in the same manner as that in Example 23 except that the compound represented by Formula (UVA-1) was changed to the compound represented by Formula (UVA-26), and the content of the compound was 0.2 parts based on 100 parts of the acrylic resin (A).

### (Example 70) Preparation of Pressure-Sensitive Adhesive Layer (10) and Pressure-Sensitive Adhesive Sheet (10)

A pressure-sensitive adhesive layer (10) and a pressure-sensitive adhesive sheet (10) were prepared in the same manner as in Example 32 except that the pressure-sensitive adhesive composition (6) was changed to the pressure-sensitive adhesive composition (16).

### (Example 71) Preparation of Pressure-Sensitive Adhesive Layer (11) and Pressure-Sensitive Adhesive Sheet (11)

A pressure-sensitive adhesive layer (11) and a pressure-sensitive adhesive sheet (11) were prepared in the same manner as in Example 32 except that the pressure-sensitive adhesive composition (6) was changed to the pressure-sensitive adhesive composition (17).

<Absorbance Measurement and Absorbance Retention Measurement of Pressure-Sensitive Adhesive Sheet>

The absorbance and the absorbance retention were measured in the same manner as in <Measurement of Absorbance of Pressure-Sensitive Adhesive Sheet> and <Measurement of Absorbance Retention of Pressure-Sensitive Adhesive Sheet> described above except that the pressure-sensitive adhesive sheets (10) to (11) were used instead of the pressure-sensitive adhesive sheet (1). The results are shown in Table 6.

TABLE 6

| | | | | A (395)/ | Absorbance |
|---|---|---|---|---|---|
| | Compounds | A (395) | A (430) | A (430) | retention |
| Example 70 | Formula (UVA-23) | 0.78 | 0.001 | 778.0 | 99.6 |
| Example 71 | Formula (UVA-26) | 1.15 | 0.309 | 3.7 | 62.2 |

### (Example 72) Preparation of Pressure-Sensitive Adhesive Composition (18)

<Preparation of Acrylic Resin (A-2)>

In a reaction container equipped with a cooling tube, a nitrogen introduction tube, a thermometer, and a stirrer, a mixed solution of 81.8 parts of ethyl acetate as a solvent, 96 parts of butyl acrylate, 3 parts of 2-hydroxyethyl acrylate, and 1 part of acrylic acid as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 1.40 million as measured by GPC. The Mw/Mn was 4.8. This is referred to as an acrylic resin (A-2).

<Preparation of Pressure-Sensitive Adhesive Composition (18)>

To 100 parts of solid content of the ethyl acetate solution (resin concentration: 20%) of the acrylic resin (A-2) synthesized above, 0.5 parts of a crosslinking agent (ethyl acetate solution of trimethylolpropane adduct of tolylene diisocyanate (solid concentration: 75%), produced by Tosoh Corporation, trade name “Coronate L”), 0.3 parts of a silane compound (1,6-bis(trimethoxysilyl) hexane, produced by Shin-Etsu Chemical Co., Ltd., trade name “KBM 3066”), and 3 parts of a compound represented by Formula (UVA-6) were mixed, and ethyl acetate was further added so that the solid content concentration was 14% to obtain a pressure-sensitive adhesive composition (18). The blending amount of the crosslinking agent is the number of parts by mass as an active component.

### (Example 73) Preparation of Pressure-Sensitive Adhesive Composition (19)

<Preparation of Acrylic Resin (A-3)>

In a reaction container equipped with a cooling tube, a nitrogen introduction tube, a thermometer, and a stirrer, a mixed solution of 81.8 parts of ethyl acetate as a solvent, 60 parts of methyl acrylate, 10 parts of 2-hydroxyethyl acrylate, 10 parts of acrylic acid, and 20 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 92 million as measured by GPC. The Mw/Mn was 7.8. This is referred to as an acrylic resin (A-3).

<Preparation of Pressure-Sensitive Adhesive Composition (19)>

A pressure-sensitive adhesive composition (19) was obtained in the same manner as that in Example 72 except that the acrylic resin (A-3) synthesized above was used instead of the acrylic resin (A-2).

### (Example 74) Preparation of Pressure-Sensitive Adhesive Composition (20)

<Preparation of Acrylic Resin (A-4)>

In a reaction container equipped with a cooling tube, a nitrogen introduction tube, a thermometer, and a stirrer, a mixed solution of 81.8 parts of ethyl acetate as a solvent, 10 parts of butyl acrylate, 60 parts of methyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 94 million as measured by GPC. The Mw/Mn was=8.5. This is referred to as an acrylic resin (A-4).

<Preparation of Pressure-Sensitive Adhesive Composition (20)>

A pressure-sensitive adhesive composition (20) was obtained in the same manner as that in Example 72 except that the acrylic resin (A-4) synthesized above was used instead of the acrylic resin (A-2).

### (Example 75) Preparation of Pressure-Sensitive Adhesive Composition (21)

<Preparation of Acrylic Resin (A-5)>

In a reaction container equipped with a cooling tube, a nitrogen introduction tube, a thermometer, and a stirrer, a mixed solution of 81.8 parts of ethyl acetate as a solvent, 20 parts of butyl acrylate, 50 parts of methyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 91 million as measured by GPC. This is referred to as an acrylic resin (A-5).

<Preparation of Pressure-Sensitive Adhesive Composition (21)>

A pressure-sensitive adhesive composition (21) was obtained in the same manner as that in Example 72 except that the acrylic resin (A-5) synthesized above was used instead of the acrylic resin (A-2).

### (Example 76) Preparation of Pressure-Sensitive Adhesive Composition (22)

<Preparation of Acrylic Resin (A-6)>

In a reaction container equipped with a cooling tube, a nitrogen introduction tube, a thermometer, and a stirrer, a mixed solution of 81.8 parts of ethyl acetate as a solvent, 50 parts of butyl acrylate, 10 parts of methyl acrylate, 10 parts of 2-hydroxyethyl acrylate, 10 parts of acrylic acid, and 20 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 120 million as measured by GPC. This is referred to as an acrylic resin (A-6).

<Preparation of Pressure-Sensitive Adhesive Composition (22)>

A pressure-sensitive adhesive composition (22) was obtained in the same manner as that in Example 72 except that the acrylic resin (A-6) synthesized above was used instead of the acrylic resin (A-2).

### (Example 77) Preparation of Pressure-Sensitive Adhesive Composition (23)

<Preparation of Acrylic Resin (A-7)>

In a reaction container equipped with a cooling tube, a nitrogen introduction tube, a thermometer, and a stirrer, a mixed solution of 81.8 parts of ethyl acetate as a solvent, 60 parts of butyl acrylate, 10 parts of methyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 118 million as measured by GPC. This is referred to as an acrylic resin (A-7).

<Preparation of Pressure-Sensitive Adhesive Composition (23)>

A pressure-sensitive adhesive composition (23) was obtained in the same manner as that in Example 72 except that the acrylic resin (A-7) synthesized above was used instead of the acrylic resin (A-2).

### (Example 78) Preparation of Pressure-Sensitive Adhesive Composition (24)

<Preparation of Acrylic Resin (A-8)>

In a reaction container equipped with a cooling tube, a nitrogen introduction tube, a thermometer, and a stirrer, a mixed solution of 81.8 parts of ethyl acetate as a solvent, 70 parts of butyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 110 million as measured by GPC. This is referred to as an acrylic resin (A-8).

<Preparation of Pressure-Sensitive Adhesive Composition (24)>

A pressure-sensitive adhesive composition (23) was obtained in the same manner as that in Example 72 except that the acrylic resin (A-8) synthesized above was used instead of the acrylic resin (A-2).

<Evaluation of Crystal Precipitation (Bleeding Resistance) of Pressure-Sensitive Adhesive Layer>

The pressure-sensitive adhesive composition (18) was applied to a release-treated surface of a separator film [trade name “PLR-382190” available from Lintec Corporation] made of a release-treated polyethylene terephthalate film using an applicator, and dried at 100° C. for 1 minute to prepare a pressure-sensitive adhesive layer. A separate film was further laminated on the other surface of the pressure-sensitive adhesive layer to obtain a pressure-sensitive adhesive layer with a separate film on both sides. The thickness of the obtained pressure-sensitive adhesive layer was 15 μm.

The obtained pressure-sensitive adhesive layer with a separate film on both sides was cured for 7 days under the conditions of a temperature of 23° C. and a relative humidity of 65%. Using the pressure-sensitive adhesive layer with a separate film on both sides after curing, the presence or absence of in-plane crystal precipitation of the compound was confirmed using a microscope. A case where there was no crystal precipitation was evaluated as a, and a case where there was crystal precipitation was evaluated as b. The evaluation results are shown in the column of “after curing” in Table 7.

The obtained pressure-sensitive adhesive layer with a separate film on both sides was stored under air at a temperature of 40° C. for 1 month. Using the pressure-sensitive adhesive layer with a separate film on both sides after storage, the presence or absence of in-plane crystal precipitation of the compound was confirmed using a microscope. A case where there was no crystal precipitation was evaluated as a, and a case where there was crystal precipitation was evaluated as b. The evaluation results are shown in the column of “40° C. 1 M” in Table 7.

The presence or absence of crystal precipitation was confirmed in the same manner except that the pressure-sensitive adhesive composition (18) was changed to the pressure-sensitive adhesive composition (19) to the pressure-sensitive adhesive composition (24). The results are shown in Table 7.

TABLE 7

| | Pressure- | | | |
|---|---|---|---|---|
| | sensitive | | Crystal precipitation | |
| | adhesive | Acrylic | After | 40° C. |
| | composition | resin | curing | 1M |
| Example 72 | (18) | (A-2) | b | — |
| Example 73 | (19) | (A-3) | a | a |
| Example 74 | (20) | (A-4) | a | a |
| Example 75 | (21) | (A-5) | a | a |
| Example 76 | (22) | (A-6) | a | b |
| Example 77 | (23) | (A-7) | a | b |
| Example 78 | (24) | (A-8) | a | b |

(Example 79) Preparation of Pressure-Sensitive Adhesive Layer (12) and Pressure-Sensitive Adhesive Sheet (12)

The obtained pressure-sensitive adhesive composition (18) was applied to a release-treated surface of a separator film [trade name “PLR-382190” available from Lintec Corporation] made of a release-treated polyethylene terephthalate film using an applicator, and dried at 100° C. for 1 minute to prepare a pressure-sensitive adhesive layer (12). The thickness of the obtained pressure-sensitive adhesive layer was 15 μm.

The obtained pressure-sensitive adhesive layer (12) was bonded to a cycloolefin film not containing an ultraviolet absorber of 23 μm by a laminator, and then aged for 7 days under conditions of a temperature of 23° C. and a relative humidity of 65% to obtain a pressure-sensitive adhesive sheet (12).

### (Example 80) Preparation of Pressure-Sensitive Adhesive Layer (13) and Pressure-Sensitive Adhesive Sheet (13)

A pressure-sensitive adhesive layer (13) and a pressure-sensitive adhesive sheet (13) were prepared in the same manner as in Example 79 except that the pressure-sensitive adhesive composition (18) was changed to the pressure-sensitive adhesive composition (19).

### (Example 81) Preparation of Pressure-Sensitive Adhesive Layer (14) and Pressure-Sensitive Adhesive Sheet (14)

A pressure-sensitive adhesive layer (14) and a pressure-sensitive adhesive sheet (14) were prepared in the same manner as in Example 79 except that the pressure-sensitive adhesive composition (18) was changed to the pressure-sensitive adhesive composition (20).

### (Example 82) Preparation of Pressure-Sensitive Adhesive Layer (15) and Pressure-Sensitive Adhesive Sheet (15)

A pressure-sensitive adhesive layer (15) and a pressure-sensitive adhesive sheet (15) were prepared in the same manner as in Example 79 except that the pressure-sensitive adhesive composition (18) was changed to the pressure-sensitive adhesive composition (21).

### (Example 83) Preparation of Pressure-Sensitive Adhesive Layer (16) and Pressure-Sensitive Adhesive Sheet (16)

A pressure-sensitive adhesive layer (16) and a pressure-sensitive adhesive sheet (16) were prepared in the same manner as in Example 79 except that the pressure-sensitive adhesive composition (18) was changed to the pressure-sensitive adhesive composition (22).

### (Example 84) Preparation of Pressure-Sensitive Adhesive Layer (17) and Pressure-Sensitive Adhesive Sheet (17)

A pressure-sensitive adhesive layer (17) and a pressure-sensitive adhesive sheet (17) were prepared in the same manner as in Example 79 except that the pressure-sensitive adhesive composition (18) was changed to the pressure-sensitive adhesive composition (23).

### (Example 85) Preparation of Pressure-Sensitive Adhesive Layer (18) and Pressure-Sensitive Adhesive Sheet (18)

A pressure-sensitive adhesive layer (18) and a pressure-sensitive adhesive sheet (18) were prepared in the same manner as in Example 79 except that the pressure-sensitive adhesive composition (18) was changed to the pressure-sensitive adhesive composition (24).

<Measurement of Absorbance Retention of Pressure-Sensitive Adhesive Sheet>

The obtained pressure-sensitive adhesive sheet (12) was cut into a size of 30 mm×30 mm, the separate film was peeled off, and the pressure-sensitive adhesive layer (12) and alkali-free glass [trade name “EAGLE XG” produced by Corning Incorporated] were bonded to each other to prepare a sample (5). The absorbance of the prepared sample (5) in the wavelength range of 300 to 800 nm was measured every 1 nm step using a spectrophotometer (UV-2450: manufactured by Shimadzu Corporation). The measured absorbances at a wavelength of 400 nm were taken as the absorbances at a wavelength of 400 nm of the pressure-sensitive adhesive sheet (12). The results are shown in Table 8. Both the cycloolefin film alone and the non-alkali glass alone have zero absorbance at a wavelength of 400 nm.

The sample (5) after the absorbance measurement was charged into a Sunshine weathermeter (manufactured by Suga Test Instruments Co., Ltd.) for 150 hours under the conditions of a temperature of 63° C. and a relative humidity of 50% RH to perform a weather resistance test. The absorbance of the sample (5) taken out was measured in the same manner as described above. From the measured absorbance, the absorbance retention of the sample at a wavelength of 400 nm was determined based on the following formula. The results are shown in Table 8. A value of the absorbance retention closer to 100 indicates that the light selective absorption function is not deteriorated and good weather resistance is obtained.

The absorbance retention was also determined when the sample (5) was charged into a Sunshine weathermeter (manufactured by Suga Test Instruments Co., Ltd.) for 225 hours under the conditions of a temperature of 63° C. and a relative humidity of 50% RH.

Absorbance retention (%)=(A(400) after durability test/A(400) before durability test)×100

The absorbance retention was measured in the same manner except that the pressure-sensitive adhesive sheet (12) was changed to the pressure-sensitive adhesive sheet (13) to the pressure-sensitive adhesive sheet (18). The results are shown in Table 8.

TABLE 8

| | Pressure- | Pressure- | | | |
|---|---|---|---|---|---|
| | sensitive | sensitive | | | |
| | adhesive | adhesive | Acrylic | Absorbance retention | |
| | sheet | composition | resin | 150 hr | 225 hr |
| Example 79 | (12) | (18) | (A-2) | 34.9 | 17.1 |
| Example 80 | (13) | (19) | (A-3) | 54.5 | 40.4 |
| Example 81 | (14) | (20) | (A-4) | 52.2 | 40.9 |
| Example 82 | (15) | (21) | (A-5) | 54 | 39.2 |
| Example 83 | (16) | (22) | (A-6) | 38.8 | 23.1 |
| Example 84 | (17) | (23) | (A-7) | 53.3 | 35 |
| Example 85 | (18) | (24) | (A-8) | 51.8 | 35.4 |

### (Example 86) Preparation of Pressure-Sensitive Adhesive Sheet (19)

A pressure-sensitive adhesive sheet (19) was prepared in the same manner as that in Example 79 except that the cycloolefin film not containing an ultraviolet absorber of 23 μm was changed to a cycloolefin film containing an ultraviolet absorber of 23 μm.

### (Example 87) Preparation of Pressure-Sensitive Adhesive Sheet (20)

A pressure-sensitive adhesive sheet (20) was prepared in the same manner as that in Example 80 except that the cycloolefin film not containing an ultraviolet absorber of 23 μm was changed to a cycloolefin film containing an ultraviolet absorber of 23 μm.

### (Example 88) Preparation of Pressure-Sensitive Adhesive Sheet (21)

A pressure-sensitive adhesive sheet (21) was prepared in the same manner as that in Example 81 except that the cycloolefin film not containing an ultraviolet absorber of 23 μm was changed to a cycloolefin film containing an ultraviolet absorber of 23 μm.

### (Example 89) Preparation of Pressure-Sensitive Adhesive Sheet (22)

A pressure-sensitive adhesive sheet (22) was prepared in the same manner as that in Example 82 except that the cycloolefin film not containing an ultraviolet absorber of 23 μm was changed to a cycloolefin film containing an ultraviolet absorber of 23 μm.

### (Example 90) Preparation of Pressure-Sensitive Adhesive Sheet (23)

A pressure-sensitive adhesive sheet (23) was prepared in the same manner as that in Example 83 except that the cycloolefin film not containing an ultraviolet absorber of 23 μm was changed to a cycloolefin film containing an ultraviolet absorber of 23 μm.

### (Example 91) Preparation of Pressure-Sensitive Adhesive Sheet (24)

A pressure-sensitive adhesive sheet (24) was prepared in the same manner as that in Example 84 except that the cycloolefin film not containing an ultraviolet absorber of 23 μm was changed to a cycloolefin film containing an ultraviolet absorber of 23 μm.

### (Example 92) Preparation of Pressure-Sensitive Adhesive Sheet (25)

A pressure-sensitive adhesive sheet (25) was prepared in the same manner as that in Example 85 except that the cycloolefin film not containing an ultraviolet absorber of 23 μm was changed to a cycloolefin film containing an ultraviolet absorber of 23 μm.

<Measurement of Absorbance Retention of Pressure-Sensitive Adhesive Sheet>

The obtained pressure-sensitive adhesive sheet (19) was cut into a size of 30 mm×30 mm, the separate film was peeled off, and the pressure-sensitive adhesive layer (19) and alkali-free glass [trade name “EAGLE XG” produced by Corning Incorporated] were bonded to each other to prepare a sample (6). The absorbance of the prepared sample (5) in the wavelength range of 300 to 800 nm was measured every 1 nm step using a spectrophotometer (UV-2450: manufactured by Shimadzu Corporation). The measured absorbances at a wavelength of 405 nm were taken as the absorbances at a wavelength of 405 nm of the pressure-sensitive adhesive sheet (19). The results are shown in Table 9. The absorbance at a wavelength of 405 nm of the alkali-free glass alone is 0.

The sample (6) after the absorbance measurement was charged into a Sunshine weathermeter (manufactured by Suga Test Instruments Co., Ltd.) for 150 hours under the conditions of a temperature of 63° C. and a relative humidity of 50% RH to perform a weather resistance test. The absorbance of the sample (5) taken out was measured in the same manner as described above. From the measured absorbance, the absorbance retention of the sample at a wavelength of 405 nm was determined based on the following formula. The results are shown in Table 9. A value of the absorbance retention closer to 100 indicates that the light selective absorption function is not deteriorated and good weather resistance is obtained.

The absorbance retention was also determined when the sample (6) was charged into a Sunshine weathermeter (manufactured by Suga Test Instruments Co., Ltd.) for 225 hours under the conditions of a temperature of 63° C. and a relative humidity of 50% RH.

Absorbance retention (%)=(A(405) after durability test/A(405) before durability test)×100

The absorbance retention was measured in the same manner except that the pressure-sensitive adhesive sheet (19) was changed to the pressure-sensitive adhesive sheet (20) to the pressure-sensitive adhesive sheet (25). The results are shown in Table 9.

TABLE 9

| | Pressure- | Pressure- | | | |
|---|---|---|---|---|---|
| | sensitive | sensitive | | | |
| | adhesive | adhesive | Acrylic | Absorbance retention | |
| | sheet | composition | resin | 150 hr | 225 hr |
| Example 86 | (19) | (18) | (A-2) | 19.2 | 33.5 |
| Example 87 | (20) | (19) | (A-3) | 83.3 | 80.2 |
| Example 88 | (21) | (20) | (A-4) | 71.1 | 68.3 |
| Example 89 | (22) | (21) | (A-5) | 71 | 68.4 |
| Example 90 | (23) | (22) | (A-6) | 66.6 | 62.8 |
| Example 91 | (24) | (23) | (A-7) | 84.5 | 82.8 |
| Example 92 | (25) | (24) | (A-8) | 83.9 | 81.7 |

### Example 93

<Preparation of Resin Composition for Spectacle Lens>

40 parts of xylylene diisocyanate, 60 parts of trimethylolpropane tris(thioglycolate), 1.6 parts of a compound represented by Formula (UVA-6), 0.2 parts of a release agent (trade name: ZELEC-UN, available from Sigme-Aldrich), and 0.03 parts of dibutyldichlorotin were mixed and stirred. The obtained mixture was allowed to stand in a vacuum dryer for 1 hour and degassed. The obtained mixture was poured into a glass mold and heated at 120° C. for 1 hour. Only a resin plate was peeled off to prepare a resin plate having a thickness of 2 mm and 3 cm×3 cm.

<Measurement of Absorbance Retention of Resin Plate>

The absorbance of the resin plate obtained above in a wavelength range of 300 to 800 nm was measured every 1 nm step by using a spectrophotometer (UV-2450: manufactured by Shimadzu Corporation).

The resin plate after the measurement was charged into a Sunshine weathermeter (manufactured by Suga Test Instruments Co., Ltd.) for 75 hours under the conditions of a temperature of 63° C. and a relative humidity of 50% RH to perform a weather resistance test. The absorbance of the resin plate taken out was measured in the same manner as described above. From the measured absorbance, the absorbance retention of the sample at a wavelength of 420 nm was determined based on the following formula. The results are shown in Table 10. A value of the absorbance retention closer to 100 indicates that the light selective absorption function is not deteriorated and good weather resistance is obtained.

Note that the spectacle lens is required to have a good absorbance retention at a wavelength of 420 nm in order to efficiently cut light of blue light that tends to have a negative effect on health. As the value of A (420)/A (480) is larger, the blue light can be cut with less coloring.

Absorbance retention (%)=(A(420) after durability test/A(420) before durability test)×100

TABLE 10

| | | | | A (420)/ | Absorbance |
|---|---|---|---|---|---|
| | Compounds | A (420) | A (480) | A (480) | retention |
| Example 93 | Formula (UVA-6) | 3.26 | 0.049 | 66.5 | 100 |

The novel compound having a merocyanine skeleton of the present invention has high absorption selectivity to short-wavelength visible light having a wavelength of 380 to 400 nm. In addition, the compound of the present invention has a high absorbance retention even after a weather resistance test, and has good weather resistance.

1. A compound having a molecular weight of 3000 or less and a partial structure represented by Formula (X): 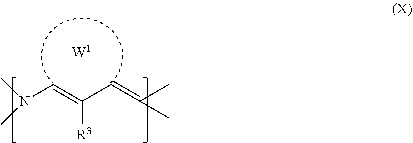
[in Formula (X), a ring W¹ represents a ring structure having at least one double bond as a constituent element of the ring and having no aromaticity;
R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—; and
R^{1A}, R^{2A}, R^{3A}, R^{4A}, R^{5A}, R^{6A}, R^{7A}, R^{8A}, R^{9A}, R^{10A}, and R^{11A} each independently represent a hydrogen atom or an alkyl group having 1 to 6 carbon atoms].

2. The compound according to claim 1, wherein the compound having a molecular weight of 3000 or less and a partial structure represented by Formula (X) is any of compounds from a compound represented by Formula (I) to a compound represented by Formula (VIII): 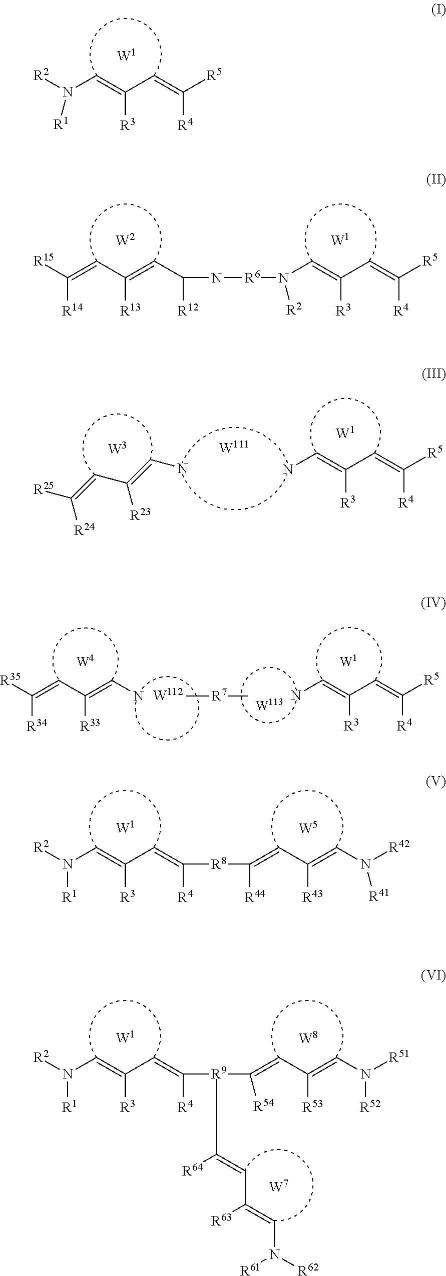 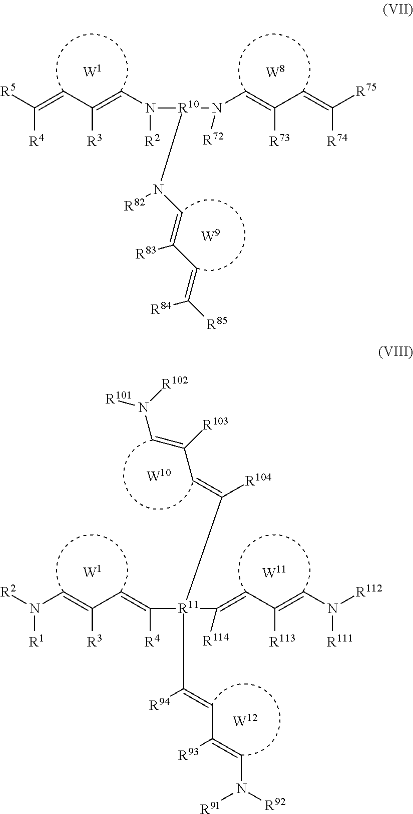
[in Formula (I) to Formula (VIII),
the ring W and R³ have the same meaning as described above;
a ring W², a ring W³, a ring W⁴, a ring W⁵, a ring W⁶, a ring W⁷, a ring W⁸, a ring W⁹, a ring W¹⁰, a ring Wu, and a ring W² each independently represent a ring structure having at least one double bond as a constituent element of the ring;
a ring W¹¹¹ represents a ring having at least two nitrogen atoms as constituent elements;
a ring W¹¹² and a ring W¹¹³ each independently represent a ring having at least one nitrogen atom as a constituent element;
R¹, R⁴¹, R⁵¹, R⁶¹, R⁹¹, R¹⁰¹, R¹¹¹, R², R¹², R⁴², R⁵², R⁶², R⁷², R⁸², R⁹², R¹⁰², and R¹¹² each independently represent a hydrogen atom, a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{12A}—, —SO₂—, —CO—, —O—, —COO—, —OCO—, —CONR^{13A}—, —NR^{14A}—CO—, —S—, —SO—, —CF₂— or —CHF—;
R¹³, R²³, R³³, R⁴³, R⁵³, R⁶³, R⁷³, R⁸³, R⁹³, R¹⁰³, and R¹¹³ each independently represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—;
R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, and R^{14A} each independently represent a hydrogen atom or an alkyl group having 1 to 6 carbon atoms;
R⁴, R¹⁴, R²⁴, R³⁴, R⁴⁴, R⁵⁴, R⁶⁴, R⁷⁴, R⁸⁴, R⁹⁴, R¹⁰⁴, R¹¹⁴, R⁵, R¹⁵, R²⁵, R³⁵, R⁷⁵, and R⁸⁵ each independently represent an electron-withdrawing group;
R¹ and R² may be bonded to each other to form a ring;
R⁴¹ and R⁴² may be bonded to each other to form a ring;
R⁵¹ and R⁵² may be bonded to each other to form a ring;
R⁶¹ and R⁶² may be bonded to each other to form a ring;
R⁹¹ and R⁹² may be bonded to each other to form a ring;
R¹⁰¹ and R¹⁰² may be bonded to each other to form a ring;
R¹¹¹ and R¹¹² may be bonded to each other to form a ring;
R² and R³ may be bonded to each other to form a ring;
R¹² and R¹³ may be bonded to each other to form a ring;
R⁴² and R⁴³ may be bonded to each other to form a ring;
R⁵² and R⁵³ may be bonded to each other to form a ring;
R⁶² and R⁶³ may be bonded to each other to form a ring;
R⁷² and R⁷³ may be bonded to each other to form a ring;
R⁸² and R⁸³ may be bonded to each other to form a ring;
R⁹² and R⁹³ may be bonded to each other to form a ring;
R¹⁰² and R¹⁰³ may be bonded to each other to form a ring;
R¹¹² and R¹¹³ may be bonded to each other to form a ring;
R⁴ and R⁵ may be bonded to each other to form a ring;
R¹⁴ and R¹⁵ may be bonded to each other to form a ring;
R²⁴ and R²⁵ may be bonded to each other to form a ring;
R³⁴ and R³⁵ may be bonded to each other to form a ring;
R⁷⁴ and R⁷⁵ may be bonded to each other to form a ring;
R⁸⁴ and R⁸⁵ may be bonded to each other to form a ring;
R⁶ and R⁸ each independently represent a divalent linking group;
R⁷ represents a single bond or a divalent linking group;
R⁹ and R¹⁰ each independently represent a trivalent linking group; and
R¹¹ represents a tetravalent linking group].

3. The compound according to claim 2, wherein at least one selected from R⁴ and R⁵ is a nitro group, a cyano group, a halogen atom, —OCF₃, —SCF₃, —SF₅, —SF₃, a fluoroalkyl group, a fluoroaryl group, —CO—O—R²²², —SO₂—R²²², or —CO—R²²² (R²²² represents a hydrogen atom, an alkyl group having 1 to 25 carbon atoms which may have a substituent, or an aromatic hydrocarbon group having 6 to 18 carbon atoms which may have a substituent).

4. The compound according to claim 2, wherein at least one selected from R⁴ and R⁵ is a nitro group, a cyano group, a fluorine atom, a chlorine atom, —OCF₃, —SCF₃, a fluoroalkyl group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent).

5. The compound according to claim 2, wherein at least one selected from R⁴ and R⁵ is a cyano group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent).

6. The compound according to claim 2, wherein at least one selected from R⁴ and R⁵ is a cyano group.

7. The compound according to claim 2, wherein R⁴ is a cyano group, R⁵ is a cyano group, —CO—O—R²²², or —SO₂—R²²² (R²²² represents a hydrogen atom, an alkyl group having 1 to 25 carbon atoms which may have a substituent, or an aromatic hydrocarbon group having 6 to 18 carbon atoms which may have a substituent).

8. The compound according to claim 2, wherein both R⁴ and R⁵ are a cyano group.

9. The compound according to claim 2, wherein R¹ and R² are each independently an aliphatic hydrocarbon group having 1 to 25 carbon atoms which may have a substituent.

10. The compound according to claim 2, wherein R¹ and R² are linked to each other to form a ring.

11. The compound according to claim 10, wherein the ring formed by linking R¹ and R² to each other is an aliphatic ring.

12. The compound according to claim 2, wherein the ring W², the ring W³, the ring W⁴, the ring W⁵, the ring W⁶, the ring W⁷, the ring W⁸, the ring W⁹, the ring W¹⁰, the ring W¹¹, and the ring W¹² are each independently a ring having no aromaticity.

13. The compound according to claim 2, wherein the ring W², the ring W³, the ring W⁴, the ring W⁵, the ring W⁶, the ring W⁷, the ring W⁸, the ring W⁹, the ring W¹⁰, the ring W¹¹, and the ring W¹² are each independently a 5 to 7-membered ring structure.

14. The compound according to claim 13, wherein the ring W², the ring W³, the ring W⁴, the ring W⁵, the ring W⁶, the ring W⁷, the ring W⁸, the ring W⁹, the ring W¹⁰, the ring W¹¹, and the ring W¹² are each independently a 6-membered ring structure.

15. The compound according to claim 1, wherein R³ is a nitro group, a cyano group, a halogen atom, —OCF₃, —SCF₃, —SF₅, —SF₃, a fluoroalkyl group, a fluoroaryl group, —CO—O—R^{111A}, or —SO₂—R^{112A} (R^{111A} and R^{112A} each independently represent an alkyl group having 1 to 24 carbon atoms which may have a halogen atom).

16. The compound according to claim 1, wherein R³ is a cyano group, a fluorine atom, a chlorine atom, —OCF₃, —SCF₃, a fluoroalkyl group, —CO—O—R^{111A}, or —SO₂—R^{112A} (R^{111A} and R^{112A} each independently represent an alkyl group having 1 to 24 carbon atoms which may have a halogen atom).

17. The compound according to claim 1, wherein R³ is a cyano group.

18. The compound according to claim 1, wherein the ring W¹ is a 5 to 7-membered ring.

19. The compound according to claim 18, wherein the ring W¹ is a 6-membered ring.

20. The compound according to claim 1, wherein a gram absorption coefficient ε at λmax is 0.5 or more.
(λmax represents a maximum absorption wavelength [nm] in a compound having a molecular weight of 3000 or less and a partial structure represented by Formula (X)).

21. The compound according to claim 1, which satisfies Formula (B),
ε(λmax)/ε(λmax+30 nm)≥5 (B)
[ε(λmax) represents a gram absorption coefficient at a maximum absorption wavelength [nm] in a compound having a molecular weight of 3000 or less and a partial structure represented by Formula (X); and
ε(λmax+30 nm) represents a gram absorption coefficient at a wavelength [nm] of (maximum absorption wavelength+30 nm) of a compound having a molecular weight of 3000 or less and a partial structure represented by Formula (X)].

22. A composition containing the compound according to claim 1.

23. A molded article formed from the composition according to claim 22.

24. A spectacle lens composition containing the compound according to claim 1.

25. A spectacle lens formed from the spectacle lens composition according to claim 24.

26. A method for producing a compound represented by Formula (I): 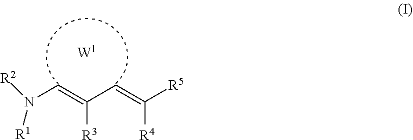
[in Formula (I), the ring W¹, R¹, R², R³, R⁴, and R⁵ have the same meaning as described above],
the method comprising a step of reacting a compound represented by Formula (I-1): 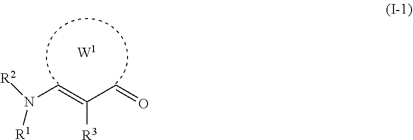
[in Formula (I-1),
a ring W¹ represents a ring structure having at least one double bond as a constituent element of the ring and having no aromaticity;
R¹ and R² each independently represent a hydrogen atom, a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{12A}—, —SO₂—, —CO—, —O—, —COO—, —OCO—, —CONR^{13A}—, —NR^{14A}—CO—, —S—, —SO—, —CF₂— or —CHF—;
R¹ and R² may be linked to each other to form a ring;
R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—;
R² and R³ may be bonded to each other to form a ring; and
R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, and R^{14A} each independently represent a hydrogen atom or an alkyl group having 1 to 6 carbon atoms], with a compound represented by Formula (I-2): 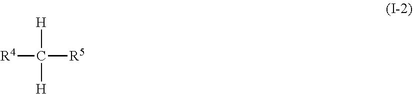
[in Formula (I-2), R⁴ and R⁵ each independently represent an electron-withdrawing group; and R⁴ and R⁵ may be bonded to each other to form a ring].

27. The production method according to claim 26, further comprising:
a step of reacting a compound represented by Formula (I-3): 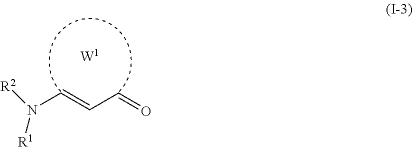
[in Formula (I-3), the ring W¹, R¹, and R² have the same meaning as described above], with a compound represented by Formula (I-4):
R³-E₁ (I-4)
[in Formula (I-4), R³ has the same meaning as described above; and E₁ represents a leaving group], to obtain the compound represented by Formula (I-1).

28. The production method according to claim 27, further comprising:
a step of reacting a compound represented by Formula (I-5): 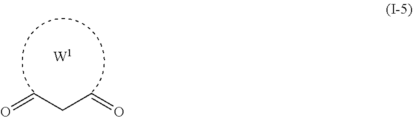
[in Formula (I-5), a ring W¹ has the same meaning as described above], with a compound represented by Formula (I-6): 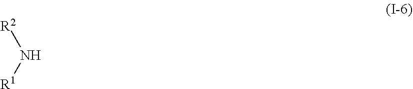
[in Formula (I-6), R¹ and R² have the same meaning as described above], to obtain the compound represented by Formula (I-3).

29. A method for producing a compound represented by Formula (I): 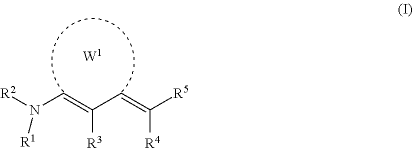
[in Formula (I), the ring W¹, R¹, R², R³, R⁴, and R⁵ have the same meaning as described above; and R² and R³ may be bonded to each other to form a ring],
the method comprising a step of reacting a compound represented by Formula (I-7): 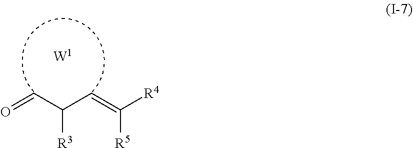
[in Formula (I-7),
a ring W¹ represents a ring structure having at least one double bond as a constituent element of the ring and having no aromaticity;
R³ represents a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —O—, —S—, —NR^{1A}—, —CO—, —CO—O—, —O—CO—, —O—CO—O—, —CONR^{2A}—, —O—CO—NR^{3A}—, —NR^{4A}—CO—, —NR^{5A}—CO—O—, —NR^{6A}—CO—NR^{7A}—, —CO—S—, —S—CO—S—, —S—CO—NR^{8A}—, —NR^{9A}—CO—S—, —CS—, —O—CS—, —CS—O—, —NR^{10A}—CS—, —NR^{11A}—CS—S—, —S—CS—, —CS—S—, —S—CS—S—, —SO—, or —SO₂—;
R^{1A}, R^{2A}, R^{3A}, R^{4A}, R^{5A}, R^{6A}, R^{7A}, R^{8A}, R^{9A}, R^{10A}, and R^{11A} each independently represent a hydrogen atom or an alkyl group having 1 to 6 carbon atoms;
R⁴ and R⁵ each independently represent an electron-withdrawing group; and
R⁴ and R⁵ may be bonded to each other to form a ring], with a compound represented by Formula (I-6): 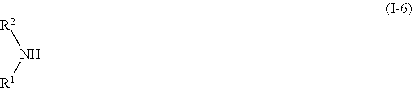
[in Formula (I-6),
R¹ and R² each independently represent a hydrogen atom, a heterocyclic group, a halogen atom, a nitro group, a cyano group, a hydroxy group, a thiol group, a carboxy group, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{12A}—, —SO₂—, —CO—, —O—, —COO—, —OCO—, —CONR^{13A}—, —N^{14A}—CO—, —S—, —SO—, —SO₂—, —CF₂— or —CHF—;
R¹ and R² may be linked to each other to form a ring;
R^{12A}, R^{13A}, and R^{14A} each independently represent a hydrogen atom or an alkyl group having 1 to 6 carbon atoms].

30. The production method according to claim 29, further comprising:
a step of reacting a compound represented by Formula (I-8): 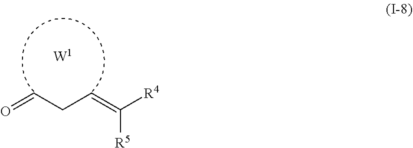
[in Formula (I-8), the ring W¹, R⁴, and R⁵ have the same meaning as described above], with a compound represented by Formula (I-4):
R³-E₁ (I-4)
[in Formula (I-4), R³ has the same meaning as described above. E₁ represents a leaving group], to obtain the compound represented by Formula (I-7).

31. The production method according to claim 30, further comprising:
a step of reacting a compound represented by Formula (I-5): 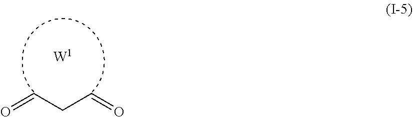
[in Formula (I-5), a ring W¹ has the same meaning as described above], with a compound represented by Formula (I-2): 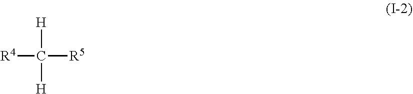
[in Formula (I-2), R⁴ and R⁵ have the same meaning as described above], to obtain the compound represented by Formula (I-8).

32. The production method according to claim 26, further comprising:
a step of reacting a compound represented by Formula (I-5-1): 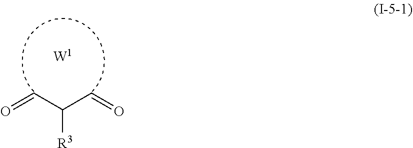
[in Formula (I-5-1), the ring W¹ and R³ have the same meaning as described above], with a compound represented by Formula (I-6): 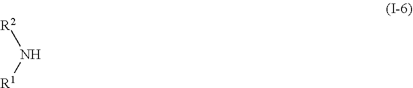
[in Formula (I-6), R¹ and R² have the same meaning as described above], to obtain the compound represented by Formula (I-1).

33. The production method according to claim 29, further comprising:
a step of reacting a compound represented by Formula (I-5-1): 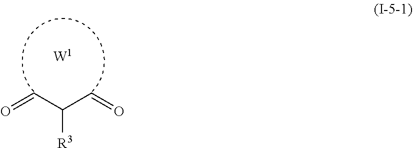
[in Formula (I-5-1), the ring W and R³ have the same meaning as described above], with a compound represented by Formula (I-2): 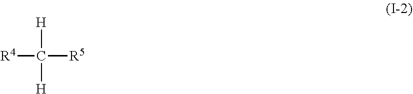
[in Formula (I-2), R⁴ and R⁵ have the same meaning as described above], to obtain the compound represented by Formula (I-7).

34. The production method according to claim 32, further comprising:
a step of reacting a compound represented by Formula (I-5): 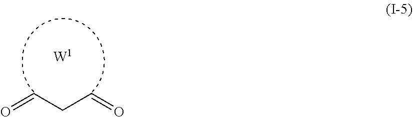
[in Formula (I-5), a ring W¹ has the same meaning as described above], with a compound represented by Formula (I-4):
R³-E₁ (I-4)
[in Formula (I-4), R³ has the same meaning as described above. E₁ represents a leaving group], to obtain the compound represented by Formula (I-5-1).
